SEQUENCE LISTING (147 sequences)

SEQ ID NO: 1 - DNA, 2679 bp, Homo sapiens
agttctcttcaagtcatgtaatcgacttttttgaattagttttcagtttcattttgttttccctaattcaagttgggaacacttcattttccccaattcaagttgggaacacttccttggtatttccttgctacatggactttagcaaatgctactttactctccttccagctactcaggaggctgaggcaggagaatcgcttgaacccgggaggcggaggttacagtgagccttttcctagttttactgttggaagcctaactcacaggagagattatgcaatacagtcctgaagtcaagggaggagagcatgtaggagaatactaaccctgcacagattgtgatggtgatgtggaatatactaaagcctagaacgcacctcctctgcatgactaatatgttctgcacaagacatgaaggcacagacagcactgtctttcttcctcattctcataacatctctgagtggatctcaaggcatattccctttggctttcttcatttatgttcctatgaatgaacaaatcgtcattggaagacttgatgaagatataattctcccttcttcatttgagaggggatccgaagtcgtaatacactggaagtatcaagatagctataaggttcacagttactacaaaggcagtgaccatttggaaagccaagatcccagatatgcaaacaggacatcccttttctataatgagattcaaaatgggaatgcgtcgctatttttcagaagagtaagccttctggacgaaggaatttacacctgctatgtaggaacagcaattcaagtgattacaaacaaagtggtgctaaaggtgggagtttttctcacacccgtgatgaagtatgaaaagaggaacacaaacagcttcttaatatgcagcgtgttaagtgtttatcctcgtccaattatcacgtggaaaatggacaacacacctatctctgaaaacaacatggaagaaacagggtctttggattctttttctattaacagcccactgaatattacaggatcaaattcatcttatgaatgtacaattgaaaattcactgctgaagcaaacatggacagggcgctggacgatgaaagatggccttcataaaatgcaaagtgaacacgtttcactctcatgtcaacctgtaaatgattatttttcaccaaaccaagacttcaaagttacttggtccagaatgaaaagtgggactttctctgtcctggcttactatctgagctcctcacaaaatacaattatcaatgaatcccgattctcatggaacaaagagctgataaaccagagtgacttctctatgaatttgatggatcttaatctttcagacagtggggaatatttatgcaatatttcttcggatgaatatactttacttaccatccacacagtgcatgtagaaccgagccaagaaacagcttcccataacaaaggcttatggattttggtgccctctgcgattttggcagcttttctgctgatttggagcgtaaaatgttgcagagcccagctagaagccaggaggagcagacaccctgctgatggagcccaacaagaaagatgttgtgtccctcctggtgagcgctgtcccagtgcacccgataatggcgaagaaaatgtgcctctttcaggaaaagtataggaaatgagagaagactgtgacaactcatgacctgcatccttaatatccagtgacttcatctcccctttcttcaccacaattccaggcaatggcctgtcggagcagacaattctaccactgcaaagagttgtaaccattttctggtatcacatttatttttcaagacatacttttcaagacatcattcactgacccactacctgcattgagtataaatgcctggatgttaaggattccaatttaactttgaaaagaactgtctcattcatttacatttctgttacagtcagcccaggaggttacagtgagctctccactaagaatctggaagaaatgcatcactaggggttgattcccaatctgatcaactgataatgggtgagagagcaggtaagagccaaagtcaccttagtggaaaggttaaaaaccagagcctggaaaccaagatgattgatttgacaaggtattttagtctagttttatatgaacggttgtatcagggtaaccaactcgatttgggatgaatcttagggcaccaaagactaagacagtatctttaagattgctagggaaaagggccctatgtgtcaggcctctgagcccaagccaagcatcgcatcccctgtgatttgcacgtatacatccagatggcctaaagtaactgaagatccacaaaagaagtaaaaatagccttaactgatgacattccaccattgtgatttgttcctgccccaccctaactgatcaatgtactttgtaatctcccccacccttaagaaggtactttgtaatcttccccacccttaagaaggttctttgtaattctccccacccttgagaatgtactttgtgagatccaccctgcccacaaaacattgctcttaacttcaccgcctaacccaaaacctataagaactaatgataatccatcacccttcgctgactctcttttcggactcagcccacctgcacccaggtgaaataaacagctttattgctcacacaaaaaaaaaaaaaaaa

SEQ ID NO: 2 - protein, 414 aa, Homo sapiens
MKAQTALSFFLILITSLSGSQGIFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNKGLWILVPSAILAAFLLIWSVKCCRAQLEARRSRHPADGAQQERCCVPPGERCPSAPDNGEENVPLSGKV

SEQ ID NO: 3 - DNA, 2488 bp, Homo sapiens
aaatcaaacgtaccttggactttactctctgagaaactcatagctgaattcaatgtttattcttatggactacttagcatttgactagacggtatgaatttctaagtaagcacatatagaactggatgcccttgtggtacatctcaaggctgatttgaaagcttgagagaccatcaagaattggatttggggaagagcatgactaatatgttctgcacaagacatgaaggcacagacagcactgtctttcttcctcattctcataacatctctgagtggatctcaaggcatattccctttggctttcttcatttatgttcctatgaatgaacaaatcgtcattggaagacttgatgaagatataattctcccttcttcatttgagaggggatccgaagtcgtaatacactggaagtatcaagatagctataaggttcacagttactacaaaggcagtgaccatttggaaagccaagatcccagatatgcaaacaggacatcccttttctataatgagattcaaaatgggaatgcgtcgctatttttcagaagagtaagccttctggacgaaggaatttacacctgctatgtaggaacagcaattcaagtgattacaaacaaagtggtgctaaaggtgggagtttttctcacacccgtgatgaagtatgaaaagaggaacacaaacagcttcttaatatgcagcgtgttaagtgtttatcctcgtccaattatcacgtggaaaatggacaacacacctatctctgaaaacaacatggaagaaacagggtctttggattctttttctattaacagcccactgaatattacaggatcaaattcatcttatgaatgtacaattgaaaattcactgctgaagcaaacatggacagggcgctggacgatgaaagatggccttcataaaatgcaaagtgaacacgtttcactctcatgtcaacctgtaaatgattatttttcaccaaaccaagacttcaaagttacttggtccagaatgaaaagtgggactttctctgtcctggcttactatctgagctcctcacaaaatacaattatcaatgaatcccgattctcatggaacaaagagctgataaaccagagtgacttctctatgaatttgatggatcttaatctttcagacagtggggaatatttatgcaatatttcttcggatgaatatactttacttaccatccacacagtgcatgtagaaccgagccaagaaacagcttcccataacaaaggcttatggattttggtgccctctgcgattttggcagcttttctgctgatttggagcgtaaaatgttgcagagcccagctagaagccaggaggagcagacaccctgctgatggagcccaacaagaaagatgttgtgtccctcctggtgagcgctgtcccagtgcacccgataatggcgaagaaaatgtgcctctttcaggaaaagtataggaaatgagagaagactgtgacaactcatgacctgcatccttaatatccagtgacttcatctcccctttcttcaccacaattccaggcaatggcctgtcggagcagacaattctaccactgcaaagagttgtaaccattttctggtatcacatttatttttcaagacatacttttcaagacatcattcactgacccactacctgcattgagtataaatgcctggatgttaaggattccaatttaactttgaaaagaactgtctcattcatttacatttctgttacagtcagcccaggaggttacagtgagctctccactaagaatctggaagaaatgcatcactaggggttgattcccaatctgatcaactgataatgggtgagagagcaggtaagagccaaagtcaccttagtggaaaggttaaaaaccagagcctggaaaccaagatgattgatttgacaaggtattttagtctagttttatatgaacggttgtatcagggtaaccaactcgatttgggatgaatcttagggcaccaaagactaagacagtatctttaagattgctagggaaaagggccctatgtgtcaggcctctgagcccaagccaagcatcgcatcccctgtgatttgcacgtatacatccagatggcctaaagtaactgaagatccacaaaagaagtaaaaatagccttaactgatgacattccaccattgtgatttgttcctgccccaccctaactgatcaatgtactttgtaatctcccccacccttaagaaggtactttgtaatcttccccacccttaagaaggttctttgtaattctccccacccttgagaatgtactttgtgagatccaccctgcccacaaaacattgctcttaacttcaccgcctaacccaaaacctataagaactaatgataatccatcacccttcgctgactctcttttcggactcagcccacctgcacccaggtgaaataaacagctttattgctcacacaaaaaaaaaaaaaaaa

SEQ ID NO: 4 - protein, 414 aa, Homo sapiens; identical to SEQ ID NO: 2

SEQ ID NO: 5 - DNA, 2419 bp, Homo sapiens
agtttactctacatcatagcagagaaaatggacaaaacacagctgttttgcatgtaggagaatactaaccctgcacagattgtgatggtgatgtggaatatactaaagcctagaacgcacctcctctgcatgactaatatgttctgcacaagacatgaaggcacagacagcactgtctttcttcctcattctcataacatctctgagtggatctcaaggcatattccctttggctttcttcatttatgttcctatgaatgaacaaatcgtcattggaagacttgatgaagatataattctcccttcttcatttgagaggggatccgaagtcgtaatacactggaagtatcaagatagctataaggttcacagttactacaaaggcagtgaccatttggaaagccaagatcccagatatgcaaacaggacatcccttttctataatgagattcaaaatgggaatgcgtcgctatttttcagaagagtaagccttctggacgaaggaatttacacctgctatgtaggaacagcaattcaagtgattacaaacaaagtggtgctaaaggtgggagtttttctcacacccgtgatgaagtatgaaaagaggaacacaaacagcttcttaatatgcagcgtgttaagtgtttatcctcgtccaattatcacgtggaaaatggacaacacacctatctctgaaaacaacatggaagaaacagggtctttggattctttttctattaacagcccactgaatattacaggatcaaattcatcttatgaatgtacaattgaaaattcactgctgaagcaaacatggacagggcgctggacgatgaaagatggccttcataaaatgcaaagtgaacacgtttcactctcatgtcaacctgtaaatgattatttttcaccaaaccaagacttcaaagttacttggtccagaatgaaaagtgggactttctctgtcctggcttactatctgagctcctcacaaaatacaattatcaatgaatcccgattctcatggaacaaagagctgataaaccagagtgacttctctatgaatttgatggatcttaatctttcagacagtggggaatatttatgcaatatttcttcggatgaatatactttacttaccatccacacagtgcatgtagaaccgagccaagaaacagcttcccataacaaaggcttatggattttggtgccctctgcgattttggcagcttttctgctgatttggagcgtaaaatgttgcagagcccagctagaagccaggaggagcagacaccctgctgatggagcccaacaagaaagatgttgtgtccctcctggtgagcgctgtcccagtgcacccgataatggcgaagaaaatgtgcctctttcaggaaaagtataggaaatgagagaagactgtgacaactcatgacctgcatccttaatatccagtgacttcatctcccctttcttcaccacaattccaggcaatggcctgtcggagcagacaattctaccactgcaaagagttgtaaccattttctggtatcacatttatttttcaagacatacttttcaagacatcattcactgacccactacctgcattgagtataaatgcctggatgttaaggattccaatttaactttgaaaagaactgtctcattcatttacatttctgttacagtcagcccaggaggttacagtgagctctccactaagaatctggaagaaatgcatcactaggggttgattcccaatctgatcaactgataatgggtgagagagcaggtaagagccaaagtcaccttagtggaaaggttaaaaaccagagcctggaaaccaagatgattgatttgacaaggtattttagtctagttttatatgaacggttgtatcagggtaaccaactcgatttgggatgaatcttagggcaccaaagactaagacagtatctttaagattgctagggaaaagggccctatgtgtcaggcctctgagcccaagccaagcatcgcatcccctgtgatttgcacgtatacatccagatggcctaaagtaactgaagatccacaaaagaagtaaaaatagccttaactgatgacattccaccattgtgatttgttcctgccccaccctaactgatcaatgtactttgtaatctcccccacccttaagaaggtactttgtaatcttccccacccttaagaaggttctttgtaattctccccacccttgagaatgtactttgtgagatccaccctgcccacaaaacattgctcttaacttcaccgcctaacccaaaacctataagaactaatgataatccatcacccttcgctgactctcttttcggactcagcccacctgcacccaggtgaaataaacagctttattgctcacacaaaaaaaaaaaaaaaa

SEQ ID NO: 6 - protein, 414 aa, Homo sapiens; identical to SEQ ID NO: 2

SEQ ID NO: 7 - DNA, 2515 bp, Homo sapiens
aaatcaaacgtaccttggactttactctctgagaaactcatagctgaattcaatgtttattcttatggactacttagcatttgactagacggtatgaatttctaagtaagcacatatagaactggatgcccttgtggtacatctcaaggctgatttgaaagcttgagagaccatcaagaattggatttggggaagagcatgtaggagaatactaaccctgcacagattgtgatggtgatgtggaatatactaaagcctagaacgcacctcctctgcatgactaatatgttctgcacaagacatgaaggcacagacagcactgtctttcttcctcattctcataacatctctgagtggatctcaaggcatattccctttggctttcttcatttatgttcctatgaatgaacaaatcgtcattggaagacttgatgaagatataattctcccttcttcatttgagaggggatccgaagtcgtaatacactggaagtatcaagatagctataaggttcacagttactacaaaggcagtgaccatttggaaagccaagatcccagatatgcaaacaggacatcccttttctataatgagattcaaaatgggaatgcgtcgctatttttcagaagagtaagccttctggacgaaggaatttacacctgctatgtaggaacagcaattcaagtgattacaaacaaagtggtgctaaaggtgggagtttttctcacacccgtgatgaagtatgaaaagaggaacacaaacagcttcttaatatgcagcgtgttaagtgtttatcctcgtccaattatcacgtggaaaatggacaacacacctatctctgaaaacaacatggaagaaacagggtctttggattctttttctattaacagcccactgaatattacaggatcaaattcatcttatgaatgtacaattgaaaattcactgctgaagcaaacatggacagggcgctggacgatgaaagatggccttcataaaatgcaaagtgaacacgtttcactctcatgtcaacctgtaaatgattatttttcaccaaaccaagacttcaaagttacttggtccagaatgaaaagtgggactttctctgtcctggcttactatctgagctcctcacaaaatacaattatcaatgaatcccgattctcatggaacaaagagctgataaaccagagtgacttctctatgaatttgatggatcttaatctttcagacagtggggaatatttatgcaatatttcttcggatgaatatactttacttaccatccacacagtgcatgtagaaccgagccaagaaacagcttcccataacaaaggcttatggattttggtgccctctgcgattttggcagcttttctgctgatttggagcgtaaaatgttgcagagaaagatgttgtgtccctcctggtgagcgctgtcccagtgcacccgataatggcgaagaaaatgtgcctctttcaggaaaagtataggaaatgagagaagactgtgacaactcatgacctgcatccttaatatccagtgacttcatctcccctttcttcaccacaattccaggcaatggcctgtcggagcagacaattctaccactgcaaagagttgtaaccattttctggtatcacatttatttttcaagacatacttttcaagacatcattcactgacccactacctgcattgagtataaatgcctggatgttaaggattccaatttaactttgaaaagaactgtctcattcatttacatttctgttacagtcagcccaggaggttacagtgagctctccactaagaatctggaagaaatgcatcactaggggttgattcccaatctgatcaactgataatgggtgagagagcaggtaagagccaaagtcaccttagtggaaaggttaaaaaccagagcctggaaaccaagatgattgatttgacaaggtattttagtctagttttatatgaacggttgtatcagggtaaccaactcgatttgggatgaatcttagggcaccaaagactaagacagtatctttaagattgctagggaaaagggccctatgtgtcaggcctctgagcccaagccaagcatcgcatcccctgtgatttgcacgtatacatccagatggcctaaagtaactgaagatccacaaaagaagtaaaaatagccttaactgatgacattccaccattgtgatttgttcctgccccaccctaactgatcaatgtactttgtaatctcccccacccttaagaaggtactttgtaatcttccccacccttaagaaggttctttgtaattctccccacccttgagaatgtactttgtgagatccaccctgcccacaaaacattgctcttaacttcaccgcctaacccaaaacctataagaactaatgataatccatcacccttcgctgactctcttttcggactcagcccacctgcacccaggtgaaataaacagctttattgctcacacaaaaaaaaaaaaaaaa

SEQ ID NO: 8 - protein, 397 aa, Homo sapiens
MKAQTALSFFLILITSLSGSQGIFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNKGLWILVPSAILAAFLLIWSVKCCRERCCVPPGERCPSAPDNGEENVPLSGKV

SEQ ID NO: 9 - DNA, 2304 bp, Homo sapiens
aaatcaaacgtaccttggactttactctctgagaaactcatagctgaattcaatgtttattcttatggactacttagcatttgactagacggtatgaatttctaagtaagcacatatagaactggatgcccttgtggtacatctcaaggctgatttgaaagcttgagagaccatcaagaattggatttggggaagagcatgtaggagaatactaaccctgcacagattgtgatggtgatgtggaatatactaaagcctagaacgcacctcctctgcatgactaatatgttctgcacaagacatgaaggcacagacagcactgtctttcttcctcattctcataacatctctgagtggatctcaagaagagtaagccttctggacgaaggaatttacacctgctatgtaggaacagcaattcaagtgattacaaacaaagtggtgctaaaggtgggagtttttctcacacccgtgatgaagtatgaaaagaggaacacaaacagcttcttaatatgcagcgtgttaagtgtttatcctcgtccaattatcacgtggaaaatggacaacacacctatctctgaaaacaacatggaagaaacagggtctttggattctttttctattaacagcccactgaatattacaggatcaaattcatcttatgaatgtacaattgaaaattcactgctgaagcaaacatggacagggcgctggacgatgaaagatggccttcataaaatgcaaagtgaacacgtttcactctcatgtcaacctgtaaatgattatttttcaccaaaccaagacttcaaagttacttggtccagaatgaaaagtgggactttctctgtcctggcttactatctgagctcctcacaaaatacaattatcaatgaatcccgattctcatggaacaaagagctgataaaccagagtgacttctctatgaatttgatggatcttaatctttcagacagtggggaatatttatgcaatatttcttcggatgaatatactttacttaccatccacacagtgcatgtagaaccgagccaagaaacagcttcccataacaaaggcttatggattttggtgccctctgcgattttggcagcttttctgctgatttggagcgtaaaatgttgcagagcccagctagaagccaggaggagcagacaccctgctgatggagcccaacaagaaagatgttgtgtccctcctggtgagcgctgtcccagtgcacccgataatggcgaagaaaatgtgcctctttcaggaaaagtataggaaatgagagaagactgtgacaactcatgacctgcatccttaatatccagtgacttcatctcccctttcttcaccacaattccaggcaatggcctgtcggagcagacaattctaccactgcaaagagttgtaaccattttctggtatcacatttatttttcaagacatacttttcaagacatcattcactgacccactacctgcattgagtataaatgcctggatgttaaggattccaatttaactttgaaaagaactgtctcattcatttacatttctgttacagtcagcccaggaggttacagtgagctctccactaagaatctggaagaaatgcatcactaggggttgattcccaatctgatcaactgataatgggtgagagagcaggtaagagccaaagtcaccttagtggaaaggttaaaaaccagagcctggaaaccaagatgattgatttgacaaggtattttagtctagttttatatgaacggttgtatcagggtaaccaactcgatttgggatgaatcttagggcaccaaagactaagacagtatctttaagattgctagggaaaagggccctatgtgtcaggcctctgagcccaagccaagcatcgcatcccctgtgatttgcacgtatacatccagatggcctaaagtaactgaagatccacaaaagaagtaaaaatagccttaactgatgacattccaccattgtgatttgttcctgccccaccctaactgatcaatgtactttgtaatctcccccacccttaagaaggtactttgtaatcttccccacccttaagaaggttctttgtaattctccccacccttgagaatgtactttgtgagatccaccctgcccacaaaacattgctcttaacttcaccgcctaacccaaaacctataagaactaatgataatccatcacccttcgctgactctcttttcggactcagcccacctgcacccaggtgaaataaacagctttattgctcacacaaaaaaaaaaaaaaaa

SEQ ID NO: 10 - protein, 350 aa, Homo sapiens
MVMWNILKPRTHLLCMTNMFCTRHEGTDSTVFLPHSHNISEWISRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNKGLWILVPSAILAAFLLIWSVKCCRAQLEARRSRHPADGAQQERCCVPPGERCPSAPDNGEENVPLSGKV

SEQ ID NO: 11 - DNA, 1723 bp, Homo sapiens
tgctgctgaactgagctggggcgcagccgcctgtctgcaccggcagcaccatgtcgctcatggtcgtcagcatggcgtgtgttgggttcttcttgctggaggggccctggccacatgtgggtggtcaggacaagcccttcctctctgcctggcccggcactgtggtgtctgaaggacaacatgtgactcttcagtgtcgctctcgtcttgggtttaatgaattcagtctgtccaaagaagacgggatgcctgtccctgagctctacaacagaatattccggaacagctttctcatgggccctgtgaccccagcacatgcagggacctacagatgttgcagttcacacccacactcccccactgggtggtcggcacccagcaaccctgtggtgatcatggtcacaggagtccacagaaaaccttccctcctggcccacccaggtcccctggtgaaatcaggagagacggtcatcctgcaatgttggtcagatgtcaggtttgagcgcttccttctgcacagagaggggatcactgaggaccccttgcgcctcgttggacagctccacgatgcgggttcccaggtcaactattccatgggtcccatgacacctgcccttgcagggacctacagatgctttggttctgtcactcacttaccctatgagttgtcggctcccagtgaccctctggacatcgtggtcgtaggtctatatgggaaaccttctctctcagcccagccgggccccacggttcaggcaggagagaatgtgaccttgtcctgcagctcccggagcttgtttgacatttaccatctatccagggaggcggaggccggtgaacttaggctcactgcagtgctgagggtcaatggaacattccaggccaacttccctctgggccctgtgacccacggagggaactacagatgcttcggctctttccgtgccctgccccatgcgtggtcagacccgagtgacccactgcccgtttctgtcacaggtaactccagaaacctgcacgttctgattgggacctcagtggtcatcatcccctttgctatcctcctcttctttctccttcatcgctggtgtgccaacaaaaagaatgctgttgtaatggaccaagagcctgcagggaacagaacagtgaacagggaggactctgatgaacaagaccctcaggaggtgacatacgcacagttgaatcactgcgttttcacacagagaaaaatcactcgcccttctcagaggcccaagacacccccaacagataccagcgtgtaacacggaacttccaaatgctgagcgcagatccaaagttgtcttctgtccactagcaccacagtcaggccttgatgggatcttctagggagacaatagccctgtctcaaaaccgggttgccagctcccatgtaccagcagctggactctgaaggcgtgagtctgcatcttagggcatcgctcttcctcacaccacgaatctgaacatgcctctctcttgcttacaaatgtctaaggtccccactgcctgctggagagaaaacacacttgcttagcccacaattctccatttcacttgacccctgcccacctctccaacctaactggcttacttcctagtctacttgaggctgcgatcacactgaggaactcacaattccaaacatataagaggctccctcttaacacggcacttagatacgtgctattccacctttcctcag

SEQ ID NO: 12 - protein, 410 aa, Homo sapiens
MSLMVVSMACVGFFLLEGPWPHVGGQDKPFLSAWPGTVVSEGQHVTLQCRSRLGFNEFSLSKEDGMPVPELYNRIFRNSFLMGPVTPAHAGTYRCCSSHPHSPTGWSAPSNPVVIMVTGVHRKPSLLAHPGPLVKSGETVILQCWSDVRFERFLLHREGITEDPLRLVGQLHDAGSQVNYSMGPMTPALAGTYRCFGSVTHLPYELSAPSDPLDIVVVGLYGKPSLSAQPGPTVQAGENVTLSCSSRSLFDIYHLSREAEAGELRLTAVLRVNGTFQANFPLGPVTHGGNYRCFGSFRALPHAWSDPSDPLPVSVTGNSRNLHVLIGTSVVIIPFAILLFFLLHRWCANKKNAVVMDQEPAGNRTVNREDSDEQDPQEVTYAQLNHCVFTQRKITRPSQRPKTPPTDTSV

SEQ ID NO: 13 - protein, 25 aa, Artificial Sequence
  Description of Artificial Sequence Synthetic peptide
QVQLQQSGAELMKPGASVKISCKAT

SEQ ID NO: 14 - protein, 7 aa, Artificial Sequence
  Description of Artificial Sequence Synthetic peptide
GYTFSNY

SEQ ID NO: 15 - protein, 19 aa, Artificial Sequence
  Description of Artificial Sequence Synthetic peptide
WIEWIKQRPGHGLEWIGEI

SEQ ID NO: 16 - protein, 6 aa, Artificial Sequence
  Description of Artificial Sequence Synthetic peptide
LPGSGS

SEQ ID NO: 17 - protein, 41 aa, Artificial Sequence
  Description of Artificial Sequence Synthetic polypeptide
TNYNEKFKGKATFTAATSSNTAYMQLSSLTSEDSAVYYCAR

SEQ ID NO: 18 - protein, 14 aa, Artificial Sequence
  Description of Artificial Sequence Synthetic peptide
RVPDYYGSRYYFDY

SEQ ID NO: 19 - protein, 11 aa, Artificial Sequence
  Description of Artificial Sequence Synthetic peptide
WGQGTTLTVSS

SEQ ID NO: 20 - protein, 23 aa, Artificial Sequence
  Description of Artificial Sequence Synthetic peptide
NIVMTQSPKSMSMSVGERVTLSC

SEQ ID NO: 21 - protein, 11 aa, Artificial Sequence
  Description of Artificial Sequence Synthetic peptide
KASENVGIYVS

SEQ ID NO: 22 - protein, 15 aa, Artificial Sequence
  Description of Artificial Sequence Synthetic peptide
WYQQKPEQSPKLLIY

SEQ ID NO: 23 - protein, 7 aa, Artificial Sequence
  Description of Artificial Sequence Synthetic peptide
GASNRYT

SEQ ID NO: 24 - protein, 32 aa, Artificial Sequence
  Description of Artificial Sequence Synthetic polypeptide
GVPDRFTGSGSATDFTLTISSVQAEDLADYHC

SEQ ID NO: 25 - protein, 9 aa, Artificial Sequence
  Description of Artificial Sequence Synthetic peptide
GQSYNYPFT

SEQ ID NO: 26 - protein, 10 aa, Artificial Sequence
  Description of Artificial Sequence Synthetic peptide
FGSGTKLEIK

SEQ ID NO: 27 - protein, 123 aa, Artificial Sequence
  Description of Artificial Sequence Synthetic polypeptide
QVQLQQSGAELMKPGASVKISCKATGYTFSNYWIEWIKQRPGHGLEWIGEILPGSGSTNYNEKFKGKATFTAATSSNTAYMQLSSLTSEDSAVYYCARRVPDYYGSRYYFDYWGQGTTLTVSS

SEQ ID NO: 28 - DNA, 369 bp, Artificial Sequence
  Description of Artificial Sequence Synthetic polynucleotide
caggttcagctgcagcagtctggagctgagctgatgaagcctggggcctcagtgaagatatcctgcaaggctactggctacacattcagtaactactggatagagtggataaagcagaggcctggacatggccttgagtggattggagagattttacctggaagtggtagtactaactacaatgagaagttcaagggcaaggccacattcactgcagctacatcctccaacacagcctacatgcaactcagcagcctgacatctgaggactctgccgtctattactgtgcaagaagggtccctgattactacggtagtaggtactactttgactactggggccaaggcaccactctcacagtctcctca

SEQ ID NO: 29 - protein, 107 aa, Artificial Sequence
  Description of Artificial Sequence Synthetic polypeptide
NIVMTQSPKSMSMSVGERVTLSCKASENVGIYVSWYQQKPEQSPKLLIYGASNRYTGVPDRFTGSGSATDFTLTISSVQAEDLADYHCGQSYNYPFTFGSGTKLEIK

SEQ ID NO: 30 - DNA, 321 bp, Artificial Sequence
  Description of Artificial Sequence Synthetic polynucleotide
aacattgtaatgacccaatctcccaaatccatgtccatgtcagtaggagagagggtcaccttgagctgcaaggccagtgagaatgtgggtatttacgtatcctggtatcaacagaaaccagagcagtctcctaaactactaatatacggggcatccaaccgatacactggggtccccgatcgcttcacaggcagtggatctgcaacagatttcactctgaccatcagcagtgtgcaggctgaagaccttgcagattatcactgtggacagagttacaactatccattcacgttcggctcggggacaaagttggaaatcaaa

SEQ ID NO: 31 - protein, 25 aa, Artificial Sequence
  Description of Artificial Sequence Synthetic peptide
EVQLQQSGPELVKPGASVKMSCKAS

SEQ ID NO: 32 - protein, 7 aa, Artificial Sequence
  Description of Artificial Sequence Synthetic peptide
GYTFTSY

SEQ ID NO: 33 - protein, 19 aa, Artificial Sequence
  Description of Artificial Sequence Synthetic peptide
VMHWLKQKPGQGLEWIAYI

SEQ ID NO: 34 - protein, 6 aa, Artificial Sequence
  Description of Artificial Sequence Synthetic peptide
NPYNDG

SEQ ID NO: 35 - protein, 41 aa, Artificial Sequence
  Description of Artificial Sequence Synthetic polypeptide
TKYNENFKGKATLTSDKSSSTAYMELSSLTSEDSAVYYCAR

SEQ ID NO: 36 - protein, 11 aa, Artificial Sequence
  Description of Artificial Sequence Synthetic peptide
PSMVTAAWFAY

SEQ ID NO: 37 - protein, 11 aa, Artificial Sequence
  Description of Artificial Sequence Synthetic peptide
WGQGTLVTVSA

SEQ ID NO: 38 - protein, 23 aa, Artificial Sequence
  Description of Artificial Sequence Synthetic peptide
DVVMTQTPLSLPVSLGDQASISC

SEQ ID NO: 39 - protein, 16 aa, Artificial Sequence
  Description of Artificial Sequence Synthetic peptide
RSSQNLAHSNGDTYLH

SEQ ID NO: 40 - protein, 15 aa, Artificial Sequence
  Description of Artificial Sequence Synthetic peptide
WYLQKPGQSPKLLIY

SEQ ID NO: 41 - protein, 7 aa, Artificial Sequence
  Description of Artificial Sequence Synthetic peptide
KVSNRFS

SEQ ID NO: 42 - protein, 32 aa, Artificial Sequence
  Description of Artificial Sequence Synthetic polypeptide
GVPDRFSGSGSGTDFTLKISRVEAEDLGVYFC

SEQ ID NO: 43 - protein, 9 aa, Artificial Sequence
  Description of Artificial Sequence Synthetic peptide
SQTTHVLWT

SEQ ID NO: 44 - protein, 10 aa, Artificial Sequence
  Description of Artificial Sequence Synthetic peptide
FGGGTKLEIK

SEQ ID NO: 45 - protein, 120 aa, Artificial Sequence
  Description of Artificial Sequence Synthetic polypeptide
EVQLQQSGPELVKPGASVKMSCKASGYTFTSYVMHWLKQKPGQGLEWIAYINPYNDGTKYNENFKGKATLTSDKSSSTAYMELSSLTSEDSAVYYCARPSMVTAAWFAYWGQGTLVTVSA

SEQ ID NO: 46 - DNA, 360 bp, Artificial Sequence
  Description of Artificial Sequence Synthetic polynucleotide
gaggtccagctgcagcagtctggacctgagctggtaaagcctggggcttcagtgaagatgtcctgcaaggcttctggatacacattcactagctatgtcatgcactggttgaagcagaagcctgggcagggccttgagtggattgcatatattaatccttacaatgatggtactaagtacaatgagaacttcaaaggcaaggccacactgacttcagacaaatcctccagcacagcctacatggagctcagcagcctgacctctgaggactctgcggtctattactgtgcaagacctagtatggtaactgccgcctggtttgcttactggggccaagggactctggtcactgtctctgca

SEQ ID NO: 47 - protein, 112 aa, Artificial Sequence
  Description of Artificial Sequence Synthetic polypeptide
DVVMTQTPLSLPVSLGDQASISCRSSQNLAHSNGDTYLHWYLQKPGQSPKLLIYKVSNRFSGVPDRFSGSGSGTDFTLKISRVEAEDLGVYFCSQTTHVLWTFGGGTKLEIK

SEQ ID NO: 48 - DNA, 336 bp, Artificial Sequence
  Description of Artificial Sequence Synthetic polynucleotide
gatgttgtgatgacccaaactccactctccctgcctgtcagtcttggagatcaagcctccatctcctgcagatctagtcagaaccttgcacacagtaatggagacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaactacacatgttttgtggacgttcggtggaggcaccaagctggaaatcaaa

SEQ ID NO: 49 - protein, 25 aa, Artificial Sequence
  Description of Artificial Sequence Synthetic peptide
DVKLVESGGGLVKPGGSLKLSCAAS

SEQ ID NO: 50 - protein, 7 aa, Artificial Sequence
  Description of Artificial Sequence Synthetic peptide
GFTFSSY

SEQ ID NO: 51 - protein, 19 aa, Artificial Sequence
  Description of Artificial Sequence Synthetic peptide
CMSWVRQTPEKRLEWVATI

SEQ ID NO: 52 - protein, 6 aa, Artificial Sequence
  Description of Artificial Sequence Synthetic peptide
SSGGNY

SEQ ID NO: 53 - protein, 41 aa, Artificial Sequence
  Description of Artificial Sequence Synthetic polypeptide
IYYPDSVKGRFTISRDIAKNTLYLQMSSLKSEDTAMYYCTR

SEQ ID NO: 54 - protein, 10 aa, Artificial Sequence
  Description of Artificial Sequence Synthetic peptide
GGYDERYFDY

SEQ ID NO: 55 - protein, 11 aa, Artificial Sequence
  Description of Artificial Sequence Synthetic peptide
WGQGTILTVSS

SEQ ID NO: 56 - protein, 23 aa, Artificial Sequence
  Description of Artificial Sequence Synthetic peptide
DIQMTQSPASLSASVGETVTITC

SEQ ID NO: 57 - protein, 11 aa, Artificial Sequence
  Description of Artificial Sequence Synthetic peptide
RASENIYSYLA

SEQ ID NO: 58 - protein, 15 aa, Artificial Sequence
  Description of Artificial Sequence Synthetic peptide
WYQQKQGKSPQLLVY

SEQ ID NO: 59 - protein, 7 aa, Artificial Sequence
  Description of Artificial Sequence Synthetic peptide
NAKTLTE

SEQ ID NO: 60 - protein, 32 aa, Artificial Sequence
  Description of Artificial Sequence Synthetic polypeptide
GMPSRFSGSGSGTQFSLRINSLQPEDFGSYYC

SEQ ID NO: 61 - protein, 9 aa, Artificial Sequence
  Description of Artificial Sequence Synthetic peptide
QHHHGTPRT

SEQ ID NO: 62 - protein, 119 aa, Artificial Sequence
  Description of Artificial Sequence Synthetic polypeptide
DVKLVESGGGLVKPGGSLKLSCAASGFTFSSYCMSWVRQTPEKRLEWVATISSGGNYIYYPDSVKGRFTISRDIAKNTLYLQMSSLKSEDTAMYYCTRGGYDERYFDYWGQGTILTVSS

SEQ ID NO: 63 - DNA, 357 bp, Artificial Sequence
  Description of Artificial Sequence Synthetic polynucleotide
gacgtgaagttggtggagtctgggggaggcttagtgaaacctggagggtccctgaaactctcctgtgcagcctctggattcactttcagttcctattgcatgtcttgggttcgccagactccggagaagaggctggagtgggtcgcaaccattagtagtggtggtaattacatctactatccggacagtgtgaagggccgattcaccatctccagagacattgccaagaacaccctgtacctgcaaatgagcagtctgaagtctgaggacacagccatgtattactgtacaagagggggttacgacgagaggtactttgactactggggccaaggcaccattctcacagtctcctca

SEQ ID NO: 64 - protein, 107 aa, Artificial Sequence
  Description of Artificial Sequence Synthetic polypeptide
DIQMTQSPASLSASVGETVTITCRASENIYSYLAWYQQKQGKSPQLLVYNAKTLTEGMPSRFSGSGSGTQFSLRINSLQPEDFGSYYCQHHHGTPRTFGGGTKLEIK

SEQ ID NO: 65 - DNA, 321 bp, Artificial Sequence
  Description of Artificial Sequence Synthetic polynucleotide
gacatccagatgactcagtctccagcctccctttctgcttctgtgggagaaactgtcaccatcacatgtcgagcaagtgaaaatatttacagttatttagcatggtatcagcagaaacagggaaaatctcctcagctcctggtctataatgcaaaaaccttaacagaaggtatgccatcaaggttcagtggcagtggatcaggtacacagttttctctgaggatcaatagcctgcagcctgaagattttgggagttattactgtcaacatcatcatggtactcctcggacgttcggtggaggcaccaagttggaaatcaaa

SEQ ID NO: 66 - protein, 25 aa, Artificial Sequence
  Description of Artificial Sequence Synthetic peptide
ELKLEESGGGLVQPGGSMKLSCVAS

SEQ ID NO: 67 - protein, 7 aa, Artificial Sequence
  Description of Artificial Sequence Synthetic peptide
GFTFSNY

SEQ ID NO: 68 - protein, 19 aa, Artificial Sequence
  Description of Artificial Sequence Synthetic peptide
WMNWVRQSPEKGLEWIAEI

SEQ ID NO: 69 - protein, 8 aa, Artificial Sequence
  Description of Artificial Sequence Synthetic peptide
RLKSNNYA

SEQ ID NO: 70 - protein, 41 aa, Artificial Sequence
  Description of Artificial Sequence Synthetic polypeptide
THYAESVKGRFTISRDDSKSSVYLQMNNLRAEDTGIFYCTP

SEQ ID NO: 71 - protein, 8 aa, Artificial Sequence
  Description of Artificial Sequence Synthetic peptide
FYGSSFPY

SEQ ID NO: 72 - protein, 23 aa, Artificial Sequence
  Description of Artificial Sequence Synthetic peptide
DIQMTQTTSSLSASLGDRVTISC

SEQ ID NO: 73 - protein, 11 aa, Artificial Sequence
  Description of Artificial Sequence Synthetic peptide
RASQDISNYLN

SEQ ID NO: 74 - protein, 15 aa, Artificial Sequence
  Description of Artificial Sequence Synthetic peptide
WYQQKPDGTVKLLIY

SEQ ID NO: 75 - protein, 7 aa, Artificial Sequence
  Description of Artificial Sequence Synthetic peptide
YTSILQL

SEQ ID NO: 76 - protein, 32 aa, Artificial Sequence
  Description of Artificial Sequence Synthetic polypeptide
GVPPRFSGSGSGTDYSLTISNLEQEDIATYFC

SEQ ID NO: 77 - protein, 9 aa, Artificial Sequence
  Description of Artificial Sequence Synthetic peptide
QQGNTLPFT

SEQ ID NO: 78 - protein, 10 aa, Artificial Sequence
  Description of Artificial Sequence Synthetic peptide
FGAGTKVELK

SEQ ID NO: 79 - protein, 119 aa, Artificial Sequence
  Description of Artificial Sequence Synthetic polypeptide
ELKLEESGGGLVQPGGSMKLSCVASGFTFSNYWMNWVRQSPEKGLEWIAEIRLKSNNYATHYAESVKGRFTISRDDSKSSVYLQMNNLRAEDTGIFYCTPFYGSSFPYWGQGTLVTVSA

SEQ ID NO: 80 - DNA, 357 bp, Artificial Sequence
  Description of Artificial Sequence Synthetic polynucleotide
gaactgaagcttgaggagtctggaggaggcttggtgcaacctggaggatccatgaaactctcctgtgttgcctctggattcactttcagtaactactggatgaactgggtccgccagtctccagagaaggggcttgaatggattgctgaaattagattgaaatctaataattatgcaacacattatgcggagtctgtgaaagggaggttcaccatctcaagagatgattccaaaagtagtgtctacctacagatgaacaacttaagagctgaagacactggcattttttactgtacccccttctacggtagttcttttccttactggggccaagggactctggtcactgtctctgca

SEQ ID NO: 81 - protein, 107 aa, Artificial Sequence
  Description of Artificial Sequence Synthetic polypeptide
DIQMTQTTSSLSASLGDRVTISCRASQDISNYLNWYQQKPDGTVKLLIYYTSILQLGVPPRFSGSGSGTDYSLTISNLEQEDIATYFCQQGNTLPFTFGAGTKVELK

SEQ ID NO: 82 - DNA, 321 bp, Artificial Sequence
  Description of Artificial Sequence Synthetic polynucleotide
gatatccagatgacacagactacatcgtccctgtctgcctctctgggagacagagtcaccatcagttgcagggcaagtcaggacattagcaattacttaaactggtatcagcagaaaccagatggaactgttaaactccttatctattacacatcaatattacaattaggagtcccgccaaggttcagtggcagtgggtctggaacagattattctctcaccattagcaacctggagcaagaagatattgccacttacttttgccaacagggtaatacgcttccgttcacgttcggtgctgggaccaaggtggagctgaaa

SEQ ID NO: 83 - protein, 25 aa, Artificial Sequence
  Description of Artificial Sequence Synthetic peptide
DVQLVESGGGLVQPGGSRKLSCAAS

SEQ ID NO: 84 - protein, 7 aa, Artificial Sequence
  Description of Artificial Sequence Synthetic peptide
GFTFSYF

SEQ ID NO: 85 - protein, 19 aa, Artificial Sequence
  Description of Artificial Sequence Synthetic peptide
GMHWVRRAPEKGLEWVAFI

SEQ ID NO: 86 - protein, 6 aa, Artificial Sequence
  Description of Artificial Sequence Synthetic peptide
SSDSTT

SEQ ID NO: 87 - protein, 41 aa, Artificial Sequence
  Description of Artificial Sequence Synthetic polypeptide
IYYTDTVKGRFTISRDNPKNTLFLQMTSLRSEDTAIYYCAR

SEQ ID NO: 88 - protein, 8 aa, Artificial Sequence
  Description of Artificial Sequence Synthetic peptide
FYDGAMDY

SEQ ID NO: 89 - protein, 11 aa, Artificial Sequence
  Description of Artificial Sequence Synthetic peptide
WGQGTSVTVST

SEQ ID NO: 90 - protein, 23 aa, Artificial Sequence
  Description of Artificial Sequence Synthetic peptide
DIVMTQSQKFMSTSVGDRVSITC

SEQ ID NO: 91 - protein, 11 aa, Artificial Sequence
  Description of Artificial Sequence Synthetic peptide
KASQSVRTAVA

SEQ ID NO: 92 - protein, 15 aa, Artificial Sequence
  Description of Artificial Sequence Synthetic peptide
WYQQKPGQSPKVLIY

SEQ ID NO: 93 - protein, 7 aa, Artificial Sequence
  Description of Artificial Sequence Synthetic peptide
LASNRHT

SEQ ID NO: 94 - protein, 32 aa, Artificial Sequence
  Description of Artificial Sequence Synthetic polypeptide
GVPDRFTGSGSGTDFTLTISNVQSEDLADYFC

SEQ ID NO: 95 - protein, 9 aa, Artificial Sequence
  Description of Artificial Sequence Synthetic peptide
LQHWNYPLT

SEQ ID NO: 96 - protein, 10 aa, Artificial Sequence
  Description of Artificial Sequence Synthetic peptide
FGAGTKLELK

SEQ ID NO: 97 - protein, 117 aa, Artificial Sequence
  Description of Artificial Sequence Synthetic polypeptide
DVQLVESGGGLVQPGGSRKLSCAASGFTFSYFGMHWVRRAPEKGLEWVAFISSDSTTIYYTDTVKGRFTISRDNPKNTLFLQMTSLRSEDTAIYYCARFYDGAMDYWGQGTSVTVST

SEQ ID NO: 98 - DNA, 351 bp, Artificial Sequence
  Description of Artificial Sequence Synthetic polynucleotide
gatgtgcagctggtggagtctgggggaggcttagtgcagcctggagggtcccggaaactctcctgtgcagcctctggattcactttcagttactttggaatgcactgggttcgtcgggctccagagaaggggctggagtgggtcgcattcattagtagtgacagtactaccatctactatacagacacagtgaagggccgattcaccatctccagagacaatcccaagaacaccctgttcctgcaaatgaccagtctaaggtctgaggacacggccatatattactgtgcaagattttacgacggtgctatggactactggggtcaaggaacctcagtcaccgtctccaca

SEQ ID NO: 99 - protein, 107 aa, Artificial Sequence
  Description of Artificial Sequence Synthetic polypeptide
DIVMTQSQKFMSTSVGDRVSITCKASQSVRTAVAWYQQKPGQSPKVLIYLASNRHTGVPDRFTGSGSGTDFTLTISNVQSEDLADYFCLQHWNYPLTFGAGTKLELK

SEQ ID NO: 100 - DNA, 321 bp, Artificial Sequence
  Description of Artificial Sequence Synthetic polynucleotide
gacattgtgatgacccagtctcaaaaattcatgtccacatcagtaggagacagggtcagcatcacctgcaaggccagtcagagtgttcgtactgctgtagcctggtatcaacagaaaccagggcagtctcctaaagtattgatttacttggcatccaaccggcacactggagtccctgatcgcttcacaggcagtggatctgggacagacttcactctcaccattagcaatgtgcaatctgaagacctggcagattatttctgtcttcaacattggaattatcctctcacgttcggtgctgggaccaagctggagctgaaa

SEQ ID NO: 101 - DNA, 321 bp, Artificial Sequence
  Description of Artificial Sequence Synthetic polynucleotide
gacattgtgatgacccagtctcaaaaattcatgtccacatcagtaggagacagggtcagcatcacctgcaaggccagtcagagtgttcgtactgctgtagcctggtatcaacagaaaccaggacagtctcctaaagtattgatttacttggcatccaaccggcacactggagtccctgatcgcttcacaggcagtggatctgggacagacttcactctcaccattagcaatgtgcaatctgaagacctggcagattatttctgtcttcaacattggaattatcctctcacgttcggtgctgggaccaagctggagctgaaa

SEQ ID NO: 102 - protein, 25 aa, Artificial Sequence
  Description of Artificial Sequence Synthetic peptide
QVQLQQSGAELVRPGTSVKVSCKAS

SEQ ID NO: 103 - protein, 7 aa, Artificial Sequence
  Description of Artificial Sequence Synthetic peptide
GYAFNNY

SEQ ID NO: 104 - protein, 19 aa, Artificial Sequence
  Description of Artificial Sequence Synthetic peptide
LIEWVKQRPGQGLEWIGVI

SEQ ID NO: 105 - protein, 6 aa, Artificial Sequence
  Description of Artificial Sequence Synthetic peptide
NPGSGG

SEQ ID NO: 106 - protein, 41 aa, Artificial Sequence
  Description of Artificial Sequence Synthetic polypeptide
TNDNEKFRGKATLTADTSSSTAYMQLTSLTSDDSAVYFCAR

SEQ ID NO: 107 - protein, 13 aa, Artificial Sequence
  Description of Artificial Sequence Synthetic peptide
GNYYGTRDWYFDV

SEQ ID NO: 108 - protein, 11 aa, Artificial Sequence
  Description of Artificial Sequence Synthetic peptide
WGAGTTVTVSS

SEQ ID NO: 109 - protein, 23 aa, Artificial Sequence
  Description of Artificial Sequence Synthetic peptide
QIVLTQSPAIMSASLGARVTMTC

SEQ ID NO: 110 - protein, 12 aa, Artificial Sequence
  Description of Artificial Sequence Synthetic peptide
TASSSVSSSSLH

SEQ ID NO: 111 - protein, 15 aa, Artificial Sequence
  Description of Artificial Sequence Synthetic peptide
WYQQKPGSSPKLWIY

SEQ ID NO: 112 - protein, 7 aa, Artificial Sequence
  Description of Artificial Sequence Synthetic peptide
SISSLAS

SEQ ID NO: 113 - protein, 32 aa, Artificial Sequence
  Description of Artificial Sequence Synthetic polypeptide
GVPARFSGSGSGTSYSLTISSLEAEDAATYYC

SEQ ID NO: 114 - protein, 9 aa, Artificial Sequence
  Description of Artificial Sequence Synthetic peptide
HQYHRSPPT

SEQ ID NO: 115 - protein, 122 aa, Artificial Sequence
  Description of Artificial Sequence Synthetic polypeptide
QVQLQQSGAELVRPGTSVKVSCKASGYAFNNYLIEWVKQRPGQGLEWIGVINPGSGGTNDNEKFRGKATLTADTSSSTAYMQLTSLTSDDSAVYFCARGNYYGTRDWYFDVWGAGTTVTVSS

SEQ ID NO: 116 - DNA, 366 bp, Artificial Sequence
  Description of Artificial Sequence Synthetic polynucleotide
caggtccagctgcagcagtctggagctgagctggtaaggcctgggacttcagtgaaggtgtcctgcaaggcttctggatacgccttcaataattacttgatagagtgggtaaagcagaggcctggacagggccttgagtggattggagtgatcaatcctggaagtggtggtactaacgacaatgagaagttcaggggcaaggctaccctgactgcagacacatcctccagcactgcctacatgcagctcaccagcctgacatctgatgactctgcggtctatttctgtgcaagagggaattactacggtactcgcgactggtacttcgatgtctggggcgcagggaccacggtcaccgtctcctca

SEQ ID NO: 117 - protein, 108 aa, Artificial Sequence
  Description of Artificial Sequence Synthetic polypeptide
QIVLTQSPAIMSASLGARVTMTCTASSSVSSSSLHWYQQKPGSSPKLWIYSISSLASGVPARFSGSGSGTSYSLTISSLEAEDAATYYCHQYHRSPPTFGGGTKLEIK

SEQ ID NO: 118 - DNA, 324 bp, Artificial Sequence
  Description of Artificial Sequence Synthetic polynucleotide
caaattgttctcacccagtctccagcaatcatgtctgcatctctaggggcacgggtcaccatgacctgcactgccagctcaagtgtaagttccagttccttgcactggtaccagcagaagccaggatcctcccccaaactctggatttatagtatatccagcctggcttctggagtcccagctcgcttcagtggcagtgggtctgggacctcttactctctcacaatcagcagcttggaggctgaagatgctgccacttattactgccaccagtatcatcgctccccaccgacgttcggtggaggcaccaagctggaaatcaaa

SEQ ID NO: 119 - protein, 25 aa, Artificial Sequence
  Description of Artificial Sequence Synthetic peptide
KVQLQQSGAELVRPGTSVKVSCKAS

SEQ ID NO: 120 - protein, 7 aa, Artificial Sequence
  Description of Artificial Sequence Synthetic peptide
GYAFTNY

SEQ ID NO: 121 - protein, 41 aa, Artificial Sequence
  Description of Artificial Sequence Synthetic polypeptide
TNDNEKFRDKATLTADKSSSTAYMQLSSLTSDDSAVYFCAR

SEQ ID NO: 122 - protein, 13 aa, Artificial Sequence
  Description of Artificial Sequence Synthetic peptide
GNYYGTTDWYFDV

SEQ ID NO: 123 - protein, 23 aa, Artificial Sequence
  Description of Artificial Sequence Synthetic peptide
QIVLTQSPAIMSASLGERVTMTC

SEQ ID NO: 124 - protein, 12 aa, Artificial Sequence
  Description of Artificial Sequence Synthetic peptide
TASASVSSSSLH

SEQ ID NO: 125 - protein, 15 aa, Artificial Sequence
  Description of Artificial Sequence Synthetic peptide
WYQQKPGSPPKFWIY

SEQ ID NO: 126 - protein, 7 aa, Artificial Sequence
  Description of Artificial Sequence Synthetic peptide
STTNLAS

SEQ ID NO: 127 - protein, 32 aa, Artificial Sequence
  Description of Artificial Sequence Synthetic polypeptide
GVPTRFSGSGSGTSYSLTISNMEAEDAATYYC

SEQ ID NO: 128 - protein, 122 aa, Artificial Sequence
  Description of Artificial Sequence Synthetic polypeptide
KVQLQQSGAELVRPGTSVKVSCKASGYAFTNYLIEWVKQRPGQGLEWIGVINPGSGGTNDNEKFRDKATLTADKSSSTAYMQLSSLTSDDSAVYFCARGNYYGTTDWYFDVWGAGTTVTVSS

SEQ ID NO: 129 - DNA, 366 bp, Artificial Sequence
  Description of Artificial Sequence Synthetic polynucleotide
aaggtccagctgcagcagtctggagctgagctggtaaggcctgggacttcagtgaaggtgtcctgcaaggcttctggatacgccttcactaattatttgatagagtgggtaaagcagaggcctggacagggccttgagtggattggagtgattaatcctggaagtggtggtactaatgacaatgagaagttcagggacaaggcaacattgactgcagacaaatcctccagcactgcctacatgcagctcagcagcctgacatctgatgactctgcggtctatttctgtgcaagagggaattactacggtactaccgactggtacttcgatgtctggggcgcagggaccacggtcaccgtctcctca

SEQ ID NO: 130 - protein, 108 aa, Artificial Sequence
  Description of Artificial Sequence Synthetic polypeptide
QIVLTQSPAIMSASLGERVTMTCTASASVSSSSLHWYQQKPGSPPKFWIYSTTNLASGVPTRFSGSGSGTSYSLTISNMEAEDAATYYCHQYHRSPPTFGGGTKLEIK

SEQ ID NO: 131 - DNA, 324 bp, Artificial Sequence
  Description of Artificial Sequence Synthetic polynucleotide
caaattgttctcacccagtctccagcaatcatgtctgcatctctaggggaacgggtcaccatgacctgcactgccagcgcaagtgtaagttccagctccttgcactggtaccagcagaagccaggatccccccccaaattctggatttatagcacaaccaacctggcttctggagtcccaactcgcttcagtggcagtgggtctgggacctcttactctctcacaatcagcaacatggaggctgaagatgctgccacttattactgccaccagtatcatcgttccccaccgacgttcggtggaggcaccaagctggaaatcaaa

SEQ ID NO: 132 - DNA, 20 bp, Homo sapiens
gctactctctctttctggcc

SEQ ID NO: 133 - DNA, 20 bp, Artificial Sequence
  Description of Artificial Sequence Synthetic primer
gtaacccgttgaaccccatt

SEQ ID NO: 134 - DNA, 20 bp, Artificial Sequence
  Description of Artificial Sequence Synthetic primer
ccatccaatcggtagtagcg

SEQ ID NO: 135 - DNA, 19 bp, Artificial Sequence
  Description of Artificial Sequence Synthetic primer
agaagacgggatgcctgtc

SEQ ID NO: 136 - DNA, 22 bp, Artificial Sequence
  Description of Artificial Sequence Synthetic primer
gtgaactgcaacatctgtaggt

SEQ ID NO: 137 - DNA, 22 bp, Artificial Sequence
  Description of Artificial Sequence Synthetic primer
tacaaaggcagtgaccatttgg

SEQ ID NO: 138 - DNA, 23 bp, Artificial Sequence
  Description of Artificial Sequence Synthetic primer
aggtgtaaattccttcgtccaga

SEQ ID NO: 139 - DNA, 21 bp, Artificial Sequence
  Description of Artificial Sequence Synthetic primer
tggcatttgctgaacgcattt

SEQ ID NO: 140 - DNA, 22 bp, Artificial Sequence
  Description of Artificial Sequence Synthetic primer
tgcagccaggtctaattgtttt

SEQ ID NO: 141 - protein, 449 aa, Artificial Sequence
  Description of Artificial Sequence Synthetic polypeptide
EVKLVESGGGLVQPGGSLKLSCAASGFTFSSFTLSWVRQTPEKRLEWVAYISYGGADTYYPDTVKGRFTIFRDNAKNTLYLQMSSLKSEDTARYYCARHGPYGKGDYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 142 - protein, 218 aa, Artificial Sequence
  Description of Artificial Sequence Synthetic polypeptide
DIVLTQSPASLAVSLGQRATISCRASESVDDFGISFMNWFQQKPGQPPKLLIFAASKQGSGVPARFSGSGSGTDFSLNIHPMEEDDTAMYFCQQSKEVPWTFGGVTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 443 aa, Artificial Sequence
  Description of Artificial Sequence Synthetic polypeptide
DVQLVESGGGLVQPGGSRKLSCAASGFTFSYFGMHWVRRAPEKGLEWVAFISSDSTTIYYTDTVKGRFTISRDNPKNTLFLQMTSLRSEDTAIYYCARFYDGAMDYWGQGTSVTVSTASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 144 - protein, 214 aa, Artificial Sequence
  Description of Artificial Sequence Synthetic polypeptide
DIVMTQSQKFMSTSVGDRVSITCKASQSVRTAVAWYQQKPGQSPKVLIYLASNRHTGVPDRFTGSGSGTDFTLTISNVQSEDLADYFCLQHWNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 487 aa, Artificial Sequence
  Description of Artificial Sequence Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGEVKLVESGGGLVQPGGSLKLSCAASGFTFSSFTLSWVRQTPEKRLEWVAYISYGGADTYYPDTVKGRFTIFRDNAKNTLYLQMSSLKSEDTARYYCARHGPYGKGDYYAMDYWGQGTSVTVSSGGGGSGGGGSGGGGSDIVLTQSPASLAVSLGQRATISCRASESVDDFGISFMNWFQQKPGQPPKLLIFAASKQGSGVPARFSGSGSGTDFSLNIHPMEEDDTAMYFCQQSKEVPWTFGGVTKLEIK

SEQ ID NO: 146 - protein, 477 aa, Artificial Sequence
  Description of Artificial Sequence Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGDVQLVESGGGLVQPGGSRKLSCAASGFTFSYFGMHWVRRAPEKGLEWVAFISSDSTTIYYTDTVKGRFTISRDNPKNTLFLQMTSLRSEDTAIYYCARFYDGAMDYWGQGTSVTVSTGGGGSGGGGSGGGGSDIVMTQSQKFMSTSVGDRVSITCKASQSVRTAVAWYQQKPGQSPKVLIYLASNRHTGVPDRFTGSGSGTDFTLTISNVQSEDLADYFCLQHWNYPLTFGAGTKLELK

SEQ ID NO: 147 - protein, 696 aa, Artificial Sequence
  Description of Artificial Sequence Synthetic polypeptide
EVKLVESGGGLVQPGGSLKLSCAASGFTFSSFTLSWVRQTPEKRLEWVAYISYGGADTYYPDTVKGRFTIFRDNAKNTLYLQMSSLKSEDTARYYCARHGPYGKGDYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGAGGGGDVQLVESGGGLVQPGGSRKLSCAASGFTFSYFGMHWVRRAPEKGLEWVAFISSDSTTIYYTDTVKGRFTISRDNPKNTLFLQHTSLRSEDTAIYYCARFYDGAMDYWGQGTSVTVSTGGGGSGGGGSGGGGSDIVMTQSQKFMSTSVGDRVSITCKASQSVRTAVAWYQQKPGQSPKVLIYLASNRHTGVPDRFTGSGSGTDFTLTISNVQSEDLADYFCLQHWNYPLTFGAGTKLEL

(19) United States
(12) Patent Application Publication
(10) Pub. No.: US 2025/0388702 A1
(43) Pub. Date: Dec. 25, 2025

(54) ANTI-KIR3DL3 ANTIBODIES AND USES THEREOF

(71) Applicant: Dana-Farber Cancer Institute, Inc., Boston, MA (US)

(72) Inventors: Gordon J. Freeman, Brookline, MA (US); Antonio R. Arulanandam, Winchester, MA (US)

(21) Appl. No.: 17/766,287

(22) PCT Filed: Oct. 2, 2020
(86) PCT No.: PCT/US2020/054063

§ 371 Date: Apr. 4, 2022

Related U.S. Application Data
(60) Provisional application No. 62/910,594, filed on Oct. 4, 2019.

(51) Int. Cl. C07K 16/46; A61P 35/00; C12N 15/63; G01N 33/574

(52) U.S. Cl. CPC C07K 16/468; A61P 35/00; C12N 15/63; G01N 33/57484; C07K 2317/14; C07K 2317/24; C07K 2317/31; C07K 2317/565; G01N 2800/52

(57) ABSTRACT

The present disclosure is based, in part, on the discovery of monoclonal antibodies, and antigen-binding fragments thereof, that specifically bind to KIR3DL3; bispecific antibodies and antigen-binding fragments thereof, that bind to KIR3DL3 and PD-1; as well as immunoglobulins, polypeptides, nucleic acids thereof, and methods of using such antibodies for prognostic, immunomodulatory, and therapeutic purposes.

### CROSS-REFERENCE TO RELATED APPLICATIONS

This application is the U.S. national phase of International Patent Application No. PCT/US2020/054063, filed on 2 Oct. 2020, which claims the benefit of priority to U.S. Provisional Application Ser. No. 62/910,594, filed on 4 Oct. 2019; the entire contents of each of said applications are incorporated herein in their entirety by this reference.

### STATEMENT OF RIGHTS

This invention was made with government support under W81XWH-18-1-0578 awarded by the Medical Research and Development Command, and P50 CA206963, P50 CA101942, and AI056299 awarded by the National Institutes of Health. The government has certain rights in the invention.

### SEQUENCE LISTING

The present specification makes reference to a Sequence Listing (submitted electronically as a .txt file named “DFS-27701_Sequence_Listing” on Apr. 4, 2022). The .txt file was generated on Nov. 17, 2020 and is 120,293 bytes in size. The entire contents of the Sequence Listing are herein incorporated by reference.

### BACKGROUND OF THE INVENTION

Immune checkpoints, such as CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, butyrophilins, and A2aR, and many more, negatively regulate immune response progression based on complex and combinatorial interactions between numerous inputs. Inhibitors of immune checkpoints can modulate immune responses in some subjects, but immune checkpoint expression and interactions with natural binding partners vary between subjects and within tissues of a subject. A significant percentage of patients do not respond to this treatment and the many patients that do respond eventually develop resistance. Thus, there is a critical unmet need to find additional immune pathways that are non-redundant with the PD-1 pathway.

HERV-H LTR-associating 2 (HHLA2, also known as B7-H5, B7-H7) is a B7 family member that modulates T-cell functions. HHLA2 is broadly expressed in a variety of tumors (e.g., solid and hematologic cancers including primary human renal cell carcinoma (RCC)) and antigen presenting cells and has been implicated as both an activating and inhibitory ligand for T cells. HHLA2 was identified as a specific ligand for TMIGD2 (CD28H, IGPR-1) and the HHLA2/TMIGD2 interaction selectively costimulates human T-cell growth and cytokine production via an AKT-dependent signaling cascade (Zhu et al. (2013) Nat. Comm. 4:2043; Janakiram et al. (2015) Clin. Cancer Res. 21:2359-2366). TMIGD2 expressed in naive T cells is an activating receptor for HHLA2 and transduces co-stimulatory signals following T cell antigen receptor (TCR) engagement. TMIGD2 is downregulated following repeated TCR stimulation. It is possible that a putative inhibitory receptor for HHLA2 is upregulated on activated T cells to modulate T cell activation.

### SUMMARY OF THE INVENTION

Prior to the present disclosure, the existence of an uncharacterized receptor for HHLA2 on activated T cells that exerts a coinhibitory function was suggested by several studies (Zhao et al. (2013) Proc. Natl. Acad. Sci. USA 110:9879-9884; Xiao and Freeman et al. (2015) Clin. Cancer Res. 21:2201-2203; Wang et al. (2014) J. Immunol. 192:126.11). It was discovered that HHLA2 binds KIR3DL3, a receptor on T cells and NK cells, and that a consequence of the HHLA2-KIR3DL3 interaction is inhibition of T cell and NK cell activation (PCT/US2019/026034). Accordingly, the present disclosure encompasses the recognition that the KIR3DL3 receptor is a candidate for cancer immunotherapy, and provided herein are compositions and methods for targeting KIR3DL3 to modulate immune response.

The present disclosure is based, at least in part, on the discovery that agents (e.g., antibodies) target KIR3DL3 can block specifically the HHLA2-KIR3DL3 interaction and can be used in methods to modulate immune response. Importantly, it is presented herein that targeting KIR3DL3 does not disrupt the overall function of HHLA2, which also includes activating immune response via its interaction with TMIGD2. Accordingly, the present disclosure provides the important and surprising finding that targeting KIR3DL3 provides the specificity of blocking only the immune inhibitory function of HHLA2, thereby eliciting an effective immune response (e.g., against cancer cells), without downregulating the immune activating function of HHLA2. Development of agents that specifically block the immune inhibitory activity of the HHLA2 pathway and preserve its stimulatory function represents a new approach to immune checkpoint blockade in patients with cancer (e.g., hematologic cancer and solid tumors, including clear cell renal cell carcinoma (ccRCC)).

The present disclosure is also based, at least in part, on the discovery that agents that target both KIR3DL3 and PD-1 can be used to modulate immune response and/or treat cancer. In some embodiments, KIR3DL3 x PD-1 bispecific antibodies described herein are useful as checkpoint immunotherapies, such as to activate T and NK cells in tumors. In some embodiments, a KIR3DL3 x PD-1 bispecific antibody is additive or synergistic with PD-1 or PD-L1 or other checkpoint immunotherapy. Furthermore, HHLA2 and/or KIR3DL3 expression in the tumor is a useful biomarker for determining the responsiveness to KIR3DL3 mAb and/or KIR3DL3 x PD-1 bispecific antibody checkpoint blockade.

A panel of exemplary, representative anti-KIR3DL3 human monoclonal antibodies (mAbs) is described herein as immune checkpoint inhibitor agents. Blocking and non-blocking anti-KIR3DL3 mAbs were identified, and the anti-KIR3DL3 mAbs that block HHLA2 binding to KIR3DL3 were shown to be checkpoint inhibitor antibodies in T cell and NK cell assays.

In one aspect, a monoclonal antibody, or antigen-binding fragment thereof, comprising a) a heavy chain sequence with at least about 95% identity to a heavy chain sequence selected from the group consisting of the sequences listed in Tables 2, 7, and 8; and/or b) a light chain sequence with at least about 95% identity to a light chain sequence selected from the group consisting of the sequences listed in Tables 2, 7, and 8, is provided.

In another aspect, a monoclonal antibody, or antigen-binding fragment thereof, comprising a) one, two, or three heavy chain CDR sequences each with at least about 95% identity to a heavy chain CDR sequence selected from the group consisting of the sequences listed in Tables 2, 7, and 8; and/or b) one, two, or three light chain CDR sequences each with at least about 95% identity to a light chain CDR sequence selected from the group consisting of the sequences listed in Tables 2, 7, and 8, is provided.

In still another aspect, a monoclonal antibody, or antigen-binding fragment thereof, comprising a) a heavy chain sequence selected from the group consisting of the sequences listed in Tables 2, 7, and 8; and/or b) a light chain sequence selected from the group consisting of the sequences listed in Tables 2, 7, and 8, is provided. In yet another aspect, a monoclonal antibody, or antigen-binding fragment thereof, comprising a) one, two, or three heavy chain CDR sequences each selected from the group consisting of the sequences listed in Tables 2, 7, and 8; and/or b) one, two, or three light chain CDR sequences each selected from the group consisting the sequences listed in Tables 2, 7, and 8, is provided.

Numerous embodiments are further provided that can be applied to any aspect encompassed by the present disclosure as described herein. For example, in one embodiment, a monoclonal antibody, or antigen-binding fragment thereof, is chimeric, humanized, composite, murine, or human. In another embodiment, a monoclonal antibody, or antigen-binding fragment thereof, is (a) detectably labeled, (b) conjugated to a cytotoxic agent, optionally a chemotherapeutic agent, a biologic agent, a toxin, and/or a radioactive isotope, (c) comprises an effector domain, (d) comprises an Fc domain, and/or (e) is selected from the group consisting of Fv, Fav, F(ab′)2), Fab′, dsFv, scFv, sc (Fv)2, and diabodies fragments. In still another embodiment, a monoclonal antibody, or antigen-binding fragment thereof, is obtainable from hybridoma deposited under deposit accession number In yet another embodiment, a monoclonal antibody, or antigen-binding fragment thereof, inhibits binding of HHLA2 to KIR3DL3. KIR3DL3 mAbs that block HHLA2 binding to KIR3DL3 in T cell activation assays were shown to be checkpoint blockers. In another embodiment, a monoclonal antibody, or antigen-binding fragment thereof, specifically binds KIR3DL3.

A panel of exemplary, representative bispecific antibodies that bind to KIR3DL3 and PD-1 is described herein as immune checkpoint inhibitor agents.

In one aspect, presented herein is a bispecific antibody, or antigen-binding fragment thereof, comprising: a) a heavy chain sequence with at least about 95% identity to a heavy chain sequence selected from the group consisting of the sequences listed in Tables 2 and 7-9; and/or b) a light chain sequence with at least about 95% identity to a light chain sequence selected from the group consisting of the sequences listed in Tables 2 and 7-9.

In another aspect, a bispecific antibody, or antigen-binding fragment thereof, comprising: a) one, two, or three heavy chain CDR sequences each with at least about 95% identity to a heavy chain CDR sequence selected from the group consisting of the sequences listed in Tables 2 and 7-9; and/or b) one, two, or three light chain CDR sequences each with at least about 95% identity to a light chain CDR sequence selected from the group consisting of the sequences listed in Tables 2 and 7-9, is provided.

In still another aspect, a bispecific antibody, or antigen-binding fragment thereof, comprising: a) a heavy chain sequence selected from the group consisting of the sequences listed in Tables 2 and 7-9; and/or b) a light chain sequence selected from the group consisting of the sequences listed in Tables 2 and 7-9, is provided.

In yet another aspect, a bispecific antibody, or antigen-binding fragment thereof, comprising: a) one, two, or three heavy chain CDR sequences each selected from the group consisting of the sequences listed in Tables 2 and 7-9; and/or b) one, two, or three light chain CDR sequences each selected from the group consisting the sequences listed in Tables 2 and 7-9, is provided.

Numerous embodiments are provided that can be applied to any aspect encompassed by the present disclosure as described herein. For example, in one embodiment, a a bispecific antibody, or antigen-binding fragment thereof, is chimeric, humanized, composite, murine, or human. In another embodiment, a bispecific antibody, or antigen-binding fragment thereof, is (a) detectably labeled, (b) conjugated to a cytotoxic agent, optionally a chemotherapeutic agent, a biologic agent, a toxin, and/or a radioactive isotope, (c) comprises an effector domain, (d) comprises an Fc domain, and/or (e) is selected from the group consisting of Fv, Fav, F(ab′)2), Fab′, dsFv, scFv, sc (Fv)2, and diabodies fragments. In still another embodiment, a bispecific antibody, or antigen-binding fragment thereof, is obtainable from hybridoma deposited under deposit accession number In yet another embodiment, a bispecific antibody, or antigen-binding fragment thereof, inhibits the binding of (a) HHLA2 to KIR3DL3, and (b) PD-1 to PD-L1 and/or PD-L2. A bispecific antibody that binds to both KIR3DL3 and PD-1 were shown to be checkpoint blockers. In another embodiment, a bispecific antibody, or antigen-binding fragment thereof, specifically binds KIR3DL3 and PD-1. In still another embodiment, bispecific antibody, or antigen-binding fragment thereof, comprises a) a heavy chain sequence listed in Table 9; and/or b) a light chain sequence listed in Table 9.

In another aspect, immunoglobulin heavy and/or light chains selected from the group consisting of immunoglobulin heavy and light chain sequences listed in Tables 2 and 7-9, are provided.

In still another aspect, an isolated nucleic acid molecule that (a) encodes an immunoglobulin heavy chain, an immunoglobulin light chain, and/or a monoclonal antibody, or antigen-binding fragment thereof, encompassed by the present disclosure described herein; and/or (b) hybridizes, under stringent conditions, with the complement of a nucleic acid encoding a polypeptide selected from the group consisting of polypeptide sequences listed in Tables 2 and 7-9, or a sequence with at least about 95% homology to a nucleic acid encoding a polypeptide selected from the group consisting of the polypeptide sequences listed in Tables 2 and 7-9, is provided.

In yet another aspect, a vector comprising an isolated nucleic acid described herein, is provided.

In another aspect, host cells comprising an isolated nucleic acid described herein, comprises a vector described herein, express an antibody, or antigen-binding fragment thereof, described herein, or are accessible under deposit accession number are provided.

In still another aspect, a device or kit comprising at least one antibody, or antigen-binding fragment thereof, (e.g., a monoclonal antibody, a bispecific antibody, or antigen-binding fragment thereof) described herein, a device or kit optionally comprising a label to detect at least one antibody, or antigen-binding fragment thereof, or a complex comprising a antibody, or antigen-binding fragment thereof, is provided.

In yet another aspect, a method of producing at least one antibody, or antigen-binding fragment thereof, (e.g., a monoclonal antibody, a bispecific antibody, or antigen-binding fragment thereof) described herein, which method comprises steps of: (i) culturing a transformed host cell which has been transformed by a nucleic acid comprising a sequence encoding at least one in accordance with the present disclosure under conditions suitable to allow expression of said antibody, or antigen-binding fragment thereof; and (ii) recovering an expressed antibody, or antigen-binding fragment thereof, is provided.

In another aspect, a method of detecting presence or level of an KIR3DL3 polypeptide comprising detecting said polypeptide in a sample by use of at least one antibody, or antigen-binding fragment thereof, (e.g., a monoclonal antibody, a bispecific antibody, or antigen-binding fragment thereof) described herein. In one embodiment, at least one antibody, or antigen-binding fragment thereof, forms a complex with a KIR3DL3 polypeptide and a complex is detected in a form of an enzyme linked immunosorbent assay (ELISA), radioimmunoassay (RIA), immunochemically, Western blot, or using an intracellular flow assay.

In still another aspect, a method of predicting responsiveness to a therapy targeting KIR3DL3, the method comprising: a) determining a level of KIR3DL3 and/or HHLA2 in a subject sample using at least one antibody, or antigen-binding fragment thereof, (e.g., a monoclonal antibody, a bispecific antibody, or antigen-binding fragment thereof) described herein; b) determining a level of KIR3DL3 and/or HHLA2 in a sample from at least one control subject having good responsiveness to a therapy targeting KIR3DL3, usin at least one antibody, or antigen-binding fragment thereof, described herein; and c) comparing the level of KIR3DL3 and/or HHLA2 in the subject sample and in the sample from the control subject; wherein a same or higher level of KIR3DL3 and/or HHLA2 in the subject sample as compared to the level in the sample from the at least one control subject is an indication that the subject will be responsive to therapy, is provided. In one embodiment, a therapy targets KIR3DL3 using at least one antibody, or antigen-binding fragment thereof, (e.g., a monoclonal antibody, a bispecific antibody, or antigen-binding fragment thereof) described herein.

In yet another aspect, a method of predicting responsiveness to a therapy targeting KIR3DL3 using at least one antibody, or antigen-binding fragment thereof, (e.g., a monoclonal antibody, a bispecific antibody, or antigen-binding fragment thereof) described herein, the method comprising: a) determining a level of KIR3DL3 and/or HHLA2 in a subject sample; b) determining a level of KIR3DL3 and/or HHLA2 in a sample from at least one control subject having good responsiveness to a therapy targeting KIR3DL3; and c) comparing the level of KIR3DL3 and/or HHLA2 in the subject sample and in the sample from the control subject; wherein the same or higher level of KIR3DL3 and/or HHLA2 in the subject sample as compared to the level in the sample from the at least one control subject is an indication that the subject will be responsive to the therapy, is provided.

As described above, certain embodiments are applicable to any method described herein. For example, in one embodiment, a sample is a portion of a single sample obtained from at least one subject or portions of pooled samples obtained from at least one subject. In another embodiment, therapy blocks an interaction and/or signaling between (a) HHLA2 and KIR3DL3; and/or (b) PD-1 and PD-L1 and/or PD-L2. In still another embodiment, a sample comprises cells (e.g., T cells or natural killer (NK) cells, serum, peritumoral tissue, and/or intratumoral tissue obtained from a subject).

In yet another aspect, a method of treating a subject afflicted with cancer comprising administering to a subject at least one antibody, or antigen-binding fragment thereof, (e.g., a monoclonal antibody, a bispecific antibody, or antigen-binding fragment thereof) described herein, is provided.

As described above, certain embodiments are applicable to any method described herein. For example, in one embodiment, at least one antibody, or antigen-binding fragment thereof, (e.g., a monoclonal antibody, a bispecific antibody, or antigen-binding fragment thereof) described herein (a) reduces proliferating cancer cell numbers in the cancer; (b) reduces volume or size of a tumor of the cancer; and/or (c) activates a T cell and/or an NK cell. In another embodiment, at least one antibody, or antigen-binding fragment thereof, (e.g., a monoclonal antibody, a bispecific antibody, or antigen-binding fragment thereof) described herein is administered in a pharmaceutically acceptable formulation. In still another embodiment, a method described herein further comprising administering to a subject a therapeutic agent or regimen for treating cancer. In yet another embodiment, a method described herein, further comprising administering to a subject an additional therapy selected from the group consisting of immunotherapy, checkpoint blockade, cancer vaccines, chimeric antigen receptors (e.g., a CAR targeting CD19), chemotherapy, radiation, target therapy, and surgery. In another embodiment, cancer cells and/or tumor immune infiltrating cells in a subject express HHLA2. In still another embodiment, a cancer is selected from the group consisting of adenocarcinoma, chronic myelogenous leukemia (CML), lung cancer, renal cancer, pancreatic cancer, colorectal cancer, acute myeloid leukemia, head and neck carcinoma, liver cancer, ovarian cancer, prostate cancer, uterine cancer, gliomas, glioblastoma, neuroblastoma, breast cancer, pancreatic ductal carcinoma, thymoma, B-CLL, leukemia, B cell lymphoma, and a cancer infiltrated with immune cells expressing a receptor to HHLA2. In yet another embodiment, a cancer is selected from the group consisting of lung cancer, renal cancer, pancreatic cancer, colorectal cancer, acute myeloid leukemia (AML), head and neck carcinoma, liver cancer, ovarian cancer, prostate cancer, and uterine cancer. In another embodiment, a subject is an animal model of cancer. In still another embodiment, an animal model is a mouse model, optionally wherein the mouse model is a humanized mouse model. In yet another embodiment, a subject is a mammal, such as a humanized mouse or a human.

In another aspect, a method of modulating an immune response using at least one anti-KIR3DL3 antibody, or antigen-binding thereof, described herein, is provided. For example, in one embodiment, at least one anti-KIR3DL3 antibody, or antigen-binding fragment thereof, described herein inhibits or disrupts the interaction between HHLA2 and its binding inhibitor receptor, KIR3DL3. In another embodiment, at least one anti-KIR3DL3 antibody, or antigen-binding fragment thereof, described herein is conjugated to a cytotoxic agent (e.g., a chemotherapeutic agent, a biologic agent, a toxin, and/or a radioactive isotope). In still another embodiment, an immune response is downregulated. In another embodiment, an immune response is upregulated. In yet another embodiment, an interaction between (a) HHLA2 and KIR3DL3; and/or (b) PD-1 and PD-L1 and/or PD-L2 is blocked. In another embodiment, an anti-KIR3DL3 antibody, or antigen-binding fragment thereof, is a checkpoint inhibitor of T cell activation for cancer immunotherapy. In still another embodiment, modulating an immune response comprises modulating a T cell function or NK cell function (e.g., cytotoxicity, such as against cancer cells like cancer cells expressing HHLA2). In yet another embodiment, a cancer cancer is selected from the group consisting of adenocarcinoma, chronic myelogenous leukemia (CML), lung cancer, renal cancer, pancreatic cancer, colorectal cancer, acute myeloid leukemia, head and neck carcinoma, liver cancer, ovarian cancer, prostate cancer, uterine cancer, gliomas, glioblastoma, neuroblastoma, breast cancer, pancreatic ductal carcinoma, thymoma, B-CLL, leukemia, B cell lymphoma, and a cancer infiltrated with immune cells expressing a receptor to HHLA2. In another embodiment, a cancer is selected from the group consisting of lung cancer, renal cancer, pancreatic cancer, colorectal cancer, acute myeloid leukemia (AML), head and neck carcinoma, liver cancer, ovarian cancer, prostate cancer, and uterine cancer. In still another embodiment, a method further comprises administering to a subject an additional therapy selected from the group consisting of immunotherapy, checkpoint blockade, cancer vaccines, chimeric antigen receptors (e.g., a CAR targeting CD19), chemotherapy, radiation, target therapy, and surgery. In yet another embodiment, an immune response is modulated in an animal model of cancer (e.g., a mouse model and/or a humanized animal model). In another embodiment, an immune response is modulated in a mammal, such as a humanized mouse or a human.

For any figure showing a bar histogram, curve, or other data associated with a legend, the bars, curve, or other data presented from left to right for each indication correspond directly and in order to the boxes from top to bottom, or from left to right, of the legend.

### BRIEF DESCRIPTION OF FIGURES

FIG. 1A-FIG. 1B show results of an expression screen identifying KIR3DL3 as a receptor for HHLA2. FIG. 1A shows cell microarray analysis results using soluble HHLA2-mIgG2a (HHLA2-Ig) to bind the indicated cell surface receptors individually expressed in HEK293 cells. HHLA2-Ig is shown to bind to TMIGD2, KIR3DL3, and control (FCGR2A) but not to other members of the KIR family, PD-1, PD-L1 or HHLA2. FIG. 1B shows flow cytometry analysis of HHLA2-Ig or control Ig binding to control 300.19 cells or 300.19 cells stably expressing KIR3DL3, TMIGD2, or HHLA2 using the indicated concentrations of HHLA2-Ig or isotype control (from 0.1 μg/mL to 160 μg/mL).

FIG. 2A-FIG. 2D show identification and characterization of KIR3DL3 as a second receptor for HHLA2. FIG. 2A shows duplicate microarray slides of cells expressing 384 human receptors and co-expressing GFP, identifying KIR3DL3 as a receptor for HHLA2-Ig (upper panel) and showing GFP expression as a control for transfection and for spot localization (lower panel). FIG. 2B shows cell microarray analysis results shown in FIG. 1A using soluble HHLA2-mIgG2a (HHLA2-Ig) to bind the indicated cell surface receptors individually expressed in HEK293 cells. HHLA2-Ig is shown to bind to TMIGD2, KIR3DL3, and control (FCGR2A), but not to other members of the KIR family, PD-1, PD-L1 or HHLA2. FIG. 2C shows transfection control (GFP expression) results of receptor array shown in FIG. 2B (and in FIG. 1A). FIG. 2D shows positive control treatment restuls of receptor array. Cell microarray analysis using soluble PD-1-Ig and PD-L1-Ig incubated with the same panel of over-expressed receptors as in FIG. 2B and FIG. 1A shows binding to FCGR2A, PD-1 and PD-L1 but none of the KIR. PD-L1 spots that do not bind PD-1-Ig are alternatively spliced isoforms.

FIG. 3A-FIG. 3E show characterization of a panel of KIR3DL3 and HHLA2 mAbs. Flow cytometry analysis of binding of (FIG. 3A) KIR3DL3 mAbs to 300.19 cells expressing KIR3DL3. FIG. 3B shows capacity of KIR3DL3 mAbs to block binding of HHLA2-Ig to 300.19 cells expressing KIR3DL3. FIG. 3C shows HHLA2 mAbs binding to 300.19 cells expressing HHLA2 with 2C4, 2G2 and 6F10 showing strongest binding and less binding with 6D10. FIG. 3D shows capacity of HHLA2 mAbs to block binding of HHLA2-Ig to 300.19 cells expressing KIR3DL3 with 2C4, 2G2, and 6F10 showing strongest binding. FIG. 3E shows capacity of HHLA2 mAbs 2G2 and 6F10 to block binding of HHLA2-Ig to 300.19 cells expressing TMIGD2.

FIG. 4A-FIG. 4C show HHLA2-mIgG2a binding to KIR3DL3 and TMIGD2. FIG. 4A shows normalized binding data of FIG. 1B. HHLA2-mIgG2a binding to KIR3DL3 (blue) or TMIGD2 (cyan) or control HHLA2 (red) transfected or parental 300.19 cells (green). FIG. 4B and FIG. 4C show HHLA2-mIgG2a or isotype control (10 μg/ml) binding to KIR3DL3-transfected 293T cells (FIG. 4B) or TMIGD2-transfected 293T cells (FIG. 4C) by flow cytometry.

FIG. 5 shows binding data for anti-KIR3DL3 mAbs on KIR3DL3 transfected 300.19 mouse pre-B cell leukemic cell line by flow cytometry.

FIG. 6 shows binding data for anti-KIR3DL3 mAbs on KIR3DL3 by Western blotting. In particular, Western blot analysis results of KIR3DL3 mAbs using Jurkat cells transfected with KIR3DL3 are shown.

FIG. 7 shows KIR3DL3 expression in Jurkat parental cells, Jurkat transfected with KIR3DL3, NK-92 cells, and NK-92-MI cells. Lysates were blotted with anti-KIR3DL3 mAb 574.1F12 at 5 μg/ml.

FIG. 8 shows single cell RNA sequencing analysis of KIR3DL3 expression as assessed in a publicly available data base (see EMBL-EBI database available on the World Wide Web at ebi.ac.uk/gxa/sc/home and corresponding publication titled “Reconstructing the human first trimester fetal-maternal interface using single cell transcriptomics” available on the World Wide Web at biorxiv.org/content/10.1101/429589v1). KIR3DL3 expression is indicated on the right panel as blue dots. Black boxes highlight decidual NK cells where most KIR3DL3 expression is noted.

FIG. 9 shows anti-KIR3DL3 mAb blockade of HHLA2 binding to KIR3DL3.

FIG. 10A-FIG. 10D show KIR3DL3 expression on activated human T-cells and NK92-MI cells. FIG. 10A shows results of T cells purified from whole blood of 4 normal donors, activated with CD3/CD28 antibody tetramers, and subjected to FACS analysis performed in duplicate at indicated days to assess KIR3DL3 expression in gated CD3+CD4+ and CD3+CD8+ T cells. Representative FACS plots showing KIR3DL3 expression at day 0 (unactivated) (FIG. 10B) and day 21 post-activation (FIG. 10C). FIG. 10D shows KIR3DL3 expression on NK92-MI (left panel), but minimally on NK-92 cells (right panel).

FIG. 11A-FIG. 11C show that KIR3DL3 is an inhibitory receptor in T cells and T cell activation is enhanced by HHLA2/KIR3DL3 blockade. FIG. 11A shows results of Jurkat IL-2-reporter T cells expressing KIR3DL3 co-cultured with CHO cells expressing anti-CD3 scFV, CHO cells co-expressing anti-CD3 scFV and HHLA2, or untransfected CHO cells in the presence or absence of CD28 mAb as indicated. Luciferase activity is represented as relative light units (RLU). FIG. 11B and FIG. 11C shows results of Jurkat IL-2-reporter T cells expressing KIR3DL3 co-cultured with CHO cells co-expressing anti-CD3 scFV and HHLA2 in the presence of CD28 mAb and HHLA2 mAbs (FIG. 11B) or KIR3DL3 mAbs (FIG. 11C). Fold activation of IL-2 reporter luciferase activity is presented as mean±S.D. (n>3; **** P≤0.0001).

FIG. 12 shows that HHLA2/TMIGD2 interaction enhances T cell activation. Jurkat T cells expressing TMIGD2 and bearing a NFAT promoter linked to luciferase were co-cultured with anti-CD3 scFV CHO cells or HHLA2-anti-CD3 scFV CHO cells and Luciferase activity (RLU) was assayed. Quantifications are presented as mean±S.D. (n>3; *** P≤0.001).

FIG. 13 shows anti-KIR3DL3 mAb enhancement of IL-2 promoter driven Luciferase expression in Jurkat-KIR3DL3 T cells in response to anti-CD3-scFV and HHLA2-mediated signals.

FIG. 14 shows anti-HHLA2 mAb enhancement of IL-2 promoter driven luciferase expression in Jurkat-KIR3DL3 T cells in response to anti-CD3-scFV and HHLA2-mediated signals.

FIG. 15A-FIG. 15D show KIR3DL3-CD19-CAR-T cell cytotoxicity against HeLa tumors expressing CD19 and HHLA2. FIG. 15A shows KIR3DL3/CAR-19 expression plasmid and lentivirus production. In particular, FIG. 15A shows a schematic diagram of the PMC456-Ef1a expression plasmid. FIG. 15B shows generation and expansion of KIR3DL3/CD19-CAR-T cells. In particular, FIG. 15B shows FACS profile of KIR3DL3/CAR-19 T cells (PMC456 Cells). FIG. 15C shows generation of stable HeLa-CD19 and HeLa-CD19+KIR3DL3 expressing cells. In particular, FIG. 15C shows a FACS profile of HeLa-CD19 and HeLa-CD19-KIR3DL3 tumor cells. FIG. 15D shows that HHLA2 mAb enhances KIR3DL3 CD19-CAR-T cell cytotoxicity against HHLA2+CD19 transfected HeLa tumor cells.

FIG. 16A-FIG. 16C show cytotoxicity assay of NK92 cells expressing KIR3DL3 against HeLa tumor target cells expressing HHLA2 or not. FIG. 16A shows KIR3DL3 expression plasmid and lentivirus production. In particular, FIG. 16A shows a schematic diagram of the PMC579 KIR3DL3 expression plasmid. FIG. 16B shows derivation of KIR3DL3 transduced NK92 cells. In particular FIG. 16B shows KIR3DL3/NK92 FACS profile. FIG. 16C shows derivation of HHLA2-transfected or transduced K562 and HeLa cells respectively. In particular, FIG. 16C shows FACS profile of HHLA2-transfected K562 cells and HHLA2 transduced HeLa tumor cells.

FIG. 17A-FIG. 17C shows NK92 cytotoxicity against HeLa alone and HeLa transduced HHLA2 expressing tumor target cells. FIG. 17A shows inhibition of NK92 cytotoxicity by KIR3DL3-HHLA2 interaction/pathway. FIG. 17B shows enhancement of NK92-KIR3DL3 cytotoxicity by HHLA2 mAbs and KIR3DL3 mAbs. FIG. 17C shows a schematic diagram of certain cytotoxicity assays.

FIG. 18 shows Beta2-microglobulin and HHLA2 expression in Raji-B2M KO and HHLA2 transfected Raji-B2M KO by flow cytometry.

FIG. 19A-FIG. 19E show that KIR3DL3 is an inhibitory receptor in NK cells and NK cytotoxicity is enhanced by HHLA2/KIR3DL3 blockade. FIG. 19A shows NK92-MI cytotoxicity on Raji cells harboring a B2M deletion (Raji-B2M KO cells) and Raji-B2M KO cells expressing HHLA2. FIG. 19B and FIG. 19C show NK92-MI cytotoxicity on Raji-B2M KO cells expressing HHLA2 at indicated E/T ratios in presence of 10 μg/ml of KIR3DL3 antibodies (FIG. 19B) or HHLA2 antibodies (FIG. 19C) and isotype controls. FIG. 19D shows results of NK92-MI cells incubated with Raji B2M KO cells or with Raji B2M KO cells overexpressing HHLA2 at indicated E: T ratios. Degranulation was measured as % CD107a positive cells of CD56+ population. Controls were effector cells alone or effector cells with PMA/ION, which leads to total degranulation. FIG. 19E shows enhanced degranulation of NK92-MI cells targeting Raji B2M KO cells overexpressing HHLA2 in the presence of KIR3DL3 mAb (1G7) as compared to isotype control. Quantifications are presented as mean±S.D. (N>3; P≥0.05; * P≤0.05; ** P≤0.01; *** P<0.001; **** P≤0.0001).

FIG. 20 shows that HHLA2 expression is distinct from PD-L1 expression. FIG. 20 shows expression levels of B7 gene family members in RCC as compared to normal kidney from The Cancer Genome Atlas (TCGA) samples.

FIG. 21A-FIG. 21B show an HHLA2 pathway model. HHLA2 delivers an immune stimulatory signal via TMIGD2 in naïve T cells or NK cells. FIG. 21A shows T cell activation leading to a loss of TMIGD2 expression and gain of KIR3DL3. HHLA2 delivers an immune inhibitory signal via KIR3DL3 in activated T cells. FIG. 21B shows NK cytolytic activity regulated by inhibitory and activating receptors. Inhibitory receptors include most KIRs, CD94/NKG2A, and LILRBI, which recognize MHC class I, E, and G, respectively. Activating receptors include NKG2D, NKp30, NKp44, NKp46, CD94/NKG2C, and TMIGD2, which recognize ULBP-1, MICA, MICB, B7-H6, HLA-E, HHLA2, and others. If tumors lose MHC expression (missing self), inhibitory signal is reduced and activating signals dominate, leading to tumor lysis by NK cells. HHLA2 on tumors are an inhibitory signal, independent of MHC, that inhibits lysis by KIR3DL3-positive NK cells.

FIG. 22 shows a schematic diagram for construction of KIR3DL3 x PD-1 bispecific antibody.

FIG. 23 shows binding sensograms of KIR3DL3 and PD-1 human IgG4 and scFV antibodies in Octet assay.

### DETAILED DESCRIPTION OF THE INVENTION

HHLA2, a B7 gene family member, is broadly expressed in a variety of tumors and antigen presenting cells and has been implicated as both an activating and inhibitory ligand for T cells. TMIGD2 expressed in naive T cells is an activating receptor for HHLA2 and transduces co-stimulatory signals following T cell antigen receptor (TCR) engagement. TMIGD2 is downregulated following repeated TCR stimulation. HHLA2 binds to another receptor, KIR3DL3, that is expressed in T cells and NK cells. As is described herein, the present disclosure encompasses the recognition that, unlike the immune activating function of the HHLA2-TMIGD2 interaction, the HHLA2-KIR3DL3 interaction can inhibit immune responses, and provides an attractive target for modulation in a variety of diseases, disorders or conditions including, for example, cancer.

The present disclosure is based, at least in part, on the discovery that targeting KIR3DL3 can block specifically the HHLA2-KIR3DL3 interaction that inhibits immune response. Importantly, targeting KIR3DL3 does not disrupt the overall function of HHLA2, which also includes activating immune response via its interaction with TMIGD2. Accordingly, precisely targeting KIR3DL3 provides the specificity of blocking only the immune inhibitory function of HHLA2, thereby eliciting an effective immune response, e.g., against cancer cells, without downregulating the immune activating function of HHLA2.

The present disclosure is also based, at least in part, on the discovery that agents that target both KIR3DL3 and PD-1 can be used to modulate immune response and/or treat cancer. In some embodiments, KIR3DL3 x PD-1 bispecific antibodies described herein are checkpoint immunotherapy to activate T and NK cells in tumors. In some embodiments, a KIR3DL3 x PD-1 bispecific antibody is additive or synergistic with PD-1 or PD-L1 or other checkpoint immunotherapy. Furthermore, HHLA2 and/or KIR3DL3 expression in a tumor is a useful biomarker for determining the responsiveness to KIR3DL3 mAb and/or KIR3DL3 x PD-1 bispecific antibody checkpoint blockade.

A panel of exemplary, representative anti-KIR3DL3 human monoclonal antibodies (mAbs) is described herein as immune checkpoint inhibitor agents. Blocking and non-blocking anti-KIR3DL3 mAbs were identified, and the anti-KIR3DL3 mAbs that block HHLA2 binding to KIR3DL3 were shown to be checkpoint inhibitor antibodies in T cell and NK cell assays. The binding characteristics as well as the variable region heavy and light chain gene sequences for these candidate therapeutic anti-KIR3DL3 antibodies are described herein.

A panel of exemplary, representative bispecific antibodies, or antigen-binding fragment thereof, that binds to both KIR3DL3 and PD-1 is also described herein as immune checkpoint inhibitor agents. Targeting two immune checkpoints with non-overlapping expression provides a combination therapy with additive or synergistic anti-tumor activity.

Accordingly, the present disclosure provides monoclonal antibodies, and antigen-binding fragments thereof, that specifically bind to KIR3DL3, bispecific antibodies, and antigen-binding fragments thereof, that bind to KIR3DL3 and PD-1, as well as immunoglobulins, polypeptides, nucleic acids thereof, and methods of using such antibodies, such as for immunomodulatory and therapeutic purposes.

### I. Definitions

The articles “a” and “an” are used herein to refer to one or to more than one (i.e. to at least one) of the grammatical object of the article. By way of example, “an element” means one element or more than one element.

The term “altered amount” of a marker refers to increased or decreased copy number of a marker and/or increased or decreased nucleic acid level of a particular marker gene or genes in a sample, as compared to that of the marker in a control sample. The term “altered amount” of a marker also includes an increased or decreased protein level of a marker in a sample, as compared to the protein level of the marker in a normal, control sample.

The term “altered activity” of a marker refers to an activity of a marker which is increased or decreased in a disease state, e.g., in a biological sample, as compared to the activity of the marker in a normal, control sample. Altered activity of a marker may be the result of, for example, altered expression of the marker, altered protein level of the marker, altered structure of the marker, or, e.g., an altered interaction with other proteins involved in the same or different pathway as the marker, or altered interaction with transcriptional activators or inhibitors.

The term “altered structure” of a marker refers to the presence of mutations or allelic variants within a marker gene or maker protein, e.g., mutations which affect expression or activity of the marker, as compared to the normal or wild-type gene or protein. For example, mutations include, but are not limited to substitutions, deletions, or addition mutations. Mutations may be present in the coding or non-coding region of the marker.

The term “activating receptor” includes immune cell receptors that bind antigen, complexed antigen (e.g., in the context of MHC polypeptides), or bind to antibodies. Such activating receptors include T cell receptors (TCR), B cell receptors (BCR), cytokine receptors, LPS receptors, complement receptors, and Fc receptors.

T cell receptors are present on T cells and are associated with CD3 polypeptides. T cell receptors are stimulated by antigen in the context of MHC polypeptides (as well as by polyclonal T cell activating reagents). T cell activation via the TCR results in numerous changes, e.g., protein phosphorylation, membrane lipid changes, ion fluxes, cyclic nucleotide alterations, RNA transcription changes, protein synthesis changes, and cell volume changes.

The term “chimeric antigen receptor,” “CAR,” or “CAR-T” refers to engineered T cell receptors (TCR) having a desired antigen specificity. T lymphocytes recognize specific antigens through interaction of the T cell receptor (TCR) with short peptides presented by major histocompatibility complex (MHC) class I or II molecules. For initial activation and clonal expansion, naive T cells are dependent on professional antigen-presenting cells (APCs) that provide additional co-stimulatory signals. TCR activation in the absence of co-stimulation can result in unresponsiveness and clonal anergy. To bypass immunization, different approaches for the derivation of cytotoxic effector cells with grafted recognition specificity have been developed. CARs have been constructed that consist of binding domains derived from natural ligands or antibodies specific for cell-surface components of the TCR-associated CD3 complex. Upon antigen binding, such chimeric antigen receptors link to endogenous signaling pathways in the effector cell and generate activating signals similar to those initiated by the TCR complex. For example, a CAR targeting CD19, a protein that is highly expressed on hematologic cancer cells, has shown good clinical efficacy. Since the first reports on chimeric antigen receptors, this concept has steadily been refined and the molecular design of chimeric receptors has been optimized and routinely use any number of well-known binding domains, such as scFV, Fav, and another protein binding fragments described herein.

Generally, CARs are one type of “cell therapy” (e.g., T cell therapy) contemplated for use according to the present disclosure. Although numerous representative embodiments of agents and methods for modulating immune cell activity by modulating the KIR3DL3 pathway, such as modulating the interaction between KIR3DL3 and a KIR3DL3 natural binding partner, such as HHLA2, immune cell-based therapies and methods are also encompassed. For example, T cells engineered to have a knockout, knockdown, or increased expression of KIR3DL3 are contemplated. Similarly, immune cells or other cells engineered to have a knockout, knockdown, or increased expression of a ligand for KIR3DL3, HHLA2, are also contemplated.

B cell receptors (BCR) are present on B cells. B cell antigen receptors are a complex between membrane Ig (mIg) and other transmembrane polypeptides (e.g., Igα and Igβ). The signal transduction function of mIg is triggered by crosslinking of receptor polypeptides by oligomeric or multimeric antigens. B cells can also be activated by anti-immunoglobulin antibodies. Upon BCR activation, numerous changes occur in B cells, including tyrosine phosphorylation.

Fc receptors are found on many cells which participate in immune responses. Fc receptors (FcRs) are cell surface receptors for the Fc portion of immunoglobulin polypeptides (Igs). Among the human FcRs that have been identified so far are those which recognize IgG (designated Fcγ R), IgE (Fcε R1), IgA (Fcα), and polymerized IgM/A (Fcμα R). FcRs are found in the following cell types: Fcε R I (mast cells), Fcε R.II (many leukocytes), Fcε R (neutrophils), and Fcμα R (glandular epithelium, hepatocytes) (Hogg, N. (1988) Immunol. Today 9:185-86). The widely studied FcγRs are central in cellular immune defenses, and are responsible for stimulating the release of mediators of inflammation and hydrolytic enzymes involved in the pathogenesis of autoimmune disease (Unkeless, J. C. et al. (1988) Annu. Rev. Immunol. 6:251-81). The FcγRs provide a crucial link between effector cells and the lymphocytes that secrete Ig, since the macrophage/monocyte, polymorphonuclear leukocyte, and natural killer (NK) cell FcγRs confer an element of specific recognition mediated by IgG. Human leukocytes have at least three different receptors for IgG: h Fcγ RI (found on monocytes/macrophages), hFcγ RII (on monocytes, neutrophils, eosinophils, platelets, possibly B cells, and the K562 cell line), and Fcγ III (on NK cells, neutrophils, eosinophils, and macrophages).

With respect to T cells, transmission of a costimulatory signal to a T cell involves a signaling pathway that is not inhibited by cyclosporin A. In addition, a costimulatory signal can induce cytokine secretion (e.g., IL-2 and/or IL-10) in a T cell and/or can prevent the induction of unresponsiveness to antigen, the induction of anergy, or the induction of cell death (deletion) in the T cell.

The term “activity,” when used with respect to a polypeptide, e.g., KIR3DL3 and/or a KIR3DL3 natural binding partner, such as HHLA2, includes activities that are inherent in the structure of the protein. For example, with regard to a HHLA2 ligand, the term “activity” includes the ability to modulate immune cell inhibition by modulating an inhibitory signal in an immune cell (e.g., by engaging a natural receptor on an immune cell). Those of skill in the art will recognize that when an activating form of the HHLA2 ligand polypeptide binds to an inhibitory receptor, such as KIR3DL3, an inhibitory signal is generated in the immune cell.

The term “inhibitory signal” refers to a signal transmitted via an inhibitory receptor (e.g., KLRB1, CTLA4, PD-1, and the like) for a polypeptide on a immune cell. Such a signal antagonizes a signal via an activating receptor (e.g., via a TCR, CD3, BCR, TMIGD2, or Fc polypeptide) and can result in, e.g., inhibition of second messenger generation; an inhibition of proliferation; an inhibition of effector function in the immune cell, e.g., reduced phagocytosis, reduced antibody production, reduced cellular cytotoxicity, the failure of the immune cell to produce mediators, (such as cytokines (e.g., IL-2) and/or mediators of allergic responses); or the development of anergy.

The amount of a biomarker in a subject is “significantly” higher or lower than the normal amount of the biomarker, if the amount of the biomarker is greater or less, respectively, than the normal or control level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% of that amount. Alternatively, the amount of the biomarker in the subject can be considered “significantly” higher or lower than the normal and/or control amount if the amount is at least about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, two times, three times, four times, five times, or more, or any range in between, such as 5%-100%, higher or lower, respectively, than the normal and/or control amount of the biomarker. Such significant modulation values can be applied to any metric described herein, such as altered level of expression, altered activity, changes in cancer cell hyperproliferative growth, changes in cancer cell death, changes in biomarker inhibition, changes in test agent binding, and the like.

The term “altered level of expression” of a marker refers to an expression level or copy number of a marker in a test sample e.g., a sample derived from a subject suffering from cancer, that is greater or less than the standard error of the assay employed to assess expression or copy number, and is preferably at least twice, and more preferably three, four, five or ten or more times the expression level or copy number of the marker or chromosomal region in a control sample (e.g., sample from a healthy subject not having the associated disease) and preferably, the average expression level or copy number of the marker or chromosomal region in several control samples. The altered level of expression is greater or less than the standard error of the assay employed to assess expression or copy number, and is preferably at least twice, and more preferably three, four, five or ten or more times the expression level or copy number of the marker in a control sample (e.g., sample from a healthy subject not having the associated disease) and preferably, the average expression level or copy number of the marker in several control samples.

Unless otherwise specified here within, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The term “inactivating antibodies” refers to antibodies that do not induce the complement system.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., KIR3DL3 polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16:778). Such single chain antibodies are also intended to be encompassed within the term “antigen-binding portion” of an antibody. Any V_{H} and V_{L} sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antibodies and Hybridomas 6:93-101) and use of a cysteine residue, a marker peptide and a C-terminal polyhistidine tag to make bivalent and biotinylated scFv polypeptides (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g., humanized, chimeric, etc.). Antibodies may also be fully human. In one embodiment, antibodies encompassed by the present disclosure bind specifically or substantially specifically to KIR3DL3 polypeptides or fragments thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The terms “CDR”, and its plural “CDRs”, refer to a complementarity determining region (CDR) of which three make up the binding character of a light chain variable region (CDR-L1, CDR-L2 and CDR-L3) and three make up the binding character of a heavy chain variable region (CDR-H1, CDR-H2 and CDR-H3) on an antibody, for example. CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat, Chothia, and/or MacCallum et al., (Kabat et al., in “Sequences of Proteins of Immunological Interest,” 5th Edition, U.S. Department of Health and Human Services, 1992; Chothia et al. (1987) J. Mol. Biol. 196, 901; and MacCallum et al., J. Mol. Biol. (1996) 262, 732, each of which is incorporated by reference in its entirety).

As used herein, the term “classifying” includes “to associate” or “to categorize” a sample with a disease state. In certain instances, “classifying” is based on statistical evidence, empirical evidence, or both. In certain embodiments, the methods and systems of classifying use of a so-called training set of samples having known disease states. Once established, the training data set serves as a basis, model, or template against which the features of an unknown sample are compared, in order to classify the unknown disease state of the sample. In certain instances, classifying the sample is akin to diagnosing the disease state of the sample. In certain other instances, classifying the sample is akin to differentiating the disease state of the sample from another disease state.

As used herein, the term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

“Complement [to]” or “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. In another embodiment, all nucleotide residues of the first portion are capable of base pairing with nucleotide residues in the second portion.

As used herein, the term “composite antibody” refers to an antibody which has variable regions comprising germline or non-germline immunoglobulin sequences from two or more unrelated variable regions. Additionally, the term “composite, human antibody” refers to an antibody which has constant regions derived from human germline or non-germline immunoglobulin sequences and variable regions comprising human germline or non-germline sequences from two or more unrelated human variable regions. A composite, human antibody is useful as an effective component in a therapeutic agent according to the present disclosure since the antigenicity of the composite, human antibody in the human body is lowered.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods encompassed by the present disclosure. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods encompassed by the present disclosure are not limited to use of a specific cut-point in comparing the level of expression product in the test sample to the control.

The term “costimulate,” as used with reference to activated immune cells, includes the ability of a costimulatory polypeptide to provide a second, non-activating receptor mediated signal (a “costimulatory signal”) that induces proliferation or effector function. For example, a costimulatory signal can result in cytokine secretion, e.g., in a T cell that has received a T cell-receptor-mediated signal. Immune cells that have received a cell-receptor mediated signal, e.g., via an activating receptor are referred to herein as “activated immune cells.”

The term “costimulatory receptor” includes receptors which transmit a costimulatory signal to a immune cell, e.g., CD28. As used herein, the term “inhibitory receptors” includes receptors which transmit a negative signal to an immune cell (e.g., CTLA4, KIR3DL3 or PD-1). An inhibitory signal as transduced by an inhibitory receptor can occur even if a costimulatory receptor (such as CD28) is not present on the immune cell and, thus, is not simply a function of competition between inhibitory receptors and costimulatory receptors for binding of costimulatory polypeptides (Fallarino et al. (1998) J. Exp. Med. 188:205). Transmission of an inhibitory signal to an immune cell can result in unresponsiveness or anergy or programmed cell death in the immune cell. Preferably transmission of an inhibitory signal operates through a mechanism that does not involve apoptosis. As used herein the term “apoptosis” includes programmed cell death which can be characterized using techniques which are known in the art. Apoptotic cell death can be characterized, e.g., by cell shrinkage, membrane blebbing and chromatin condensation culminating in cell fragmentation. Cells undergoing apoptosis also display a characteristic pattern of internucleosomal DNA cleavage. Depending upon the form of the polypeptide that binds to a receptor, a signal can either be transmitted (e.g., by a multivalent form of HHLA2 and/or KIR3DL3 polypeptide) or a signal can be inhibited (e.g., by a soluble, monovalent form of a HHLA2 and/or KIR3DL3), for instance by competing with activating forms of HHLA2 and/or KIR3DL3 for binding to one or more natural binding partners. However, there are instances in which a soluble polypeptide can be stimulatory. The effects of a modulatory agent can be easily demonstrated using routine screening assays as described herein.

The term “determining a suitable treatment regimen for the subject” is taken to mean the determination of a treatment regimen (i.e., a single therapy or a combination of different therapies that are used for the prevention and/or treatment of the cancer in the subject) for a subject that is started, modified and/or ended based or essentially based or at least partially based on the results of the analysis according to the present disclosure. One example is determining whether to provide targeted therapy against a cancer to provide immunomodulatory therapy (e.g., KIR3DL3 pathway modulator therapy (e.g., modulator of the interaction between KIR3DL3 and one or more natural binding partners, such as KIR3DL3)). Another example is starting an adjuvant therapy after surgery whose purpose is to decrease the risk of recurrence, another would be to modify the dosage of a particular chemotherapy. The determination can, in addition to the results of the analysis according to the present disclosure, be based on personal characteristics of the subject to be treated. In most cases, the actual determination of the suitable treatment regimen for the subject will be performed by the attending physician or doctor.

As used herein, the term “Fc region” is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies encompassed by the present disclosure include human IgG1, IgG2 (IgG2A, IgG2B), IgG3 and IgG4.

As used herein, “Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see M. Daëron, Annu. Rev. Immunol. 15:203-234 (1997). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

A molecule is “fixed” or “affixed” to a substrate if it is covalently or non-covalently associated with the substrate such the substrate can be rinsed with a fluid (e.g. standard saline citrate, pH 7.4) without a substantial fraction of the molecule dissociating from the substrate.

As used herein, “framework” or “FR” residues are those variable-domain residues other than the CDR residues as herein defined.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

As used herein, the term “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

The terms “high,” “low,” “intermediate,” and “negative” in connection with cellular biomarker expression refers to the amount of the biomarker expressed relative to the cellular expression of the biomarker by one or more reference cells. Biomarker expression can be determined according to any method described herein including, without limitation, an analysis of the cellular level, activity, structure, and the like, of one or more biomarker genomic nucleic acids, ribonucleic acids, and/or polypeptides. In one embodiment, the terms refer to a defined percentage of a population of cells expressing the biomarker at the highest, intermediate, or lowest levels, respectively. Such percentages can be defined as the top 0.1%, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10%, 11%, 12%, 13%, 14%, 15% or more, or any range in between, inclusive, of a population of cells that either highly express or weakly express the biomarker. The term “low” excludes cells that do not detectably express the biomarker, since such cells are “negative” for biomarker expression. The term “intermediate” includes cells that express the biomarker, but at levels lower than the population expressing it at the “high” level. In another embodiment, the terms can also refer to, or in the alternative refer to, cell populations of biomarker expression identified by qualitative or statistical plot regions. For example, cell populations sorted using flow cytometry can be discriminated on the basis of biomarker expression level by identifying distinct plots based on detectable moiety analysis, such as based on mean fluorescence intensities and the like, according to well-known methods in the art. Such plot regions can be refined according to number, shape, overlap, and the like based on well-known methods in the art for the biomarker of interest. In still another embodiment, the terms can also be determined according to the presence or absence of expression for additional biomarkers.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

As used herein, the term “host cell” is intended to refer to a cell into which a nucleic acid encompassed by the present disclosure, such as a recombinant expression vector encompassed by the present disclosure, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “humanized antibody”, as used herein, is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. Humanized antibodies may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

A humanized mouse, as used herein, is a mouse carrying functioning human genes (e.g., HHLA2 and/or KIR3DL3), cells, tissues, and/or organs. Humanized mice are commonly used as small animal models in biological and medical research for human therapeutics. The nude mouse and severe combined immunodeficiency (SCID) mouse may be used for this purpose. The NCG mouse, NOG mouse and the NSG mouse may be used to engraft human cells and tissues more efficiently than other models. Such humanized mouse models may be used to model the human immune system in scenarios of health and pathology, and may enable evaluation of therapeutic candidates in an in vivo setting relevant to human physiology.

As used herein, the term “hypervariable region,” “HVR,” or “HV,” refers to the regions of an antibody-variable domain that are hypervariable in sequence and/or form structurally defined loops, and include the CDRs.

As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

As used herein, the term “immune disorder” includes immune diseases, conditions, and predispositions to, including, but not limited to, cancer, chronic inflammatory disease and disorders (including, e.g., Crohn's disease, inflammatory bowel disease, reactive arthritis, and Lyme disease), insulin-dependent diabetes, organ specific autoimmunity (including, e.g., multiple sclerosis, Hashimoto's thyroiditis, autoimmune uveitis, and Grave's disease), contact dermatitis, psoriasis, graft rejection, graft versus host disease, sarcoidosis, atopic conditions (including, e.g., asthma and allergy including, but not limited to, allergic rhinitis and gastrointestinal allergies such as food allergies), eosinophilia, conjunctivitis, glomerular nephritis, systemic lupus erythematosus, scleroderma, certain pathogen susceptibilities such as helminthic (including, e.g., leishmaniasis) and certain viral infections (including, e.g., HIV and bacterial infections such as tuberculosis and lepromatous leprosy) and malaria.

As used herein, the term “immune response” includes T cell mediated and/or B cell mediated immune responses. Exemplary immune responses include T cell responses, e.g., cytokine production, and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “immune checkpoint” refers to a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

As used herein, the term “inhibiting” and grammatical equivalents thereof refer decrease, limiting, and/or blocking a particular action, function, or interaction. In one embodiment, the term refers to reducing the level of a given output or parameter to a quantity (e.g., background staining, KIR3DL3 signaling, KIR3DL3 immunoinhibitory function, and the like) which is at least 10%, 15%, 20%, 25%, 30%, 35%, 40%, 45%, 50%, 55%, 60%, 65%, 70%, 75%, 80%, 85%, 90%, 95%, 99% or less than the quantity in a corresponding control. A reduced level of a given output or parameter need not, although it may, mean an absolute absence of the output or parameter. The invention does not require, and is not limited to, methods that wholly eliminate the output or parameter. The given output or parameter can be determined using methods well-known in the art, including, without limitation, immunohistochemical, molecular biological, cell biological, clinical, and biochemical assays, as discussed herein and in the examples. The opposite terms “promoting,” “increasing,” and grammatical equivalents thereof refer to the increase in the level of a given output or parameter that is the reverse of that described for inhibition or decrease.

As used herein, the term “interaction”, when referring to an interaction between two molecules, refers to the physical contact (e.g., binding) of the molecules with one another (e.g., binding of HHLA2 to TMIGD2 or binding of HHLA2 to KIR3DL3). Generally, such an interaction results in an activity (which produces a biological effect) of one or both of said molecules. The activity may be a direct activity of one or both of the molecules, (e.g., signal transduction). Alternatively, one or both molecules in the interaction may be prevented from binding their ligand, and thus be held inactive with respect to ligand binding activity (e.g., binding its ligand and triggering or inhibiting an immune response). To inhibit such an interaction results in the disruption of the activity of one or more molecules involved in the interaction. To enhance such an interaction is to prolong or increase the likelihood of said physical contact, and prolong or increase the likelihood of said activity.

The term “neoadjuvant therapy” refers to a treatment given before the primary treatment. Examples of neoadjuvant therapy can include chemotherapy, radiation therapy, and hormone therapy.

As used herein, the term an “isolated antibody” is intended to refer to an antibody which is substantially free of other antibodies having different antigenic specificities (e.g., an isolated antibody that specifically binds to KIR3DL3 and is substantially free of antibodies that do not bind to KIR3DL3). An isolated antibody that specifically binds to a KIR3DL3 may, however, have cross-reactivity to other KIR family proteins, respectively, from different species. For example, in some embodiments, the antibody maintains specific binding affinity for at least two species, such as human and other animals, such as non-rodent animals, or other mammal or non-mammal species. However, in some embodiments, the antibody maintains higher or indeed specific affinity and selectivity for human KIR3DL3. In addition, an isolated antibody is typically substantially free of other cellular material and/or chemicals. In one embodiment encompassed by the present disclosure, a combination of “isolated” monoclonal antibodies having different specificities to human KIR3DL3 are combined in a well-defined composition.

As used herein, an “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a target polypeptide (e.g., immunoglobulin) or fragment thereof, in which the protein is separated from cellular components of the cells from which it is isolated or recombinantly produced. In one embodiment, the language “substantially free of cellular material” includes preparations of target protein or fragment thereof, having less than about 30% (by dry weight) of non-target protein (also referred to herein as a “contaminating protein”), more preferably less than about 20% of non-target protein, still more preferably less than about 10% of non-target protein, and most preferably less than about 5% non-target protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM or IgG1) that is encoded by heavy chain constant region genes.

As used herein, the term “K_{D}” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

As used herein, a “kit” is any manufacture (e.g. a package or container) comprising at least one reagent, e.g. a probe, for specifically detecting or modulating the expression of a marker encompassed by the present disclosure. The kit may be promoted, distributed, or sold as a unit for performing the methods encompassed by the present disclosure.

A “marker” or “biomarker” is a gene or protein whose altered level of expression in a tissue or cell from its expression level in normal or healthy tissue or cell is associated with a disease state, such as cancer. A “marker nucleic acid” is a nucleic acid (e.g., mRNA, cDNA) encoded by or corresponding to a marker encompassed by the present disclosure. Such marker nucleic acids include DNA (e.g., cDNA) comprising the entire or a partial sequence of any of the nucleic acid sequences set forth in the Sequence Listing or the complement of such a sequence. The marker nucleic acids also include RNA comprising the entire or a partial sequence of any of the nucleic acid sequences set forth in the Sequence Listing or the complement of such a sequence, wherein all thymidine residues are replaced with uridine residues. A “marker protein” is a protein encoded by or corresponding to a marker encompassed by the present disclosure. A marker protein comprises the entire or a partial sequence of any of the sequences set forth in the Sequence Listing. In some embodiments, the overall KIR3DL3 or HHLA2 is used as a marker. In other embodiments, a fragment of KIR3DL3 or HHLA2 is used as a marker. The terms “protein” and “polypeptide” are used interchangeably.

As used herein, the term “modulate” includes up-regulation and down-regulation, e.g., enhancing or inhibiting a response.

The term “pre-determined” biomarker amount and/or activity measurement(s) may be a biomarker amount and/or activity measurement(s) used to, by way of example only, evaluate a subject that may be selected for a particular treatment, evaluate a response to a treatment such as one or more modulators of the KIR3DL3 pathway, such as a modulator of KIR3DL3 nd one or more natural binding partners, such as HHLA2, either alone or in combination with one or more immunotherapies, and/or evaluate the disease state. A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., cell ratios or serum biomarker normalized to the expression of housekeeping or otherwise generally constant biomarker). The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The term “predictive” includes the use of a biomarker nucleic acid and/or protein status, e.g., over- or under-activity, emergence, expression, growth, remission, recurrence or resistance of tumors before, during or after therapy, for determining the likelihood of response of a cancer to immunomodulatory therapy, such as KIR3DL3 pathway modulator therapy (e.g., modulator of the interaction between KIR3DL3 and one or more natural binding partners, such as HHLA2, either alone or in combination with one or more additional therapy, such as immunotherapy, e.g., an immune checkpoint inhibition therapy). Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC) and/or biomarker target, or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer (e.g., those responding to a particular immunomodulatory therapy (e.g., KIR3DL3 pathway modulator therapy (e.g., modulator of the interaction between KIR3DL3 and one or more natural binding partners, such as HHLA2, either alone or in combination with an immunotherapy) or those developing resistance thereto).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., solid tumors, such as lung cancer, melanoma, and renal cell carcinoma), development of one or more clinical factors, development of intestinal cancer, or recovery from the disease.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus, internally, or at the carboxyl-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. They can be, for example, at least and/or including 10, 15, 20, 25, 30, 35, 40, 45, 50, 55, 60, 65, 70, 75, 80, 85, 90, 95, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, 1200, 1220, 1240, 1260, 1280, 1300, 1320, 1340 or more long so long as they are less than the length of the full-length polypeptide. Alternatively, they can be no longer than and/or excluding such a range so long as they are less than the length of the full-length polypeptide.

The term “probe” refers to any molecule which is capable of selectively binding to a specifically intended target molecule, for example, a nucleotide transcript or protein encoded by or corresponding to a marker. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

As used herein, the term “rearranged” refers to a configuration of a heavy chain or light chain immunoglobulin locus wherein a V segment is positioned immediately adjacent to a D-J or J segment in a conformation encoding essentially a complete V_{H} and V_{L} domain, respectively. A rearranged immunoglobulin gene locus can be identified by comparison to germline DNA; a rearranged locus will have at least one recombined heptamer/nonamer homology element.

As used herein, the term “recombinant host cell” (or simply “host cell”), is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “resistance” refers to an acquired or natural resistance of a cancer sample or a mammal to an immunomodulatory therapy (i.e., being nonresponsive to or having reduced or limited response to the therapeutic treatment), such as having a reduced response to a therapeutic treatment by 5% or more, for example, 10%, 15%, 20%, 25%, 30%, 35%, 40%, 45%, 50%, 55%, 60%, 65%, 70%, 75%, 80%, 85%, 90%, 95%, 100%, or more, to 2-fold, 3-fold, 4-fold, 5-fold, 10-fold, 15-fold, 20-fold or more. The reduction in response can be measured by comparing with the same cancer sample or mammal before the resistance is acquired, or by comparing with a different cancer sample or a mammal who is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

As described above, the term “response” is generally related to for example, determining the effects on progression, efficacy, or outcome of a clinical intervention. For example, a response to therapy (e.g., KIR3DL3 pathway modulator therapy (e.g., modulator of the interaction between KIR3DL3 and one or more natural binding partners, such as HHLA2, either alone or in combination with an immunotherapy, such as an immune checkpoint inhibition therapy) relates to any response to therapy (e.g., KIR3DL3 pathway modulator therapy (e.g., modulator of the interaction between KIR3DL3 and one or more natural binding partners, such as KIR3DL3, either alone or in combination with an immunotherapy, such as an immune checkpoint inhibition therapy), and, for cancer, preferably to a change in cancer cell numbers, tumor mass, and/or volume after initiation of neoadjuvant or adjuvant chemotherapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR-CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any immunomodulatory therapy. The outcome measurement may be pathologic response to therapy given in the neoadjuvant setting. Alternatively, outcome measures, such as overall survival and disease-free survival can be monitored over a period of time for subjects following immunomodulatory therapy for whom biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months.

As used herein, the term “specific binding” refers to antibody binding to a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using human KIR3DL3 as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” The term “subject” refers to any healthy animal, mammal or human, or any animal, mammal or human afflicted with a condition of interest (e.g., cancer). The term “subject” is interchangeable with “patient.” In some embodiments, the term is intended to include living organisms in which an immune response can be elicited. Representative, non-limiting examples of subjects include humans, dogs, cats, mice, rats, and transgenic species thereof.

As used herein, the term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “tolerance” or “unresponsiveness” includes refractivity of cells, such as immune cells, to stimulation, e.g., stimulation via an activating receptor or a cytokine. Unresponsiveness can occur, e.g., because of exposure to immunosuppressants or exposure to high doses of antigen. Several independent methods can induce tolerance. One mechanism is referred to as “anergy,” which is defined as a state where cells persist in vivo as unresponsive cells rather than differentiating into cells having effector functions. Such refractivity is generally antigen-specific and persists after exposure to the tolerizing antigen has ceased. For example, anergy in T cells is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the API sequence that can be found within the enhancer (Kang et al. (1992) Science 257:1134). Another mechanism is referred to as “exhaustion.” T cell exhaustion is a state of T cell dysfunction that arises during many chronic infections and cancer. It is defined by poor effector function, sustained expression of inhibitory receptors and a transcriptional state distinct from that of functional effector or memory T cells.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a marker encompassed by the present disclosure and normal post-transcriptional processing (e.g. splicing), if any, of the RNA transcript, and reverse transcription of the RNA transcript.

As used herein, the term “T cell” includes CD4+ T cells and CD8+ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells) as well as other antigen presenting cells (e.g., keratinocytes, endothelial cells, astrocytes, fibroblasts, oligodendrocytes). Conventional T cells, also known as Tconv or Teffs, have effector functions (e.g., cytokine secretion, cytotoxic activity, anti-self-recognization, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+Teffs or CD8+Teffs, such as CD4+ helper T lymphocytes (e.g., Th0, Th1, Tfh, or Th17) and CD8+ cytotoxic T lymphocytes. As described further herein, cytotoxic T cells are CD8+T lymphocytes. “Naïve Tcons” are CD4+ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

As used herein, the term “unrearranged” or “germline configuration” in reference to a V segment refers to the configuration wherein the V segment is not recombined so as to be immediately adjacent to a D or J segment.

### II. Monoclonal Antibodies, Immunoglobulins, and Polypeptides

The present disclosure relates, in part, to isolated monoclonal antibodies or fragments thereof that are directed against KIR3DL3 (such as monoclonal antibodies and polyclonal antibodies listed herein). Such molecules, in part, are characterized in that they exhibit the ability to recognize KIR3DL3 protein in diagnostic assays, such as immunohistochemical (IHC), Western blot, intercellular flow, ELISA, and the like. Such molecules, in part, are characterized in that they exhibit the ability to inhibit KIR3DL3 binding to binding partners, such as HHLA2.

The term “HHLA2”, also known as human endogenous retrovirus-H long terminal repeat-associating protein 2, HERV-H LTR-associating 2, B7y, B7H7, B7-H5, B7-H7, refers to a member of the B7 family. HHLA2 protein has limited expression in normal human tissues but is widely expressed in human cancers. The HHLA2 protein is a membrane protein with three Ig-like domains (IgV-IgC-IgV), whereas other members of the B7 family generally have only two Ig domains (IgV-IgC). HHLA2 protein in normal human tissues is expressed in the epithelium of kidney, gut, gallbladder, and breast as well as placental trophoblast cells. In the immune system, HHLA2 protein is constitutively expressed on human monocytes/macrophages. HHLA2 regulates human T-cell functions including, for example, HHLA2 inhibits T-cell proliferation and cytokine production, and increases T-cell production and cytokine production. HHLA2 is expressed in higher levels in a wide range of human cancers from the colorectal, renal, lung, pancreas, ovary, and prostate. HHLA2 is also expressed in human cancers of thyroid, melanoma, liver, bladder, colon, kidney, breast, and esophagus.

Certain HHLA2 structures and functions, are well-known in the art as described above (see, for example, Xiao et al. (2015) Clin. Cancer Res. 21:2201-2203, Janakiram et al. (2015) Clin. Cancer Res. 21:2359-2366, Mager et al. (1999) Genomics 21:2359-2366, Flajnik et al. (2012) Immunogenet. 64:571-590, Zhao et al. (2013) Proc. Natl. Acad. Sci. U.S.A. 110:9879-9884, and Zhu et al. (2013) Nat. Commun. 4:2043).

The term “HHLA2” is intended to include fragments, variants (e.g., allelic variants), and derivatives thereof. Representative human HHLA2 cDNA and human HHLA2 protein sequences are well-known in the art and are publicly available from the National Center for Biotechnology Information (NCBI). Human HHLA2 variants include variant 1 (NM_007072.3 and NP_009003.1, which represents the longest transcript and encodes the longest isoform a), variant 2 (NM_001282556.1 and NP_001269485.1, which represents the use of an alternate promoter and differs in the 5′ UTR, compared to variant 1), vaiant 3 (NM_001282557.1 and NP_001269486.1, which represents the use of an alternate promoter and differs in the 5′ UTR, compared to variant 1), variant 4 (NM_001282558.1 and NP_001269487.1, which encodes isoform b, represents the use of an alternate promoter, differs in the 5′ UTR and lacks an alternate in-frame exon in the 3′ coding region, compared to variant 1, resulting a shorter isoform than isoform a), and variant 5 (NM_001282559.1 and NP_001269488.1, which encodes isoform c, represents the use of an alternate promoter, and has multiple differences compared to variant 2, resulting in a distinct 5′ UTR and causing translation initiation at an alternate start codon, compared to variant 1, resulting in a distinct N-terminus and a shorter isoform than isoform a). Nucleic acid and polypeptide sequences of HHLA2 orthologs in organisms other than humans are well-known and include, for example, frog HHLA2 (NM_001128644.1 and NP_001122116.1). Representative sequences of HHLA2 orthologs are presented below in Table 1.

Anti-HHLA2 antibodies suitable for detecting HHLA2 protein are well-known in the art and include, for example, antibodies Cat #: ab107119 and ab214327 (abcam), antibodies PA5-24146 and PA5-6313 (ThermoFisher Scientific), antibodies MAB80841, AF8084, FAB80841R, FAB80841T, and MAB8084 (R&D systems), antibody AP52042PU-N(Origene), antibodies NBP2-49187, MAB80842, H00011148-B01P, and NBP2-32420 (Novus Biologicals), antibody GTX51981 (GeneTex), antibody HPA055478 (Atlas Antibodies), antibodies LS-C321945, LS-C308228, LS-C246742, LS-C246743, LS-C246744, LS-C236210, and LS-C249186 (LifeSpan Biosiences), etc. Moreover, multiple siRNA, shRNA, CRISPR constructs for reducing HHLA2 expression can be found in the commercial product lists of the above-referenced companies, such as shRNA product #TL312462, TF312462, TR312462, TG312462, and TL312462V, siRNA product #SR323358 from Origene Technologies, SiRNA product #1009616, i009616a, i009616b, i009616c, i009616d, iV009616, iV009616a, iV009616b, iV009616c, iV009616d, iAAV00961600, iAAV00961601, iAAV00961602, iAAV00961603, iAAV00961604, iAAV00961605, iAAV00961606, iAAV00961607, iAAV00961608, and iAAV00961609, CRISPR product #K0950321, K0950301, K0950302, K0950303, K0950304, K0950305, K0950306, K0950307, K0950308, and K0950311 (abm), siRNA product #sc-78498, shRNA product #sc-78498-V and sc-78498-SH, CRISPR product #sc-411576, sc-411576-HDR, sc-411576-NIC, sand c-411576-NIC-2 (Santa Cruz Biotechnology), etc. It is to be noted that the term can further be used to refer to any combination of features described herein regarding HHLA2 molecules. For example, any combination of sequence composition, percentage identify, sequence length, domain structure, functional activity, etc. can be used to describe an HHLA2 molecule encompassed by the present disclosure.

The term “HHLA2 pathway” includes HHLA2 and interactions of HHLA2 with one or more of its natural binding partners, such as TMIGD2 and KIR3DL3.

The term “KIR3DL3 pathway” includes KIR3DL3 and interactions of KIR3DL3 with one or more of its natural binding partners, such as HHLA2.

The term “TMIGD2” refers to transmembrane and immunoglobulin domain containing 2, CD28H, IGPR1, and IGPR-1, which is a membrane protein having ˜10% amino acid identity with CD28, CTLA-4, ICOS, and PD-1. TMIGD2 has one extracellular IgV-like domain, a transmembrane region, and a proline-rich cytoplasmic domain with two tyrosine signaling motifs. TMIGD2 protein is constitutively expressed on all naive T cells and the majority of natural killer (NK) cells, but not on T regulatory cells or B cells. TMIGD2 expression is slowly lost with repetitive stimulation of T cells. Consistent with this, TMIGD2 is expressed on only about half of memory T cells, and TMIGD2-negative T cells have a terminally-differentiated, senescent phenotype. TMIGD2 has also been shown to be expressed in endothelial and epithelial cells and function to reduce cell migration and promote capillary tube formation during angiogenesis.

Certain TMIGD2 structures and functions are well-known in the art as described above (see, for example, Xiao et al. (2015) Clin. Cancer Res. 21:2201-2203, Janakiram et al. (2015) Clin. Cancer Res. 21:2359-2366, Zhu et al. (2013) Nat. Commun. 4:2043, and Rahimi (2012) Cell 23:1646-1656).

The term “TMIGD2” is intended to include fragments, variants (e.g., allelic variants), and derivatives thereof. Representative human TMIGD2 cDNA and human TMIGD2 protein sequences are well-known in the art and are publicly available from the National Center for Biotechnology Information (NCBI). Human TMIGD2 isoforms include isoform 1 (NM_144615.2 and NP_653216.2), isoform 2 (NM_001169126.1 and NP_001162597.1; which uses an alternate in-frame splice site in the 3′ coding region, compared to variant 1, resulting a shorter isoform, compared to isoform 1), and isoform 3 (NM_001308232.1 and NP_001295161.1, which lacks an alternate in-frame exon in the 5′ coding region compared to variant 1, resulting a shorter isoform, compared to isoform 1). Nucleic acid and polypeptide sequences of TMIGD2 orthologs in organisms other than humans are well-known and include, for example, chimpanzee TMIGD2 (XM_009434393.2 and XP_009432668.2, and XM_001138228.4 and XP_001138228.3), and cattle TMIGD2 (XM_005208980.3 and XP_005209037.1, XM_005208979.3 and XP_005209036.1, and XM_002688933.5 and XP_002688979.1). Representative sequences of TMIGD2 orthologs are presented below in Table 1.

Anti-TMIGD2 antibodies suitable for detecting TMIGD2 protein are well-known in the art and include, for example, antibodies Cat #MAB8316, MAB83162, FAB8316R, FAB83162R, FAB83162G, FAB83162N, FAB83162S, FAB83162T, FAB83162U, and FAB83162V (R&D systems), antibody TA326695 (Origene), antibodies PA5-52787, and PA5-38055 (ThermoFisher Scientific), antibodies MAB83161, and NBP1-81164 (Novus Biologicals), etc., Moreover, multiple siRNA, shRNA, CRISPR constructs for reducing TMIGD2 expression can be found in the commercial product lists of the above-referenced companies, such as shRNA product #TF317829, TG317829, TL317829, TR317829, and TL317829V, siRNA product #SR314913, and CRISPR products #KN204938, KN204938LP, KN204938RB, and KN204938BN from Origene Technologies, siRNA products #1024914, i024914a, i024914b, i024914c, i024914d, iV024914, iV024914a, iV024914b, iV024914c, iV024914d, iAAV02491400, iAAV02491401, iAAV02491402, iAAV02491403, iAAV02491404, iAAV02491405, iAAV02491406, iAfAV02491407, iAAV02491408, and iAAV02491409, and CRISPR products #K2409321, K2409301, K2409302, K2409303, K2409304, K2409305, K2409306, K2409307, K2409308, and K2409311 (Abm), siRNA product #sc-97757, shRNA products #sc-97757-SH, and sc-97757-V, and CRISPR products #sc-414261, sc-414261-HDR, sc-414261-NIC, and sc-414261-NIC-2 (Santa Cruz Biotechnology), shRNA products #SH888208, and SH874720 (Vigene Biosciences), etc., Moreover, multiple CRISPR constructs for increasing TMIGD2 expression can be found in the commercial product lists of the above-referenced companies, such as CRISPR products #K2409378, K2409377, K2409376, K2409375, K2409374, K2409373, K2409372, and K2409371 (Abm), CRISPR products #sc-414261-ACT, sc-414261-ACT-2, sc-414261-LAC, and sc-414261-LAC-2 (Santa Cruz Biotechnology), etc., It is to be noted that the term can further be used to refer to any combination of features described herein regarding TMIGD2 molecules. For example, any combination of sequence composition, percentage identify, sequence length, domain structure, functional activity, etc. can be used to describe an TMIGD2 molecule encompassed by the present disclosure.

Interactions between TMIGD2 and HHLA2 as well as their functions, are well-known in the art as described above (see, for example, Xiao et al. (2015) Clin. Cancer Res. 21:2201-2203 and Janakiram et al. (2015) Clin. Cancer Res. 21:2359-2366).

The term “KIR3DL3”, also known as Killer cell immunoglobulin-like receptor 3DL3, CD158Z, KIR3DL7, KIR44, KIRC1, KIR2DS2, killer cell immunoglobulin like receptor, three Ig domains and long cytoplasmic tail 3, refers to a member of a transmembrane glycoprotein family expressed by natural killer cells and subsets of T cells. The killer cell immunoglobulin-like receptor (KIR) genes are polymorphic and highly homologous and they are found in a cluster on chromosome 19q13.4 within the 1 Mb leukocyte receptor complex (LRC). The gene content of the KIR gene cluster varies among haplotypes, although several “framework” genes are found in all haplotypes (KIR3DL3, KIR3DP1, KIR3DL4, KIR3DL2). The KIR proteins are classified by the number of extracellular immunoglobulin domains (2D or 3D) and by whether they have a long (L) or short(S) cytoplasmic domain. KIR proteins with the long cytoplasmic domain transduce inhibitory signals upon ligand binding via an immune tyrosine-based inhibitory motif (ITIM), while KIR proteins with the short cytoplasmic domain lack the ITIM motif and instead associate with the TYRO protein tyrosine kinase binding protein to transduce activating signals. The ligands for several KIR proteins are subsets of HLA class I molecules; thus, KIR proteins are thought to play an important role in regulation of the immune response. This gene is one of the “framework” loci that is present on all haplotypes. The KIR3DL3 protein has an N-terminal signal sequence, 3 Ig domains, a transmembrane region lacking a positively charged residue, and a long cytoplasmic tail containing an immunoreceptor tyrosine-based inhibitory motif (ITIM). KIR3DL3 lacks the stalk region found in other KIRs.

Certain KIR3DL3 structures and functions, are well-known in the art as described above (see, for example, Hsu et al. (2002) Immunol Rev. 190:40-52, Trompeter et al. (2005) J. Immunol. 174:4135-4143, Trundley et al. (2006) Immunogenet. 57:904-916, and Jones et al. (2006) Immunogenet. 58:614-627).

The term “KIR3DL3” is intended to include fragments, variants (e.g., allelic variants), and derivatives thereof. Representative human KIR3DL3 cDNA and human KIR3DL3 protein sequences are well-known in the art and are publicly available from the National Center for Biotechnology Information (NCBI). For example, at least one human KIR3DL3 isoform is known: human KIR3DL3 (NM_153443.4) is encodable by the transcript (NP_703144.3). Nucleic acid and polypeptide sequences of KIR3DL3 orthologs in organisms other than humans are well-known and include, for example, chimpanzee KIR3DL3 (XM_003316679.3 and XP_003316727.3), Rhesus monkey KIR3DL3 (NM_001104552.2 and NP_001098022.1), mouse KIR3DL3 (NM_001310690.1 and NP_001297619.1, NM_177749.4 and NP_808417.2, NM_177748.2 and NP_808416.1), and rat KIR3DL3 (NM_181479.2 and NP_852144.1). Representative sequences of KIR3DL3 orthologs are presented below in Table 1.

Anti-KIR3DL3 antibodies suitable for detecting KIR3DL3 protein are well-known in the art and include, for example, antibodies Cat #: FAB8919R, MAB8919, FAB8919G, FAB8919N, FAB8919S, FAB8919T, FAB8919U, and FAB8919V (R&D systems), antibody AP52374PU-N(Origene), antibody PA5-26178 (ThermoFisher Scientific), antibodies OAAB05761, OAAF08125, OAAN04122, OACA09134, OACA09135, OACD04988, and OASG01190 (Aviva Systems Biology), etc., Moreover, multiple siRNA, shRNA, CRISPR constructs for reducing KIR3DL3 expression can be found in the commercial product lists of the above-referenced companies, such as shRNA products #TF303684, TR303684, TG303684, TL303684, TL303684V, siRNA products #SR314516, and CRISPR products #KN224383, KN224383BN, KN224383RB, and KN224383LP from Origene Technologies, siRNA products #1011627, i011627a, i011627b, i011627c, 1011627d, iV011627, iV011627a, iV011627b, iV011627c, iV011627d, iAAV01162700, iAAV01162701, iAAV01162702, iAAV01162703, iAAV01162704, iAAV01162705, iAAV01162706, iAAV01162707, iAAV01162708, and iAAV01162709, and CRISPR products #K1151421, K1151401, K1151402, K1151403, K1151404, K1151405, K1151406, K1151407, K1151408, and K1151411 (Abm), siRNA product #sc-60892, shRNA products #sc-60892-SH, and sc-60892-V, and CRISPR products #sc-406227, sc-406227-KO-2, sc-406227-HDR-2, sc-406227-NIC, and sc-406227-NIC-2 (Santa Cruz Biotechnology), etc., It is to be noted that the term can further be used to refer to any combination of features described herein regarding KIR3DL3 molecules. For example, any combination of sequence composition, percentage identify, sequence length, domain structure, functional activity, etc. can be used to describe an KIR3DL3 molecule encompassed by the present disclosure.

The term “peripheral blood cell subtypes” refers to cell types normally found in the peripheral blood including, but is not limited to, eosinophils, neutrophils, T cells, monocytes, NK cells, granulocytes, and B cells.

The term “recombinant human antibody” includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline and/or non-germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “sample” used for detecting or determining the presence or level of at least one biomarker is typically whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method encompassed by the present disclosure further comprises obtaining the sample from the individual prior to detecting or determining the presence or level of at least one marker in the sample.

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene encompassed by the present disclosure, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn, G. and Cullen, B. (2002) J. of Virology 76 (18): 9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs, shRNAs, or other RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

In addition to RNAi, genome editing can be used to modulate the copy number or genetic sequence of a biomarker of interest, such as constitutive or induced knockout or mutation of a biomarker of interest, such as a KIR3DL3 pathway component like HHLA2, TMIGD2, and/or KIR3DL3. For example, the CRISPR-Cas system can be used for precise editing of genomic nucleic acids (e.g., for creating non-functional or null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLOS One 6: e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42: e47). Such genetic strategies can use constitutive expression systems or inducible expression systems according to well-known methods in the art.

“Piwi-interacting RNA (piRNA)” is the largest class of small non-coding RNA molecules. piRNAs form RNA-protein complexes through interactions with piwi proteins. These piRNA complexes have been linked to both epigenetic and post-transcriptional gene silencing of retrotransposons and other genetic elements in germ line cells, particularly those in spermatogenesis. They are distinct from microRNA (miRNA) in size (26-31 nt rather than 21-24 nt), lack of sequence conservation, and increased complexity. However, like other small RNAs, piRNAs are thought to be involved in gene silencing, specifically the silencing of transposons. The majority of piRNAs are antisense to transposon sequences, suggesting that transposons are the piRNA target. In mammals it appears that the activity of piRNAs in transposon silencing is most important during the development of the embryo, and in both C. elegans and humans, piRNAs are necessary for spermatogenesis. piRNA has a role in RNA silencing via the formation of an RNA-induced silencing complex (RISC).

“Aptamers” are oligonucleotide or peptide molecules that bind to a specific target molecule. “Nucleic acid aptamers” are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. “Peptide aptamers” are artificial proteins selected or engineered to bind specific target molecules. These proteins consist of one or more peptide loops of variable sequence displayed by a protein scaffold. They are typically isolated from combinatorial libraries and often subsequently improved by directed mutation or rounds of variable region mutagenesis and selection. The “Affimer protein”, an evolution of peptide aptamers, is a small, highly stable protein engineered to display peptide loops which provides a high affinity binding surface for a specific target protein. It is a protein of low molecular weight, 12-14 kDa, derived from the cysteine protease inhibitor family of cystatins. Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA Apr; 9 (4): 493-501 incorporated by reference herein).

RNA interfering agents, e.g., siRNA molecules, may be administered to a patient having or at risk for having cancer, to inhibit expression of a biomarker gene which is overexpressed in cancer and thereby treat, prevent, or inhibit cancer in the subject.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. 1998. Science 282:63), and natural product extract libraries. In another embodiment, the compounds are small, organic non-peptidic compounds. In a further embodiment, a small molecule is not biosynthetic.

The term “selective modulator” or “selectively modulate” as applied to a biologically active agent refers to the agent's ability to modulate the target, such as a cell population, signaling activity, etc. as compared to off-target cell population, signaling activity, etc. via direct or interact interaction with the target. For example, an agent that selectively inhibits the interaction between KIR3DL3 and one or more natural binding partners, such as HHLA2, over another interaction between KIR3DL3 and another binding partner, and/or such interaction(s) on a cell population of interest may have an activity against the KIR3DL3 pathway modulator therapy (e.g., modulator of the interaction between KIR3DL3 and one or more natural binding partners, such as HHLA2, interaction that is at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 2× (times) or more than the agent's activity against at least one other binding partner (e.g., at least about 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, 105×, 110×, 120×, 125×, 150×, 200×, 250×, 300×, 350×, 400×, 450×, 500×, 600×, 700×, 800×, 900×, 1000×, 1500×, 2000×, 2500×, 3000×, 3500×, 4000×, 4500×, 5000×, 5500×, 6000×, 6500×, 7000×, 7500×, 8000×, 8500×, 9000×, 9500×, 10000×, or greater, or any range in between, inclusive). Such metrics are typically expressed in terms of relative amounts of agent required to reduce the interaction/activity by half.

More generally, the term “selective” refers to a preferential action or function. The term “selective” can be quantified in terms of the preferential effect in a particular target of interest relative to other targets. For example, a measured variable (e.g., modulation of Tregs/Bregs versus other cells, such as other immune cells like Tcons) can be 10%, 20%, 25%, 30%, 35%, 40%, 45%, 50%, 55%, 60%, 65%, 70%, 75%, 80%, 85%, 90%, 95%, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 70-fold, 80-fold, 90-fold, 100-fold, or greater or any range in between inclusive (e.g., 50% to 16-fold), different in a target of interest versus unintended or undesired targets. The same fold analysis can be used to confirm the magnitude of an effect in a given tissue, cell population, measured variable, measured effect, and the like, such as the Tregs: Tcons ratio, Bregs: Tcons ratio, hyperproliferative cell growth rate or volume, Tregs/Bregs proliferation rate or number, and the like.

By contrast, the term “specific” refers to an exclusionary action or function. For example, specific modulation of the HHLA2-KIR3DL3 interactions refers to the exclusive modulation of the HHLA2-KIR3DL3 interactions, and not modulation of the interaction between KIR3DL3 with another ligand. In another example, specific binding of an antibody to a predetermined antigen refers to the ability of the antibody to bind to the antigen of interest without binding to other antigens. Typically, the antibody binds with an affinity (K_{D}) of approximately less than 1×10⁻⁷ M, such as approximately less than 10⁻⁸ M, 10⁻⁹ M, 10⁻¹⁰ M, 10⁻¹¹ M, or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. In addition, K_{D} is the inverse of K_{A}. The phrases “an antibody recognizing an antigen” and “an antibody specific for an antigen” are used interchangeably herein with the term “an antibody which binds specifically to an antigen.”

The term “sensitize” means to alter cells, such as cancer cells or tumor cells, in a way that allows for more effective treatment with a therapy (e.g., KIR3DL3 pathway modulator therapy (e.g., modulator of the interaction between KIR3DL3 and one or more natural binding partners, such as HHLA2), either alone or in combination with an immunotherapy, such as an immune checkpoint inhibition therapy). In some embodiments, normal cells are not affected to an extent that causes the normal cells to be unduly injured by the therapy (e.g., KIR3DL3 pathway modulator therapy (e.g., modulator of the interaction between KIR3DL3 and one or more natural binding partners, such as HHLA2), either alone or in combination with an immunotherapy, such as an immune checkpoint inhibition therapy). An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42:2159-2164), cell death assays (Weisenthal L M, Shoemaker R H, Marsden J A, Dill P L, Baker J A, Moran E M, Cancer Res 1984; 94:161-173; Weisenthal L M, Lippman M E, Cancer Treat Rep 1985; 69:615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993:415-432; Weisenthal L M, Contrib Gynecol Obstet 1994; 19:82-90). The sensitivity or resistance may also be measured in animal by measuring the tumor size reduction over a period of time, for example, 6 months for human and 4-6 weeks for mouse. A composition or a method sensitizes response to a therapeutic treatment if the increase in treatment sensitivity or the reduction in resistance is 5% or more, for example, 10%, 15%, 20%, 25%, 30%, 35%, 40%, 45%, 50%, 55%, 60%, 65%, 70%, 75%, 80%, 85%, 90%, 95%, 100%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of an immunomodulatory can be equally applied to methods for sensitizing hyperproliferative or otherwise cancerous cells (e.g., resistant cells) to the therapy.

The term “synergistic effect” refers to the combined effect of two or more therapeutic agents, such as two or more KIR3DL3 pathway modulators, a KIR3DL3 pathway modulator and an immunotherapy, KIR3DL3 pathway modulators either alone or in combination with an immunotherapy, such as an immune checkpoint inhibition therapy, and the like, can be greater than the sum of the separate effects of the anticancer agents alone.

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically-effective amount” and “effective amount” as used herein means that amount of a compound, material, or composition comprising a compound encompassed by the present disclosure which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. Cancer cell death can be promoted by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in cancer cell numbers and/or a solid malignancy can be achieved.

The term “substantially free of chemical precursors or other chemicals” includes preparations of antibody, polypeptide, peptide or fusion protein in which the protein is separated from chemical precursors or other chemicals which are involved in the synthesis of the protein. In one embodiment, the language “substantially free of chemical precursors or other chemicals” includes preparations of antibody, polypeptide, peptide or fusion protein having less than about 30% (by dry weight) of chemical precursors or non-antibody, polypeptide, peptide or fusion protein chemicals, more preferably less than about 20% chemical precursors or non-antibody, polypeptide, peptide or fusion protein chemicals, still more preferably less than about 10% chemical precursors or non-antibody, polypeptide, peptide or fusion protein chemicals, and most preferably less than about 5% chemical precursors or non-antibody, polypeptide, peptide or fusion protein chemicals.

A “transcribed polynucleotide” or “nucleotide transcript” is a polynucleotide (e.g. an mRNA, hnRNA, cDNA, mature miRNA, pre-miRNA, pri-miRNA, miRNA*, anti-miRNA, or a miRNA binding site, or a variant thereof or an analog of such RNA or cDNA) which is complementary to or homologous with all or a portion of a mature mRNA made by transcription of a marker encompassed by the present disclosure and normal post-transcriptional processing (e.g. splicing), if any, of the RNA transcript, and reverse transcription of the RNA transcript.

The term “vector” refers to a nucleic acid capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for nucleic acid and polypeptide molecules useful in the present disclosure are well-known in the art and readily available on publicly available databases, such as the National Center for Biotechnology Information (NCBI). For example, exemplary nucleic acid and amino acid sequences derived from publicly available sequence databases are provided in Table 1 below.

TABLE 1
SEQ ID NO: 1 Human HHLA2 Variant 1 cDNA Sequence (NM 007072.3, CDS
region from position 415-1659)

| | |
|---|---|
| 1 | agttctcttc aagtcatgta atcgactttt ttgaattagt tttcagtttc attttgtttt |
| 61 | ccctaattca agttgggaac acttcatttt ccccaattca agttgggaac acttccttgg |
| 121 | tatttccttg ctacatggac tttagcaaat gctactttac tctccttcca gctactcagg |
| 181 | aggctgaggc aggagaatcg cttgaacccg ggaggcggag gttacagtga gccttttcct |
| 241 | agttttactg ttggaagcct aactcacagg agagattatg caatacagtc ctgaagtcaa |
| 301 | gggaggagag catgtaggag aatactaacc ctgcacagat tgtgatggtg atgtggaata |
| 361 | tactaaagcc tagaacgcac ctcctctgca tgactaatat gttctgcaca agacatgaag |
| 421 | gcacagacag cactgtcttt cttcctcatt ctcataacat ctctgagtgg atctcaaggc |
| 481 | atattccctt tggctttctt catttatgtt cctatgaatg aacaaatcgt cattggaaga |
| 541 | cttgatgaag atataattct cccttcttca tttgagaggg gatccgaagt cgtaatacac |
| 601 | tggaagtatc aagatagcta taaggttcac agttactaca aaggcagtga ccatttggaa |
| 661 | agccaagatc ccagatatgc aaacaggaca tcccttttct ataatgagat tcaaaatggg |
| 721 | aatgcgtcgc tatttttcag aagagtaagc cttctggacg aaggaattta cacctgctat |
| 781 | gtaggaacag caattcaagt gattacaaac aaagtggtgc taaaggtggg agtttttctc |
| 841 | acacccgtga tgaagtatga aaagaggaac acaaacagct tottaatatg cagcgtgtta |
| 901 | agtgtttatc ctcgtccaat tatcacgtgg aaaatggaca acacacctat ctctgaaaac |
| 961 | aacatggaag aaacagggtc tttggattct ttttctatta acagcccact gaatattaca |
| 1021 | ggatcaaatt catcttatga atgtacaatt gaaaattcac tgctgaagca aacatggaca |
| 1081 | gggcgctgga cgatgaaaga tggccttcat aaaatgcaaa gtgaacacgt ttcactctca |
| 1141 | tgtcaacctg taaatgatta tttttcacca aaccaagact tcaaagttac ttggtccaga |
| 1201 | atgaaaagtg ggactttctc tgtcctggct tactatctga gctcctcaca aaatacaatt |
| 1261 | atcaatgaat cccgattctc atggaacaaa gagctgataa accagagtga cttctctatg |
| 1321 | aatttgatgg atcttaatct ttcagacagt ggggaatatt tatgcaatat ttcttcggat |
| 1381 | gaatatactt tacttaccat ccacacagtg catgtagaac cgagccaaga aacagcttcc |
| 1441 | cataacaaag gcttatggat tttggtgccc tctgcgattt tggcagcttt tctgctgatt |
| 1501 | tggagcgtaa aatgttgcag agcccagcta gaagccagga ggagcagaca ccctgctgat |
| 1561 | ggagcccaac aagaaagatg ttgtgtccct cctggtgagc gctgtcccag tgcacccgat |
| 1621 | aatggcgaag aaaatgtgcc totttcagga aaagtatagg aaatgagaga agactgtgac |
| 1681 | aactcatgac ctgcatcctt aatatccagt gacttcatct cccctttctt caccacaatt |
| 1741 | ccaggcaatg gcctgtcgga gcagacaatt ctaccactgc aaagagttgt aaccattttc |
| 1801 | tggtatcaca tttatttttc aagacatact tttcaagaca tcattcactg acccactacc |
| 1861 | tgcattgagt ataaatgcct ggatgttaag gattccaatt taactttgaa aagaactgtc |
| 1921 | tcattcattt acatttctgt tacagtcagc ccaggaggtt acagtgagct ctccactaag |
| 1981 | aatctggaag aaatgcatca ctaggggttg attcccaatc tgatcaactg ataatgggtg |
| 2041 | agagagcagg taagagccaa agtcacctta gtggaaaggt taaaaaccag agcctggaaa |
| 2101 | ccaagatgat tgatttgaca aggtatttta gtctagtttt atatgaacgg ttgtatcagg |
| 2161 | gtaaccaact cgatttggga tgaatcttag ggcaccaaag actaagacag tatctttaag |
| 2221 | attgctaggg aaaagggccc tatgtgtcag gcctctgagc ccaagccaag catcgcatcc |
| 2281 | cctgtgattt gcacgtatac atccagatgg cctaaagtaa ctgaagatcc acaaaagaag |
| 2341 | taaaaatagc cttaactgat gacattccac cattgtgatt tgttcctgcc ccaccctaac |
| 2401 | tgatcaatgt actttgtaat ctcccccacc cttaagaagg tactttgtaa tcttccccac |
| 2461 | ccttaagaag gttctttgta attctcccca cccttgagaa tgtactttgt gagatccacc |
| 2521 | ctgcccacaa aacattgctc ttaacttcac cgcctaaccc aaaacctata agaactaatg |
| 2581 | ataatccatc acccttcgct gactctcttt tcggactcag cccacctgca cccaggtgaa |
| 2641 | ataaacagct ttattgctca cacaaaaaaa aaaaaaaaa |

SEQ ID NO: 2 Human HHLA2 Variant 1 Amino Acid Sequence (NP 009003.1)

| | |
|---|---|
| 1 | MKAQTALSFF LILITSLSGS QGIFPLAFFI YVPMNEQIVI GRLDEDIILP SSFERGSEVV |
| 61 | IHWKYQDSYK VHSYYKGSDH LESQDPRYAN RTSLFYNEIQ NGNASLFFRR VSLLDEGIYT |
| 121 | CYVGTAIQVI TNKVVLKVGV FLTPVMKYEK RNTNSFLICS VLSVYPRPII TWKMDNTPIS |
| 181 | ENNMEETGSL DSFSINSPLN ITGSNSSYEC TIENSLLKQT WTGRWTMKDG LHKMQSEHVS |
| 241 | LSCQPVNDYF SPNQDFKVTW SRMKSGTFSV LAYYLSSSQN TIINESRESW NKELINQSDE |
| 301 | SMNLMDLNLS DSGEYLCNIS SDEYTLLTIH TVHVEPSQET ASHNKGLWIL VPSAILAAFL |
| 361 | LIWSVKCCRA QLEARRSRHP ADGAQQERCC VPPGERCPSA PDNGEENVPL SGKV |

SEQ ID NO: 3 Human HHLA2 Variant 2 cDNA Sequence (NM 001282556.1,
CDS region from position 224-1468)

| | |
|---|---|
| 1 | aaatcaaacg taccttggac tttactctct gagaaactca tagctgaatt caatgtttat |
| 61 | tcttatggac tacttagcat ttgactagac ggtatgaatt tctaagtaag cacatataga |
| 121 | actggatgcc cttgtggtac atctcaaggc tgatttgaaa gcttgagaga ccatcaagaa |
| 181 | ttggatttgg ggaagagcat gactaatatg ttctgcacaa gacatgaagg cacagacagc |
| 241 | actgtctttc ttcctcattc tcataacatc tctgagtgga tctcaaggca tattcccttt |
| 301 | ggctttcttc atttatgttc ctatgaatga acaaatcgtc attggaagac ttgatgaaga |
| 361 | tataattctc ccttcttcat ttgagagggg atccgaagtc gtaatacact ggaagtatca |
| 421 | agatagctat aaggttcaca gttactacaa aggcagtgac catttggaaa gccaagatcc |
| 481 | cagatatgca aacaggacat cccttttcta taatgagatt caaaatggga atgcgtcgct |
| 541 | atttttcaga agagtaagcc ttctggacga aggaatttac acctgctatg taggaacagc |
| 601 | aattcaagtg attacaaaca aagtggtgct aaaggtggga gtttttctca cacccgtgat |
| 661 | gaagtatgaa aagaggaaca caaacagctt cttaatatgc agcgtgttaa gtgtttatcc |
| 721 | tcgtccaatt atcacgtgga aaatggacaa cacacctatc tctgaaaaca acatggaaga |
| 781 | aacagggtct ttggattctt tttctattaa cagcccactg aatattacag gatcaaattc |
| 841 | atcttatgaa tgtacaattg aaaattcact gctgaagcaa acatggacag ggcgctggac |
| 901 | gatgaaagat ggccttcata aaatgcaaag tgaacacgtt tcactctcat gtcaacctgt |
| 961 | aaatgattat ttttcaccaa accaagactt caaagttact tggtccagaa tgaaaagtgg |
| 1021 | gactttctct gtcctggctt actatctgag ctcctcacaa aatacaatta tcaatgaatc |
| 1081 | ccgattctca tggaacaaag agctgataaa ccagagtgac ttctctatga atttgatgga |
| 1141 | tcttaatctt tcagacagtg gggaatattt atgcaatatt tcttcggatg aatatacttt |
| 1201 | acttaccatc cacacagtgc atgtagaacc gagccaagaa acagcttccc ataacaaagg |
| 1261 | cttatggatt ttggtgccct ctgcgatttt ggcagctttt ctgctgattt ggagcgtaaa |
| 1321 | atgttgcaga gcccagctag aagccaggag gagcagacac cctgctgatg gagcccaaca |
| 1381 | agaaagatgt tgtgtccctc ctggtgagcg ctgtcccagt gcacccgata atggcgaaga |
| 1441 | aaatgtgcct ctttcaggaa aagtatagga aatgagagaa gactgtgaca actcatgacc |
| 1501 | tgcatcctta atatccagtg acttcatctc ccctttcttc accacaattc caggcaatgg |
| 1561 | cctgtcggag cagacaattc taccactgca aagagttgta accattttct ggtatcacat |
| 1621 | ttatttttca agacatactt ttcaagacat cattcactga cccactacct gcattgagta |
| 1681 | taaatgcctg gatgttaagg attccaattt aactttgaaa agaactgtct cattcattta |
| 1741 | catttctgtt acagtcagcc caggaggtta cagtgagctc tccactaaga atctggaaga |
| 1801 | aatgcatcac taggggttga ttcccaatct gatcaactga taatgggtga gagagcaggt |
| 1861 | aagagccaaa gtcaccttag tggaaaggtt aaaaaccaga gcctggaaac caagatgatt |
| 1921 | gatttgacaa ggtattttag tctagtttta tatgaacggt tgtatcaggg taaccaactc |
| 1981 | gatttgggat gaatcttagg gcaccaaaga ctaagacagt atotttaaga ttgctaggga |
| 2041 | aaagggccct atgtgtcagg cctctgagcc caagccaagc atcgcatccc ctgtgatttg |
| 2101 | cacgtataca tccagatggc ctaaagtaac tgaagatcca caaaagaagt aaaaatagcc |
| 2161 | ttaactgatg acattccacc attgtgattt gttcctgccc caccctaact gatcaatgta |
| 2221 | ctttgtaatc tcccccaccc ttaagaaggt actttgtaat cttccccacc cttaagaagg |
| 2281 | ttctttgtaa ttctccccac ccttgagaat gtactttgtg agatccaccc tgcccacaaa |
| 2341 | acattgctct taacttcacc gcctaaccca aaacctataa gaactaatga taatccatca |
| 2401 | cccttcgctg actctctttt cggactcagc ccacctgcac ccaggtgaaa taaacagctt |
| 2461 | tattgctcac acaaaaaaaa aaaaaaaa |

SEQ ID NO: 4 Human HHLA2 Variant 2 Amino Acid Sequence
(NP 001269485.1)

| | |
|---|---|
| 1 | MKAQTALSFF LILITSLSGS QGIFPLAFFI YVPMNEQIVI GRLDEDIILP SSFERGSEVV |
| 61 | IHWKYQDSYK VHSYYKGSDH LESQDPRYAN RTSLFYNEIQ NGNASLFFRR VSLLDEGIYT |
| 121 | CYVGTAIQVI TNKVVLKVGV FLTPVMKYEK RNTNSFLICS VLSVYPRPII TWKMDNTPIS |
| 181 | ENNMEETGSL DSFSINSPLN ITGSNSSYEC TIENSLLKQT WTGRWTMKDG LHKMQSEHVS |
| 241 | LSCQPVNDYF SPNQDFKVTW SRMKSGTFSV LAYYLSSSQN TIINESRESW NKELINQSDF |
| 301 | SMNLMDLNLS DSGEYLCNIS SDEYTLLTIH TVHVEPSQET ASHNKGLWIL VPSAILAAFL |
| 361 | LIWSVKCCRA QLEARRSRHP ADGAQQERCC VPPGERCPSA PDNGEENVPL SGKV |

SEQ ID NO: 5 Human HHLA2 Variant 3 cDNA Sequence (NM 001282557.1,
CDS region from position 155-1399)

| | |
|---|---|
| 1 | agtttactct acatcatagc agagaaaatg gacaaaacac agctgttttg catgtaggag |
| 61 | aatactaacc ctgcacagat tgtgatggtg atgtggaata tactaaagcc tagaacgcac |
| 121 | ctcctctgca tgactaatat gttctgcaca agacatgaag gcacagacag cactgtcttt |
| 181 | cttcctcatt ctcataacat ctctgagtgg atctcaaggc atattccctt tggctttctt |
| 241 | catttatgtt cctatgaatg aacaaatcgt cattggaaga cttgatgaag atataattct |
| 301 | cccttcttca tttgagaggg gatccgaagt cgtaatacac tggaagtatc aagatagcta |
| 361 | taaggttcac agttactaca aaggcagtga ccatttggaa agccaagatc ccagatatgc |
| 421 | aaacaggaca tcccttttct ataatgagat tcaaaatggg aatgcgtcgc tatttttcag |
| 481 | aagagtaagc cttctggacg aaggaattta cacctgctat gtaggaacag caattcaagt |
| 541 | gattacaaac aaagtggtgc taaaggtggg agtttttctc acacccgtga tgaagtatga |
| 601 | aaagaggaac acaaacagct tottaatatg cagcgtgtta agtgtttatc ctcgtccaat |
| 661 | tatcacgtgg aaaatggaca acacacctat ctctgaaaac aacatggaag aaacagggtc |
| 721 | tttggattct ttttctatta acagcccact gaatattaca ggatcaaatt catcttatga |
| 781 | atgtacaatt gaaaattcac tgctgaagca aacatggaca gggcgctgga cgatgaaaga |
| 841 | tggccttcat aaaatgcaaa gtgaacacgt ttcactctca tgtcaacctg taaatgatta |
| 901 | tttttcacca aaccaagact tcaaagttac ttggtccaga atgaaaagtg ggactttctc |
| 961 | tgtcctggct tactatctga gctcctcaca aaatacaatt atcaatgaat cccgattctc |
| 1021 | atggaacaaa gagctgataa accagagtga cttctctatg aatttgatgg atottaatct |
| 1081 | ttcagacagt ggggaatatt tatgcaatat ttcttcggat gaatatactt tacttaccat |
| 1141 | ccacacagtg catgtagaac cgagccaaga aacagcttcc cataacaaag gcttatggat |
| 1201 | tttggtgccc tctgcgattt tggcagcttt tctgctgatt tggagcgtaa aatgttgcag |
| 1261 | agcccagcta gaagccagga ggagcagaca ccctgctgat ggagcccaac aagaaagatg |
| 1321 | ttgtgtccct cctggtgagc gctgtcccag tgcacccgat aatggcgaag aaaatgtgcc |
| 1381 | tctttcagga aaagtatagg aaatgagaga agactgtgac aactcatgac ctgcatcctt |
| 1441 | aatatccagt gacttcatct cccctttctt caccacaatt ccaggcaatg gcctgtcgga |
| 1501 | gcagacaatt ctaccactgc aaagagttgt aaccattttc tggtatcaca tttatttttc |
| 1561 | aagacatact tttcaagaca tcattcactg acccactacc tgcattgagt ataaatgcct |
| 1621 | ggatgttaag gattccaatt taactttgaa aagaactgtc tcattcattt acatttctgt |
| 1681 | tacagtcagc ccaggaggtt acagtgagct ctccactaag aatctggaag aaatgcatca |
| 1741 | ctaggggttg attcccaatc tgatcaactg ataatgggtg agagagcagg taagagccaa |
| 1801 | agtcacctta gtggaaaggt taaaaaccag agcctggaaa ccaagatgat tgatttgaca |
| 1861 | aggtatttta gtctagtttt atatgaacgg ttgtatcagg gtaaccaact cgatttggga |
| 1921 | tgaatcttag ggcaccaaag actaagacag tatctttaag attgctaggg aaaagggccc |
| 1981 | tatgtgtcag gcctctgagc ccaagccaag catcgcatcc cctgtgattt gcacgtatac |
| 2041 | atccagatgg cctaaagtaa ctgaagatcc acaaaagaag taaaaatagc cttaactgat |
| 2101 | gacattccac cattgtgatt tgttcctgcc ccaccctaac tgatcaatgt actttgtaat |
| 2161 | ctcccccacc cttaagaagg tactttgtaa tottccccac ccttaagaag gttctttgta |
| 2221 | attctcccca cccttgagaa tgtactttgt gagatccacc ctgcccacaa aacattgctc |
| 2281 | ttaacttcac cgcctaaccc aaaacctata agaactaatg ataatccatc acccttcgct |
| 2341 | gactctcttt tcggactcag cccacctgca cccaggtgaa ataaacagct ttattgctca |
| 2401 | cacaaaaaaa aaaaaaaaa |

SEQ ID NO: 6 Human HHLA2 Variant 3 Amino Acid Sequence
(NP 001269486.1)

| | |
|---|---|
| 1 | MKAQTALSFF LILITSLSGS QGIFPLAFFI YVPMNEQIVI GRLDEDIILP SSFERGSEVV |
| 61 | IHWKYQDSYK VHSYYKGSDH LESQDPRYAN RTSLFYNEIQ NGNASLFFRR VSLLDEGIYT |
| 121 | CYVGTAIQVI TNKVVLKVGV FLTPVMKYEK RNTNSFLICS VLSVYPRPII TWKMDNTPIS |
| 181 | ENNMEETGSL DSFSINSPLN ITGSNSSYEC TIENSLLKQT WTGRWTMKDG LHKMQSEHVS |
| 241 | LSCQPVNDYF SPNQDFKVTW SRMKSGTFSV LAYYLSSSQN TIINESRESW NKELINQSDE |
| 301 | SMNLMDLNLS DSGEYLCNIS SDEYTLLTIH TVHVEPSQET ASHNKGLWIL VPSAILAAFL |

SEQ ID NO: 7 Human HHLA2 Variant 4 cDNA Sequence (NM 001282558.1,
CDS region from position 302-1495)

| | |
|---|---|
| 1 | aaatcaaacg taccttggac tttactctct gagaaactca tagctgaatt caatgtttat |
| 61 | tcttatggac tacttagcat ttgactagac ggtatgaatt tctaagtaag cacatataga |
| 121 | actggatgcc cttgtggtac atctcaaggc tgatttgaaa gcttgagaga ccatcaagaa |
| 181 | ttggatttgg ggaagagcat gtaggagaat actaaccctg cacagattgt gatggtgatg |
| 241 | tggaatatac taaagcctag aacgcacctc ctctgcatga ctaatatgtt ctgcacaaga |
| 301 | catgaaggca cagacagcac tgtctttctt cctcattctc ataacatctc tgagtggatc |
| 361 | tcaaggcata ttccctttgg ctttcttcat ttatgttcct atgaatgaac aaatcgtcat |
| 421 | tggaagactt gatgaagata taattctccc ttcttcattt gagaggggat ccgaagtcgt |
| 481 | aatacactgg aagtatcaag atagctataa ggttcacagt tactacaaag gcagtgacca |
| 541 | tttggaaagc caagatccca gatatgcaaa caggacatcc cttttctata atgagattca |
| 601 | aaatgggaat gcgtcgctat ttttcagaag agtaagcctt ctggacgaag gaatttacac |
| 661 | ctgctatgta ggaacagcaa ttcaagtgat tacaaacaaa gtggtgctaa aggtgggagt |
| 721 | ttttctcaca cccgtgatga agtatgaaaa gaggaacaca aacagcttct taatatgcag |
| 781 | cgtgttaagt gtttatcctc gtccaattat cacgtggaaa atggacaaca cacctatctc |
| 841 | tgaaaacaac atggaagaaa cagggtcttt ggattctttt tctattaaca gcccactgaa |
| 901 | tattacagga tcaaattcat cttatgaatg tacaattgaa aattcactgc tgaagcaaac |
| 961 | atggacaggg cgctggacga tgaaagatgg ccttcataaa atgcaaagtg aacacgtttc |
| 1021 | actctcatgt caacctgtaa atgattattt ttcaccaaac caagacttca aagttacttg |
| 1081 | gtccagaatg aaaagtggga ctttctctgt cctggcttac tatctgagct cctcacaaaa |
| 1141 | tacaattatc aatgaatccc gattctcatg gaacaaagag ctgataaacc agagtgactt |
| 1201 | ctctatgaat ttgatggatc ttaatctttc agacagtggg gaatatttat gcaatatttc |
| 1261 | ttcggatgaa tatactttac ttaccatcca cacagtgcat gtagaaccga gccaagaaac |
| 1321 | agcttcccat aacaaaggct tatggatttt ggtgccctct gcgattttgg cagcttttct |
| 1381 | gctgatttgg agcgtaaaat gttgcagaga aagatgttgt gtccctcctg gtgagcgctg |
| 1441 | tcccagtgca cccgataatg gcgaagaaaa tgtgcctctt tcaggaaaag tataggaaat |
| 1501 | gagagaagac tgtgacaact catgacctgc atccttaata tccagtgact tcatctcccc |
| 1561 | tttcttcacc acaattccag gcaatggcct gtcggagcag acaattctac cactgcaaag |
| 1621 | agttgtaacc attttctggt atcacattta tttttcaaga catacttttc aagacatcat |
| 1681 | tcactgaccc actacctgca ttgagtataa atgcctggat gttaaggatt ccaatttaac |
| 1741 | tttgaaaaga actgtctcat tcatttacat ttctgttaca gtcagcccag gaggttacag |
| 1801 | tgagctctcc actaagaatc tggaagaaat gcatcactag gggttgattc ccaatctgat |
| 1861 | caactgataa tgggtgagag agcaggtaag agccaaagtc accttagtgg aaaggttaaa |
| 1921 | aaccagagcc tggaaaccaa gatgattgat ttgacaaggt attttagtct agttttatat |
| 1981 | gaacggttgt atcagggtaa ccaactcgat ttgggatgaa tottagggca ccaaagacta |
| 2041 | agacagtatc tttaagattg ctagggaaaa gggccctatg tgtcaggcct ctgagcccaa |
| 2101 | gccaagcatc gcatccccty tgatttgcac gtatacatcc agatggccta aagtaactga |
| 2161 | agatccacaa aagaagtaaa aatagcctta actgatgaca ttccaccatt gtgatttgtt |
| 2221 | cctgccccac cctaactgat caatgtactt tgtaatctcc cccaccctta agaaggtact |
| 2281 | ttgtaatctt ccccaccctt aagaaggttc tttgtaattc tccccaccct tgagaatgta |
| 2341 | ctttgtgaga tccaccctgc ccacaaaaca ttgctcttaa cttcaccgcc taacccaaaa |
| 2401 | cctataagaa ctaatgataa tccatcaccc ttcgctgact ctcttttcgg actcagccca |
| 2461 | cctgcaccca ggtgaaataa acagctttat tgctcacaca aaaaaaaaaa aaaaa |

SEQ ID NO: 8 Human HHLA2 Variant 4 Amino Acid Sequence
(NP 001269487.1)

| | |
|---|---|
| 1 | MKAQTALSFF LILITSLSGS QGIFPLAFFI YVPMNEQIVI GRLDEDIILP SSFERGSEVV |
| 61 | IHWKYQDSYK VHSYYKGSDH LESQDPRYAN RTSLFYNEIQ NGNASLFFRR VSLLDEGIYT |
| 121 | CYVGTAIQVI TNKVVLKVGV FLTPVMKYEK RNTNSFLICS VLSVYPRPII TWKMDNTPIS |
| 181 | ENNMEETGSL DSFSINSPLN ITGSNSSYEC TIENSLLKQT WTGRWTMKDG LHKMQSEHVS |
| 241 | LSCQPVNDYF SPNQDFKVTW SRMKSGTFSV LAYYLSSSQN TIINESRESW NKELINQSDE |
| 301 | SMNLMDLNLS DSGEYLCNIS SDEYTLLTIH TVHVEPSQET ASHNKGLWIL VPSAILAAFL |
| 361 | LIWSVKCCRE RCCVPPGERC PSAPDNGEEN VPLSGKV |

SEQ ID NO: 9 Human HHLA2 Variant 5 cDNA Sequence (NM 001282559.1,
CDS region from position 232-1284)

| | |
|---|---|
| 1 | aaatcaaacg taccttggac tttactctct gagaaactca tagctgaatt caatgtttat |
| 61 | tcttatggac tacttagcat ttgactagac ggtatgaatt tctaagtaag cacatataga |
| 121 | actggatgcc cttgtggtac atctcaaggc tgatttgaaa gcttgagaga ccatcaagaa |
| 181 | ttggatttgg ggaagagcat gtaggagaat actaaccctg cacagattgt gatggtgatg |
| 241 | tggaatatac taaagcctag aacgcacctc ctctgcatga ctaatatgtt ctgcacaaga |
| 301 | catgaaggca cagacagcac tgtctttctt cctcattctc ataacatctc tgagtggatc |
| 361 | tcaagaagag taagccttct ggacgaagga atttacacct gctatgtagg aacagcaatt |
| 421 | caagtgatta caaacaaagt ggtgctaaag gtgggagttt ttctcacacc cgtgatgaag |
| 481 | tatgaaaaga ggaacacaaa cagcttctta atatgcagcg tgttaagtgt ttatcctcgt |
| 541 | ccaattatca cgtggaaaat ggacaacaca cctatctctg aaaacaacat ggaagaaaca |
| 601 | gggtctttgg attctttttc tattaacagc ccactgaata ttacaggatc aaattcatct |
| 661 | tatgaatgta caattgaaaa ttcactgctg aagcaaacat ggacagggcg ctggacgatg |
| 721 | aaagatggcc ttcataaaat gcaaagtgaa cacgtttcac tctcatgtca acctgtaaat |
| 781 | gattattttt caccaaacca agacttcaaa gttacttggt ccagaatgaa aagtgggact |
| 841 | ttctctgtcc tggcttacta tctgagctcc tcacaaaata caattatcaa tgaatcccga |
| 901 | ttctcatgga acaaagagct gataaaccag agtgacttct ctatgaattt gatggatott |
| 961 | aatctttcag acagtgggga atatttatgc aatatttctt cggatgaata tactttactt |
| 1021 | accatccaca cagtgcatgt agaaccgagc caagaaacag cttcccataa caaaggctta |
| 1081 | tggattttgg tgccctctgc gattttggca gcttttctgc tgatttggag cgtaaaatgt |
| 1141 | tgcagagccc agctagaagc caggaggagc agacaccctg ctgatggagc ccaacaagaa |
| 1201 | agatgttgtg tccctcctgg tgagcgctgt cccagtgcac ccgataatgg cgaagaaaat |
| 1261 | gtgcctcttt caggaaaagt ataggaaatg agagaagact gtgacaactc atgacctgca |
| 1321 | tccttaatat ccagtgactt catctcccct ttcttcacca caattccagg caatggcctg |
| 1381 | tcggagcaga caattctacc actgcaaaga gttgtaacca ttttctggta tcacatttat |
| 1441 | ttttcaagac atacttttca agacatcatt cactgaccca ctacctgcat tgagtataaa |
| 1501 | tgcctggatg ttaaggattc caatttaact ttgaaaagaa ctgtctcatt catttacatt |
| 1561 | tctgttacag tcagcccagg aggttacagt gagctctcca ctaagaatct ggaagaaatg |
| 1621 | catcactagg ggttgattcc caatctgatc aactgataat gggtgagaga gcaggtaaga |
| 1681 | gccaaagtca ccttagtgga aaggttaaaa accagagcct ggaaaccaag atgattgatt |
| 1741 | tgacaaggta ttttagtcta gttttatatg aacggttgta tcagggtaac caactcgatt |
| 1801 | tgggatgaat cttagggcac caaagactaa gacagtatct ttaagattgc tagggaaaag |
| 1861 | ggccctatgt gtcaggcctc tgagcccaag ccaagcatcg catcccctgt gatttgcacg |
| 1921 | tatacatcca gatggcctaa agtaactgaa gatccacaaa agaagtaaaa atagccttaa |
| 1981 | ctgatgacat tccaccattg tgatttgttc ctgccccacc ctaactgatc aatgtacttt |
| 2041 | gtaatctccc ccacccttaa gaaggtactt tgtaatcttc cccaccctta agaaggttct |
| 2101 | ttgtaattct ccccaccctt gagaatgtac tttgtgagat ccaccctgcc cacaaaacat |
| 2161 | tgctcttaac ttcaccgcct aacccaaaac ctataagaac taatgataat ccatcaccct |
| 2221 | tcgctgactc tcttttcgga ctcagcccac ctgcacccag gtgaaataaa cagctttatt |
| 2281 | gctcacacaa aaaaaaaaaa aaaa |

SEQ ID NO: 10 Human HHLA2 Variant 5 Amino Acid Sequence
(NP 001269488.1)

| | |
|---|---|
| 1 | MVMWNILKPR THLLCMTNMF CTRHEGTDST VELPHSHNIS EWISRRVSLL DEGIYTCYVG |
| 61 | TAIQVITNKV VLKVGVFLTP VMKYEKRNTN SFLICSVLSV YPRPIITWKM DNTPISENNM |
| 121 | EETGSLDSFS INSPLNITGS NSSYECTIEN SLLKQTWTGR WTMKDGLHKM QSEHVSLSCQ |
| 181 | PVNDYFSPNQ DFKVTWSRMK SGTFSVLAYY LSSSQNTIIN ESRFSWNKEL INQSDESMNL |
| 241 | MDLNLSDSGE YLCNISSDEY TLLTIHTVHV EPSQETASHN KGLWILVPSA ILAAFLLIWS |
| 301 | VKCCRAQLEA RRSRHPADGA QQERCCVPPG ERCPSAPDNG EENVPLSGKV |

SEQ ID NO: 11 Human KIR3DL3 cDNA Sequence (NM 153443.4, CDS region
from position 51-1283)

| | |
|---|---|
| 1 | tgctgctgaa ctgagctggg gcgcagccgc ctgtctgcac cggcagcacc atgtcgctca |
| 61 | tggtcgtcag catggcgtgt gttgggttct tcttgctgga ggggccctgg ccacatgtgg |
| 121 | gtggtcagga caagcccttc ctctctgcct ggcccggcac tgtggtgtct gaaggacaac |
| 181 | atgtgactct tcagtgtcgc tctcgtcttg ggtttaatga attcagtctg tccaaagaag |
| 241 | acgggatgcc tgtccctgag ctctacaaca gaatattccg gaacagcttt ctcatgggcc |
| 301 | ctgtgacccc agcacatgca gggacctaca gatgttgcag ttcacaccca cactccccca |
| 361 | ctgggtggtc ggcacccagc aaccctgtgg tgatcatggt cacaggagtc cacagaaaac |
| 421 | cttccctcct ggcccaccca ggtcccctgg tgaaatcagg agagacggtc atcctgcaat |
| 481 | gttggtcaga tgtcaggttt gagcgcttcc ttctgcacag agaggggatc actgaggacc |
| 541 | ccttgcgcct cgttggacag ctccacgatg cgggttccca ggtcaactat tccatgggtc |
| 601 | ccatgacacc tgcccttgca gggacctaca gatgctttgg ttctgtcact cacttaccct |
| 661 | atgagttgtc ggctcccagt gaccctctgg acatcgtggt cgtaggtcta tatgggaaac |
| 721 | cttctctctc agcccagccg ggccccacgg ttcaggcagg agagaatgtg accttgtcct |
| 781 | gcagctcccg gagcttgttt gacatttacc atctatccag ggaggcggag gccggtgaac |
| 841 | ttaggctcac tgcagtgctg agggtcaatg gaacattcca ggccaacttc cctctgggcc |
| 901 | ctgtgaccca cggagggaac tacagatgct tcggctcttt ccgtgccctg ccccatgcgt |
| 961 | ggtcagaccc gagtgaccca ctgcccgttt ctgtcacagg taactccaga aacctgcacg |
| 1021 | ttctgattgg gacctcagtg gtcatcatcc cctttgctat cctcctcttc tttctccttc |
| 1081 | atcgctggtg tgccaacaaa aagaatgctg ttgtaatgga ccaagagcct gcagggaaca |
| 1141 | gaacagtgaa cagggaggac tctgatgaac aagaccctca ggaggtgaca tacgcacagt |
| 1201 | tgaatcactg cgttttcaca cagagaaaaa tcactcgccc ttctcagagg cccaagacac |
| 1261 | ccccaacaga taccagcgtg taacacggaa cttccaaatg ctgagcgcag atccaaagtt |
| 1321 | gtcttctgtc cactagcacc acagtcaggc cttgatggga tcttctaggg agacaatagc |
| 1381 | cctgtctcaa aaccgggttg ccagctccca tgtaccagca gctggactct gaaggcgtga |
| 1441 | gtctgcatct tagggcatcg ctcttcctca caccacgaat ctgaacatgc ctctctcttg |
| 1501 | cttacaaatg tctaaggtcc ccactgcctg ctggagagaa aacacacttg cttagcccac |
| 1561 | aattctccat ttcacttgac ccctgcccac ctctccaacc taactggctt acttcctagt |
| 1621 | ctacttgagg ctgcgatcac actgaggaac tcacaattcc aaacatataa gaggctccct |
| 1681 | cttaacacgg cacttagata cgtgctattc cacctttcct cag |

SEQ ID NO: 12 Human KIR3DL3 Amino Acid Sequence (NP 703144.3)

| | |
|---|---|
| 1 | MSLMVVSMAC VGFFLLEGPW PHVGGQDKPF LSAWPGTVVS EGQHVTLQCR SRLGENEFSL |
| 61 | SKEDGMPVPE LYNRIFRNSF LMGPVTPAHA GTYRCCSSHP HSPTGWSAPS NPVVIMVTGV |
| 121 | HRKPSLLAHP GPLVKSGETV ILQCWSDVRF ERFLLHREGI TEDPLRLVGQ LHDAGSQVNY |
| 181 | SMGPMTPALA GTYRCFGSVT HLPYELSAPS DPLDIVVVGL YGKPSLSAQP GPTVQAGENV |
| 241 | TLSCSSRSLF DIYHLSREAE AGELRLTAVL RVNGTFQANF PLGPVTHGGN YRCFGSFRAL |
| 301 | PHAWSDPSDP LPVSVTGNSR NLHVLIGTSV VIIPFAILLE FLLHRWCANK KNAVVMDQEP |
| 361 | AGNRTVNRED SDEQDPQEVT YAQLNHCVFT QRKITRPSQR PKTPPTDTSV |

* Included in Table 1 are RNA nucleic acid molecules (e.g., thymines replaced with uridines), nucleic acid molecules encoding orthologs of the encoded proteins, as well as DNA, cDNA, or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 1, or a portion thereof. Such nucleic acid molecules can have a function of the full-length nucleic acid as described further herein.
* Included in Table 1 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 1, or a portion thereof. Such polypeptides can have a function of the full-length polypeptide as described further herein.
* Included in Table 1 are other known HHLA2 and KIR3DL3 nucleic acid and amino acid sequences.

The term “KIR3DL3 activity,” includes the ability of a KIR3DL3 polypeptide to modulate an inhibitory signal in an activated immune cell, e.g., by engaging a natural HHLA2 ligand on a cancer cell. Modulation of an inhibitory signal in an immune cell results in modulation of proliferation of, and/or cytokine secretion by, an immune cell. Thus, the term “KIR3DL3 activity” includes the ability of a KIR3DL3 polypeptide to bind its natural ligand(s), the ability to modulate immune cell inhibitory signals, and the ability to modulate the immune response.

In some embodiments, a condition such as cancer is responsive to KIR3DL3 blockade alone. In other embodiments, a condition such as cancer is responsive to KIR3DL3 blockade alone, but is significantly or synergistically more responsive when treated with KIR3DL3 blockade and at least one other therapy in combination. Many conditions responsive to KIR3DL3 blockade alone or in combination include, without limitation, melanoma (e.g., advanced or metastatic melanoma), lung cancer (e.g., non-small cell lung cancer and small cell lung cancer), breast cancer (e.g., HER-2 negative breast cancer, estrogen-receptor+/HER-2-breast cancer, and triple negative breast cancer), pancreatic cancer (e.g., pancreatic adenocarcinoma), and Hodgkin lymphoma, as well as bladder, gastric, head and neck, renal, prostate, gynecologic, colorectal, ovary, adenocarcinoma, adenocarcinoma, chronic myelogenous leukemia (CML), and hematologic cancers.

Preferred B7 polypeptides are capable of providing costimulatory or inhibitory signals to immune cells to thereby promote or inhibit immune cell responses. For example, B7 family members that bind to costimulatory receptors increase T cell activation and proliferation, while B7 family members that bind to inhibitory receptors reduce costimulation. Moreover, the same B7 family member may increase or decrease T cell costimulation. For example, when bound to a costimulatory receptor, HHLA2 can induce costimulation of immune cells or when bound to an inhibitory receptor, HHLA2 can inhibit immune cells. When bound to an inhibitory receptor, HHLA2 can transmit an inhibitory signal to an immune cell. Preferred B7 family members include HHLA2, B7-1, B7-2, B7h, PD-L1 or PD-L2 and soluble fragments or derivatives thereof. In one embodiment, B7 family members bind to one or more receptors on an immune cell, e.g., TMIGD2, KIR3DL3, CTLA4, CD28, ICOS, PD-1 and/or other receptors, and, depending on the receptor, have the ability to transmit an inhibitory signal or a costimulatory signal to an immune cell, preferably a T cell.

Modulation of a costimulatory signal results in modulation of effector function of an immune cell. Thus, the term “KIR3DL3 activity” includes the ability of a KIR3DL3 ligand polypeptide to bind its natural receptor(s) (e.g. HHLA2), the ability to modulate immune cell costimulatory or inhibitory signals, and the ability to modulate the immune response.

KIR3DL3 pathway is a negative regulator of immune function, such that modulating the interaction between KIR3DL3 and one or more natural binding partners, such as HHLA2 can modulate immune function. Thus, the agents encompassed by the present disclosure described herein that modulate the interaction between KIR3DL3 and one or more natural binding partners whether directly or indirectly, can upregulate or downregulate the immune system and, thereby, upregulate or downregulate an immune response. Agents that modulate such an interaction can do so either directly or indirectly.

Exemplary agents for upregulating an immune response include antibodies against HHLA2 or KIR3DL3 that block the interaction between HHLA2 and KIR3DL3; a non-activating form of HHLA2 or KIR3DL3 (e.g., a dominant negative polypeptide), small molecules or peptides that block the interaction between HHLA2 and KIR3DL3; fusion proteins (e.g., the extracellular portion of HHLA2 or KIR3DL3 fused to the Fc portion of an antibody or immunoglobulin) that bind to HHLA2 or KIR3DL3, respectively, and inhibit the interaction between HHLA2 and KIR3DL3; nucleic acid molecules and/or genetic modifications that block HHLA2 and/or KIR3DL3 transcription or translation; a non-activating form of a natural HHLA2 ligand, and a soluble form of a natural KIR3DL3 ligand.

In other exemplary embodiments, agents that promote the binding of a HHLA2 polypeptide to one or more natural binding partners, such as KIR3DL3 polypeptide, promote an inhibitory signal to an immune cell. Agents that modulate such an interaction can do so either directly or indirectly. Thus, in one embodiment, agents which directly enhance the interaction between HHLA2 and KIR3DL3 (HHLA2 agonists and/or KIR3DL3 agonists) can promote inhibitory signaling and downregulate an immune response. Alternatively, agents that block KIR3DL3 binding to other targets increase the effective concentration of KIR3DL3 available to bind to HHLA2. Exemplary agents for downregulating an immune response include antibodies against HHLA2 or KIR3DL3 that activate or promote the interaction between HHLA2 and KIR3DL3; small molecules or peptides that activate or promote the interaction between HHLA2 and KIR3DL3; and blocking antibodies that bind natural binding partners of HHLA2 and KIR3DL3 other than HHLA2 and KIR3DL3, respectively.

Additional agents useful in the methods encompassed by the present disclosure include antibodies, small molecules, peptides, peptidomimetics, natural ligands, and derivatives of natural ligands, that can either bind and/or activate or inhibit protein biomarkers encompassed by the present disclosure, including the biomarkers listed in Table 1, or fragments thereof; RNA interference, antisense, nucleic acid aptamers, etc. that can downregulate the expression and/or activity of the biomarkers encompassed by the present disclosure, including the biomarkers listed in Table 1, or fragments thereof.

Isolated monoclonal antibodies or fragments thereof that are directed against KIR3DL3 are provided. In some embodiments, mAbs produced by hybridomas have been deposited at the American Type Culture Collection (ATCC), in accordance with the terms of Budapest Treaty, on under deposit numbers

Since it is well-known in the art that antibody heavy and light chain CDR3 domains play a particularly important role in the binding specificity/affinity of an antibody for an antigen, the recombinant monoclonal antibodies encompassed by the present disclosure prepared as set forth above preferably comprise the heavy and light chain CDR3s of variable regions encompassed by the present disclosure (e.g., including the sequences of Table 2, or portions thereof). The antibodies further can comprise the CDR2s of variable regions encompassed by the present disclosure (e.g., including the sequences of Table 2, or portions thereof). The antibodies further can comprise the CDR1s of variable regions encompassed by the present disclosure (e.g., including the sequences of Table 2, or portions thereof). In other embodiments, the antibodies can comprise any combinations of the CDRs.

The CDR1, 2, and/or 3 regions of the engineered antibodies described above can comprise the exact amino acid sequence(s) as those of variable regions encompassed by the present disclosure (e.g., including the sequences of Table 2, or portions thereof) disclosed herein. However, the ordinarily skilled artisan will appreciate that some deviation from the exact CDR sequences may be possible while still retaining the ability of the antibody to bind KIR3DL3 effectively (e.g., conservative sequence modifications). Accordingly, in another embodiment, the engineered antibody may be composed of one or more CDRs that are, for example, 50%, 60%, 70%, 80%, 85%, 90%, 91%, 92%, 93%, 94%, 95%, 96%, 97%, 98%, 99%, or 99.5% identical to one or more CDRs encompassed by the present disclosure (e.g., including the sequences of Table 2, or portions thereof).

The structural features of known, non-human or human antibodies (e.g., a mouse or a non-rodent anti-human KIR3DL3 antibody) can be used to create structurally related human anti-human KIR3DL3 antibodies that retain at least one functional property of the antibodies encompassed by the present disclosure, such as binding of KIR3DL3. Another functional property includes inhibiting binding of the original known, non-human or human antibodies in a competition ELISA assay.

In some embodiments, monoclonal antibodies capable of binding human KIR3DL3 are provided, comprising a heavy chain wherein the variable domain comprises at least a CDR having a sequence that is at least 80%, 85%, 90%, 91%, 92%, 93%, 94%, 95%, 96%, 97%, 98%, 99%, 99.5% or 100% identical from the group of heavy chain variable domain CDRs presented in Table 2.

Similarly, monoclonal antibodies capable of binding human KIR3DL3, comprising a light chain wherein the variable domain comprises at least a CDR having a sequence that is at least 80%, 85%, 90%, 91%, 92%, 93%, 94%, 95%, 96%, 97%, 98%, 99%, 99.5% or 100% identical from the group of light chain variable domain CDRs presented in Table 2, are also provided.

Monoclonal antibodies capable of binding human KIR3DL3, comprising a heavy chain wherein the variable domain comprises at least a CDR having a sequence that is at least 80%, 85%, 90%, 91%, 92%, 93%, 94%, 95%, 96%, 97%, 98%, 99%, 99.5% or 100% identical from the group of heavy chain variable domain CDRs presented in Table 2; and comprising a light chain wherein the variable domain comprises at least a CDR having a sequence that is at least 80%, 85%, 90%, 91%, 92%, 93%, 94%, 95%, 96%, 97%, 98%, 99%, 99.5% or 100% identical from the group of light chain variable domain CDRs presented in Table 2, are also provided.

A skilled artisan will note that such percentage homology is equivalent to and can be achieved by introducing 1, 2, 3, 4, 5, 6, 7, 8, 9, 10, or more conservative amino acid substitutions within a given CDR.

The monoclonal antibodies encompassed by the present disclosure can comprise a heavy chain, wherein the variable domain comprises at least one CDR having a sequence selected from the group consisting of the heavy chain variable domain CDRs presented in Table 2 and a light chain, wherein the variable domain comprises at least one CDR having a sequence selected from the group consisting of the light chain variable domain CDRs presented in Table 2.

Such monoclonal antibodies can comprise a light chain, wherein the variable domain comprises at least one CDR having a sequence selected from the group consisting of CDR-L1, CDR-L2, and CDR-L3, as described herein; and/or a heavy chain, wherein the variable domain comprises at least one CDR having a sequence selected from the group consisting of CDR-H1, CDR-H2, and CDR-H3, as described herein. In some embodiments, the monoclonal antibodies capable of binding human KIR3DL3 comprises or consists of CDR-L1, CDR-L2, CDR-L3, CDR-H1, CDR-H2, and CDR-H3, as described herein.

The heavy chain variable domain of the monoclonal antibodies encompassed by the present disclosure can comprise or consist of the vH amino acid sequence set forth in Table 2 and/or the light chain variable domain of the monoclonal antibodies encompassed by the present disclosure can comprise or consist of the vL amino acid sequence set forth in Table 2.

The monoclonal antibodies encompassed by the present disclosure can be produced and modified by any technique well-known in the art. For example, such monoclonal antibodies can be murine or non-rodent antibodies, such as those obtainable from the hybridoma deposited on with the ATCC as deposit Similarly, such monoclonal antibodies can be chimeric, preferably chimeric mouse/human antibodies. In some embodiments, monoclonal antibodies are humanized antibodies such that the variable domain comprises human acceptor frameworks regions, and optionally human constant domain where present, and non-human donor CDRs, such as mouse or non-rodent CDRs as defined above.

The present disclosure further provides fragments of said monoclonal antibodies which include, but are not limited to, Fv, Fab, F(ab′)2, Fab′, dsFv, scFv, sc (Fv)2 and diabodies; and multispecific antibodies formed from antibody fragments. For example, a number of immunoinhibitory molecules, such as HHLA2, PD-L2, PD-L1, CTLA-4, KIR3DL3, and the like, can be detected in a bispecific or multispecific manner in order to efficiently characterize the expression of such molecules.

Other fragments of the monoclonal antibodies encompassed by the present disclosure are also contemplated. For example, individual immunoglobulin heavy and/or light chains are provided, wherein the variable domains thereof comprise at least one CDR presented in Table 2. In one embodiment, the immunoglobulin heavy chain comprises at least one CDR having a sequence that is at least 80%, 85%, 90%, 91%, 92%, 93%, 94%, 95%, 96%, 97%, 98%, 99%, 99.5% or 100% identical from the group of heavy chain or light chain variable domain CDRs presented in Table 2. In another embodiment, an immunoglobulin light chain comprises at least one CDR having a sequence that is at least 80%, 85%, 90%, 91%, 92%, 93%, 94%, 95%, 96%, 97%, 98%, 99%, 99.5% or 100% identical from the group of light chain or heavy chain variable domain CDRs described herein (e.g., presented in Table 2).

In some embodiments, the immunoglobulin heavy and/or light chain comprises a variable domain comprising at least one of CDR-L1, CDR-L2, CDR-L3, CDR-H1, CDR-H2, or CDR-H3 described herein. Such immunoglobulin heavy chains can comprise or consist of at least one of CDR-H1, CDR-H2, and CDR-H3. Such immunoglobulin light chains can comprise or consist of at least one of CDR-L1, CDR-L2, and CDR-L3.

In other embodiments, an immunoglobulin heavy and/or light chain according to the present disclosure comprises or consists of a vH or vL variable domain sequence, respectively, provided in Table 2.

The present disclosure further provides polypeptides which have a sequence selected from the group consisting of vH variable domain, vL variable domain, CDR-L1, CDR-L2, CDR-L3, CDR-H1, CDR-H2, and CDR-H3 sequences described herein.

Antibodies, immunoglobulins, and polypeptides encompassed by the present disclosure can be used in an isolated (e.g., purified) form or contained in a vector, such as a membrane or lipid vesicle (e.g. a liposome).

TABLE 2
Characteristics and sequences of representative variable regions of anti-KIR3DL3
monoclonal antibodies, including mAbs 1C7, 1D12, 1G7, 2A3, 2D8, 2F11, 2H1, 8C2, and 8F7

| mouse | | | |
|---|---|---|---|
| anti-human | Jurkat assay for | Blocking of | |
| KIR3DL3 | enhancement of | HHLA2 | |
| mAbs | IL-2 Luciferase | binding | isoytpe |
| 574.2.1C7 | enhancer | blocker | IgG1 |
| 574.2.1D12 | ND, sup | blocker | IgG2a |
| 574.2.1G7 | strong enhancer | blocker | IgG2b |
| 574.2.2A3 | ND, sup | non-blocker | IgG1 |
| 574.2.2D8 | strong enhancer | blocker | IgG2b |
| 574.2.2F11 | strong enhancer | blocker | IgG2b |
| 574.2.2H1 | strong enhancer | blocker | IgG2b |
| 574.2.8C2 | ND, sup | blocker | IgG2a |
| 574.2.8F7 | Non-enhancer | non-blocker | IgG1 |

1C7 Heavy Chain Variable (vH) and Light Chain Variable (vL) DNA and Amino Acid
Sequences*
AVS-3698HC [574.2.1C7.D2.6.5 heavy chain]

| Region | Sequence Fragment | Residues | Length |
|---|---|---|---|
| HFR1 | QVQLQQSGAELMKPGASVKISCKAT | 1-25 | 25 |
| CDR-H1 | GYTESNY | 26-32 | 7 |
| HFR2 | WIEWIKQRPGHGLEWIGEI | 33-51 | 19 |
| CDR-H2 | LPGSGS | 52-57 | 6 |
| HFR3 | TNYNEKFKGKATFTAATSSNTAYMQLSS | 58-98 | 41 |
| | LTSEDSAVYYCAR | | |
| CDR-H3 | RVPDYYGSRYYFDY | 99-112 | 14 |
| HFR4 | WGQGTTLTVSS | 113-123 | 11 |

AVS-3698LC [574.2.1C7.D2.6.5 light chain]

| Region | | Sequence Fragment | | Residues | | Length | |
|---|---|---|---|---|---|---|---|
| LFR1 | | NIVMTQSPKSMSMSVGERVTLSC | | 1-23 | | 23 | |
| CDR-L1 | | KASENVGIYVS | | 24-34 | | 11 | |
| LFR2 | | WYQQKPEQSPKLLIY | | 35-49 | | 15 | |
| CDR-L2 | | GASNRYT | | 50-56 | | ? | |
| LFR3 | | GVPDRETGSGSAIDFTLTISSVQAEDLADYHC | | 57-88 | | 32 | |
| CDR-L3 | | GQSYNYPFT | | 89-97 | | 9 | |
| LFR4 | | FGSGTKLEIK | | 98-107 | | 10 | |
| | | Client | | | | | |
| SR | AVS | Sample | LP Sample | Chain | | | |
| linked | Linked | Name | Name | type | CDR-1 | CDR-2 | CDR-3 |
| SR- | AVS- | 574.2.1C7.D2.6.5 | AVS- | HC | GYTFSNY | LPGSGS | RVPDYYGSRYY |
| 14910 | 3698 | | 3698HC.3 | | | | FDY |
| Protein sequence | | | Nucleotide sequence | | | | |
| QVQLQQSGAELMKPGASVKIS | | | CAGGTTCAGCTGCAGCAGTCTGGAGCTGAGCTGATGAAGCC | | | | |
| CKATGYTFSNYWIEWIKQRPG | | | TGGGGCCTCAGTGAAGATATCCTGCAAGGCTACTGGCTACAC | | | | |
| HGLEWIGEILPGSGSTNYNEKF | | | ATTCAGTAACTACTGGATAGAGTGGATAAAGCAGAGGCCTG | | | | |
| KGKATFTAATSSNTAYMQLSS | | | GACATGGCCTTGAGTGGATTGGAGAGATTTTACCTGGAAGTG | | | | |
| LTSEDSAVYYCARRVPDYYGS | | | GTAGTACTAACTACAATGAGAAGTTCAAGGGCAAGGCCACA | | | | |
| RYYFDYWGQGTTLTVSS | | | TTCACTGCAGCTACATCCTCCAACACAGCCTACATGCAACTC | | | | |
| | | | AGCAGCCTGACATCTGAGGACTCTGCCGTCTATTACTGTGCA | | | | |
| | | | AGAAGGGTCCCTGATTACTACGGTAGTAGGTACTACTTTGAC | | | | |
| | | | TACTGGGGCCAAGGCACCACTCTCACAGTCTCCTCA | | | | |
| | | Client | | | | | |
| SR | AVS | Sample | LP Sample | Chain | | | |
| linked | Linked | Name | Name | type | CDR-1 | CDR-2 | CDR-3 |
| SR- | AVS- | 574.2.1C7.D2.6.5 | AVS- | LC | KASENVGIYV | GASNRY | GQSYNYPF |
| 14910 | 3698 | | 3698LCP.4 | | S | T | T |

| Protein sequence | Nucleotide sequence |
|---|---|
| NIVMTQSPKSMSMSVGERVTL | AACATTGTAATGACCCAATCTCCCAAATCCATGTCCATGTCA |
| SCKASENVGIYVSWYQQKPEQ | GTAGGAGAGAGGGTCACCTTGAGCTGCAAGGCCAGTGAGAA |
| SPKLLIYGASNRYTGVPDRFTG | TGTGGGTATTTACGTATCCTGGTATCAACAGAAACCAGAGCA |
| SGSATDFTLTISSVQAEDLADY | GTCTCCTAAACTACTAATATACGGGGCATCCAACCGATACAC |
| HCGQSYNYPFTFGSGTKLEIK | TGGGGTCCCCGATCGCTTCACAGGCAGTGGATCTGCAACAG |
| | ATTTCACTCTGACCATCAGCAGTGTGCAGGCTGAAGACCTTG |
| | CAGATTATCACTGTGGACAGAGTTACAACTATCCATTCACGT |
| | TCGGCTCGGGGACAAAGTTGGAAATCAAA |

2A3 Heavy Chain Variable (vH) and Light Chain Variable (vL) DNA and Amino Acid
Sequences*
AVS-3699HC [574.2.2A3.5.9 heavy chain]

| Region | Sequence Fragment | Residues | Length |
|---|---|---|---|
| HFR1 | EVQLQQSGPEL VKPGASYKMSCKAS | 1-25 | 25 |
| CDR-H1 | GYTFTSY | 26-32 | 7 |
| HFR2 | VMHWLKQKPGQGLEWIAYI | 33-51 | 19 |
| CDR-H2 | NPYNDG | 52-57 | 6 |
| HFR3 | TKYNENFKGKATLISDKSSSTAYMELSS | 58-98 | 41 |
| | LTSEDSAVYYCAR | | |
| CDR-H3 | PSMVTAAWFAY | 99-109 | 11 |
| HFR4 | WGQGTLVTVSA | 110-120 | 11 |

AVS-3699LC [574.2.2A3.5.9 light chain]

| Region | | Sequence Fragment | | Residues | | Length | |
|---|---|---|---|---|---|---|---|
| LFR1 | | DVVMTQTPLSLPVSLGDQASISC | | 1-23 | | 23 | |
| CDR-L1 | | RSSQNLAHSNGDTYLH | | 24-39 | | 16 | |
| LFR2 | | WYLQKPGQSPELLIY | | 40-54 | | 15 | |
| CDR-L2 | | KVSNRES | | 55-61 | | 7 | |
| LFR3 | | GVPDRFSGSGSGTDFTLKISRVEAEDLGVYFC | | 62-93 | | 32 | |
| CDR-L3 | | SQTTHVLWT | | 94-102 | | 9 | |
| LFR4 | | FGGGTKLEIK | | 103-112 | | 10 | |
| | | Client | | | | | |
| SR | AVS | Sample | LP Sample | Chain | | | |
| linked | Linked | Name | Name | type | CDR-1 | CDR-2 | CDR-3 |
| SR- | AVS- | 574.2.2A3.5.9 | AVS- | HC | GYTFTS | NPYNDG | PSMVTAAWF |
| 14910 | 3699 | | 3699HC.1 | | Y | | AY |
| Protein sequence | | | Nucleotide sequence | | | | |
| EVQLQQSGPEL VKPGASVKMS | | | GAGGTCCAGCTGCAGCAGTCTGGACCTGAGCTGGTAAAGCC | | | | |
| CKASGYTFTSYVMHWLKQKPG | | | TGGGGCTTCAGTGAAGATGTCCTGCAAGGCTTCTGGATACA | | | | |
| QGLEWIAYINPYNDGTKYNENF | | | CATTCACTAGCTATGTCATGCACTGGTTGAAGCAGAAGCCT | | | | |
| KGKATLTSDKSSSTAYMELSSL | | | GGGCAGGGCCTTGAGTGGATTGCATATATTAATCCTTACAA | | | | |
| TSEDSAVYYCARPSMVTAAWF | | | TGATGGTACTAAGTACAATGAGAACTTCAAAGGCAAGGCC | | | | |
| AYWGQGTLVTVSA | | | ACACTGACTTCAGACAAATCCTCCAGCACAGCCTACATGGA | | | | |
| | | | GCTCAGCAGCCTGACCTCTGAGGACTCTGCGGTCTATTACT | | | | |
| | | | GTGCAAGACCTAGTATGGTAACTGCCGCCTGGTTTGCTTAC | | | | |
| | | | TGGGGCCAAGGGACTCTGGTCACTGTCTCTGCA | | | | |
| | | Client | | | | | |
| SR | AVS | Sample | LP Sample | Chain | | | |
| linked | Linked | Name | Name | type | CDR-1 | CDR-2 | CDR-3 |
| SR- | AVS- | 574.2.2A3.5.9 | AVS- | LC | RSSQNLAH | KVSNRF | SQTTHVL W |
| 14910 | 3699 | | 3699LCP.1 | | SNGDTYLH | S | T |

| Protein sequence | Nucleotide sequence |
|---|---|
| DVVMTQTPLSLPVSLGDQASIS | GATGTTGTGATGACCCAAACTCCACTCTCCCTGCCTGTCAGT |
| CRSSQNLAHSNGDTYLHWYLQ | CTTGGAGATCAAGCCTCCATCTCCTGCAGATCTAGTCAGAA |
| KPGQSPKLLIYKVSNRFSGVPD | CCTTGCACACAGTAATGGAGACACCTATTTACATTGGTACC |
| RFSGSGSGTDFTLKISRVEAEDL | TGCAGAAGCCAGGCCAGTCTCCAAAGCTCCTGATCTACAAA |
| GVYFCSQTTHVLWTFGGGTKL | GTTTCCAACCGATTTTCTGGGGTCCCAGACAGGTTCAGTGG |
| EIK | CAGTGGATCAGGGACAGATTTCACACTCAAGATCAGCAGA |
| | GTGGAGGCTGAGGATCTGGGAGTTTATTTCTGCTCTCAAAC |
| | TACACATGTTTTGTGGACGTTCGGTGGAGGCACCAAGCTGG |
| | AAATCAAA |

8F7 Heavy Chain Variable (vH) and Light Chain Variable (vL) DNA and Amino Acid
Sequences*
AVS-3700HC [574.2.8F7.1.7.4 heavy chain]

| Region | Sequence Fragment | Residues | Length |
|---|---|---|---|
| HFR1 | DVKLVESGGGLVKPGGSLKLSCAAS | 1-25 | 25 |
| CDR-H1 | GFTFSSY | 26-32 | 7 |
| HFR2 | CMSWVRQTPEKRLEWVATI | 33-51 | 19 |
| CDR-H2 | SSGGNY | 52-57 | 6 |
| HFR3 | IYYPDSVKGRFTISRDIAKNTLYLQMSSLK | 58-98 | 41 |
| | SEDTAMYYCTR | | |
| CDR-H3 | GGYDERYEDY | 99-108 | 10 |
| HFR4 | WGQGTILTVSS | 109-119 | 11 |

AVS-3700LC [574.2.8F7.1.7.4 light chain]

| Region | | Sequence Fragment | | Residues | | Length | |
|---|---|---|---|---|---|---|---|
| LFR1 | | DIQMTQSPASLSASVGETVTITC | | 1-23 | | 23 | |
| CDR-L1 | | RASENTYSYLA | | 24-34 | | 11 | |
| LFR2 | | WYQQKQGKSPQLLVY | | 35-49 | | 15 | |
| CDR-L2 | | NAKTLTE | | 50-56 | | 7 | |
| LFR3 | | GMPSRFSGSGSGTQFSLRINSLQPEDFGSYYC | | 57-88 | | 32 | |
| CDR-L3 | | QHHHGTPRT | | 89-97 | | 9 | |
| LFR4 | | FGGGTKLEIK | | 98-107 | | 10 | |
| | | Client | | | | | |
| SR | AVS | Sample | LP Sample | Chain | | | |
| linked | Linked | Name | Name | type | CDR-1 | CDR-2 | CDR-3 |
| SR- | AVS- | 574.2.8F7.1.7.4 | AVS- | HC | GFTFSSY | SSGGN | GGYDERYFD |
| 14910 | 3700 | | 3700HC.2 | | | Y | Y |
| Protein sequence | | | Nucleotide sequence | | | | |
| DVKLVESGGGLVKPGGSLKLSC | | | GACGTGAAGTTGGTGGAGTCTGGGGGAGGCTTAGTGAAAC | | | | |
| AASGFTFSSYCMSWVRQTPEKR | | | CTGGAGGGTCCCTGAAACTCTCCTGTGCAGCCTCTGGATTC | | | | |
| LEWVATISSGGNYIYYPDSVKG | | | ACTTTCAGTTCCTATTGCATGTCTTGGGTTCGCCAGACTCCG | | | | |
| RFTISRDIAKNTLYLQMSSLKSE | | | GAGAAGAGGCTGGAGTGGGTCGCAACCATTAGTAGTGGTG | | | | |
| DTAMYYCTRGGYDERYFDYW | | | GTAATTACATCTACTATCCGGACAGTGTGAAGGGCCGATTC | | | | |
| GQGTILTVSS | | | ACCATCTCCAGAGACATTGCCAAGAACACCCTGTACCTGCA | | | | |
| | | | AATGAGCAGTCTGAAGTCTGAGGACACAGCCATGTATTACT | | | | |
| | | | GTACAAGAGGGGGTTACGACGAGAGGTACTTTGACTACTGG | | | | |
| | | | GGCCAAGGCACCATTCTCACAGTCTCCTCA | | | | |
| | | Client | | | | | |
| SR | AVS | Sample | LP Sample | Chain | | | |
| linked | Linked | Name | Name | type | CDR-1 | CDR-2 | CDR-3 |
| SR- | AVS- | 574.2.8F7.1.7.4 | AVS- | LC | RASENIYSYL | NAKTLT | QHHHGTPR |
| 14910 | 3700 | | 3700LCP.3 | | A | E | T |

| Protein sequence | Nucleotide sequence |
|---|---|
| DIQMTQSPASLSASVGETVTITC | GACATCCAGATGACTCAGTCTCCAGCCTCCCTTTCTGCTTCT |
| RASENIYSYLAWYQQKQGKSP | GTGGGAGAAACTGTCACCATCACATGTCGAGCAAGTGAAA |
| QLLVYNAKTLTEGMPSRFSGSG | ATATTTACAGTTATTTAGCATGGTATCAGCAGAAACAGGGA |
| SGTQFSLRINSLQPEDFGSYYCQ | AAATCTCCTCAGCTCCTGGTCTATAATGCAAAAACCTTAAC |
| HHHGTPRTFGGGTKLEIK | AGAAGGTATGCCATCAAGGTTCAGTGGCAGTGGATCAGGTA |
| | CACAGTTTTCTCTGAGGATCAATAGCCTGCAGCCTGAAGAT |
| | TTTGGGAGTTATTACTGTCAACATCATCATGGTACTCCTCGG |
| | ACGTTCGGTGGAGGCACCAAGTTGGAAATCAAA |

1G7 Heavy Chain Variable (vH) and Light Chain Variable (vL) DNA and Amino Acid
Sequences*
AVS-3701HC [574.2.1G7.9.9 heavy chain]

| Region | Sequence Fragment | Residues | Length |
|---|---|---|---|
| HFR1 | ELKLEESGGGLVQPGGSMKLSCVAS | 1-25 | 25 |
| CDR-H1 | GFTFSNY | 26-32 | 7 |
| HFR2 | WMNWVRQSPEKGLEWIAEI | 33-51 | 19 |
| CDR-H2 | RIKSNNYA | 52-59 | 8 |
| HFR3 | THYAESVKGRFTISRDDSKSSVYLQMNNLR | 60-100 | 41 |
| | AEDTGIFYCTP | | |
| CDR-H3 | FYGSSFPY | 101-108 | 8 |
| HFR4 | WGQGTLVTVSA | 109-119 | 11 |

AVS-3701LC [574.2.1G7.9.9 light chain]

| Region | | Sequence Fragment | | Residues | | Length | |
|---|---|---|---|---|---|---|---|
| LFR1 | | DIQMTQTTSSLSASLGDRVTISC | | 1-23 | | 23 | |
| CDR-L1 | | RASQDISNYLN | | 24-34 | | 11 | |
| LFR2 | | WYQQKPDGTVKLLIY | | 35-49 | | 15 | |
| CDR-L2 | | YTSILQL | | 50-56 | | 7 | |
| LFR3 | | GVPPRESGSGSGTDYSLTISNLEQEDIATYFC | | 57-88 | | 32 | |
| CDR-L3 | | QQGNTLPFT | | 89-97 | | 9 | |
| LFR4 | | FGAGTKVELK | | 98-107 | | 10 | |
| | | Client | | | | | |
| SR | AVS | Sample | LP Sample | Chain | | | |
| linked | Linked | Name | Name | type | CDR-1 | CDR-2 | CDR-3 |
| SR- | AVS- | 574.2.1G7.9.9 | AVS- | HC | GFTFSNY | RLKSNNY | FYGSSFPY |
| 14910 | 3701 | | 3701HCT.2 | | | A | |
| Protein sequence | | | Nucleotide sequence | | | | |
| ELKLEESGGGLVQPGGSMKLSCV | | | GAACTGAAGCTTGAGGAGTCTGGAGGAGGCTTGGTGCAA | | | | |
| ASGFTFSNYWMNWVRQSPEKGL | | | CCTGGAGGATCCATGAAACTCTCCTGTGTTGCCTCTGGAT | | | | |
| EWIAEIRLKSNNYATHYAESVKG | | | TCACTTTCAGTAACTACTGGATGAACTGGGTCCGCCAGTC | | | | |
| RFTISRDDSKSSVYLQMNNLRAE | | | TCCAGAGAAGGGGCTTGAATGGATTGCTGAAATTAGATT | | | | |
| DTGIFYCTPFYGSSFPYWGQGTLV | | | GAAATCTAATAATTATGCAACACATTATGCGGAGTCTGTG | | | | |
| TVSA | | | AAAGGGAGGTTCACCATCTCAAGAGATGATTCCAAAAGT | | | | |
| | | | AGTGTCTACCTACAGATGAACAACTTAAGAGCTGAAGAC | | | | |
| | | | ACTGGCATTTTTTACTGTACCCCCTTCTACGGTAGTTCTTT | | | | |
| | | | TCCTTACTGGGGCCAAGGGACTCTGGTCACTGTCTCTGCA | | | | |
| | | Client | | | | | |
| SR | AVS | Sample | LP Sample | Chain | | | |
| linked | Linked | Name | Name | type | CDR-1 | CDR-2 | CDR-3 |
| SR- | AVS- | 574.2.1G7.9.9 | AVS- | LC | RASQDISN | YTSILQL | QQGNTLPFT |
| 14910 | 3701 | | 3701LCP.2 | | YLN | | |

| Protein sequence | Nucleotide sequence |
|---|---|
| DIQMTQTTSSLSASLGDRVTISC | GATATCCAGATGACACAGACTACATCGTCCCTGTCTGCCTC |
| RASQDISNYLNWYQQKPDGTV | TCTGGGAGACAGAGTCACCATCAGTTGCAGGGCAAGTCAG |
| KLLIYYTSILQLGVPPRFSGSGS | GACATTAGCAATTACTTAAACTGGTATCAGCAGAAACCAGA |
| GTDYSLTISNLEQEDIATYFCQQ | TGGAACTGTTAAACTCCTTATCTATTACACATCAATATTACA |
| GNTLPFTFGAGTKVELK | ATTAGGAGTCCCGCCAAGGTTCAGTGGCAGTGGGTCTGGAA |
| | CAGATTATTCTCTCACCATTAGCAACCTGGAGCAAGAAGAT |
| | ATTGCCACTTACTTTTGCCAACAGGGTAATACGCTTCCGTTC |
| | ACGTTCGGTGCTGGGACCAAGGTGGAGCTGAAA |

2D8 Heavy Chain Variable (vH) and Light Chain Variable (vL) DNA and Amino Acid
Sequences*
AVS-3702HC [574.2.2D8.6.9 heavy chain]

| Region | Sequence Fragment | Residues | Length |
|---|---|---|---|
| HFR1 | DVQLVESGGGLVQPGGSRKLSCAAS | 1-25 | 25 |
| CDR-H1 | GFTFSYF | 26-32 | 7 |
| HFR2 | GMHWVRRAPEKGLEWVAFI | 33-51 | 19 |
| CDR-H2 | SSDSTT | 52-57 | 6 |
| HFR3 | IYYTDTVKGRFTISRDNPKNTLFLQMTS | 58-98 | 41 |
| | LRSEDTAIYYCAR | | |
| CDR-H3 | FYDGAMDY | 99-106 | 8 |
| HFR4 | WGQGISVTVST | 107-117 | 11 |

AVS-3702LC [574.2.2D8.6.9 light chain]

| Region | | Sequence Fragment | | Residues | | Length | |
|---|---|---|---|---|---|---|---|
| LFR1 | | DIVMTQSQKFMSTSVGDRVSITC | | 1-23 | | 23 | |
| CDR-L1 | | KASQSVRTAVA | | 24-34 | | 11 | |
| LFR2 | | WYQQKPGQSPKVLIY | | 35-49 | | 15 | |
| CDR-L2 | | LASNRHT | | 50-56 | | 7 | |
| LFR3 | | GVPDRFTGSGSGTDFTLTISNVQSEDLADYFC | | 57-88 | | 32 | |
| CDR-L3 | | LQHWNYPLT | | 89-97 | | 9 | |
| LFR4 | | FGAGTKLELK | | 98-107 | | 10 | |
| | | Client | | | | | |
| SR | AVS | Sample | LP Sample | Chain | | | |
| linked | Linked | Name | Name | type | CDR-1 | CDR-2 | CDR-3 |
| SR- | AVS-3702 | 574.2.2D8.6.9 | AVS- | HC | GFTFSYF | SSDSTT | FYDGAM |
| 14910 | | | 3702HC.2 | | | | DY |
| Protein sequence | | | Nucleotide sequence | | | | |
| DVQLVESGGGLVQPGGSRKLSCAAS | | | GATGTGCAGCTGGTGGAGTCTGGGGGAGGCTTAGTGC | | | | |
| GFTFSYFGMHWVRRAPEKGLEWVAF | | | AGCCTGGAGGGTCCCGGAAACTCTCCTGTGCAGCCTC | | | | |
| ISSDSTTIYYTDTVKGRFTISRDNPKNT | | | TGGATTCACTTTCAGTTACTTTGGAATGCACTGGGTTC | | | | |
| LFLQMTSLRSEDTAIYYCARFYDGAM | | | GTCGGGCTCCAGAGAAGGGGCTGGAGTGGGTCGCATT | | | | |
| DYWGQGTSVTVST | | | CATTAGTAGTGACAGTACTACCATCTACTATACAGAC | | | | |
| | | | ACAGTGAAGGGCCGATTCACCATCTCCAGAGACAATC | | | | |
| | | | CCAAGAACACCCTGTTCCTGCAAATGACCAGTCTAAG | | | | |
| | | | GTCTGAGGACACGGCCATATATTACTGTGCAAGATTT | | | | |
| | | | TACGACGGTGCTATGGACTACTGGGGTCAAGGAACCT | | | | |
| | | | CAGTCACCGTCTCCACA | | | | |
| | | Client | | | | | |
| SR | AVS | Sample | LP Sample | Chain | | | |
| linked | Linked | Name | Name | type | CDR-1 | CDR-2 | CDR-3 |
| SR- | AVS-3702 | 574.2.2D8.6.9 | AVS- | LC | KASQS | LASNRHT | LQHWNYP |
| 14910 | | | 3702LCP.5 | | VRTAV | | LT |
| | | | | | A | | |

| Protein sequence | Nucleotide sequence |
|---|---|
| DIVMTQSQKFMSTSVGDRVSITCKAS | GACATTGTGATGACCCAGTCTCAAAAATTCATGTCCA |
| QSVRTAVAWYQQKPGQSPKVLIYLA | CATCAGTAGGAGACAGGGTCAGCATCACCTGCAAGG |
| SNRHTGVPDRFTGSGSGTDFTLTISNV | CCAGTCAGAGTGTTCGTACTGCTGTAGCCTGGTATCA |
| QSEDLADYFCLQHWNYPLTFGAGTK | ACAGAAACCAGGGCAGTCTCCTAAAGTATTGATTTAC |
| LELK | TTGGCATCCAACCGGCACACTGGAGTCCCTGATCGCT |
| | TCACAGGCAGTGGATCTGGGACAGACTTCACTCTCAC |
| | CATTAGCAATGTGCAATCTGAAGACCTGGCAGATTAT |
| | TTCTGTCTTCAACATTGGAATTATCCTCTCACGTTCGG |
| | TGCTGGGACCAAGCTGGAGCTGAAA |

2F11 Heavy Chain Variable (vH) and Light Chain Variable (vL) DNA and Amino
Acid Sequences*
AVS-3703HC [574.2.2F11.2.7.4 heavy chain]

| Region | Sequence Fragment | Residues | Length |
|---|---|---|---|
| HFR1 | DVQLVESGGGLVQPGGSRKLSCAAS | 1-25 | 25 |
| CDR-H1 | GFTFSYF | 26-32 | 7 |
| HFR2 | GMHWVRRAPEKGLEWVAFI | 33-51 | 19 |
| CDR-H2 | SSDSTT | 52-57 | 6 |
| HFR3 | IYYTDTVKGRFTISRDNPKNTLFLQMTS | 58-98 | 41 |
| | LRSEDTAIYYCAR | | |
| CDR-H3 | FYDGAMDY | 99-106 | 8 |
| HFR4 | WGQGTSVTVST | 107-117 | 11 |

AVS-3703LC [574.2.2F11.2.7.4 light chain]

| Region | | Sequence Fragment | | Residues | | Length | |
|---|---|---|---|---|---|---|---|
| LFR1 | | DIVMTQSQKFMSTSVGDRVSITC | | 1-23 | | 23 | |
| CDR-L1 | | KASQSVRTAVA | | 24-34 | | 11 | |
| LFR2 | | WYQQKPGQSPKVLIY | | 35-49 | | 15 | |
| CDR-L2 | | LASNRHT | | 50-56 | | 7 | |
| LFR3 | | GVPDRFTGSGSGTDFTLTISNVQSEDLADYFC | | 57-88 | | 32 | |
| CDR-L3 | | LQHWNYPLT | | 89-97 | | 9 | |
| LFR4 | | FGAGTKLELK | | 98-107 | | 10 | |
| | | Client | | | | | |
| SR | AVS | Sample | LP Sample | Chain | | | |
| linked | Linked | Name | Name | type | CDR-1 | CDR-2 | CDR-3 |
| SR-14910 | AVS- | 574.2.2F11.2.7.4 | AVS- | HC | GFTFSYF | SSDSTT | FYDGAM |
| | 3703 | | 3703HCT.2 | | | | DY |
| Protein sequence | | | Nucleotide sequence | | | | |
| DVQLVESGGGLVQPGGSRKLSCAAS | | | GATGTGCAGCTGGTGGAGTCTGGGGGAGGCTTAGTGC | | | | |
| GFTFSYFGMHWVRRAPEKGLEWVAF | | | AGCCTGGAGGGTCCCGGAAACTCTCCTGTGCAGCCTC | | | | |
| ISSDSTTIYYTDTVKGRFTISRDNPKNT | | | TGGATTCACTTTCAGTTACTTTGGAATGCACTGGGTTC | | | | |
| LFLQMTSLRSEDTAIYYCARFYDGAM | | | GTCGGGCTCCAGAGAAGGGGCTGGAGTGGGTCGCATT | | | | |
| DYWGQGTSVTVST | | | CATTAGTAGTGACAGTACTACCATCTACTATACAGAC | | | | |
| | | | ACAGTGAAGGGCCGATTCACCATCTCCAGAGACAATC | | | | |
| | | | CCAAGAACACCCTGTTCCTGCAAATGACCAGTCTAAG | | | | |
| | | | GTCTGAGGACACGGCCATATATTACTGTGCAAGATTT | | | | |
| | | | TACGACGGTGCTATGGACTACTGGGGTCAAGGAACCT | | | | |
| | | | CAGTCACCGTCTCCACA | | | | |
| | | Client | | | | | |
| SR | AVS | Sample | LP Sample | Chain | | | |
| linked | Linked | Name | Name | type | CDR-1 | CDR-2 | CDR-3 |
| SR- | AVS- | 574.2.2F11.2.7.4 | AVS- | LC | KASQSVR | LASNRH | LQHWNYP |
| 14910 | 3703 | | 3703LCT.2 | | TAVA | T | LT |

| Protein sequence | Nucleotide sequence |
|---|---|
| DIVMTQSQKFMSTSVGDRVSITCKA | GACATTGTGATGACCCAGTCTCAAAAATTCATGTCCAC |
| SQSVRTAVAWYQQKPGQSPKVLIY | ATCAGTAGGAGACAGGGTCAGCATCACCTGCAAGGCCA |
| LASNRHTGVPDRFTGSGSGTDFTLT | GTCAGAGTGTTCGTACTGCTGTAGCCTGGTATCAACAG |
| ISNVQSEDLADYFCLQHWNYPLTFG | AAACCAGGGCAGTCTCCTAAAGTATTGATTTACTTGGC |
| AGTKLELK | ATCCAACCGGCACACTGGAGTCCCTGATCGCTTCACAG |
| | GCAGTGGATCTGGGACAGACTTCACTCTCACCATTAGC |
| | AATGTGCAATCTGAAGACCTGGCAGATTATTTCTGTCTT |
| | CAACATTGGAATTATCCTCTCACGTTCGGTGCTGGGACC |
| | AAGCTGGAGCTGAAA |

2H1 Heavy Chain Variable (vH) and Light Chain Variable (vL) DNA and Amino Acid
Sequences*
AVS-3704HC [574.2.2H1.11.6 heavy chain]

| Region | Sequence Fragment | Residues | Length |
|---|---|---|---|
| HFR1 | DVQLVESGGGLVQPGGSRKLSCAAS | 1-25 | 25 |
| CDR-H1 | GFTFSYF | 26-32 | 7 |
| HFR2 | GMHWVRRAPEKGLEWVAFI | 33-51 | 19 |
| CDR-H2 | SSDSTT | 52-57 | 6 |
| HFR3 | IYYTDTVKGRFTISRDNPKNTLFLQMTS | 58-98 | 41 |
| | LRSEDTAIYYCAR | | |
| CDR-H3 | FYDGAMDY | 99-106 | 8 |
| HFR4 | WGQGTSVTVST | 107-117 | 11 |

AVS-3704LC [574.2.2H1.11.6 light chain]

| Region | | Sequence Fragment | | Residues | | Length | |
|---|---|---|---|---|---|---|---|
| LFR1 | | DIVMTQSQKFMSTSVGDRVSITC | | 1-23 | | 23 | |
| CDR-L1 | | KASQSVRTAVA | | 24-34 | | 11 | |
| LFR2 | | WYQQKPGQSPKVLIY | | 35-49 | | 15 | |
| CDR-L2 | | LASNRHT | | 50-56 | | 7 | |
| LFR3 | | GVPDRFTGSGSGTDFTLTISNVQSEDLADYFC | | 57-88 | | 32 | |
| CDR-L3 | | LQHWNYPLT | | 89-97 | | 9 | |
| LFR4 | | FGAGTKLELK | | 98-107 | | 10 | |
| | | Client | | | | | |
| SR | AVS | Sample | LP Sample | Chain | | | |
| linked | Linked | Name | Name | type | CDR-1 | CDR-2 | CDR-3 |
| SR-14910 | AVS- | 574.2.2H1.11.6 | AVS- | HC | GFTFSYF | SSDSTT | FYDGAMDY |
| | 3704 | | 3704HCT.1 | | | | |
| Protein sequence | | | Nucleotide sequence | | | | |
| DVQLVESGGGLVQPGGSRKLSCA | | | GATGTGCAGCTGGTGGAGTCTGGGGGAGGCTTAGTGCAG | | | | |
| ASGFTFSYFGMHWVRRAPEKGLE | | | CCTGGAGGGTCCCGGAAACTCTCCTGTGCAGCCTCTGGA | | | | |
| WVAFISSDSTTIYYTDTVKGRFTIS | | | TTCACTTTCAGTTACTTTGGAATGCACTGGGTTCGTCGGG | | | | |
| RDNPKNTLFLQMTSLRSEDTAIYY | | | CTCCAGAGAAGGGGCTGGAGTGGGTCGCATTCATTAGTA | | | | |
| CARFYDGAMDYWGQGTSVTVST | | | GTGACAGTACTACCATCTACTATACAGACACAGTGAAGG | | | | |
| | | | GCCGATTCACCATCTCCAGAGACAATCCCAAGAACACCC | | | | |
| | | | TGTTCCTGCAAATGACCAGTCTAAGGTCTGAGGACACGG | | | | |
| | | | CCATATATTACTGTGCAAGATTTTACGACGGTGCTATGG | | | | |
| | | | ACTACTGGGGTCAAGGAACCTCAGTCACCGTCTCCACA | | | | |
| | | Client | | | | | |
| SR | AVS | Sample | LP Sample | Chain | | | |
| linked | Linked | Name | Name | type | CDR-1 | CDR-2 | CDR-3 |
| SR- | AVS- | 574.2.2H1.11.6 | AVS- | LC | KASQSV | LASNRH | LQHWNYPL |
| 14910 | 3704 | | 3704LCP.5 | | RTAVA | T | T |

| Protein sequence | Nucleotide sequence |
|---|---|
| DIVMTQSQKFMSTSVGDRVSITCK | GACATTGTGATGACCCAGTCTCAAAAATTCATGTCCACA |
| ASQSVRTAVAWYQQKPGQSPKVL | TCAGTAGGAGACAGGGTCAGCATCACCTGCAAGGCCAG |
| IYLASNRHTGVPDRFTGSGSGTDF | TCAGAGTGTTCGTACTGCTGTAGCCTGGTATCAACAGAA |
| TLTISNVQSEDLADYFCLQHWNYP | ACCAGGACAGTCTCCTAAAGTATTGATTTACTTGGCATC |
| LTFGAGTKLELK | CAACCGGCACACTGGAGTCCCTGATCGCTTCACAGGCAG |
| | TGGATCTGGGACAGACTTCACTCTCACCATTAGCAATGT |
| | GCAATCTGAAGACCTGGCAGATTATTTCTGTCTTCAACA |
| | TTGGAATTATCCTCTCACGTTCGGTGCTGGGACCAAGCT |
| | GGAGCTGAAA |

1D12 Heavy Chain Variable (vH) and Light Chain Variable (vL) DNA and Amino
Acid Sequences*
AVS-3705HC [574.2.1D12.1.6 heavy chain]

| Region | Sequence Fragment | Residues | Length |
|---|---|---|---|
| HFR1 | QVQLQQSGAELVRPGTSVKVSCKAS | 1-25 | 25 |
| CDR-H1 | GYAFNNY | 26-32 | 7 |
| HFR2 | LIEWVKQRPGQGLEWIGVI | 33-51 | 19 |
| CDR-H2 | NPGSGG | $2-57 | 6 |
| HFR3 | TNDNEKFRGKAILTADTSSSTAYMQLTS | 58-98 | 41 |
| | LTSDDSAVYFCAR | | |
| CDR-H3 | GNYYGTRDWYFDV | 99-111 | 13 |
| HFR4 | WGAGTTVTVSS | 112-122 | 11 |

AVS-3705LC [574.2.1D12.1.6 light chain]

| Region | | Sequence Fragment | | Residues | | Length | |
|---|---|---|---|---|---|---|---|
| LFR1 | | QIVLTQSPAIMSASLGARVTMTC | | 1-23 | | 23 | |
| CDR-L1 | | TASSSVSSSSLH | | 24-35 | | 12 | |
| LFR2 | | WYQQKPGSSPKLWIY | | 36-50 | | 15 | |
| CDR-L2 | | SESSLAS | | 51-57 | | 7 | |
| LFR3 | | GVPARFSGSGSGTSYSLTISSLEAEDAATYYC | | 58-89 | | 32 | |
| CDR-L3 | | HQYHRSPPT | | 90-98 | | 9 | |
| L.FR4 | | FGGGTKLEIK | | 99-108 | | 10 | |
| | | Client | | | | | |
| SR | AVS | Sample | LP Sample | Chain | | | |
| linked | Linked | Name | Name | type | CDR-1 | CDR-2 | CDR-3 |
| SR-14910 | AVS- | 574.2.1D12.1.6 | AVS- | HC | GYAFNN | NPGSG | GNYYGTR |
| | 3705 | | 3705HCT.2 | | Y | G | DWYFDV |
| Protein sequence | | | Nucleotide sequence | | | | |
| QVQLQQSGAELVRPGTSVKVSCKASG | | | CAGGTCCAGCTGCAGCAGTCTGGAGCTGAGCTGGTA | | | | |
| YAFNNYLIEWVKQRPGQGLEWIGVINP | | | AGGCCTGGGACTTCAGTGAAGGTGTCCTGCAAGGCT | | | | |
| GSGGTNDNEKFRGKATLTADTSSSTA | | | TCTGGATACGCCTTCAATAATTACTTGATAGAGTGGG | | | | |
| YMQLTSLTSDDSAVYFCARGNYYGTR | | | TAAAGCAGAGGCCTGGACAGGGCCTTGAGTGGATTG | | | | |
| DWYFDVWGAGTTVTVSS | | | GAGTGATCAATCCTGGAAGTGGTGGTACTAACGACA | | | | |
| | | | ATGAGAAGTTCAGGGGCAAGGCTACCCTGACTGCAG | | | | |
| | | | ACACATCCTCCAGCACTGCCTACATGCAGCTCACCA | | | | |
| | | | GCCTGACATCTGATGACTCTGCGGTCTATTTCTGTGC | | | | |
| | | | AAGAGGGAATTACTACGGTACTCGCGACTGGTACTT | | | | |
| | | | CGATGTCTGGGGCGCAGGGACCACGGTCACCGTCTC | | | | |
| | | | CTCA | | | | |
| | | Client | | | | | |
| SR | AVS | Sample | LP Sample | Chain | | | |
| linked | Linked | Name | Name | type | CDR-1 | CDR-2 | CDR-3 |
| SR-14910 | AVS-3705 | 574.2.1D12.1.6 | AVS- | LC | TASSSV | SISSLA | HQYHRSPP |
| | | | 3705LCP.2 | | SSSSLH | S | T |

| Protein sequence | Nucleotide sequence |
|---|---|
| QIVLTQSPAIMSASLGARVTMTCTASSS | CAAATTGTTCTCACCCAGTCTCCAGCAATCATGTCT |
| VSSSSLHWYQQKPGSSPKLWIYSISSLA | GCATCTCTAGGGGCACGGGTCACCATGACCTGCACT |
| SGVPARFSGSGSGTSYSLTISSLEAEDAA | GCCAGCTCAAGTGTAAGTTCCAGTTCCTTGCACTGG |
| TYYCHQYHRSPPTFGGGTKLEIK | TACCAGCAGAAGCCAGGATCCTCCCCCAAACTCTGG |
| | ATTTATAGTATATCCAGCCTGGCTTCTGGAGTCCCA |
| | GCTCGCTTCAGTGGCAGTGGGTCTGGGACCTCTTAC |
| | TCTCTCACAATCAGCAGCTTGGAGGCTGAAGATGCT |
| | GCCACTTATTACTGCCACCAGTATCATCGCTCCCCA |
| | CCGACGTTCGGTGGAGGCACCAAGCTGGAAATCAA |
| | A |

8C2 Heavy Chain Variable (vH) and Light Chain Variable (vL) DNA and Amino Acid
Sequences*
AVS-3706HC [574.2.8C2.12.3.10 heavy chain]

| Region | Sequence Fragment | Residues | Length |
|---|---|---|---|
| HFR1 | KYQLQQSGAEL VRPGTSVKVSCKAS | 1-25 | 25 |
| CDR-H1 | GYAFTNY | 26-32 | 7 |
| HFR2 | LIEWVKQRPGQGLEWIGVI | 33-51 | 19 |
| CDR-H2 | NPGSGG | 52-57 | 6 |
| HFR3 | INDNEKFRDKATLTADKSSSTAYMQLSS | 58-98 | 41 |
| | LTSDDSAVYFCAR | | |
| CDR-H3 | GNYYGTTDWYFDV | 99-111 | 13 |
| HFR4 | WGAGTTVTVSS | 112-122 | 11 |

AVS-3706LC [574.2.8C2.12.3.10 light chain]

| Region | | Sequence Fragment | | Residues | | Length | |
|---|---|---|---|---|---|---|---|
| LFR1 | | QIVLTQSPAIMSASLGERVTMTC | | 1-23 | | 23 | |
| CDR-L1 | | TASASVSSSSLH | | 24-35 | | 12 | |
| LFR2 | | WYQQKPGSPPKFWIY | | 36-50 | | 15 | |
| CDR-L2 | | STINLAS | | 51-57 | | 7 | |
| LFR3 | | GVPTRESGSGSGTSYSLTISNMEAEDAATYYC | | 58-89 | | 32 | |
| CDR-L3 | | HQYHRSPPT | | 90-98 | | 9 | |
| LFR4 | | FGGGTKLEIK | | 99-108 | | 10 | |
| | | Client | | | | | |
| SR | AVS | Sample | LP Sample | Chain | | | |
| linked | Linked | Name | Name | type | CDR-1 | CDR-2 | CDR-3 |
| SR- | AVS- | 574.2.8C2.12.3.10 | AVS- | HC | GYAFTN | NPGSG | GNYYGTT |
| 14910 | 3706 | | 3706HCT.1 | | Y | G | DWYFDV |
| Protein sequence | | | Nucleotide sequence | | | | |
| KVQLQQSGAELVRPGTSVKVSCK | | | AAGGTCCAGCTGCAGCAGTCTGGAGCTGAGCTGGTAAG | | | | |
| ASGYAFTNYLIEWVKQRPGQGLE | | | GCCTGGGACTTCAGTGAAGGTGTCCTGCAAGGCTTCTGG | | | | |
| WIGVINPGSGGTNDNEKFRDKATL | | | ATACGCCTTCACTAATTATTTGATAGAGTGGGTAAAGCA | | | | |
| TADKSSSTAYMQLSSLTSDDSAVY | | | GAGGCCTGGACAGGGCCTTGAGTGGATTGGAGTGATTAA | | | | |
| FCARGNYYGTTDWYFDVWGAGT | | | TCCTGGAAGTGGTGGTACTAATGACAATGAGAAGTTCAG | | | | |
| TVTVSS | | | GGACAAGGCAACATTGACTGCAGACAAATCCTCCAGCA | | | | |
| | | | CTGCCTACATGCAGCTCAGCAGCCTGACATCTGATGACT | | | | |
| | | | CTGCGGTCTATTTCTGTGCAAGAGGGAATTACTACGGTA | | | | |
| | | | CTACCGACTGGTACTTCGATGTCTGGGGCGCAGGGACCA | | | | |
| | | | CGGTCACCGTCTCCTCA | | | | |
| | | Client | | | | | |
| SR | AVS | Sample | LP Sample | Chain | | | |
| linked | Linked | Name | Name | type | CDR-1 | CDR-2 | CDR-3 |
| SR- | AVS- | 574.2.8C2.12.3.10 | AVS- | LC | TASAS | STTNLAS | HQYHRSPP |
| 14910 | 3706 | | 3706LCP.1 | | VSSSSL | | T |
| | | | | | H | | |

| Protein sequence | Nucleotide sequence |
|---|---|
| QIVLTQSPAIMSASLGERVTMTCT | CAAATTGTTCTCACCCAGTCTCCAGCAATCATGTCTGCAT |
| ASASVSSSSLHWYQQKPGSPPKFW | CTCTAGGGGAACGGGTCACCATGACCTGCACTGCCAGCG |
| IYSTTNLASGVPTRFSGSGSGTSYS | CAAGTGTAAGTTCCAGCTCCTTGCACTGGTACCAGCAGA |
| LTISNMEAEDAATYYCHQYHRSPP | AGCCAGGATCCCCCCCCAAATTCTGGATTTATAGCACAA |
| TFGGGTKLEIK | CCAACCTGGCTTCTGGAGTCCCAACTCGCTTCAGTGGCA |
| | GTGGGTCTGGGACCTCTTACTCTCTCACAATCAGCAACA |
| | TGGAGGCTGAAGATGCTGCCACTTATTACTGCCACCAGT |
| | ATCATCGTTCCCCACCGACGTTCGGTGGAGGCACCAAGC |
| | TGGAAATCAAA |

*CDR definitions and protein sequence numbering according to Kabat.
*Included in Table 2 are RNA nucleic acid molecules (e.g., thymines replaced with uridines), nucleic acid molecules encoding orthologs of the encoded proteins, as well as DNA, cDNA, or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 2, or a portion thereof. Such nucleic acid molecules can have a function of the full-length nucleic acid as described further herein.

### III. Nucleic Acids, Vectors, and Recombinant Host Cells

A further aspect encompassed by the present disclosure relates to nucleic acid sequences encoding monoclonal antibodies and fragments thereof, immunoglobulins, and polypeptides encompassed by the present disclosure.

Typically, a nucleic acid is a DNA or RNA molecule, which may be included in any suitable vector, such as a plasmid, cosmid, episome, artificial chromosome, phage or a viral vector.

Vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further aspect encompassed by the present disclosure relates to a cell which has been transfected, infected or transformed by a nucleic acid and/or a vector according to the present disclosure. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids encompassed by the present disclosure may be used to produce a recombinant polypeptide encompassed by the present disclosure in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

The present disclosure also relates to methods of producing a recombinant host cell expressing an antibody or a polypeptide encompassed by the present disclosure according to the present disclosure, said method comprising the steps consisting of (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody or polypeptide. Such recombinant host cells can be used for the production of antibodies and polypeptides as described herein.

In another aspect, the present disclosure provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides encompassed by the present disclosure can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences. Optionally, polynucleotides of this invention will encode at least a portion of an antibody encoded by the polynucleotides described herein. The polynucleotides of this invention embrace nucleic acid sequences that can be employed for selective hybridization to a polynucleotide encoding an antibody encompassed by the present disclosure. See, e.g., Ausubel, supra; Colligan, supra, each entirely incorporated herein by reference.

### IV. Methods of Producing Antibodies

Antibodies and fragments thereof, immunoglobulins, and polypeptides encompassed by the present disclosure may be produced by any technique known in the art, such as, without limitation, any chemical, biological, genetic or enzymatic technique, either alone or in combination.

Knowing the amino acid sequence of the desired sequence, one skilled in the art can readily produce said antibodies or polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies and other polypeptides encompassed by the present disclosure can be synthesized by recombinant DNA techniques as is well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

In particular, the present disclosure further relates to a method of producing an antibody or a polypeptide encompassed by the present disclosure, which method comprises the steps consisting of: (i) culturing a transformed host cell according to the present disclosure under conditions suitable to allow expression of said antibody or polypeptide; and (ii) recovering the expressed antibody or polypeptide.

Antibodies and other polypeptides encompassed by the present disclosure may be suitably separated from the culture medium by conventional immunoglobulin purification procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Chimeric antibodies (e.g., mouse-human chimeras or non-rodent-human chimeras) encompassed by the present disclosure can be produced by obtaining nucleic sequences encoding V_{L} and V_{H} domains as previously described, constructing a human chimeric antibody expression vector by inserting them into an expression vector for animal cell having genes encoding human antibody CH and human antibody CL, and expressing the coding sequence by introducing the expression vector into an animal cell. The CH domain of a human chimeric antibody can be any region which belongs to human immunoglobulin, such as the IgG class or a subclass thereof, such as IgG1, IgG2, IgG3 and IgG4. Similarly, the CL of a human chimeric antibody can be any region which belongs to Ig, such as the kappa class or lambda class. Chimeric and humanized monoclonal antibodies, comprising both human and non-human portions, which can be made using standard recombinant DNA techniques, are within the scope encompassed by the present disclosure. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332. In some embodiments, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743. Humanized antibodies encompassed by the present disclosure can be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell. The humanized antibody expression vector may be either of a type in which a gene encoding an antibody heavy chain and a gene encoding an antibody light chain exists on separate vectors or of a type in which both genes exist on the same vector (tandem type).

Methods for producing humanized antibodies based on conventional recombinant DNA and gene transfection techniques are well-known in the art (See, e.g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

Similarly, bispecific or multispecific antibodies described herein can be made according to standard procedures. For example, triomas and hybrid hybridomas are two examples of cell lines that can secrete bispecific or multispecific antibodies. Examples of bispecific and multispecific antibodies produced by a hybrid hybridoma or a trioma are disclosed in U.S. Pat. No. 4,474,893. Such antibodies can also be constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986) Immunol. Today 7:241). Alternatively, such antibodies can also be generated by making heterohybridomas by fusing hybridomas or other cells making different antibodies, followed by identification of clones producing and co-assembling the desired antibodies. They can also be generated by chemical or genetic conjugation of complete immunoglobulin chains or portions thereof such as Fab and Fv sequences. The antibody component can bind to a polypeptide or a fragment thereof of one or more biomarkers encompassed by the present disclosure, including one or more immunoinhibitory biomarkers described herein.

In addition, methods for producing antibody fragments are well-known. For example, Fab fragments encompassed by the present disclosure can be obtained by treating an antibody which specifically reacts with human KIR3DL3 with a protease such as papain. Also, Fabs can be produced by inserting DNA encoding Fabs of the antibody into a vector for prokaryotic expression system, or for eukaryotic expression system, and introducing the vector into a procaryote or eucaryote (as appropriate) to express the Fabs.

Similarly, F(ab′)2 fragments encompassed by the present disclosure can be obtained treating an antibody which specifically reacts with KIR3DL3 with a protease, pepsin. Also, the F(ab′)2 fragment can be produced by binding Fab′ described below via a thioether bond or a disulfide bond.

Fab′ fragments encompassed by the present disclosure can be obtained treating F(ab′)2 which specifically reacts with human KIR3DL3 with a reducing agent, dithiothreitol. Also, the Fab′ fragments can be produced by inserting DNA encoding a Fab′ fragment of the antibody into an expression vector for prokaryote, or an expression vector for eukaryote, and introducing the vector into a prokaryote or eukaryote (as appropriate) to perform its expression.

In addition, scFvs encompassed by the present disclosure can be produced by obtaining cDNA encoding the V_{H} and V_{L} domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well-known technology called CDR grafting may be used, which involves selecting the complementary determining regions (CDRs) from a donor scFv fragment, and grafting them onto a human scFv fragment framework of known three dimensional structure (see, e.g., WO98/45322; WO 87/02671; U.S. Pat. Nos. 5,859,205; 5,585,089; 4,816,567; EP0173494).

V. Modification of Antibodies. Immunoglobulins, and Polypeptides

Amino acid sequence modification(s) of the antibodies described herein are contemplated. For example, it may be desirable to improve the binding affinity and/or other biological properties of the antibody. It is known that when a humanized antibody is produced by simply grafting only CDRs in V_{H} and V_{L} of an antibody derived from a non-human animal in FRs of the V_{H} and V_{L} of a human antibody, the antigen binding activity is reduced in comparison with that of the original antibody derived from a non-human animal. It is considered that several amino acid residues of the V_{H} and V_{L} of the non-human antibody, not only in CDRs but also in FRs, are directly or indirectly associated with the antigen binding activity. Hence, substitution of these amino acid residues with different amino acid residues derived from FRs of the V_{H} and V_{L} of the human antibody would reduce binding activity and can be corrected by replacing the amino acids with amino acid residues of the original antibody derived from a non-human animal.

Modifications and changes may be made in the structure of the antibodies encompassed by the present disclosure, and in the DNA sequences encoding them, and still obtain a functional molecule that encodes an antibody and polypeptide with desirable characteristics. For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences encompassed by the present disclosure, or corresponding DNA sequences which encode said polypeptides, without appreciable loss of their biological activity.

In some embodiments, amino acid changes may be achieved by changing codons in the DNA sequence to encode conservative substitutions based on conservation of the genetic code. Specifically, there is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code (see genetic code chart above).

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

Another type of amino acid modification of the antibodies encompassed by the present disclosure may be useful for altering the original glycosylation pattern of the antibody to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N-or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr H. et al. (1987) and by Edge, A S. et al. (1981). Enzymatic cleavage of carbohydrate moieties on antibodies can be achieved by the use of a variety of endo- and exo-glycosidases as described by Thotakura, N R. et al. (1987).

Other modifications can involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Conjugation of antibodies or other proteins encompassed by the present disclosure with heterologous agents can be made using a variety of bifunctional protein coupling agents including but not limited to N-succinimidyl (2-pyridyldithio) propionate (SPDP), succinimidyl (N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6 diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

In another aspect, the present disclosure features antibodies that specifically bind KIR3DL3 conjugated to a therapeutic moiety, such as a cytotoxin, a drug, and/or a radioisotope. When conjugated to a cytotoxin, these antibody conjugates are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). An antibody of the present disclosure can be conjugated to a radioisotope, e.g., radioactive iodine, to generate cytotoxic radiopharmaceuticals for treating a related disorder, such as a cancer.

Conjugated anti-KIR3DL3 antibodies can be used, inter alia, diagnostically or prognostically to monitor polypeptide levels in tissue as part of a clinical testing procedure, e.g., to determine the efficacy of a given treatment regimen or to select patients most likely to response to an immunotherapy. For example, cells can be permeabilized in a flow cytometry assay to allow antibodies that bind KIR3DL3 to target its recognized intracellular epitope and allow detection of the binding by analyzing signals emanating from the conjugated molecules. Detection can be facilitated by coupling (i e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include 1251, 1311, 35S, or 3H. As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or indocyanine (Cy5)) to the antibody, as well as indirect labeling of the antibody by reactivity with a detectable substance.

The antibody conjugates encompassed by the present disclosure can be used to modify a given biological response. The chemical moiety is not to be construed as limited to classical chemical agents. For example, the drug moiety may be a protein or polypeptide possessing a desired biological activity. Such proteins may include, for example, a tumor necrosis factor or interferon-. gamma.; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other cytokines or growth factors.

Techniques for conjugating such therapeutic moiety to antibodies are well-known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243 56 (Alan R. Liss, Inc. 1985); Hellstrom et al., “Antibodies For Drug Delivery”, in Controlled Drug Delivery (2nd Ed.), Robinson et al. (eds.), pp. 623 53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475 506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303 16 (Academic Press 1985), and Thorpe et al., “The Preparation And Cytotoxic Properties Of Antibody-Toxin Conjugates”, Immunol. Rev., 62:119 58 (1982).

In some embodiments, conjugations can be made using a “cleavable linker” facilitating release of the cytotoxic agent or growth inhibitory agent in a cell. For example, an acid-labile linker, peptidase-sensitive linker, photolabile linker, dimethyl linker or disulfide-containing linker (See e.g. U.S. Pat. No. 5,208,020) may be used. Alternatively, a fusion protein comprising the antibody and a growth inhibitory agent may be made, by recombinant techniques or peptide synthesis. The length of DNA may comprise respective regions encoding the two portions of the conjugate either adjacent one another or separated by a region encoding a linker peptide which does not destroy the desired properties of the conjugate.

### VI. Uses and Methods

The anti-KIR3DL3 antibodies, immunoglobulins, polypeptides, and nucleic acids encompassed by the present disclosure described herein can be useful for a variety of uses, such as KIR3DL3 detection methods, therapeutic purposes (e.g., therapeutic, prophylactic, and immunomodulatory) either alone or in combination with other therapeutics, and the like. In addition, the anti-KIR3DL3 antibodies, immunoglobulins, polypeptides, and nucleic acids encompassed by the present disclosure described herein can be used in numerous predictive medicine assays based on detection of KIR3DL3 levels. For example, the present disclosure provides for prognostic (or predictive) assays for determining whether an individual will be responsive to a certain therapy (e.g., a therapy targeting KIR3DL3). As described herein, a KIR3DL3 polypeptide or fragment thereof encompassed by the present disclosure has one or more of the following activities: 1) binds to and/or modulates the activity of its natural binding partner(s), such as HHLA2; 2) modulates intra- or intercellular signaling, such as co-immunoinhibitory signaling; 3) modulates activation of T cells or NK cells; 4) modulates the immune response of an organism, e.g., a mammalian organism, such as a mouse, a non-rodent animal, or human; and 5) modulates immune cell anergy.

The present disclosure also provides for detection of KIR3DL3 as a means to identify agents that transduce a KIR3DL3 signal. Agents that transduce a KIR3DL3 signal would attenuate immune responses and might be useful in autoimmune diseases, asthma, and for the establishment of tolerance.

In any method described herein, KIR3DL3 can be detected either alone or in combination with the expression of other molecules, such as other immune checkpoint and/or costimulatory molecules. Combinatorial detection (e.g., sequentially or simultaneously) of several molecules can provide useful information regarding synergies of therapeutic intervention and/or personalized, higher-resolution diagnoses of disorder subtypes. In some embodiments, KIR3DL3 is combinatorially detected with one more markers.

### 1. Therapeutic Methods and Uses

In some embodiments, antibodies, fragments or immunoconjugates encompassed by the present disclosure (e.g., anti-KIR3DL3 antibodies) are useful for treating any disorder (e.g., a cancer) associated with aberrant or undesired activation of KIR3DL3. In certain embodiments, the treatment is of a mammal, such as a human. Such antibodies encompassed by the present disclosure may be used alone or in combination with any suitable agent or appropriate therapy to treat the disorder of interest. For example, therapeutic synergies are believed to become manifested when treating a cell with a therapy comprising anti-KIR3DL3 mAbs and another immune checkpoint inhibitors or cell therapies, such as CAR.

The antibodies or fragments thereof encompassed by the present disclosure described herein are useful in modulating the immune response by preventing or disrupting the interaction between KIR3DL3 and its natural ligand, HHLA2. Similarly, the antibodies or fragments thereof described herein are useful in treating diseases, e.g., cancer by increasing immune response and T cell and/or NK cell activity against cancer cells. Thus, an object encompassed by the present disclosure relates to a method for modulating immune response and/or treating a disorder associated with aberrant KIR3DL3 activation comprising administering a subject in need thereof with a therapeutically effective amount of an antibody, fragment thereof encompassed by the present disclosure.

Upregulation of immune responses can be in the form of enhancing an existing immune response or eliciting an initial immune response. For instance, enhancing an immune response using the subject compositions and methods is useful in cases of improving an immunological defense against cancer and infections with microbes (e.g., bacteria, viruses, or parasites). For example, upregulation or enhancement of an immune response function, as described herein, is useful in the induction of tumor immunity.

In another embodiment, the immune response can be stimulated by the methods described herein, such that preexisting tolerance, clonal deletion, and/or exhaustion (e.g., T cell exhaustion) is overcome. For example, immune responses against antigens to which a subject cannot mount a significant immune response, e.g., to an autologous antigen, such as a tumor specific antigens can be induced by administering appropriate agents described herein that upregulate the imimune response. In one embodiment, an autologous antigen, such as a tumor-specific antigen, can be coadministered. In another embodiment, an immune response can be stimulated against an antigen (e.g., an autologous antigen) to treat a neurological disorder. In another embodiment, the subject agents can be used as adjuvants to boost responses to foreign antigens in the process of active immunization.

In certain instances, it may be desirable to further administer other agents that upregulate immune responses, for example, forms of other B7 family members that transduce signals via costimulatory receptors, in order to further augment the immune response. Also, agents that upregulate an immune response can be used prophylactically in vaccines against various polypeptides (e.g., polypeptides derived from pathogens). Immunity against a pathogen (e.g., a virus) can be induced by vaccinating with a viral protein along with an agent that upregulates an immune response, in an appropriate adjuvant.

Additionally or alternatively, in some embodiments, the antibodies and the antigen-binding fragments encompassed by the present disclosure are useful for therapeutic applications, in addition to diagnostic, prognostic, and prevention applications (such as treating, and delaying the onset or progression of the diseases), to inhibit diseases that upregulate the immune reaction, for example, asthma, autoimmune diseases (glomerular nephritis, arthritis, dilated cardiomyopathy-like disease, ulceous colitis, Sjogren syndrome, Crohn disease, systemic erythematodes, chronic rheumatoid arthritis, multiple sclerosis, psoriasis, allergic contact dermatitis, polymyosiis, pachyderma, periarteritis nodosa, rheumatic fever, vitiligo vulgaris, insulin dependent diabetes mellitus, Behcet disease, Hashimoto disease, Addison disease, dermatomyositis, myasthenia gravis, Reiter syndrome, Graves' disease, anaemia perniciosa, Goodpasture syndrome, sterility disease, chronic active hepatitis, pemphigus, autoimmune thrombopenic purpura, and autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus, erythematosis, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, some cases of lymphopenia, mixed connective tissue disease, pemphigoid, pemphigus vulgaris, pernicious anema, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, ulcerative colitis and Wegener's granulomatosis).

Similarly, the antibodies and the antigen-binding fragments encompassed by the present disclosure are useful for therapeutic applications, in addition to diagnostic, prognostic, and prevention applications (such as treating, and delaying the onset or progression of the diseases) for persistent infectious disease (e.g., viral infectious diseases including HPV, HBV, hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, and influenza virus. Other antigens associated with pathogens that can be used as described herein are antigens of various parasites, includes malaria, preferably malaria peptide based on repeats of NANP. In addition, bacterial, fungal and other pathogenic diseases are included, such as Aspergillus, Brugia, Candida, Chlamydia, Coccidia, Cryptococcus, Dirofilaria, Gonococcus, Histoplasma, Leishmania, Mycobacterium, Mycoplasma, Paramecium, Pertussis, Plasmodium, Pneumococcus, Pneumocystis, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum; or, a fungus, such as, e.g., Paracoccidioides brasiliensis; or other pathogen, e.g., Plasmodium falciparum. Also included are National Institute of Allergy and Infectious Diseases (NIAID) priority pathogens. These include Category A agents, such as variola major (smallpox), Bacillus anthracis (anthrax), Yersinia pestis (plague), Clostridium botulinum toxin (botulism), Francisella tularensis (tularaemia), filoviruses (Ebola hemorrhagic fever, Marburg hemorrhagic fever), arenaviruses (Lassa (Lassa fever), Junin (Argentine hemorrhagic fever) and related viruses); Category B agents, such as Coxiella burnetti (Q fever), Brucella species (brucellosis), Burkholderia mallei (glanders), alphaviruses (Venezuelan encephalomyelitis, eastern & western equine encephalomyelitis), ricin toxin from Ricinus communis (castor beans), epsilon toxin of Clostridium perfringens; Staphylococcus enterotoxin B, Salmonella species, Shigella dysenteriae, Escherichia coli strain 0157: H7, Vibrio cholerae, Cryptosporidium parvum; Category C agents, such as nipah virus, hantaviruses, tickborne hemorrhagic fever viruses, tickborne encephalitis viruses, yellow fever, and multidrug-resistant tuberculosis; helminths, such as Schistosoma and Taenia; and protozoa, such as Leishmania (e.g., L. mexicana) and Plasmodium.

In some embodiments, antibodies or the antigen-binding fragments encompassed by the present disclosure are useful for therapeutic applications, in addition to prognostic and prevention applications, regarding induction of immunological tolerance, organ graft rejection, graft-versus-host disease (GVHD), allergic disease, and diseases caused by attenuation of immune reactions mediated by KIR3DL3.

In the context of the invention, the term “treating” or “treatment”, as used herein, means reversing, alleviating, or inhibiting the progress of the disorder or condition to which such term applies, or one or more symptoms of such disorder or condition. By the term “treating cancer” as used herein is meant the inhibition of the growth and/or proliferation of cancer cells. Preferably such treatment also leads to the regression of tumor growth (i.e., the decrease in size of a measurable tumor). Most preferably, such treatment leads to the complete regression of the tumor.

Therapeutic formulations comprising one or more antibodies encompassed by the present disclosure are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. The antibody composition may be formulated, dosed, and administered in any fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The therapeutic dose can be at least about 0.001 μg/kg body weight, 0.005 μg/kg body weight, 0.01 μg/kg body weight, at least about 0.05 μg/kg body weight; at least about 0.1 μg/kg body weight, at least about 0.5 μg/kg body weight, at least about 1 μg/kg body weight, at least about 2.5 μg/kg body weight, at least about 5 μg/kg body weight, at least about 50 μg/kg body weight, or at least about 100 μg/kg body weight. It will be understood by one of skill in the art that such guidelines will be adjusted for the molecular weight of the active agent, e.g. in the use of antibody fragments, or in the use of antibody conjugates. The dosage may also be varied for localized administration, e.g. intranasal, inhalation, etc., or for systemic administration, e.g. i.m., i.p., i.v., and the like.

The composition need not be, but is optionally formulated with one or more agents that potentiate activity, or that otherwise increase the therapeutic effect.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride;
benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The compositions described herein can be administered by any suitable means, including parenteral, subcutaneous, intraperitoneal, intrapulmonary, and intranasal. Parenteral infusions include intramuscular, intravenous, intraarterial, intraperitoneal, or subcutaneous administration. In addition, the compositions can be suitably administered by pulse infusion, particularly with declining doses of the antibody.

For the prevention or treatment of disease, the appropriate dosage of antibody will depend on the type of disease to be treated, as defined above, the severity and course of the disease, whether the antibody is administered for preventive purposes, previous therapy, the patient's clinical history and response to the antibody, and the discretion of the attending physician. The antibody is suitably administered to the patient at one time or over a series of treatments.

Agents which directly block the interaction between KIR3DL3 and HHLA2, such as an anti-HHLA2 antibody, an anti-KIR3DL3 antibody, an anti-KIR3DL3/anti-immune checkpoint bispecific antibody (e.g., anti-KIR3DL3/PD-1 bispecific antibody), and the like, can prevent the KIR3DL3 signaling and its downstream immune responses. Alternatively, agents that indirectly block the interaction between KIR3DL3 and HHLA2 can prevent the KIR3DL3 signaling and its downstream immune responses. For example, in some embodiments, a soluble form of KIR3DL3, such as an extracellular domain of KIR3DL3, by binding to HHLA2, can indirectly reduce the effective concentration of HHLA2 available to bind to KIR3DL3 on cell surface. Exemplary agents include monospecific or bispecific blocking antibodies against KIR3DL3 and/or HHLA2 that block the interaction between the receptor and ligand(s); a non-activating form of HHLA2 and/or KIR3DL3 (e.g., a dominant negative or soluble polypeptide), small molecules or peptides that block the interaction between KIR3DL3 and HHLA2; fusion proteins (e.g. the extracellular portion of HHLA2 and/or KIR3DL3, fused to the Fc portion of an antibody or immunoglobulin) that bind to KIR3DL3 and/or HHLA2 and inhibit the interaction between the receptor and ligand(s); a non-activating form of a natural KIR3DL3 and/or HHLA2, and a soluble form of a natural KIR3DL3 and/or HHLA2.

In some embodiments, anti-KIR3DL3 antibody therapy or combinations of therapies (e.g., one or more anti-KIR3DL3 antibody therapy in combination with one or more additional anti-cancer therapies, such as another immune checkpoint inhibitor) can be administered. Combination therapies can comprise, for example, one or more chemotherapeutic agents and radiation, one or more chemotherapeutic agents and immunotherapy, or one or more chemotherapeutic agents, radiation and chemotherapy, each combination of which can be with anti-immune checkpoint therapy. In addition, any representative embodiment of an agent to modulate a particular target can be adapted to any other target described herein and below by the ordinarily skilled artisan (e.g., direct and indirect KIR3DL3 inhibitors described herein can be applied to other immune checkpoint inhibitors and/or monospecific antibodies, bispecific antibodies, non-activating forms, small molecules, peptides, interfering nucleic acids, and the like).

Thus, the therapeutic agents encompassed by the present disclosure can be used alone or can be administered in combination therapy with, e.g., chemotherapeutic agents, hormones, antiangiogens, CAR, radiolabelled compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre-or post-conventional therapy. For example, agents encompassed by the present disclosure can be administered with a therapeutically effective dose of chemotherapeutic agent. In another embodiment, agents encompassed by the present disclosure are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

The anti-KIR3DL3 agents can also be administered in combination with targeted therapy, e.g., immunotherapy. Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

The term “targeted therapy” refers to administration of agents that selectively interact with a chosen biomolecule, for example, to thereby treat cancer. For example, targeted therapy regarding the inhibition of immune checkpoint inhibitor is useful in combination with the methods encompassed by the present disclosure. The term “immune checkpoint inhibitor” means a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, TMIDG2, KIR3DL3, and A2aR (see, for example, WO 2012/177624). Inhibition of one or more immune checkpoint inhibitors can block or otherwise neutralize inhibitory signaling to thereby upregulate an immune response in order to more efficaciously treat cancer.

Immunotherapy is one form of targeted therapy that may comprise, for example, the use of one or more cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer. As described above, immunotherapy against immune checkpoint targets, such as HHLA2, KIR3DL3, and the like, are useful.

In some embodiments, immunotherapy may comprise one or more adoptive cell-based immunotherapies. Well-known adoptive cell-based immunotherapeutic modalities, including, without limitation, Irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, patient-specific neoantigen vaccines, and the like.

In some embodiments, immunotherapy may comprise one or more non-cell-based immunotherapies. In some embodiments, compositions comprising antigens with or without vaccine-enhancing adjuvants are used. Such compositions exist in many well-known forms, such as peptide compositions, oncolytic viruses, recombinant antigen comprising fusion proteins, and the like. In still another embodiment, immunomodulatory interleukins, such as IL-2, IL-6, IL-7, IL-12, IL-17, IL-23, and the like, as well as modulators thereof (e.g., blocking antibodies or more potent or longer lasting forms) are used. In yet another embodiment, immunomodulatory cytokines, such as interferons, G-CSF, imiquimod, TNFalpha, and the like, as well as modulators thereof (e.g., blocking antibodies or more potent or longer lasting forms) are used. In another embodiment, immunomodulatory chemokines, such as CCL3, CCL26, and CXCL7, and the like, as well as modulators thereof (e.g., blocking antibodies or more potent or longer lasting forms) are used. In another embodiment, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

In some embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-kB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), 13C (indole-3-carbinol)/DIM (di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet another embodiment, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

In some embodiments, nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, agents and therapies other than immunotherapy can be used with in combination with an anti-KIR3DL3 antibodies to stimulate an immune response to thereby treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well-known in the art.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6 (5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et.al. Experimental Hematology, Volume 31, Number 6, June 2003, pp. 446-454 (9); Herceg Z.; Wang Z.-Q. Mutation Research/Fundamental and Molecular Mechanisms of Mutagenesis, Volume 477, Number 1, 2 Jun. 2001, pp. 97-110 (14)). Poly (ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

The duration and/or dose of treatment with therapies may vary according to the particular therapeutic agent or combination thereof. An appropriate treatment time for a particular cancer therapeutic agent will be appreciated by the skilled artisan. The present disclosure contemplates the continued assessment of optimal treatment schedules for each cancer therapeutic agent, where the phenotype of the cancer of the subject as determined by the methods encompassed by the present disclosure is a factor in determining optimal treatment doses and schedules.

Any means for the introduction of a polynucleotide into mammals, human or non-human, or cells thereof may be adapted to the practice of this invention for the delivery of the various constructs encompassed by the present disclosure into the intended recipient. In one embodiment encompassed by the present disclosure, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. The preferred colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner, et al., Ann NY Acad Sci 126-139, 1995). Formulation of DNA, e.g. with various lipid or liposome materials, may then be effected using known methods and materials and delivered to the recipient mammal. See, e.g., Canonico et al, Am J Respir Cell Mol Biol 10:24-29, 1994; Tsan et al, Am J Physiol 268; Alton et al., Nat Genet. 5:135-142, 1993 and U.S. Pat. No. 5,679,647 by Carson et al.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Naked DNA or DNA associated with a delivery vehicle, e.g., liposomes, can be administered to several sites in a subject (see below).

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

Nucleic acids encoding a protein or nucleic acid of interest may be in a plasmid or viral vector, or other vector as is known in the art. Such vectors are well-known and any can be selected for a particular application. In one embodiment encompassed by the present disclosure, the gene delivery vehicle comprises a promoter and a demethylase coding sequence. Preferred promoters are tissue-specific promoters and promoters which are activated by cellular proliferation, such as the thymidine kinase and thymidylate synthase promoters. Other preferred promoters include promoters which are activatable by infection with a virus, such as the a-and β-interferon promoters, and promoters which are activatable by a hormone, such as estrogen. Other promoters which can be used include the Moloney virus LTR, the CMV promoter, and the mouse albumin promoter. A promoter may be constitutive or inducible.

In another embodiment, naked polynucleotide molecules may be used as gene delivery vehicles, as described in WO 90/11092 and U.S. Pat. No. 5,580,859. Such gene delivery vehicles can be either growth factor DNA or RNA and, in certain embodiments, are linked to killed adenovirus. Curiel et al., Hum. Gene. Ther. 3:147-154, 1992. Other vehicles which can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise one or more viral sequences such as a viral origin of replication or packaging signal. These viral sequences can be selected from viruses such as astrovirus, coronavirus, orthomyxovirus, papovavirus, paramyxovirus, parvovirus, picornavirus, poxvirus, retrovirus, togavirus or adenovirus. In a preferred embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present disclosure, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that can be used to deliver a polynucleotide encompassed by the present disclosure have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth,; Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Preferred viruses include an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al. (1990) J.Virol., 64:642-650).

In other embodiments, target DNA in the genome can be manipulated using well-known methods in the art. For example, the target DNA in the genome can be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis.

In other embodiments, recombinant biomarker polypeptides, and fragments thereof, can be administered to subjects. In some embodiments, fusion proteins can be constructed and administered which have enhanced biological properties. In addition, the biomarker polypeptides, and fragment thereof, can be modified according to well-known pharmacological methods in the art (e.g., pegylation, glycosylation, oligomerization, etc.) in order to further enhance desirable biological activities, such as increased bioavailability and decreased proteolytic degradation.

### 2 Assays and Screening Methods

Another aspect encompassed by the present disclosure relates to screening assays, including non-cell based assays and xenograft animal model assays. In one embodiment, the assays provide a method for identifying agents that modulate KIR3DL3 signaling, such as in a human or an animal model assay, in order to identify agents that reduce KIR3DL3 signaling thereby increasing immune responses and/or identify agents that increase KIR3DL3 signaling thereby decreasing immune responses.

In one embodiment, the present disclosure relates to assays for screening test agents which bind to, or modulate the biological activity of, at least one biomarker described herein (e.g., in the tables, figures, examples, or otherwise in the specification), such as HHLA2, TMIGD2, and KIR3DL3. In one embodiment, a method for identifying such an agent entails determining the ability of the agent to modulate, e.g. inhibit, the at least one biomarker described herein.

In one embodiment, an assay is a cell-free or cell-based assay, comprising contacting at least one biomarker described herein, with a test agent, and determining the ability of the test agent to modulate (e.g., inhibit) the enzymatic activity of the biomarker, such as by measuring direct binding of substrates or by measuring indirect parameters as described below.

For example, in a direct binding assay, a biomarker protein (or their respective target polypeptides or molecules) can be coupled with a radioisotope or enzymatic label such that binding can be determined by detecting the labeled protein or molecule in a complex. For example, the targets can be labeled with 1251, 35S, 14C, or 3H, either directly or indirectly, and the radioisotope detected by direct counting of radioemmission or by scintillation counting. Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies described herein can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene. In an alternative embodiment, determining the ability of the agent to modulate the interaction between the biomarker and a substrate or a biomarker and its natural binding partner can be accomplished by determining the ability of the test agent to modulate the activity of a polypeptide or other product that functions downstream or upstream of its position within the signaling pathway (e.g., feedback loops). Such feedback loops are well-known in the art (see, for example, Chen and Guillemin (2009) Int. J. Tryptophan Res. 2:1-19).

KIR3DL3 status can be measured using the anti-KIR3DL3 antibodies described herein. A reduction in KIR3DL3 binding to HHLA2 indicates that the agent inhibits KIR3DL3 activity/signaling and identifies an agent as useful for inhibiting KIR3DL3 activity/signaling and for increasing immune responses. By contrast, an increase in KIR3DL3 binding to HHLA2 indicates that the agent promotes KIR3DL3 activity/signaling and identifies an agent as useful for promoting KIR3DL3 activity/signaling and for reducing immune responses.

The present disclosure further pertains to novel agents identified by the above-described screening assays. Accordingly, it is within the scope of this invention to further use an agent identified as described herein, such as in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

One aspect encompassed by the present disclosure relates to screening assays, including non-cell based assays and xenograft animal model assays. In one embodiment, the assays provide a method for identifying whether a cancer is likely to respond to anti-KIR3DL3 antibody therapy, such as in a human by using a xenograft animal model assay, and/or whether an agent can inhibit the growth of or kill a cancer cell that is unlikely to respond to anti-KIR3DL3 antibody therapy.

### 3. Prophylactic Methods

In one aspect, the present disclosure provides methods for preventing in a subject, a disease or condition associated with an unwanted or less than desirable immune response. Subjects at risk for a disease that would benefit from treatment with the claimed agents or methods can be identified, for example, by any or a combination of diagnostic or prognostic assays known in the art. Administration of a prophylactic agent can occur prior to the manifestation of symptoms associated with an unwanted or less than desirable immune response. The appropriate agent used for treatment (e.g. antibodies, peptides, fusion proteins or small molecules) can be determined based on clinical indications and can be identified, e.g., using screening assays described herein.

### 4. Prognostic Assays

The detection methods described herein can furthermore be utilized to identify subjects that will respond to a certain therapy, such as a therapy targeting KIR3DL3 for modulating the activity and/or interaction with a binding partner, such as HHLA2. Similarly, the prognostic assays described herein can be used to determine whether a subject can be administered an agent (e.g., an agonist, antagonist, peptidomimetic, polypeptide, peptide, nucleic acid, small molecule, or other drug candidate) to treat such a disorder associated with too much or too little KIR3DL3 activity. For example, such methods can be used to determine whether a subject can be effectively treated with one or a combination of agents. Thus, the present disclosure provides methods for determining whether a subject can be effectively treated with one or more agents for treating a disorder associated with too much or too little KIR3DL3 activity, in which a test sample is obtained and KIR3DL3 is detected. A test sample may be a biological sample obtained from a subject of interest. The test sample may be obtained from a subject of interest. For example, the sample may be a biological fluid (e.g., cerebrospinal fluid or serum), cell sample, or tissue, such as a histopathological slide of the tumor microenvironment, peritumoral area, and/or intratumoral area. In some embodiments, a test sample may comprise cells expressing mature membrane-bound KIR3DL3 and/or KIR3DL3 fragments.

The methods described herein may be performed, for example, by utilizing pre-packaged diagnostic kits comprising at least one antibody reagent described herein, which may be conveniently used, e.g., in clinical settings to prognose patients exhibiting symptoms or family history of a disease or illness involving KIR3DL3.

Furthermore, any cell type or tissue in which KIR3DL3 is expressed may be utilized in the prognostic assays described herein.

Another aspect of the present disclosure includes uses of the compositions and methods described herein for association and/or stratification analyses in which the KIR3DL3 in biological samples from individuals with a disorder associated with too much or too little KIR3DL3 activity, are analyzed and the information is compared to that of controls (e.g., individuals who do not have the disorder; controls may be also referred to as “healthy” or “normal” individuals or at early timepoints in a given time lapse study) who are preferably of similar age and race. Alternatively, the controls may be individuals who are afflicted with disorders with too much or too little KIR3DL3 activity, who have responded well to a therapy targeting KIR3DL3, e.g., a therapy that modulates the activity of KIR3DL3 or interaction of KIR3DL3 with one or more of its binding partners. Since, in some embodiments, the appropriate selection of patients and controls is useful for association and/or stratification studies, it may be desirable to have include a pool of individuals with well-characterized phenotypes is extremely desirable. Different study designs may be used for stratification studies (Modern Epidemiology, Lippincott Williams & Wilkins (1998), 609-622).

### VII. Pharmaceutical Compositions

Agents that modulate (e.g., inhibit or promote) the interaction between KIR3DL3 and one or more natural binding partners, such as HHLA2, including, e.g., blocking antibodies, peptides, fusion proteins, or small molecules, can be incorporated into pharmaceutical compositions suitable for administration to a subject. Such pharmaceutical compositions can further include additional components and/or therapeutic agents, such as those described herein. Pharmaceutical compositions typically comprise one or more agent(s) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary active compounds can also be incorporated into the compositions.

A pharmaceutical composition encompassed by the present disclosure is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor E L™ (BASF, Parsippany, NJ) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, modulatory agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms encompassed by the present disclosure are dictated by, and directly dependent on, the unique characteristics of the active compound, the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method encompassed by the present disclosure, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The above described modulating agents may be administered it he form of expressible nucleic acids which encode said agents. Such nucleic acids and compositions in which they are contained, are also encompassed by the present disclosure. For instance, the nucleic acid molecules encompassed by the present disclosure can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### VIII. Administration of Agents

The immune modulating agents encompassed by the present disclosure are administered to subjects in a biologically compatible form suitable for pharmaceutical administration in vivo, to either enhance or suppress immune cell mediated immune responses. By “biologically compatible form suitable for administration in vivo” is meant a form of the protein to be administered in which any toxic effects are outweighed by the therapeutic effects of the protein. Administration of an agent as described herein can be in any pharmacological form including a therapeutically active amount of an agent alone or in combination with a pharmaceutically acceptable carrier.

Administration of a therapeutically active amount of the therapeutic composition encompassed by the present disclosure is defined as an amount effective, at dosages and for periods of time necessary, to achieve the desired result. For example, a therapeutically active amount of an agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

The agents or the invention described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions of agents suitable for injectable use include sterile aqueous solutions (where water soluble) or dispersions and sterile powders for the extemporaneous preparation of sterile injectable solutions or dispersion. In all cases the composition will preferably be sterile and must be fluid to the extent that easy syringeability exists. It will preferably be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating an agent encompassed by the present disclosure (e.g., an antibody, peptide, fusion protein or small molecule) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the agent plus any additional desired ingredient from a previously sterile-filtered solution thereof.

When the agent is suitably protected, as described above, the protein can be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well-known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. “Dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms encompassed by the present disclosure are dictated by, and directly dependent on, (a) the unique characteristics of the active compound and the particular therapeutic effect to be achieved, and (b) the limitations inherent in the art of compounding such an active compound for the treatment of sensitivity in individuals.

In one embodiment, an agent encompassed by the present disclosure is an antibody. As defined herein, a therapeutically effective amount of antibody (i.e., an effective dosage) ranges from about 0.001 to 100 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays.

As described above, in some embodiments, agents for administration are cell-based. Cell-based agents have an immunocompatibility relationship to a subject host and any such relationship is contemplated for use according to the present disclosure. For example, the cells, such as adoptive T cells, can be syngeneic. The term “syngeneic” can refer to the state of deriving from, originating in, or being members of the same species that are genetically identical, particularly with respect to antigens or immunological reactions. These include identical twins having matching MHC types. Thus, a “syngeneic transplant” refers to transfer of cells from a donor to a recipient who is genetically identical to the donor or is sufficiently immunologically compatible as to allow for transplantation without an undesired adverse immunogenic response (e.g., such as one that would work against interpretation of immunological screen results described herein).

A syngeneic transplant can be “autologous” if the transferred cells are obtained from and transplanted to the same subject. An “autologous transplant” refers to the harvesting and reinfusion or transplant of a subject's own cells or organs. Exclusive or supplemental use of autologous cells may eliminate or reduce many adverse effects of administration of the cells back to the host, particular graft versus host reaction.

A syngeneic transplant can be “matched allogeneic” if the transferred cells are obtained from and transplanted to different members of the same species yet have sufficiently matched major histocompatibility complex (MHC) antigens to avoid an adverse immunogenic response. Determining the degree of MHC mismatch may be accomplished according to standard tests known and used in the art. For instance, there are at least six major categories of MHC genes in humans, identified as being important in transplant biology. HLA-A, HLA-B, HLA-C encode the HLA class I proteins while HLA-DR, HLA-DQ, and HLA-DP encode the HLA class II proteins. Genes within each of these groups are highly polymorphic, as reflected in the numerous HLA alleles or variants found in the human population, and differences in these groups between individuals is associated with the strength of the immune response against transplanted cells. Standard methods for determining the degree of MHC match examine alleles within HLA-B and HLA-DR, or HLA-A, HLA-B and HLA-DR groups. Thus, tests may be made of at least 4, and even 5 or 6 MHC antigens within the two or three HLA groups, respectively. In serological MHC tests, antibodies directed against each HLA antigen type are reacted with cells from one subject (e.g., donor) to determine the presence or absence of certain MHC antigens that react with the antibodies. This is compared to the reactivity profile of the other subject (e.g., recipient). Reaction of the antibody with an MHC antigen is typically determined by incubating the antibody with cells, and then adding complement to induce cell lysis (i.e., lymphocytotoxicity testing). The reaction is examined and graded according to the amount of cells lysed in the reaction (see, for example, Mickelson and Petersdorf (1999) Hematopoietic Cell Transplantation, Thomas, E. D. et al. eds., pg 28-37, Blackwell Scientific, Malden, Mass.). Other cell-based assays include flow cytometry using labeled antibodies or enzyme linked immunoassays (ELISA). Molecular methods for determining MHC type are well-known and generally employ synthetic probes and/or primers to detect specific gene sequences that encode the HLA protein. Synthetic oligonucleotides may be used as hybridization probes to detect restriction fragment length polymorphisms associated with particular HLA types (Vaughn (2002) Method. Mol. Biol. MHC Protocol. 210:45-60). Alternatively, primers may be used for amplifying the HLA sequences (e.g., by polymerase chain reaction or ligation chain reaction), the products of which may be further examined by direct DNA sequencing, restriction fragment polymorphism analysis (RFLP), or hybridization with a series of sequence specific oligonucleotide primers (SSOP) (Petersdorf et al. (1998) Blood 92:3515-3520; Morishima et al. (2002) Blood 99:4200-4206; and Middleton and Williams (2002) Method. Mol. Biol. MHC Protocol. 210:67-112).

A syngeneic transplant can be “congenic” if the transferred cells and cells of the subject differ in defined loci, such as a single locus, typically by inbreeding. The term “congenic” refers to deriving from, originating in, or being members of the same species, where the members are genetically identical except for a small genetic region, typically a single genetic locus (i.e., a single gene). A “congenic transplant” refers to transfer of cells or organs from a donor to a recipient, where the recipient is genetically identical to the donor except for a single genetic locus. For example, CD45 exists in several allelic forms and congenic mouse lines exist in which the mouse lines differ with respect to whether the CD45.1 or CD45.2 allelic versions are expressed.

By contrast, “mismatched allogeneic” refers to deriving from, originating in, or being members of the same species having non-identical major histocompatibility complex (MHC) antigens (i.e., proteins) as typically determined by standard assays used in the art, such as serological or molecular analysis of a defined number of MHC antigens, sufficient to elicit adverse immunogenic responses. A “partial mismatch” refers to partial match of the MHC antigens tested between members, typically between a donor and recipient. For instance, a “half mismatch” refers to 50% of the MHC antigens tested as showing different MHC antigen type between two members. A “full” or “complete” mismatch refers to all MHC antigens tested as being different between two members.

Similarly, in contrast, “xenogeneic” refers to deriving from, originating in, or being members of different species, e.g., human and rodent, human and swine, human and chimpanzee, etc. A “xenogeneic transplant” refers to transfer of cells or organs from a donor to a recipient where the recipient is a species different from that of the donor.

In addition, cells can be obtained from a single source or a plurality of sources (e.g., a single subject or a plurality of subjects). A plurality refers to at least two (e.g., more than one). In still another embodiment, the non-human mammal is a mouse. The animals from which cell types of interest are obtained may be adult, newborn (e.g., less than 48 hours old), immature, or in utero. Cell types of interest may be primary cancer cells, cancer stem cells, established cancer cell lines, immortalized primary cancer cells, and the like. In certain embodiments, the immune systems of host subjects can be engineered or otherwise elected to be immunological compatible with transplanted cancer cells. For example, in one embodiment, the subject may be “humanized” in order to be compatible with human cancer cells. The term “immune-system humanized” refers to an animal, such as a mouse, comprising human HSC lineage cells and human acquired and innate immune cells, survive without being rejected from the host animal, thereby allowing human hematopoiesis and both acquired and innate immunity to be reconstituted in the host animal. Acquired immune cells include T cells and B cells. Innate immune cells include macrophages, granulocytes (basophils, eosinophils, neutrophils), DCs, NK cells and mast cells. Representative, non-limiting examples include SCID-hu, Hu-PBL-SCID, Hu-SRC-SCID, NSG (NOD-SCID IL2r-gamma (null) lack an innate immune system, B cells, T cells, and cytokine signaling), NOG (NOD-SCID IL2r-gamma (truncated)), BRG (BALB/c-Rag2 (null) IL2r-gamma (null)), and H2dRG (Stock-H2d-Rag2 (null) IL2r-gamma (null)) mice (see, for example, Shultz et al. (2007) Nat. Rev. Immunol. 7:118; Pearson et al. (2008) Curr. Protocol. Immunol. 15:21; Brehm et al. (2010) Clin. Immunol. 135:84-98; McCune et al. (1988) Science 241:1632-1639, U.S. Pat. No. 7,960,175, and U.S. Pat. Publ. 2006/0161996), as well as related null mutants of immune-related genes like Rag1 (lack B and T cells), Rag2 (lack B and T cells), TCR alpha (lack T cells), perforin (CD8+ T cells lack cytotoxic function), FoxP3 (lack functional CD4+T regulatory cells), IL2rg, or Prfl, as well as mutants or knockouts of HHLA2, KIR3DL3, TMIGD2, PD-1, PD-L1, Tim3, and/or 2B4, allow for efficient engraftment of human immune cells in and/or provide compartment-specific models of immunocompromised animals like mice (see, for example, PCT Publ. WO2013/062134). In addition, NSG-CD34+ (NOD-SCID IL2r-gamma (null) CD34+) humanized mice are useful for studying human gene and tumor activity in animal models like mice.

As used herein, “obtained” from a biological material source means any conventional method of harvesting or partitioning a source of biological material from a donor. For example, biological material may obtained from a solid tumor, a blood sample, such as a peripheral or cord blood sample, or harvested from another body fluid, such as bone marrow or amniotic fluid. Methods for obtaining such samples are well-known to the artisan. In the present disclosure, the samples may be fresh (i.e., obtained from a donor without freezing). Moreover, the samples may be further manipulated to remove extraneous or unwanted components prior to expansion. The samples may also be obtained from a preserved stock. For example, in the case of cell lines or fluids, such as peripheral or cord blood, the samples may be withdrawn from a cryogenically or otherwise preserved bank of such cell lines or fluid. Such samples may be obtained from any suitable donor.

The obtained populations of cells may be used directly or frozen for use at a later date. A variety of mediums and protocols for cryopreservation are known in the art. Generally, the freezing medium will comprise DMSO from about 5-10%, 10⁻⁹⁰% serum albumin, and 50-90% culture medium. Other additives useful for preserving cells include, by way of example and not limitation, disaccharides such as trehalose (Scheinkoniget al. (2004) Bone Marrow Transplant. 34:531-536), or a plasma volume expander, such as hetastarch (i.e., hydroxyethyl starch). In some embodiments, isotonic buffer solutions, such as phosphate-buffered saline, may be used. An exemplary cryopreservative composition has cell-culture medium with 4% HSA, 7.5% dimethyl sulfoxide (DMSO), and 2% hetastarch. Other compositions and methods for cryopreservation are well-known and described in the art (see, e.g., Broxmeyer et al. (2003) Proc. Natl. Acad. Sci. U.S.A. 100:645-650). Cells are preserved at a final temperature of less than about −135° C. Cells can be administered at 0.1×10⁶, 0.2×10⁶, 0.3×10⁶, 0.4×10⁶, 0.5×10⁶, 0.6 ×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0 ×10⁸, or more, or any range in between or any value in between, cells per kilogram of subject body weight. The number of cells transplanted may be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted. In some embodiment, transplantation of at least about 0.1×10⁶, 0.5×10⁶, 1.0×10⁶, 2.0×10⁶, 3.0×10⁶, 4.0×10⁶, or 5.0×10⁶ total cells relative to an average size mouse is effective.

Cells can also be administered before, concurrently with, or after, other anti-cancer agents.

As described above, administration of agents like cells may be accomplished using methods generally known in the art, including, but not limited to, administration by intravascular, intracerebral, parenteral, intraperitoneal, intravenous, epidural, intraspinal, intrasternal, intra-articular, intra-synovial, intrathecal, intra-arterial, intracardiac, or intramuscular, intravenous, subcutaneous, specific tissue (e.g., focal transplantation), femur bone marrow cavity, spleen, renal capsule of fetal liver modes, and the like.

Engraftment of transplanted cells may be assessed by any of various methods, such as, but not limited to, tumor volume, cytokine levels, time of administration, flow cytometric analysis of cells of interest obtained from the subject at one or more time points following transplantation, and the like. For example, a time-based analysis of waiting 1, 2, 3, 4, 5, 6, 7, 8, 9, 10, 11, 12, 13, 14, 15, 16, 17, 18, 19, 20, 21, 22, 23, 24, 25, 26, 27, 28 days or can signal the time for tumor harvesting. Any such metrics are variables that can be adjusted according to well-known parameters in order to determine the effect of the variable on a response to anti-cancer immunotherapy. In addition, the transplanted cells can be co-transplanted with other agents, such as cytokines, extracellular matrices, cell culture supports, and the like.

### IX. Subjects

In some embodiments, the subject for whom the present disclosure (e.g., anti-KIR3DL3 antibodies, or antigen-binding fragment thereof) is used for therapy or immunomodulation, is a mammal (e.g., mouse, humanized mouse, rat, primate, non-human mammal, domestic animal, such as a dog, cat, cow, horse, and the like), and is preferably a human. In another embodiment, the subject is an animal model of cancer. For example, the animal model can be an orthotopic xenograft animal model of a human-derived cancer.

In another embodiment of the methods encompassed by the present disclosure, the subject has not undergone treatment, such as chemotherapy, radiation therapy, targeted therapy, and/or anti-immune checkpoint therapy. In still another embodiment, the subject has undergone treatment, such as chemotherapy, radiation therapy, targeted therapy, and/or anti-immune checkpoint therapy.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

The methods encompassed by the present disclosure can be used to determine the responsiveness to KIR3DL3 therapy and/or treat many different cancers in subjects such as those described herein.

### X. Sample Collection, Preparation and Separation

In some embodiments, biomarker amount and/or activity measurement(s) in a sample from a subject is compared to a predetermined control (standard) sample. The sample from the subject is typically from a diseased tissue, such as cancer cells or tissues. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects. In some embodiments, the biomarker amount and/or activity measurement(s) from a subject is compared to a pre-determined level. This pre-determined level is typically obtained from normal samples. As described herein, a “pre-determined” biomarker amount and/or activity measurement(s) may be a biomarker amount and/or activity measurement(s) used to, by way of example only, evaluate a subject that may be selected for treatment (e.g., based on the number of genomic mutations and/or the number of genomic mutations causing non-functional proteins for DNA repair genes), evaluate a response to an anti-KIR3DL3 antibody therapy, and/or evaluate a response to an anti-KIR3DL3 antibody therapy with one or more additional anti-cancer therapies. A pre-determined biomarker amount and/or activity measurement(s) may be determined in populations of patients with or without cancer. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements.

In another embodiment, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of anti-cancer therapy. Post-treatment biomarker measurement can be made at any time after initiation of anti-cancer therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of anti-cancer therapy, and even longer toward indefinitely for continued monitoring. Treatment can comprise anti-cancer therapy, such as a therapeutic regimen comprising one or more anti-KIR3DL3 antibodies alone or in combination with other anti-cancer agents, such as with immune checkpoint inhibitors.

The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments encompassed by the present disclosure the change of biomarker amount and/or activity measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

Biological samples can be collected from a variety of sources from a patient including a body fluid sample, cell sample, or a tissue sample comprising nucleic acids and/or proteins. “Body fluids” refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. In another embodiment, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present disclosure. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, and concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

Ultracentrifugation is a method for removing undesired polypeptides from a sample. Ultracentrifugation is the centrifugation of a sample at about 15,000-60,000 rpm while monitoring with an optical system the sedimentation (or lack thereof) of particles. Electrodialysis is a procedure which uses an electromembrane or semipermable membrane in a process in which ions are transported through semi-permeable membranes from one solution to another under the influence of a potential gradient. Since the membranes used in electrodialysis may have the ability to selectively transport ions having positive or negative charge, reject ions of the opposite charge, or to allow species to migrate through a semipermable membrane based on size and charge, it renders electrodialysis useful for concentration, removal, or separation of electrolytes. Separation and purification in the present disclosure may include any procedure known in the art, such as capillary electrophoresis (e.g., in capillary or on-chip) or chromatography (e.g., in capillary, column or on a chip). Electrophoresis is a method which can be used to separate ionic molecules under the influence of an electric field. Electrophoresis can be conducted in a gel, capillary, or in a microchannel on a chip. Examples of gels used for electrophoresis include starch, acrylamide, polyethylene oxides, agarose, or combinations thereof. A gel can be modified by its cross-linking, addition of detergents, or denaturants, immobilization of enzymes or antibodies (affinity electrophoresis) or substrates (zymography) and incorporation of a pH gradient. Examples of capillaries used for electrophoresis include capillaries that interface with an electrospray.

Capillary electrophoresis (CE) is preferred for separating complex hydrophilic molecules and highly charged solutes. CE technology can also be implemented on microfluidic chips. Depending on the types of capillary and buffers used, CE can be further segmented into separation techniques such as capillary zone electrophoresis (CZE), capillary isoelectric focusing (CIEF), capillary isotachophoresis (cITP) and capillary electrochromatography (CEC). An embodiment to couple CE techniques to electrospray ionization involves the use of volatile solutions, for example, aqueous mixtures containing a volatile acid and/or base and an organic such as an alcohol or acetonitrile.

Capillary isotachophoresis (cITP) is a technique in which the analytes move through the capillary at a constant speed but are nevertheless separated by their respective mobilities. Capillary zone electrophoresis (CZE), also known as free-solution CE (FSCE), is based on differences in the electrophoretic mobility of the species, determined by the charge on the molecule, and the frictional resistance the molecule encounters during migration which is often directly proportional to the size of the molecule. Capillary isoelectric focusing (CIEF) allows weakly-ionizable amphoteric molecules, to be separated by electrophoresis in a pH gradient. CEC is a hybrid technique between traditional high performance liquid chromatography (HPLC) and CE. Separation and purification techniques used in the present disclosure include any chromatography procedures known in the art. Chromatography can be based on the differential adsorption and elution of certain analytes or partitioning of analytes between mobile and stationary phases. Different examples of chromatography include, but not limited to, liquid chromatography (LC), gas chromatography (GC), high performance liquid chromatography (HPLC), etc.

### XI. Biomarker Polypeptides

Another aspect encompassed by the present disclosure pertains to the use of biomarker proteins and biologically active portions thereof. In one embodiment, the native polypeptide corresponding to a marker can be isolated from cells or tissue sources by an appropriate purification scheme using standard protein purification techniques. In another embodiment, polypeptides corresponding to a marker encompassed by the present disclosure are produced by recombinant DNA techniques. Alternative to recombinant expression, a polypeptide corresponding to a marker encompassed by the present disclosure can be synthesized chemically using standard peptide synthesis techniques.

Biologically active portions of a biomarker polypeptide include polypeptides comprising amino acid sequences sufficiently identical to or derived from a biomarker protein amino acid sequence described herein, but which includes fewer amino acids than the full length protein, and exhibit at least one activity of the corresponding full-length protein. Typically, biologically active portions comprise a domain or motif with at least one activity of the corresponding protein. A biologically active portion of a protein encompassed by the present disclosure can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide encompassed by the present disclosure.

Preferred polypeptides have an amino acid sequence of a biomarker protein encoded by a nucleic acid molecule described herein. Other useful proteins are substantially identical (e.g., at least about 40%, preferably 50%, 60%, 70%, 75%, 80%, 83%, 85%, 88%, 90%, 91%, 92%, 93%, 94%, 95%, 96%, 97%, 98%, or 99%) to one of these sequences and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity= # of identical positions/total # of positions (e.g., overlapping positions) x100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules encompassed by the present disclosure. BLAST protein searches can be performed with the XBLAST program, score=50, wordlength=3 to obtain amino acid sequences homologous to a protein molecules encompassed by the present disclosure. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The present disclosure also provides chimeric or fusion proteins corresponding to a biomarker protein. As used herein, a “chimeric protein” or “fusion protein” comprises all or part (preferably a biologically active part) of a polypeptide corresponding to a marker encompassed by the present disclosure operably linked to a heterologous polypeptide (i.e., a polypeptide other than the polypeptide corresponding to the marker). Within the fusion protein, the term “operably linked” is intended to indicate that the polypeptide encompassed by the present disclosure and the heterologous polypeptide are fused in-frame to each other. The heterologous polypeptide can be fused to the amino-terminus or the carboxyl-terminus of the polypeptide encompassed by the present disclosure.

One useful fusion protein is a GST fusion protein in which a polypeptide corresponding to a marker encompassed by the present disclosure is fused to the carboxyl terminus of GST sequences. Such fusion proteins can facilitate the purification of a recombinant polypeptide encompassed by the present disclosure.

In another embodiment, the fusion protein contains a heterologous signal sequence, immunoglobulin fusion protein, toxin, or other useful protein sequence. Chimeric and fusion proteins encompassed by the present disclosure can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide encompassed by the present disclosure can be cloned into such an expression vector such that the fusion moiety is linked in-frame to the polypeptide encompassed by the present disclosure.

A signal sequence can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the present disclosure pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present disclosure also pertains to variants of the biomarker polypeptides described herein. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a biomarker protein which function as either agonists (mimetics) or as antagonists can be identified by screening combinatorial libraries of mutants, e.g., truncation mutants, of the protein encompassed by the present disclosure for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides encompassed by the present disclosure from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker encompassed by the present disclosure can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein encompassed by the present disclosure (Arkin and Yourvan, 1992, Proc. Natl. Acad. Sci. USA 89:7811-7815; Delgrave et al., 1993, Protein Engineering 6 (3): 327-331).

An isolated polypeptide or a fragment thereof (or a nucleic acid encoding such a polypeptide) corresponding to one or more biomarkers encompassed by the present disclosure, including the biomarkers listed in Table 1 or fragments thereof, can be used as an immunogen to generate antibodies that bind to said immunogen, using standard techniques for polyclonal and monoclonal antibody preparation according to well-known methods in the art. An antigenic peptide comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein).

In one embodiment, an antibody binds substantially specifically to KIR3DL3 and inhibits or blocks its function, such as by interrupting its interaction with HHLA2.

For example, a polypeptide immunogen typically is used to prepare antibodies by immunizing a suitable subject (e.g., rabbit, goat, mouse, humanized mouse or other mammal) with the immunogen. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique (originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well-known (see generally Kenneth, R. H. in Monoclonal Antibodies: A New Dimension In Biological Analyses, Plenum Publishing Corp., New York, New York (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically. In some embodiments, the immunization is performed in a cell or animal host that has a knockout of a target antigen of interest (e.g., does not produce the antigen prior to immunization).

Any of the many well-known protocols used for fusing lymphocytes and immortalized cell lines can be applied for the purpose of generating a monoclonal antibody against one or more biomarkers encompassed by the present disclosure, including the biomarkers listed in Table 1, or a fragment thereof (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation encompassed by the present disclosure with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, MD. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody encompassed by the present disclosure are detected by screening the hybridoma culture supernatants for antibodies that bind a given polypeptide, e.g., using a standard ELISA assay.

As an alternative to preparing monoclonal antibody-secreting hybridomas, a monoclonal specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

The structural features of non-human or human antibodies (e.g., a rat anti-mouse/anti-human antibody) can be used to create structurally related human antibodies that retain at least one functional property of the antibodies encompassed by the present disclosure, such as binding to KIR3DL3. Another functional property includes inhibiting binding of the original known, non-human or human antibodies in a competition ELISA assay.

In some embodiments, monoclonal antibodies capable of binding and inhibiting/blocking KIR3DL3 are provided, comprising a heavy chain wherein the variable domain comprises at least a CDR having a sequence that is at least 80%, 85%, 90%, 91%, 92%, 93%, 94%, 95%, 96%, 97%, 98%, 99%, 99.5% or 100% identical from the group of heavy chain variable domain CDRs presented herein or otherwise publicly available.

Similarly, monoclonal antibodies binding and inhibiting/blocking KIR3DL3, comprising a light chain wherein the variable domain comprises at least a CDR having a sequence that is at least 80%, 85%, 90%, 91%, 92%, 93%, 94%, 95%, 96%, 97%, 98%, 99%, 99.5% or 100% identical from the group of light chain variable domain CDRs presented herein or otherwise publicly available, are also provided.

Monoclonal antibodies capable of binding and inhibiting/blocking KIR3DL3, comprising a heavy chain wherein the variable domain comprises at least a CDR having a sequence that is at least 80%, 85%, 90%, 91%, 92%, 93%, 94%, 95%, 96%, 97%, 98%, 99%, 99.5% or 100% identical from the group of heavy chain variable domain CDRs presented herein or otherwise publicly available; and comprising a light chain wherein the variable domain comprises at least a CDR having a sequence that is at least 80%, 85%, 90%, 91%, 92%, 93%, 94%, 95%, 96%, 97%, 98%, 99%, 99.5% or 100% identical from the group of light chain variable domain CDRs presented herein or otherwise publicly available, are also provided.

A skilled artisan will note that such percentage homology is equivalent to and can be achieved by introducing 1, 2, 3, 4, 5, 6, 7, 8, 9, 10, or more conservative amino acid substitutions within a given CDR.

Additionally, fully human antibodies could be made against biomarkers encompassed by the present disclosure, including the biomarkers listed in Table 1, or fragments thereof. Fully human antibodies can be made in mice that are transgenic for human immunoglobulin genes, e.g. according to Hogan, et al., “Manipulating the Mouse Embryo: A Laboratory Manuel,” Cold Spring Harbor Laboratory. Briefly, transgenic mice are immunized with purified immunogen. Spleen cells are harvested and fused to myeloma cells to produce hybridomas. Hybridomas are selected based on their ability to produce antibodies which bind to the immunogen. Fully human antibodies would reduce the immunogenicity of such antibodies in a human.

In one embodiment, an antibody for use in the instant invention is a bispecific or multispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986) Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a polypeptide or a fragment thereof of one or more biomarkers encompassed by the present disclosure, including one or more biomarkers listed in Table 1, or a fragment thereof. In one embodiment, the bispecific antibody could specifically bind to both a polypeptide or a fragment thereof and its natural binding partner(s) or a fragment(s) thereof.

In another aspect of this invention, peptides or peptide mimetics can be used to antagonize the activity of one or more biomarkers encompassed by the present disclosure, including one or more biomarkers listed in Table 1, or a fragment(s) thereof. In one embodiment, variants of one or more biomarkers listed in Table 1 which function as a modulating agent for the respective full length protein, can be identified by screening combinatorial libraries of mutants, e.g., truncation mutants, for antagonist activity. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad. Sci. USA 89:7811-7815; Delagrave et al. (1993) Protein Eng. 6 (3): 327-331). In one embodiment, cell based assays can be exploited to analyze a variegated polypeptide library. For example, a library of expression vectors can be transfected into a cell line which ordinarily synthesizes one or more biomarkers encompassed by the present disclosure, including one or more biomarkers listed in Table 1, or a fragment thereof. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences disclosed herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well-known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments encompassed by the present disclosure. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides disclosed herein can be used therapeutically to treat disease, e.g., by altering costimulation in a patient.

Peptidomimetics (Fauchere (1986) Adv. Drug Res. 15:29; Veber and Freidinger (1985) TINS p.392; and Evans et al. (1987) J. Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of:-CH2NH—, -CH2S-, -CH2-CH2-, -CH═CH-(cis and trans), -COCH2-, -CH (OH) CH2—, and—CH2SO-, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (-CH2NH—, CH2CH2-); Spatola, A. F. et al. (1986) Life Sci. 38:1243-1249 (-CH2-S); Hann, M. M. (1982) J. Chem. Soc. Perkin Trans. I. 307-314 (-CH-CH—, cis and trans); Almquist, R. G. et al. (190) J. Med. Chem. 23:1392-1398 (-COCH2-); Jennings-White, C. et al. (1982) Tetrahedron Lett. 23:2533 (-COCH2-); Szelke, M. et al. European Appln. EP 45665 (1982) CA: 97:39405 (1982) (-CH(OH)CH2-); Holladay, M. W. et al. (1983) Tetrahedron Lett. (1983) 24:4401-4404 (-C(OH) CH2-); and Hruby, V. J. (1982) Life Sci. (1982) 31:189-199 (-CH2—S—); each of which is incorporated herein by reference. A particularly preferred non-peptide linkage is-CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivatization (e.g., labeling) of peptidomimetics should not substantially interfere with the desired biological or pharmacological activity of the peptidomimetic.

Also encompassed by the present disclosure are small molecules which can modulate (either enhance or inhibit) interactions, e.g., between biomarkers described herein or listed in Table 1 and their natural binding partners. The small molecules encompassed by the present disclosure can be obtained using any of the numerous approaches in combinatorial library methods known in the art, including: spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.). Compounds can be screened in cell based or non-cell based assays. Compounds can be screened in pools (e.g. multiple compounds in each testing sample) or as individual compounds.

The invention also relates to chimeric or fusion proteins of the biomarkers encompassed by the present disclosure, including the biomarkers listed in Table 1, or fragments thereof. As used herein, a “chimeric protein” or “fusion protein” comprises one or more biomarkers encompassed by the present disclosure, including one or more biomarkers listed in Table 1, or a fragment thereof, operatively linked to another polypeptide having an amino acid sequence corresponding to a protein which is not substantially homologous to the respective biomarker. In a preferred embodiment, the fusion protein comprises at least one biologically active portion of one or more biomarkers encompassed by the present disclosure, including one or more biomarkers listed in Table 1, or fragments thereof. Within the fusion protein, the term “operatively linked” is intended to indicate that the biomarker sequences and the non-biomarker sequences are fused in-frame to each other in such a way as to preserve functions exhibited when expressed independently of the fusion. The “another” sequences can be fused to the N-terminus or C-terminus of the biomarker sequences, respectively.

Such a fusion protein can be produced by recombinant expression of a nucleotide sequence encoding the first peptide and a nucleotide sequence encoding the second peptide. The second peptide may optionally correspond to a moiety that alters the solubility, affinity, stability or valency of the first peptide, for example, an immunoglobulin constant region. In another preferred embodiment, the first peptide consists of a portion of a biologically active molecule (e.g. the extracellular portion of the polypeptide or the ligand binding portion). The second peptide can include an immunoglobulin constant region, for example, a human Cγ1 domain or Cγ4 domain (e.g., the hinge, CH2 and CH3 regions of human IgCγ 1, or human IgCγ4, see e.g., Capon et al. U.S. Pat. Nos. 5,116,964; 5,580,756; 5,844,095 and the like, incorporated herein by reference). Such constant regions may retain regions which mediate effector function (e.g. Fc receptor binding) or may be altered to reduce effector function. A resulting fusion protein may have altered solubility, binding affinity, stability and/or valency (i.e., the number of binding sites available per polypeptide) as compared to the independently expressed first peptide, and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well-known in the art. Protein and peptides can be isolated from cell culture media, host cells, or both using techniques known in the art for purifying proteins and peptides. Techniques for transfecting host cells and purifying proteins and peptides are known in the art.

Preferably, a fusion protein encompassed by the present disclosure is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992).

Particularly preferred Ig fusion proteins include the extracellular domain portion or variable region-like domain of one or more biomarker listed in Table 1, coupled to an immunoglobulin constant region (e.g., the Fc region). The immunoglobulin constant region may contain genetic modifications which reduce or eliminate effector activity inherent in the immunoglobulin structure. For example, DNA encoding the extracellular portion of a polypeptide of interest can be joined to DNA encoding the hinge, CH2 and CH3 regions of human IgGγ1 and/or IgGγ4 modified by site directed mutagenesis, e.g., as taught in WO 97/28267.

In another embodiment, the fusion protein contains a heterologous signal sequence at its N-terminus. In certain host cells (e.g., mammalian host cells), expression and/or secretion of a polypeptide can be increased through use of a heterologous signal sequence.

The fusion proteins encompassed by the present disclosure can be used as immunogens to produce antibodies in a subject. Such antibodies may be used to purify the respective natural polypeptides from which the fusion proteins were generated, or in screening assays to identify polypeptides which inhibit the interactions between one or more biomarkers polypeptide or a fragment thereof and its natural binding partner(s) or a fragment(s) thereof.

The modulatory agents described herein (e.g., antibodies, small molecules, peptides, fusion proteins, or small nucleic acids) can be incorporated into pharmaceutical compositions and administered to a subject in vivo. The compositions may contain a single such molecule or agent or any combination of agents described herein. “Single active agents” described herein can be combined with other pharmacologically active compounds (“second active agents”) known in the art according to the methods and compositions provided herein.

The production and use of biomarker nucleic acid and/or biomarker polypeptide molecules described herein can be facilitated by using standard recombinant techniques. In some embodiments, such techniques use vectors, preferably expression vectors, containing a nucleic acid encoding a biomarker polypeptide or a portion of such a polypeptide. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the present disclosure is intended to include such other forms of expression vectors, such as viral vectors (e.g., replication defective retroviruses, adenoviruses and adeno-associated viruses), which serve equivalent functions.

The recombinant expression vectors encompassed by the present disclosure comprise a nucleic acid encompassed by the present disclosure in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, CA (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors encompassed by the present disclosure can be introduced into host cells to thereby produce proteins or peptides, including fusion proteins or peptides, encoded by nucleic acids as described herein.

The recombinant expression vectors for use in the present disclosure can be designed for expression of a polypeptide corresponding to a marker encompassed by the present disclosure in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, MA) and pRIT5 (Pharmacia, Piscataway, NJ) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology: Methods in Enzymology vol. 185, Academic Press, San Diego, CA, 1991). Target biomarker nucleic acid expression from the pTrc vector relies on host RNA polymerase transcription from a hybrid trp-lac fusion promoter. Target biomarker nucleic acid expression from the pET 11d vector relies on transcription from a T7 gn10-lac fusion promoter mediated by a co-expressed viral RNA polymerase (T7 gn1). This viral polymerase is supplied by host strains BL21 (DE3) or HMS174 (DE3) from a resident prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman, p. 119-128, In Gene Expression Technology: Methods in Enzymology vol. 185, Academic Press, San Diego, CA, 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences encompassed by the present disclosure can be carried out by standard DNA synthesis techniques.

In another embodiment, the expression vector is a yeast expression vector. Examples of vectors for expression in yeast S. cerevisiae include pYepSecl (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, CA), and pPicZ (Invitrogen Corp, San Diego, CA).

Alternatively, the expression vector is a baculovirus expression vector. Baculovirus vectors available for expression of proteins in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid encompassed by the present disclosure is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The present disclosure further provides a recombinant expression vector comprising a DNA molecule cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide encompassed by the present disclosure. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes (see Weintraub et al., 1986, Trends in Genetics, Vol. 1 (1)).

Another aspect encompassed by the present disclosure pertains to host cells into which a recombinant expression vector encompassed by the present disclosure has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

### XII. Analyzing Biomarker Nucleic Acids and Polypeptides

Biomarker nucleic acids and/or biomarker polypeptides can be analyzed according to the methods described herein and techniques known to the skilled artisan to identify such genetic or expression alterations useful for the present disclosure including, but not limited to, 1) an alteration in the level of a biomarker transcript or polypeptide, 2) a deletion or addition of one or more nucleotides from a biomarker gene, 4) a substitution of one or more nucleotides of a biomarker gene, 5) aberrant modification of a biomarker gene, such as an expression regulatory region, and the like.

a. Methods for Detection of Copy Number

Methods of evaluating the copy number of a biomarker nucleic acid are well-known to those of skill in the art. The presence or absence of chromosomal gain or loss can be evaluated simply by a determination of copy number of the regions or markers identified herein.

Methods of evaluating the copy number of a biomarker locus include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the biomarker gene copy number in a sample involves a Southern Blot. In a Southern Blot, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well-known in the art to detect RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152:649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-I DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well-known in the art (see, e.g., U.S. Pat. Nos. 6,335,167; 6,197,501; 5,830,645; and 5,665,549 and Albertson (1984) EMBO J. 3:1227-1234; Pinkel (1988) Proc. Natl. Acad. Sci. USA 85:9138-9142; EPO Pub. No. 430,402; Methods in Molecular Biology, Vol. 33: In situ Hybridization Protocols, Choo, ed., Humana Press, Totowa, N. J. (1994), etc.) In another embodiment, the hybridization protocol of Pinkel, et al. (1998) Nature Genetics 20:207-211, or of Kallioniemi (1992) Proc. Natl Acad Sci USA 89:5321-5325 (1992) is used.

In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well-known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods encompassed by the present disclosure. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4:560, Landegren, et al. (1988) Science 241:1077, and Barringer et al. (1990) Gene 89:117), transcription amplification (Kwoh, et al. (1989) Proc. Natl. Acad. Sci. USA 86:1173), self-sustained sequence replication (Guatelli, et al. (1990) Proc. Nat. Acad. Sci. USA 87:1874), dot PCR, and linker adapter PCR, etc.

Loss of heterozygosity (LOH) and major copy proportion (MCP) mapping (Wang, Z. C., et al. (2004) Cancer Res 64 (1): 64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93; Li et al., (2008) MBC Bioinform. 9, 204-219) may also be used to identify regions of amplification or deletion.

b. Methods for Detection of Biomarker Nucleic Acid Expression

Biomarker expression may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In preferred embodiments, activity of a particular gene is characterized by a measure of gene transcript (e.g. mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In another embodiment, detecting or determining expression levels of a biomarker and functionally similar homologs thereof, including a fragment or genetic alteration thereof (e.g., in regulatory or promoter regions thereof) comprises detecting or determining RNA levels for the marker of interest. In one embodiment, one or more cells from the subject to be tested are obtained and RNA is isolated from the cells. In a preferred embodiment, a sample of breast tissue cells is obtained from the subject.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278:1481; Emmert-Buck et al. (1996) Science 274:998; Fend et al. (1999) Am. J. Path. 154:61 and Murakami et al. (2000) Kidney Int. 58:1346). For example, Murakami et al., supra, describe isolation of a cell from a previously immunostained tissue section. It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly (A)+RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, NY).

In a preferred embodiment, the RNA population is enriched in marker sequences. Enrichment can be undertaken, e.g., by primer-specific cDNA synthesis, or multiple rounds of linear amplification based on cDNA synthesis and template-directed in vitro transcription (see, e.g., Wang et al. (1989) PNAS 86, 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope encompassed by the present disclosure to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4:80-84 (1994). Real time PCR may also be used.

Other known amplification methods which can be utilized herein include but are not limited to the so-called “NASBA” or “3SR” technique described in PNAS USA 87:1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42:9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Many techniques are known in the state of the art for determining absolute and relative levels of gene expression, commonly used techniques suitable for use in the present disclosure include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a subject may be hybridized to a solid surface comprising biomarker DNA. Positive hybridization signal is obtained with the sample containing biomarker transcripts. Methods of preparing DNA arrays and their use are well-known in the art (see, e.g., U.S. Pat. Nos: 6,618,6796; 6,379,897; 6,664,377; 6,451,536; 548,257; US20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. patent application No. 20030215858).

To monitor mRNA levels, for example, mRNA is extracted from the biological sample to be tested, reverse transcribed, and fluorescently-labeled cDNA probes are generated. The microarrays capable of hybridizing to marker cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize marker mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, 32P and ³⁵S. Labeling with radioisotopes may be achieved, whether the probe is synthesized chemically or biologically, by the use of suitably labeled bases.

In one embodiment, the biological sample contains polypeptide molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting marker polypeptide, mRNA, genomic DNA, or fragments thereof, such that the presence of the marker polypeptide, mRNA, genomic DNA, or fragments thereof, is detected in the biological sample, and comparing the presence of the marker polypeptide, mRNA, genomic DNA, or fragments thereof, in the control sample with the presence of the marker polypeptide, mRNA, genomic DNA, or fragments thereof in the test sample.

c. Methods for Detection of Biomarker Protein Expression

The activity or level of a biomarker protein can be detected and/or quantified by detecting or quantifying the expressed polypeptide. The polypeptide can be detected and quantified by any of a number of means well-known to those of skill in the art. Aberrant levels of polypeptide expression of the polypeptides encoded by a biomarker nucleic acid and functionally similar homologs thereof, including a fragment or genetic alteration thereof (e.g., in regulatory or promoter regions thereof) are associated with the likelihood of response of a cancer to anti-KIR3DL3 antibody therapy. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference). Preferred are binder-ligand immunoassay methods including reacting antibodies with an epitope or epitopes and competitively displacing a labeled polypeptide or derivative thereof. In certain embodiments, the antibodies listed in table 2 are used to detect and/or quantify the biomarkers listed in table 1.

For example, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope-or enzyme-labeled anti-biomarker protein antibody is allowed to react with the system, and radioactivity or the enzyme assayed (ELISA-sandwich assay). Other conventional methods may also be employed as suitable.

In some embodiments, antibodies encompassed by the present disclosure, can be used in any one of well-known immunoassay forms, including, without limitation, a radioimmunoassay, a Western blot assay, an immunofluorescence assay, an enzyme immunoassay, an immunoprecipitation assay, a chemiluminescence assay, an immunohistochemical assay, a dot blot assay, or a slot blot assay. General techniques to be used in performing the various immunoassays noted above and other variations of the techniques, such as in situ proximity ligation assay (PLA), fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA), ELISA, etc. alone or in combination or alternatively with NMR, MALDI-TOF, LC-MS/MS, are known to those of ordinary skill in the art.

Such reagents can also be used to monitor protein levels in a cell or tissue, e.g., white blood cells or lymphocytes, as part of a clinical testing procedure, e.g., in order to monitor an optimal dosage of an inhibitory agent. Detection can be facilitated by coupling (e.g., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring biomarker protein levels comprises the steps of: contacting a biological specimen with an antibody or variant (e.g., fragment) thereof which selectively binds the biomarker protein, and detecting whether said antibody or variant thereof is bound to said sample and thereby measuring the levels of the biomarker protein.

Enzymatic and radiolabeling of biomarker protein and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect biomarker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-biomarker protein antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of biomarker protein, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-biomarker protein antibodies (e.g., listed in table 2), such as intrabodies, may also be used for imaging purposes, for example, to detect the presence of biomarker protein in cells and tissues of a subject. Suitable labels include radioisotopes, iodine (¹²⁵I, ¹²¹I), carbon (¹⁴C), sulphur (³⁵S), tritium (³H), indium (¹¹² In), and technetium (⁹⁹mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the subject, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99. The labeled antibody or antibody fragment will then preferentially accumulate at the location of cells which contain biomarker protein. The labeled antibody or antibody fragment can then be detected using known techniques.

Antibodies that may be used to detect biomarker protein include any antibody (e.g., listed in table 2), whether natural or synthetic, full length or a fragment thereof, monoclonal or polyclonal, that binds sufficiently strongly and specifically to the biomarker protein to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the biomarker protein relative to other proteins, such as related proteins.

Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′)2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10:1455-1460 (1992), regarding primatized antibody, and Ladner et al., U.S. Pat. No. 4,946,778 and Bird, R. E. et al., Science, 242:423-426 (1988)) regarding single-chain antibodies. Antibodies produced from a library, e.g., phage display library, may also be used.

In some embodiments, agents that specifically bind to a biomarker protein other than antibodies are used, such as peptides. Peptides that specifically bind to a biomarker protein can be identified by any means known in the art. For example, specific peptide binders of a biomarker protein can be screened for using peptide phage display libraries.

d. Methods for Detection of Biomarker Structural Alterations

The following illustrative methods can be used to identify the presence of a structural alteration in a biomarker nucleic acid and/or biomarker polypeptide molecule in order to, for example, identify HHLA2 or KIR3DL3 that is overexpressed, overfunctional, and the like.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a biomarker nucleic acid such as a biomarker gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a biomarker gene under conditions such that hybridization and amplification of the biomarker gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

In an alternative embodiment, mutations in a biomarker nucleic acid from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in biomarker nucleic acid can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, biomarker genetic mutations can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such biomarker genetic mutations can be identified in a variety of contexts, including, for example, germline and somatic mutations.

In yet another embodiment, any of a variety of sequencing reactions known in the art can be used to directly sequence a biomarker gene and detect mutations by comparing the sequence of the sample biomarker with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad Sci. USA 74:5463. It is also contemplated that any of a variety of automated sequencing procedures can be utilized when performing the diagnostic assays (Naeve (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a biomarker gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type biomarker sequence with potentially mutant RNA or DNA obtained from a tissue sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with SI nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in biomarker cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a biomarker sequence, e.g., a wild-type biomarker treated with a DNA mismatch repair enzyme, and the cleavage products, if any, can be detected from electrophoresis protocols or the like (e.g., U.S. Pat. No. 5,459,039.)

In other embodiments, alterations in electrophoretic mobility can be used to identify mutations in biomarker genes. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat. Res. 285:125-144 and Hayashi (1992) Genet. Anal. Tech. Appl. 9:73-79). Single-stranded DNA fragments of sample and control biomarker nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

### XIII. Clincal Efficacy

Clinical efficacy can be measured by any method known in the art. For example, the response to a therapy, such as anti-KIR3DL3 antibody therapy, relates to any respons, such as of cancer, e.g., a tumor, to a therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to anti-immune checkpoint therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any anti-immune checkpoint therapy. The outcome measurement may be pathologic response to therapy given in the neoadjuvant setting. Alternatively, outcome measures, such as overall survival and disease-free survival can be monitored over a period of time for subjects following anti-immune checkpoint therapy for whom biomarker measurement values are known. In certain embodiments, the same doses of anti-immune checkpoint agents are administered to each subject. In related embodiments, the doses administered are standard doses known in the art for anti-immune checkpoint agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of an anti-immune checkpoint therapy can be determined using methods such as those described in the Examples section.

### XIV. Kits

In addition, the present disclosure also encompasses kits for detecting the presence of a KIR3DL3 polypeptide, or fragments thereof, in a biological sample. For example, the kit can comprise a labeled compound or agent capable of detecting a KIR3DL3 polypeptide, or fragments thereof, in a biological sample; means for determining the amount of the KIR3DL3 polypeptide, or fragments thereof, in the sample; and means for comparing the amount of the KIR3DL3 polypeptide, or fragments thereof, in the sample with a standard. Alternatively, the kit can comprise one or more anti-KIR3DL3 antibodies and/or anti-HHLA2 antibodies (e.g., those described herein) for use in prognostic, therapeutic, and/or immunomodulatory methods. The compound or agent can be packaged in a suitable container. Kits encompassed by the present disclosure can contain an antibody coupled to a solid support, e.g., a tissue culture plate or beads (e.g., sepharose beads).

A kit can include additional components to facilitate the particular application for which the kit is designed. For example, kits can be provided which contain antibodies for detection and quantification of KIR3DL3 in vitro, e.g. in an ELISA or a Western blot. Additional, exemplary agents that kits can contain include means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or KIR3DL3 protein standards). A kit may additionally include buffers and other reagents recognized for use in a method of the disclosed invention. Non-limiting examples include agents to reduce non-specific binding, such as a carrier protein or a detergent. A kit encompassed by the present disclosure can also include instructional materials disclosing or describing the use of the kit or an antibody of the disclosed invention in a method of the disclosed invention as provided herein.

This invention is further illustrated by the following examples which should not be construed as limiting. The contents of all references, patents and published patent applications cited throughout this application, as well as the Figures, are incorporated herein by reference.

### Examples

### Example 1: Materials and Methods

### Expression Screen to Identify Novel Receptors for HHLA2

Cell microarray technology was used to identify novel HHLA2 receptors (Retrogenix, High Peak, UK). A library of ˜5500 full length cDNA clones covering more than 3,500 different plasma membrane proteins was arrayed in duplicate across 13 microarray slides (“slide sets”). Human HEK293 cells were grown above the cDNA clones and reverse-transfected. An expression vector (pIRES-hEGFR-IRES-ZsGreen1) was spotted in quadruplicate on every slide and was used to ensure that a minimal threshold of transfection efficiency had been achieved or exceeded on every slide. The resultant cell microarrays were evaluated for binding to soluble human HHLA2-mIgG2a fusion protein.

Human HHLA2-mIgG2a fusion protein was added to fixed cell microarray slides at a 20 μg/ml concentration, and binding interactions were detected with an AF647 labeled anti-mouse IgG detection antibody. Two replicate slides were screened for each of the 13 slide sets. Fluorescent images were analyzed and quantitated (for transfection efficiency) using ImageQuant software (GE). A protein ‘hit’ was defined as duplicate spots showing a raised signal compared to background levels. This was achieved by visual inspection using the images gridded on the ImageQuant software.

In order to determine which hit(s), if any, were reproducible and specific to human HHLA2, vectors encoding all library screening hits, plus appropriate control KIR receptors, were arrayed and expressed in HEK293 cells on new slides. Identical slides were screened with soluble HHLA2-mIgG2a, using the doses and incubation conditions used in the library screens or appropriate positive and negative control treatments (n=2 slides per treatment).

### KIR3DL3 Antibody Specificity Assay

Replicate microarray slides expressing a complete KIR family cDNA panel were fixed and blocked with buffer containing PBS/0.5% BSA and incubated for 1 hour at room temperature with individual KIR3DL3 mAb hybridoma supernatants at 1:5, 1:25 and 1:250 dilutions in PBS/0.1% BSA. Cell arrays were washed with PBS and incubated for 1 hour at room temperature in PBS/0.1% BSA containing AF647-conjugated goat anti-mouse IgG (H+L) (Life Technologies, A21235). Slides were washed with PBS, dried, and imaged for ZsGreen1 and AF647 fluorescence. HHLA2-mIgG2a (20 μg/ml) was used as a positive control to detect binding to KIR3DL3 and TMIGD2 on the spotted array.

### Receptor Binding and Blocking Assay

KIR3DL3 mAbs were pre-incubated with KIR3DL3 transfected 300.19 cells for 30 minutes at 4° C. HHLA2-mouse IgG2a (mutated at IgG2a L235E, E318A, K320A, K322A) (25 μl of 10 μg/ml) was added and incubation continued for 30 minutes at 4° C. Cells were washed and binding of HHLA2-mouse IgG2a was detected with 5 μg/ml Alexa 647 conjugated 298.6F8 mAb (mouse antibody specific for mouse IgG2a mutated at L235E, E318A, K320A, K322A). EC50 and IC50 analysis were conducted using GraphPad Prism® 8.

### Cell Lines and Cell Culture

Raji cell line (ATCC CCL-86), Jurkat (clone E6-1) (ATCC TIB-152), CHO-K1 (ATCC CCL-61), A498 (ATCC HTB-44), 786-0 (ATCC CRL-1932) and K562 (ATCC CCL-243) and NK-92 MI (ATCC CRL-2408) cells were obtained from American Type Culture Collection (ATCC, Manassas, VA, USA). 786-0, A498, Raji, Jurkat and K562 cells were incubated at 37° C. with 5% CO₂ in RPMI1640 medium (Life Technologies A10491-01) supplemented with 10% fetal bovine serum (FBS, Life Technologies 26140-079), 1% penicillin/streptomycin (Hyclone SV30010.01). NK-92 MI cells were cultured at 37° C. with 5% CO₂ using X-VIVO 15TM serum-free Hematopoietic Cell Medium (Lonza 04-418Q) supplemented with 10% FBS (Life Technologies 26140-079) and supplemented with 10% human serum (Sigma Aldrich H3667-100ML), 1% penicillin/streptomycin (Hyclone SV30010.01). Where indicated, cells were treated with IFN-Y (R&D #285-IF/CF; 10 ng/ml), IL-10 (R&D #1064-IF/CF; 10 ng/ml) or TGF-β1 (R&D #4454-BH; 10 ng/ml).

TMIGD2-NFAT-Jurkat stable cell line culture was supplemented with 1000 μg/ml of Geneticin™ (Life Technologies 11811031), and 200 μg/ml hygromycin (Invitrogen 10687010) to ensure recombinant expression of TMIGD2 and NFAT reporter is maintained. KIR3DL3-IL2-Jurkat stable cell line culture was supplemented with 1000 μg/ml of Geneticin™, and 0.25 μg/ml puromycin (InvivoGen ant-pr-1) to ensure expression of KIR3DL3 and IL-2 reporter is maintained. K562 HHLA2 stable cell line was supplemented with 1 μg/mL of puromycin. CHO cells were maintained in F12-K (Hyclone SH30526.01) medium supplemented with 10% FBS and 1% penicillin/streptomycin. HHLA2-anti-CD3 scFV-CHO stable cell line culture was supplemented with 1000 μg/ml of Geneticin™, and 500 μg/ml hygromycin to ensure recombinant expression of HHLA2 and TCR activator is maintained.

### Activation and Culture of Human T and NK Cells

RosetteSep™ Human T Cell Enrichment Cocktail (Stemcell #15021) was used to isolate T cells by negative selection from the blood of healthy donors. T cells were activated using ImmunoCult™ Human CD3/CD28 T Cell Activator tetramers following the manufacturer's recommended protocol (Stemcell 10971) and cultured using ImmunoCult™-XF T Cell Expansion Medium (Stemcell 10981) in the presence of 100 U/ml of IL-2 (Peprotech #200-02). At the indicated times, T cells were stained with the following antibodies: Alexa 647 1G7 antibody (KIR3DL3 antibody) or Alexa 647 Mouse IgG2b, K isotype control (Biolegend #400330) at 5 μg/ml, BV785 anti human CD3 (Biolegend #344842), PE/Cyanine7 anti-human CD8 Antibody (Biolegend #344712), PE/Cyanine7 anti-human CD4 Antibody (Biolgend #317414), PE/Cyanine7 Mouse IgG2b, K isotype (Biolegend #400325), PE/Cyanine7 Mouse IgG1, K isotype (Biolegend #400125) and BV785 Mouse IgG1, K isotype (Biolegend #400169).

NK-92 (ATCC CRL-2407) and NK-92 MI (ATCC CRL-2408) cells were obtained from American Type Culture Collection (ATCC, Manassas, VA, USA). NK-92 MI cells were cultured at 37° C. with 5% CO₂ using X-VIVO 15TM serum-free Hematopoietic Cell Medium (Lonza 04-418Q) supplemented with 10% FBS (Life Technologies 26140-079) and supplemented with 10% human serum (Sigma Aldrich H3667-100ML), 1% penicillin/streptomycin (Hyclone SV30010.01).

### Plasmid Construction

The TCR activator, a membrane-anchored chimeric antibody, was constructed by fusing the single chain variable fragment (scFv) of the human CD3 mAb OKT3 (Kipriyanov et al. 1997, PEDS 10:445-453) to the C-terminal domain (113-220) of mouse CD8a (accession number: NP_001074579.1). The DNA sequence encoding TCR activator was synthesized and inserted into pIRES-hyg3 vector (ClonTech) to make resulting construct TCRa_pIREShyg3. The DNA sequence of human HHLA2 (accession number: NM_009003), TMIGD2 (accession number: NM_144615) and KIR3DL3 (Genbank accession number BC143802.1 corresponding to KIR3DL3*00402 allele) were synthesized and inserted into pIRES-Hyg3 or pIRES-Neo3 individually to make HHLA2_pIRESneo3, TMIGD2_pIREShyg3 and KIR3DL3_pIREShyg3. NFAT reporter contains a firefly luciferase gene under the control of four copies of NFAT response element followed by a minimal promoter. IL2 reporter contains a firefly luciferase gene under the control of an endogenous IL2 promoter. The DNA sequence encoding the reporters was inserted into pcDNA 3.1 to generate NFAT-Luc-pcDNA and IL2-Luc-pcDNA.

### Generation of Stable Cell Lines

Jurkat cells (clone E6-1) were co-transfected sequentially with NFAT_Luc_pcDNA and TMIGD2_pIREShyg3 by electroporation. Stable clones were generated by hygromycin (200 μg/ml) and G418 (1000 μg/ml) double selection and limiting dilution. The chosen stable cell clone was maintained with complete cell culture medium supplemented with hygromycin and G418. Jurkat cells (clone E6-1) were co-transfected sequentially with IL2_Luc_pcDNA and KIR3DL3_puro by electroporation. Stable clones were generated by puromycin (0.25 μg/ml) and G418 (1000 μg/ml) double selection and limiting dilution. The chosen stable cell clone was maintained with complete cell culture medium supplemented with puromycin and G418. CHO-K1 cells were co-transfected sequentially with TCRa_pIREShyg3 and HHLA2_pIRESneo3 by Lipofectamine 2000 (Invitrogen). Stable clones were generated by hygromycin and G418 double selection and limiting dilution. The chosen stable cell clone was maintained with complete cell culture medium supplemented with hygromycin and G418.

sgRNA targeting the human β2-microglobulin gene (5′-GCTACTCTCTCTTTCTGGCC) (World Wide Web at addgene.org/84381/) was ordered from Synthego with 2′-O-methyl 3′ phosphorothionate modification in the first and last 3 nucleotides. 2 μl of 66.88 μM of Cas9 Nuclease (Aldevron, SpyFi Cas9 Nuclease 9214-0.25 MG) was mixed with 3 μl of 100 μM sgRNA. Cas9 Nuclease and sgRNA were incubated at room temperature for 20 minutes. Raji cells were electroporated with Cas9 RNP using Lonza 4D Nucleofector following Lonza recommended setup for Raji cells. Raji cells were cultured for 48 hours, then sorted for β2-microglobulin negativity using human β2-microglobulin antibody (Biolegend 395711). B2-microglobulin negative cells were cultured and re-sorted multiple times until a pure β2-microglobulin negative cells population was obtained.

### Reporter Assays

### TMIGD2_NFAT_Jurkat Reporter Activity:

HHLA2-TCR-CHO and TCR-CHO cells were seeded at 2×10⁴ cells/well density in CHOK1 growth medium in a white opaque bottom 96-well plate and incubated overnight at 37° C. with 5% CO₂. The next day, medium was removed and cells were incubated with HHLA2 antibody in 50 μl Jurkat cell medium for one hour before the addition of TMIGD2 NFAT Jurkat reporter cell line (clone #62) at 4-5×10⁴ cells/well in 50 μl Jurkat cell medium. The culture was incubated for 3-6 hours. Luciferase signal was produced by adding 100 μl ONE-Step™ Luciferase Assay System (BPS Bioscience 60690), according to manufacturer's protocol and luminescence measured in a luminometer.

### KIR3DL3_IL2_Jurkat Reporter Activity

HHLA2-TCR-CHO (clone #28) or TCR-CHO cells were seeded at 2×10⁴ cells/well density in CHO-K1 growth medium in a white opaque bottom 96-well plate and incubated overnight at 37° C. with 5% CO₂. The next day, the medium was removed and cells were incubated with HHLA2 or KIR3DL3 antibodies in 50 μl Jurkat cell medium for one hour before the addition of KIR3DL3_IL2_Jurkat reporter cell line (Clone #2-12) at 4-5×10⁴ cells/well in 50 μl Jurkat cell medium plus CD28 antibody at a final concentration of 1 μg/mL in 100 μL assay mixture per well (clone 9.3, BioXcell BE0248). Luciferase signal was produced by adding 100 μl ONE-Step™ Luciferase Assay System (BPS Bioscience, 60690), according to manufacturer's protocol and luminescence measured in a luminometer.

### Antibody Generation

HHLA2 mAbs: BALB/c mice were primed with 50 μg of recombinant HHLA2-mIg2a in complete Freund's adjuvant by subcutaneous injection followed by 3-4 rounds of boosting with 50 μg of recombinant HHLA2-mIgG2a followed by denatured HHLA2-mIgG2a in incomplete Freund's adjuvant by intraperitoneal injection. Spleen and lymph node cells from mice that showed the highest HHLA2 antibody titers were fused to SP2/0 myeloma cells and hybridoma supernatants were screened by flow cytometry on HHLA2 transfected and parental 300.19 cells.

KIR3DL3 mAbs: BALB/c mice were primed by intramuscular injection with cardiotoxin (50 μl of 10 mM) and immunized with 100 ug plasmid containing the KIR3DL3 cDNA by the same route of injection. Two additional rounds of cardiotoxin pretreatment and boosting with KIR3DL3 plasmid DNA and KIR3DL3 transfected NIH-3T3 cells were conducted. Spleen and lymph node cells from mice that showed the highest KIR3DL3 antibody titers were fused to SP2/0 myeloma cells and hybridoma supernatants were screened by flow cytometry on KIR3DL3 transfected and parental 300.19 cells.

### NK Cytotoxicity Assay

NK92-MI cells cytotoxicity was determined using a KILR detection kit (Eurofins/Discoverx 97-0001M). Using the manufacturer's recommendation, the target cells, K562 cells were infected with KILR Retroparticles (KILR® Retroparticles for Adherent & Suspension Cells (G418), Eurofins/Discoverx 97-0006). Infected cells were selected in 500 μg/ml G418. NK92-MI (effector) cells were co-cultured in a 96-well plate with 104 K562 target cells at an effector-to-target (E: T) ratios of 1:1, 3:1 and 5:1 at 37° C. for 4 h. Cell lysis was detected on a luminometer 1 h after adding 100 μl KILR detection reagent to the effector cells or target cells at room temperature. Where indicated, NK92-MI (effector) cells were incubated with HHLA2 or KIR3DL3 blocking antibodies at 0.1, 1.0 and 10 μg/ml concentrations and appropriate isotype controls and co-cultured with K562 (target) cells at a fixed effector-to-target (E: T) ratio of 3:1 at 37° C. for 4 h. The percentage specific lysis was calculated as follows:

Specific lysis (%)= [optical density (OD) experimental group-OD target cell natural release control]/(OD target cell maximum release control-OD target cell natural release control)×100. Maximum release was measured after treatment with lysis agent provided by manufacturer.

Raji and HHLA-2 transfected Raji cells (B2-microglobulin Knock out) were stained with 1 μM Calcein A M (BioLegend 425201) in PBS for 20 minutes at room temperature then washed twice with PBS and resuspended in RPMI complete medium at 2×106/ml. 5 ×10⁴ Raji target cells and NK-92 MI effector cells at 1:1, 2:1 and 3:1 E/T ratios were cultured either in the presence of HHLA2 or KIR3DL3 antibodies or isotype controls for 4 hours in round bottom 96 well plates at 37° C. with 5% CO₂. Plates were then put on ice and analyzed by flow cytometry. Analysis was performed using FlowJo. Percent cell lysis was calculated as follows: % cytotoxicity=% calcein AM+target cells in the absence of effectors (100%)-% calcein AM+target cells in the presence of effectors (E/T ratio+1).

### CD107 Degranulation Assay

Raji β2-microglobulin KO and HHLA-2 transfected Raji β2-microglobulin KO were co-cultured with NK-92 MI for 3 hours at effector to target ratios (E: T) of 1:1, 2:1 and 3:1 in round-bottom 96 well plate at 37° C. with 5% CO₂. Anti-KIR3DL3 antibody (1G7) and mouse IgG2b Isotype were used at 10 μg/ml. Antibodies BV421 anti human CD107a (Biolegend #328625), APC anti-human CD56 (Biolegend #362503), APC Mouse IgG1, K Isotype Ctrl (Biolegend #400121) and BV421 Mouse IgG1, K Isotype Ctrl (Biolegend #400157) were used at 1:100 dilution. Monensin Solution (1,000×) (Biolegend #420701) was used at 1:1000 dilution and Cell Stimulation Cocktail, PMA/Ionomycin (Biolegend #423301) was at 1:500 dilution. Subsequently the plate was put on ice and cells were analyzed for CD107a expression on CD56 gated cells using flow cytometry and FlowJo software.

### Derivation of HHLA2 Transfected K562 and Raji Beta-2 Microglobulin Knockout Target Cells:

K562 cells were incubated at 37° C. with 5% CO₂ in RPMI1640 medium (Life Technologies A10491-01) supplemented with 10% fetal bovine serum (FBS, Life Technologies 26140-079), 1% Penicillin/Streptomycin (Hyclone SV30010.01).

K562 cells were electroporated (300 volts, 1600 uFarads) with 50 μg of Mlu I linearized HHLA2 cDNA in the pEF-Puro vector, selected for puromycin resistance, stained with PE-conjugated HHLA2 mab 6F10, sorted and single cell cloned. K562 HHLA2 stable cell line was cultured in above indicated RPMI1640 media supplemented with 1 μg/mL of puromycin.

### Western Blot Analysis

Protein lysates were prepared with RIPA buffer with protease inhibitor cocktail per manufacturer's instructions (Thermo Scientific; complete Ultra tablets, mini, EDTA-free, Roche). Lysates were loaded into a single wide lane 4-15% gradient mini-Protean TGX gel (Biorad) and transferred by a semidry method. Membranes were blocked with 12% non-fat milk and 1% normal goat serum in Tris-buffered saline with Tween20 (TBST) for 1 hour at room temperature. The membrane was washed with TBST and incubated with anti-KIR3DL3 mAb, 574.1F12, at 5 μg/ml in TBST and 1% BSA at 4° C. overnight in a multi-well-mini-blotter. Membranes were washed with TBST three times at room temperature and incubated with secondary antibody (1:4000, HRP-conjugated goat anti-mouse IgG, Southern Biotech) in TBST, 6% non-fat milk and 0.5% normal goat serum for 30 min. After 3 additional washes with TBST, a 1:1 ratio of ECL substrate: enhancer was added to the membrane (SuperSignal West Pico Stable Peroxide Solution, Supersignal West Pico Luminol/Enhancer Solution, ThermoScientific) and imaged on Hyblot CL autoradiography film (Denville Scientific).

### RNA Extraction and Quantitative Real-Time PCR

Total RNA was extracted from cell pellets using Purelink RNA mini Kit (ThermoFisher). cDNA synthesis was performed with the High Capacity RNA-to-cDNA Kit (Applied Biosystems, ThermoFisher) according to the manufacturer's recommendations. RT-PCR was performed using Power SYBR™ Green PCR Master Mix according to the manufacturer's recommendations. Primers used included:

18S:
fw 5′-GTAACCCGTTGAACCCCATT-3′;
5′-CCATCCAATCGGTAGTAGCG-3′
KIR3DL3:
fw 5′-AGAAGACGGGATGCCTGTC-3′;
rv 5′-GTGAACTGCAACATCTGTAGGT-3′
HHLA2:
fw 5′-TACAAAGGCAGTGACCATTTGG-3′;
rv 5′-AGGTGTAAATTCCTTCGTCCAGA-3′
PD-L1 (CD274):
fw 5′-TGGCATTTGCTGAACGCATTT-3′;
rv 5′-TGCAGCCAGGTCTAATTGTTTT-3′

18S was used as internal control for each sample. Relative mRNA levels were determined by the 2-ΔΔCT formula, and experiments were repeated three times.

Analysis of mRNA Expression in ccRCC Patients

TCGA RSEM RNASeqV2 data for TCGA Kidney Clear Cell patients was downloaded from the TCGA GDAC at World Wide Web at gdac.broadinstitute.org/runs/stddata_2016_01_28/data/KIRC/20160128/gdac.broadinstitute.org_KIRC.Merge_rnaseqv2_illuminahiseq_rnaseqv2 unc_edu_Level_3_RSEM_genes_data. Level_3.2016012800.0.0.tar.gz). To derive Transcripts per Million (TPM), the “scaled_estimate” output value was multiplied by 106. These TPM values were transformed to log 2 (TPM+1) and tumor and normal expression was compared for each gene using a Wilcoxon rank sum test.

### Cytometry:

Cells were analyzed on a Gallios Flow Cytometer (Configuration: 488 nm, 561 nm, 405 nm, 355 nm, and 635 nm) or a BD Fortessa Flow Cytometer. Data were analyzed with FlowJo or with Kaluza Software. For each experiment, 10,000 to 20,000 cells were analyzed.

### Statistics:

Data were analyzed using GraphPad Prism® Software 7, unless otherwise indicated. All data are represented as mean±S.D. (error bars) except where stated otherwise (see Figure legends). Student's t test and two-way ANOVA were used as indicated in the legends (non-significant (ns), P>0.05; * P≤0.05; ** P<0.01; *** P<0.001; **** P<0.0001).

### Example 2: Identification and Characterization of KIR3DL3 as Second Receptor for HHLA2

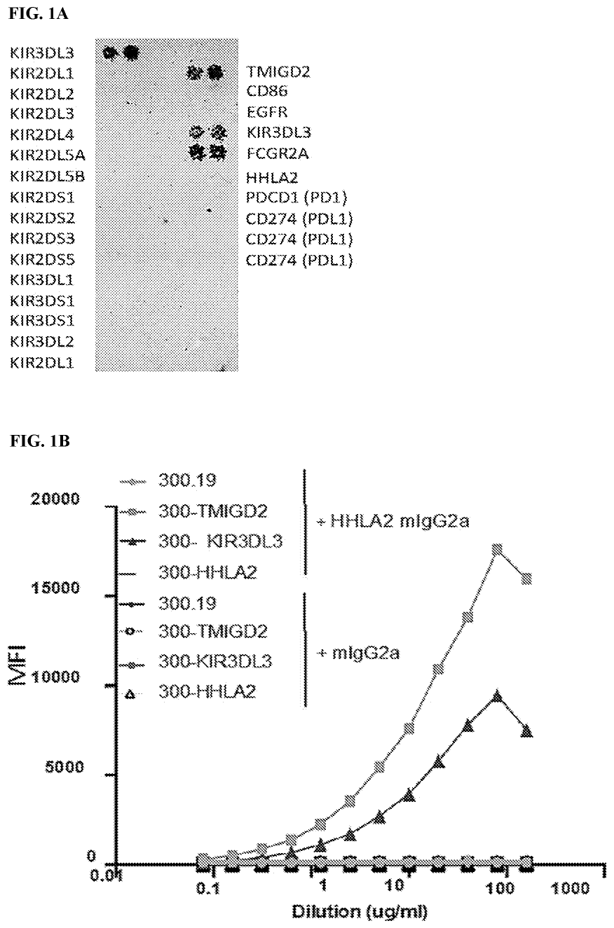

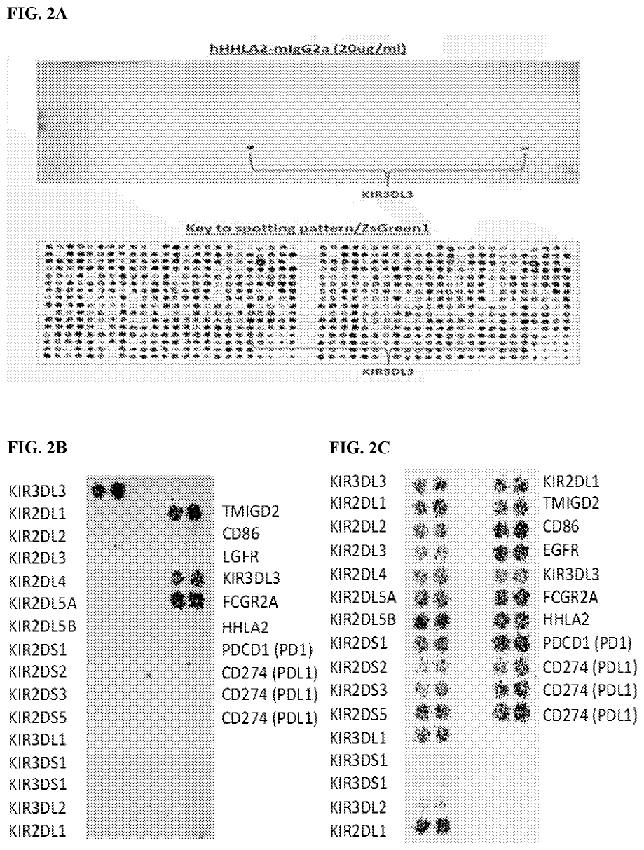

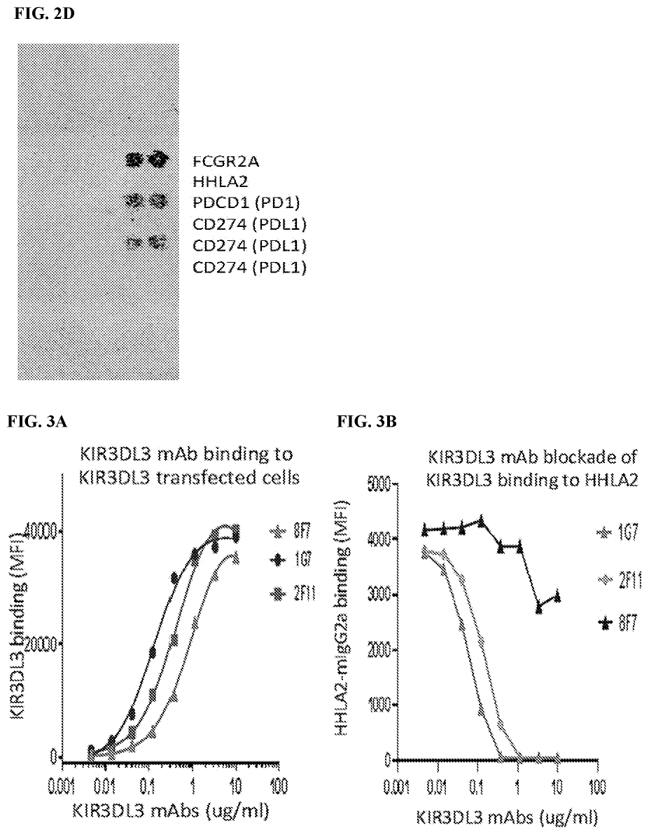

In order to identify an inhibitory receptor for HHLA2, a receptor screen was performed using soluble human HHLA2-mIgG2a fusion protein (HHLA2-Ig) on a library of ˜5500 cell surface receptors each expressed individually in HEK293 cells on glass slides. The screen identified KIR3DL3 as a positive signal for HHLA2 binding (FIGS. 1A and 2A). As expected, HHLA2-Ig also bound to TMIGD2 and the Fc receptor, FCGR2A but not to EGFR, PD-1 or PD-L1 (FIG. 1A). KIR3DL3 is a member of the KIR gene family whose ligand has not yet been described. Other members of the KIR family were present in the 5500 cDNA screen that only identified KIR3DL3, but to confirm the specificity, we individually tested HHLA2-Ig binding to KIR3DL3 and other members of the KIR gene family. HHLA2-Ig bound only to KIR3DL3 and not to other members of the KIR family (FIGS. 1A and 2A).

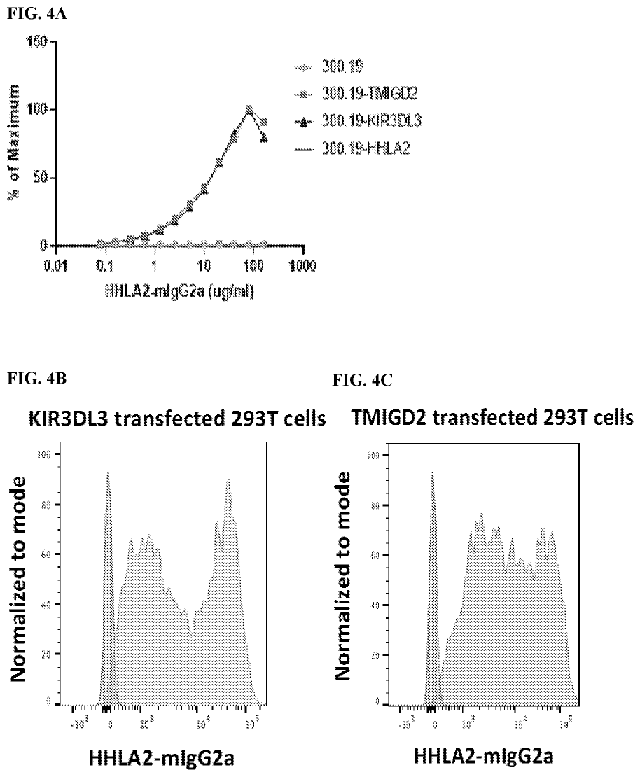

To confirm the HHLA2/KIR3DL3 and HHLA2/TMIGD2 interactions, the binding of HHLA2-Ig to cells expressing TMIGD2 and KIR3DL3 was tested. KIR3DL3 and TMIGD2 were stably overexpressed in the 300.19 mouse pre-B cell leukemic cell line and transfected cells were incubated with HHLA2-mIgG2a and analyzed by flow cytometry. It was discovered herein a dose dependent binding of HHLA2 to its two receptors, KIR3DL3 and TMIGD2 with very similar 50% maximal binding levels indicating similar binding affinities for these two receptors (FIGS. 1B and 4A). There was no binding to untransfected 300.19 cells or to HHLA2 itself (300.19 cells transfected with HHLA2). Similar specific binding was observed with KIR3DL3 and TMIGD2 transiently transfected 293T cells (FIGS. 4B and 4C).

### Example 3: Derivation of KIR3DL3 Monoclonal Antibodies

A number of anti-KIR3DL3 monoclonal antibodies were generated and analyzed. Briefly, human KIR3DL3 cDNA plasmid and KIR3DL3 transfected 3T3 or 300.19 cells were produced and utilized to immunize mice for the derivation of KIR3DL3 mouse monoclonal antibodies. Five mice (Balb/c; C57Bl/6; Swiss-Webster), 4-6 weeks old, were obtained from Charles River Laboratories (Wilmington, MA). All animals were acquired and maintained according to the guidelines of the Institutional Animal Care and Use Committee of Harvard Standing Committee on Animals. Mice were primed in the tibialis muscle with a pre-injection of 50 μl of 10 mM cardiotoxin (Naja nigricollis venom; Latoxan Laboratories, France) five days prior to an intramuscular injection of plasmid DNA. The mice were anesthetized and 100 micrograms of cDNA suspended in Dulbecco's phosphate buffered saline (PBS; GIBCO, Grand Island, NY) was injected into both tibialis muscles (50 μl each). The cardiotoxin pre-treatment and cDNA boost was repeated on days 14 and day 28. Five weeks later the mice were immunized with KIR3DL3 transfected 300.19 cells in PBS. Two weeks later the mice were immunized with KIR3DL3 transfected NIH-3T3 cells in PBS. Ten days later mice were bled and serum KIR3DL3 mAb titers were evaluated on KIR3DL3 transfected cells by flow cytometry. Balb/C mouse animal #2 that showed the highest serum KIR3DL3 mAb titers was selected for fusion. Five weeks after the previous immunization, Mouse #2 was boosted with KIR3DL3 transfected NIH-3T3 cells in PBS and fused 4 days later. The harvested spleen and lymph nodes were made into a cell suspension and then washed with DMEM. The spleen/lymph node cells were counted and mixed with SP 2/0 myeloma cells that are incapable of secreting either heavy or light chain immunoglobulin chains (Kearney et al. (1979) J Immunol 123:1548-1550 and Kilpatrick et al. (1997) Hybridoma 16:381-389) using a spleen: myeloma ratio of 2:1. Cells were fused with polyethylene glycol 1450 in eight 96-well tissue culture plates in HAT selection medium according to standard procedures (Kohler and Milstein (1975) Nature 256:495-497). Between 10 and 21 days after fusion, hybridoma colonies became visible and culture supernatants were harvested and then screened for KIR3DL3 binding by flow cytometry on 300.19 cells transfected with KIR3DL3 cDNA and for lack of reactivity on untransfected 300.19 cells.

The binding characteristics of derived KIR3DL3 mAb, e.g., binding affinity, are summarized in Table 3.

### Example 4: Binding and Selectivity of KIR3DL3 mAb

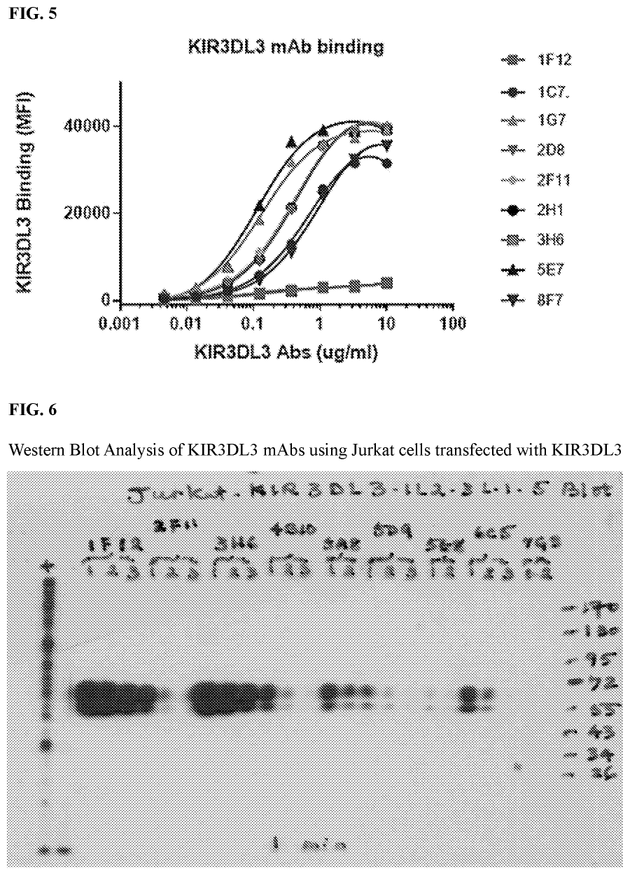

FIG. 5 shows that KIR3DL3 mAbs bind to KIR3DL3-positive cells. The indicated concentrations of KIR3DL3 mAbs were incubated with KIR3DL3 transfected 300.19 pre-B cells for 30 minutes at 4° C. KIR3DL3 mAb binding to transfected 300.19 cells was detected with 10 μg/ml of PE-labeled goat anti-mouse IgG (H+L). Accordingly, KIR3DL3 mAbs bind to KIR3DL3 expressed by cells.

Table 4 shows that most KIR3DL3 mAbs bind specifically to KIR3DL3 but not the other members of KIR family. Some KIR3DL3 mAbs also showed weak to medium binding to KIR3DL1, KIR2DL5A, and/or KIR2DL5B. A panel of cDNA plasmids encoding KIR family genes were spotted on glass slides containing human HEK293 cells and reverse transfected into the HEK293 cells to express cell surface KIR molecules. KIR3DL3 mAb hybridoma supernatants at dilutions of 1:5, 1:25 and 1:250 were incubated on the KIR molecule panel and binding was detected using AlexaFlour647 labeled anti-mouse IgG (H+L) antibody followed by imaging for fluorescence (Table 4). HHLA2-mIgG2a (20 μg/ml) was used as a positive control to detect binding to KIR3DL3 and TMIGD2 on the spotted array.

FIG. 6 shows that KIR3DL3 mAb binds KIR3DL3 on Western blots. Protein lysates of KIR3DL3 transfected Jurkat cells were prepared with RIPA buffer per manufacturer's instructions (Thermo Scientific), and protease inhibitor cocktail was added to the buffer (complete Ultra tablets, mini, EDTA-free, Roche) prior to lysate preparation. Protein lysates were made from Jurkat cells stably transfected with human KIR3DL3. Seven hundred ug of lysate was loaded into a single wide lane 4-15% gradient mini-Protean TGX gel (Biorad) and transferred by a semidry method. Membranes were blocked with 12% non-fat milk and 1% normal goat serum in Tris-buffered saline with Tween20 (TBST) for 1 hour at room temperature. The membrane was washed with TBST and incubated with the primary antibody (final dilutions of 1 to 10, 1 to 30, and 1 to 90 of hybridoma supernatant anti-KIR3DL3 mAb in TBST and 1% BSA at 4° C. overnight in a multi-well-mini-blotter. Membranes were washed with TBST three times at room temperature and incubated with secondary antibody (1:4000, HRP-conjugated goat anti-mouse IgG, Southern Biotech) in TBST, 6% non-fat milk and 0.5% normal goat serum for 30 min. After 3 additional washes with TBST, a 1:1 ratio of ECL substrate: enhancer was added to the membrane (SuperSignal West Pico Stable Peroxide Solution, Supersignal West Pico Luminol/Enhancer Solution, ThermoScientific) and imaged on Hyblot CL autoradiography film (Denville Scientific).

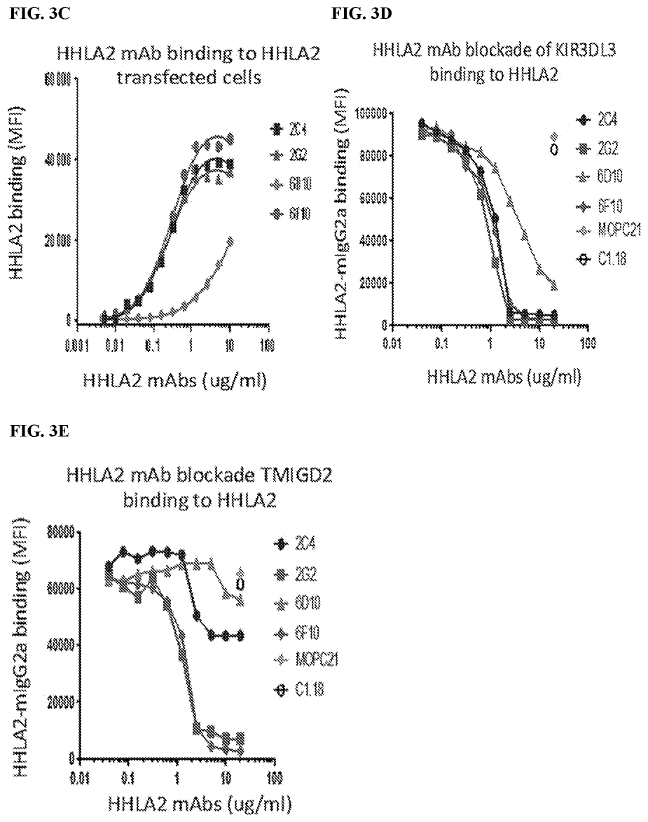

FIG. 3A-FIG. 3E and Table 5 show additional characterization of the binding of the antibodies to antigen and capacity to block HHLA2 binding to either KIR3DL3 or TMIGD2. Dose dependent binding of KIR3DL3 and HHLA2 antibodies was observed to KIR3DL3 and HHLA2 transfected 300.19 cells, respectively (FIGS. 3A and 3C). All HHLA2 antibodies except 6D10 showed high affinity binding to their antigen. Blocking and non-blocking antibodies were identified. HHLA2 antibodies 2G2 and 6F10 blocked the interaction of HHLA2 with both KIR3DL3 and TMIGD2, while 2C4 and 6D10 antibodies only blocked the HHLA2/KIR3DL3 interaction but not the HHLA2/TMIGD2 interaction (FIGS. 3D and 3E, Table 5). KIR3DL3 mAbs 1G7, 2F11, and 8F7 showed potent binding to KIR3DL3 in a dose-dependent manner (FIG. 3A). 1G7 and 2F11 blocked the interaction of KIR3DL3 with HHLA2 while 8F7 only weakly blocked the interaction (FIG. 3B and Table 5). All KIR3DL3 antibodies specifically bound KIR3DL3 and not to any other KIR family members except the 2F11 antibody, which also showed weak binding to KIR2DL5A (Table 4).

### Example 5: KIR3DL3 mAb Blockade of HHLA2 Binding to KIR3DL3

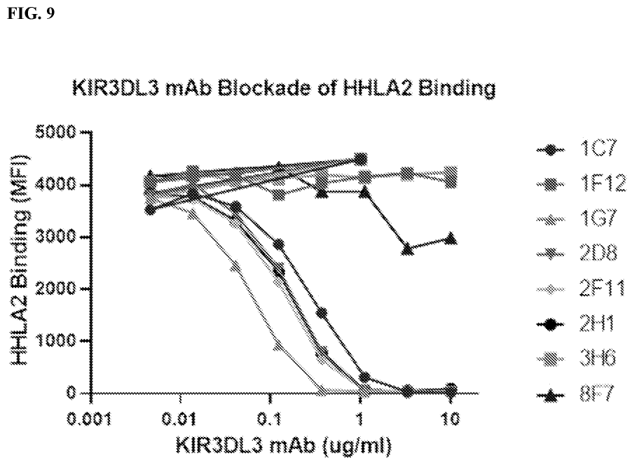

FIG. 9 shows KIR3DL3 mAb blocks binding of HHLA2 to KIR3DL3. The indicated concentrations of KIR3DL3 mAbs were pre-incubated with KIR3DL3 transfected 300.19 cells for 30 minutes at 4° C. HHLA2-mouse IgG2a (mutated at IgG2a L235E, E318A, K320A, K322A) (25 μl of 10 μg/ml) was added and incubation continued for 30 minutes at 4° C. Cells were washed and binding of HHLA2-mouse IgG2a was detected with 5 μg/ml Alexa647 conjugated 298.6F8 mAb (mouse antibody specific for mouse IgG2a mutated at L235E, E318A, K320A, K322A). EC50 and IC50 analysis were conducted using GraphPad Prism®.

TABLE 3
Summary of KIR3DL3 mAb Characteristics

| | | | | Blockade of | | | |
|---|---|---|---|---|---|---|---|
| | | | | KIR3DL3 | Stimulation of Jurkat IL-2 | | |
| KIR3DL3 | | Binding to | | binding to | Luciferase Activation by | | |
| mAb | | KIR3DL3 | Selectivity | HHLA2 | KIR3DL3 mAb blockade | | Western |
| 574.2 | | Kd | for | IC50 | EC50 | Fold | Blot of |
| series | Isotype | (ug/ml) | KIR3DL3 | (ug/ml) | (ug/ml) | Activation | KIR3DL3 |
| 1C7 | IgG1 | 0.77 | 3DL3 | 0.32 | 77.0 | 8.2 | ND |
| 1F12 | IgG2b | 0.1 | 3DL3 | Non-blocker | ND | ND | ND |
| 1G7 | IgG2b | 0.13 | 3DL3 | 0.06 | 0.37 | 9.9 | ND |
| 2D8 | IgG2b | 0.46 | 3DL3, 2DL5A | 0.21 | 1.3 | 14.9 | ND |
| 2F11 | IgG2b | 0.43 | 3DL3, 2DL5A | 0.16 | 1.25 | 17.1 | Weak |
| 2H1 | IgG2b | 0.42 | 3DL3, 2DL5A | 0.19 | 1.79 | 16.2 | ND |
| 3H6 | IgG2b | 0.14 | 3DL3 | Non-blocker | ND | ND | Strong |
| 5E7 | IgG2a | 0.11 | 3DL3 | ND | ND | 8.0 | ND |
| 8F7 | IgG1 | 1.0 | 3DL3 | 27.5 | No activation | 1.2 | ND |

ND: Not determined
Anti-KIR3DL3 mAb 1F12 was first choice in immunohistochemistry of human kidney tumor tissues and 3H6 was second choice.

TABLE 4
KIR3DL3 mAb Binding Selectivity for KIR3DL3 versus other KIR family members

| KIR3DL3 | | | | | | | | | | | | | | | |
|---|---|---|---|---|---|---|---|---|---|---|---|---|---|---|---|
| mAb | Isotype | 3DL3 | 3DL1 | 3DL2 | 2DL1 | 2DL2 | 2DL3 | 2DL4 | 2DL5A | 2DL5B | 2DS1 | 2DS2 | 2DS3 | 2DS5 | 3DS1 |
| 1C7 | IgG1 | +++ | − | − | − | − | − | − | − | − | − | − | − | − | − |
| 1D12 | IgG2a | +++ | − | − | − | − | − | − | + | | − | − | − | − | − |
| 1F12 | IgG2b | +++ | − | − | − | − | − | − | − | − | − | − | − | − | − |
| 1G7 | IgG2b | +++ | − | − | − | − | − | − | − | − | − | − | − | − | − |
| 2D8 | IgG2b | +++ | − | − | − | − | − | − | + | − | − | − | − | − | − |
| 2F11 | IgG2b | +++ | − | − | − | − | − | − | + | − | − | − | − | − | − |
| 2H1 | IgG2b | +++ | − | − | − | − | − | − | + | − | − | − | − | − | − |
| 3H6 | IgG2b | +++ | − | − | − | − | − | − | − | − | − | − | − | − | − |
| 5A7 | IgG1 | +++ | − | − | − | − | − | − | − | − | − | − | − | − | − |
| 5E7 | IgG2a | +++ | − | − | − | − | − | − | − | − | − | − | − | − | − |
| 6C5 | IgG2a | +++ | − | − | − | − | − | − | − | − | − | − | − | − | − |
| 7F1 | IgG2a | +++ | +/− | − | − | − | − | − | − | − | − | − | − | − | − |
| 7H2 | IgG1 | +++ | − | − | − | − | − | − | − | − | − | − | − | − | − |
| 8C2 | IgG2a | +++ | − | − | − | − | − | − | ++ | + | − | − | − | − | − |
| 8F7 | IgG1 | +++ | − | − | − | − | − | − | − | − | − | − | − | − | − |

Strong Binding: +++
Medium Binding: ++
Weak/Medium Binding: +
Very Weak Binding: +/−
No binding above background: −

In Table 4, replicate microarray slides expressing the KIR family cDNA panel (as in FIG. 1A, FIG. 2B, and FIG. 2C) were fixed and blocked with buffer containing PBS/0.5% BSA and incubated for 1 hour at room temperature with individual KIR3DL3 mAb hybridoma supernatants at 1:5, 1:25 and 1:250 dilutions in PBS/0.1% BSA. Cell arrays were washed with PBS and incubated for 1 hour at room temperature in PBS/0.1% BSA containing AF647-conjugated goat anti-mouse IgG (H+L) (Life Technologies, A21235). Slides were washed with PBS, dried, and imaged for ZsGreen1 and AF647 fluorescence.

### Example 6: KIR3DL3 is Induced on Some T Cells Following T Cell Activation and is Expressed on NK92-MI Cells

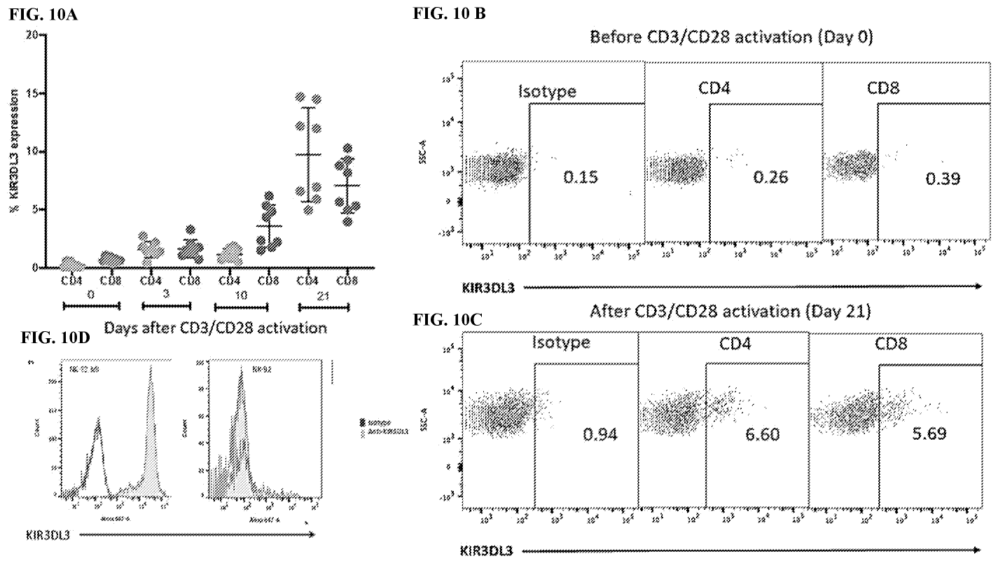

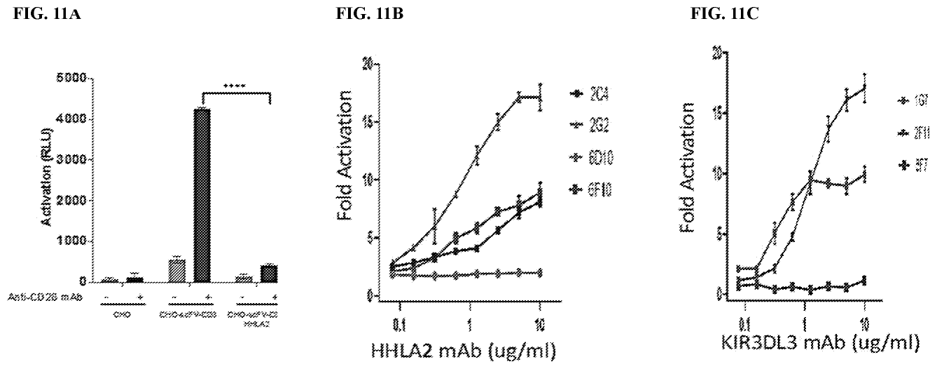

Previously published studies showed that TMIGD2 is expressed on naïve T and NK cells and downmodulated with activation. Some NK cell receptors such as NKG2A and KIR receptors can be expressed on CD8 lymphocytes. The expression of KIR3DL3 on T cells was assessed. T cells were purified from donor whole blood, activated with CD3/CD28 antibody tetramers and KIR3DL3 expression was determined in gated CD3+CD4+ and CD3+CD8+ T cells (FIG. 10A). Minimal KIR3DL3 expression (0.26% of CD4+ cells and 0.39% of CD8+ cells) was noted at day 0 (unactivated) (FIG. 10B) and there was an increase in KIR3DL3 expression in both CD4+ and CD8+ cells on days 3 and 10 which peaked on day 21 post activation (6.60% of CD4+ cells and 5.69% of CD8+ cells) (FIG. 10C).

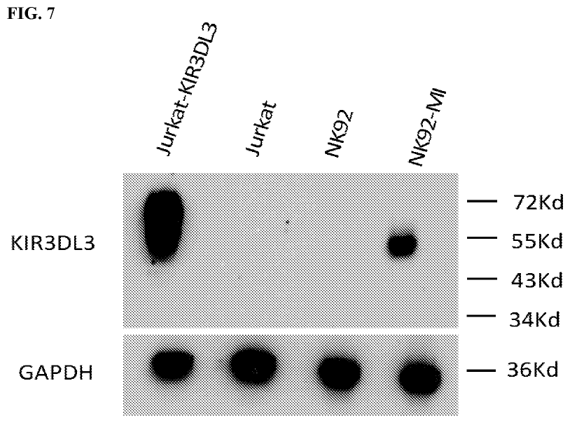

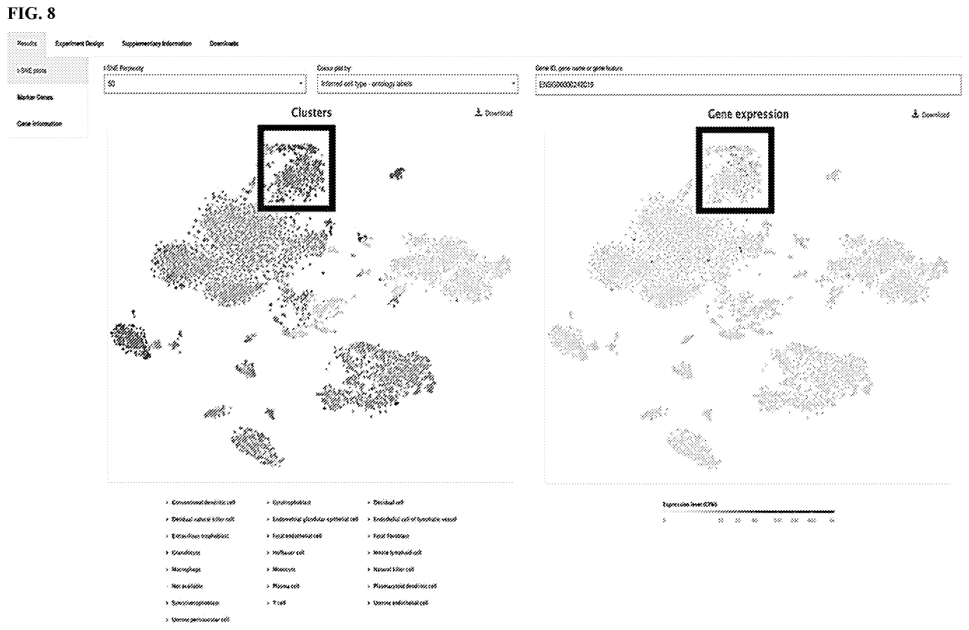

An NK cell line, NK92-MI, which endogenously expresses KIR3DL3, was identified herein. NK92-MI was derived from the original NK92 by transfection of an IL-2 cDNA and overexpresses IL-2, obviating the need for exogenous application of IL-2. KIR3DL3 was expressed on a minority of parental NK92 cells but was confirmed in NK92-MI cells by flow cytometry and Western blot (FIG. 10D and FIG. 7). In addition, KIR3DL3 expression has been reported on decidual NK cells and it was confirmed herein this by analyzing single cell RNA data in publicly accessible databases (FIG. 8).

### Example 7: KIR3DL3 is an Immunoinhibitory Checkpoint Receptor for HHLA2 and Blocking Antibodies Enhance T Cell and NK Activity

The cytoplasmic domain of KIR3DL3 contains one ITIM motif and is predicted to have an immunoinhibitory function. To assess the function of the HHLA2/KIR3DL3 interaction, Jurkat T cells expressing KIR3DL3 and an IL-2 promoter containing NFAT, AP-1 and NFKB response elements driving a luciferase reporter gene were co-cultured with CHO cells stably expressing cell surface anti-CD3 scFV alone or in combination with HHLA2. Soluble anti-CD28 mAb was added to provide a strong costimulatory signal and the luciferase activity was assessed. As expected, a CD3 signal was necessary for T cell activation and a CD28 costimulatory signal significantly increased IL-2 reporter activity (FIG. 11A). It was observed herein that an HHLA2-mediated signal through KIR3DL3 resulted in a significant decrease of T cell activation as measured by IL-2 reporter activity. These results indicate that KIR3DL3 is an inhibitory checkpoint receptor.

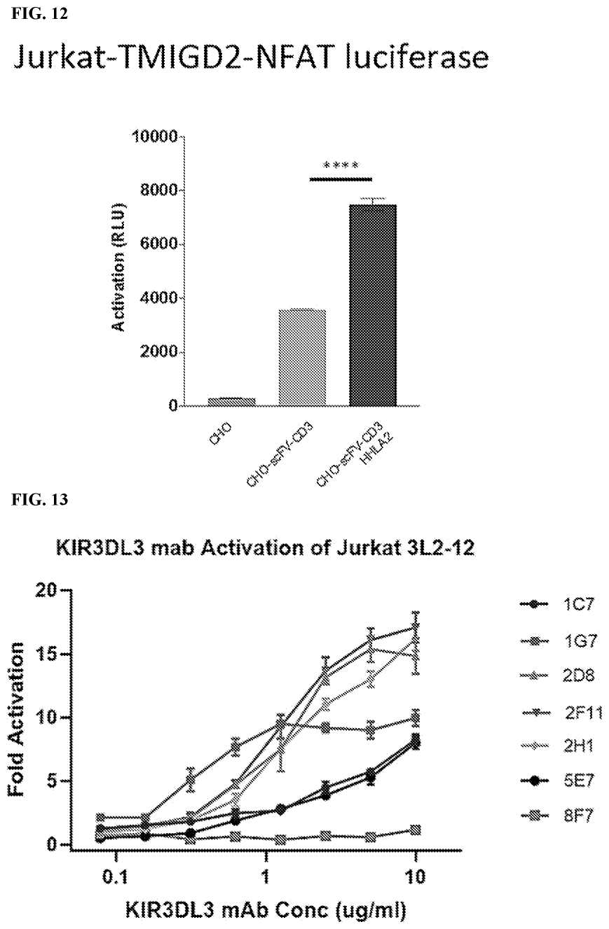

In order to assess the effect of TMIGD2 signaling following HHLA2 engagement and anti-CD3 mAb activation, TMIGD2 was overexpressed in Jurkat cells expressing the NFAT response element driving the luciferase reporter gene. When TMIGD2-transfected Jurkat cells were co-cultured with CHO cells stably expressing cell surface anti-CD3 scFV alone or in combination with HHLA2, we observed that HHLA2 increased T cell activation over CD3 signaling alone (FIG. 12), consistent with TMIGD2's reported co-stimulatory activity.

To assess the functional consequences of blocking the HHLA2/KIR3DL3 interaction, both receptor blocking and non-blocking HHLA2 and KIR3DL3 antibodies were evaluated in the KIR3DL3 Jurkat reporter gene assay. Consistent with their receptor blocking activity, the blocking HHLA2 mAbs 2C4, 2G2 and 6F10 enhanced IL-2 reporter activity whereas the weak blocker 6D10 did not (FIG. 11B). Similarly, KIR3DL3 mAbs 1G7 and 2F11 enhanced IL-2 reporter activity and T cell activation consistent with their receptor blocking activity whereas the weak blocker 8F7 did not (FIG. 11C and Table 5). The 2F11 mAb showed higher fold induction of IL-2 reporter activity compared to 1G7 although the binding avidity of 1G7 was higher.

### Example 8: KIR3DL3 mAb or HHLA2 mAb Promotes T Cell Activation

FIG. 13 shows that KIR3DL3 mAb enhances IL-2 promoter driven Luciferase expression in Jurkat-KIR3DL3 T cells in response to anti-CD3-scFV and HHLA2-mediated signals. CHO-anti-CD3 scFv-HHLA2 cells or CHO-anti-CD3 scFV cells were seeded in a 96 well plate at a concentration of 3.5×10⁴ in 0.15 ml media, with well A1 as a negative control containing just 0.15 of media. The cells were incubated overnight at 37° C. with 5% CO₂. The next day, eight serial two-fold dilutions of anti-KIR3DL3 or control MOPC21 antibodies were added to the appropriate wells. One row had no antibody added as another negative control. After antibodies were added, the plates were incubated for thirty minutes at 37° C. with 5% CO₂. Next, 25 μl of anti-CD28 antibody at 16 μg/mL was added to the appropriate wells. The negative control row had no anti-CD28 added. Jurkat cells expressing KIR3DL3 and an IL-2 promoter driven Luciferase gene were harvested, spun down, washed, and resuspended at a concentration of 2×10⁶/ml in media. Finally, 50,000 Jurkat cells in 25 μl of media were added to each well (except the negative control A1), and the wells were mixed. The final volume of all additions was 100 μl. The plates were incubated for 6 hours at 37° C. with 5% CO₂. After the six-hour incubation period, reagents from BPS Bioscience's ONE-Step™ Luciferase Assay System were thawed and mixed in the appropriate ratio according to the manufacturer's instructions in an aluminum foil covered tube (the reagents are light-sensitive). One hundred μl of the reagent mixture was added to each well, and the plates were then rocked on an orbital shaker for 15 minutes at room temperature. Immediately after the plates were rocked, they were read on a luminometer.

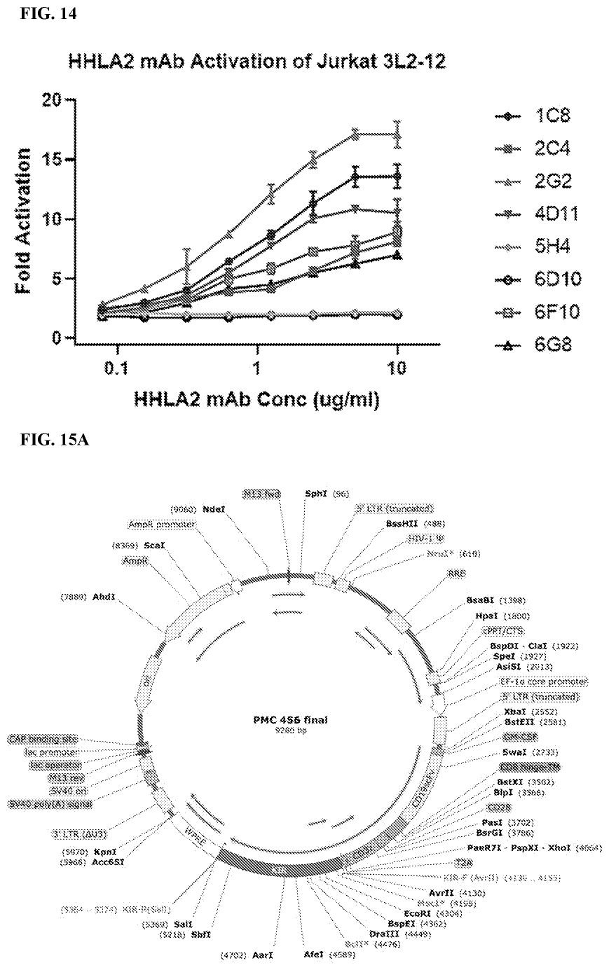

FIG. 14 shows that HHLA2 mAb enhances IL-2 promoter driven Luciferase expression in Jurkat-KIR3DL3 T cells in response to anti-CD3-scFV and HHLA2-mediated signals. CHO-anti-CD3 scFv-HHLA2 cells or CHO-anti-CD3 scFV cells were seeded in a 96 well plate at a concentration of 3.5×10⁴ in 0.15 ml media, with well A1 as a negative control containing just 0.15 of media. The cells were incubated overnight at 37° C. with 5% CO₂. The next day, eight serial two-fold dilutions of anti-HHLA2 or control MOPC21 antibodies were added to the appropriate wells. One row had no antibody added as another negative control. After antibodies were added, the plates were incubated for thirty minutes at 37° C. with 5% CO₂. Next, 25 μl of anti-CD28 antibody at 16 μg/mL was added to the appropriate wells. The negative control row had no anti-CD28 added. Jurkat cells expressing KIR3DL3 and an IL-2 promoter driven Luciferase gene were harvested, spun down, washed, and resuspended at a concentration of 2×10⁶/ml in media. Finally, 50,000 Jurkat cells in 25 μl of media were added to each well (except the negative control A1), and the wells were mixed. The final volume of all additions was 100 μl. The plates were incubated for 6 hours at 37° C. with 5% CO₂. After the six-hour incubation period, reagents from BPS Bioscience's ONE-Step™ Luciferase Assay System were thawed and mixed in the appropriate ratio according to the manufacturer's instructions in an aluminum foil covered tube (the reagents are light-sensitive). One hundred μl of the reagent mixture was added to each well, and the plates were then rocked on an orbital shaker for 15 minutes at room temperature. Immediately after the plates were rocked, they were read on a luminometer.

### Example 9: Blocking the HHLA2-KIR3DL3 Interaction Enhances CD19 CAR-T Cell Cytotoxicity Against Tumor Cells

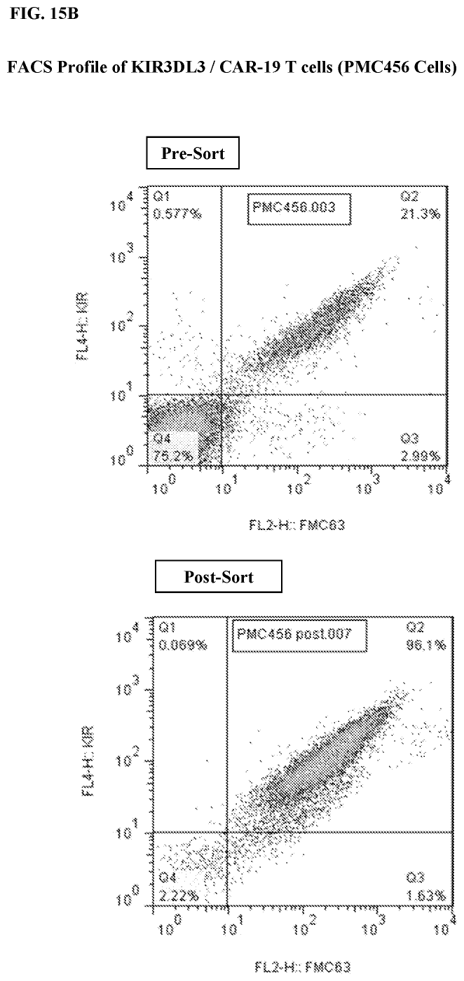

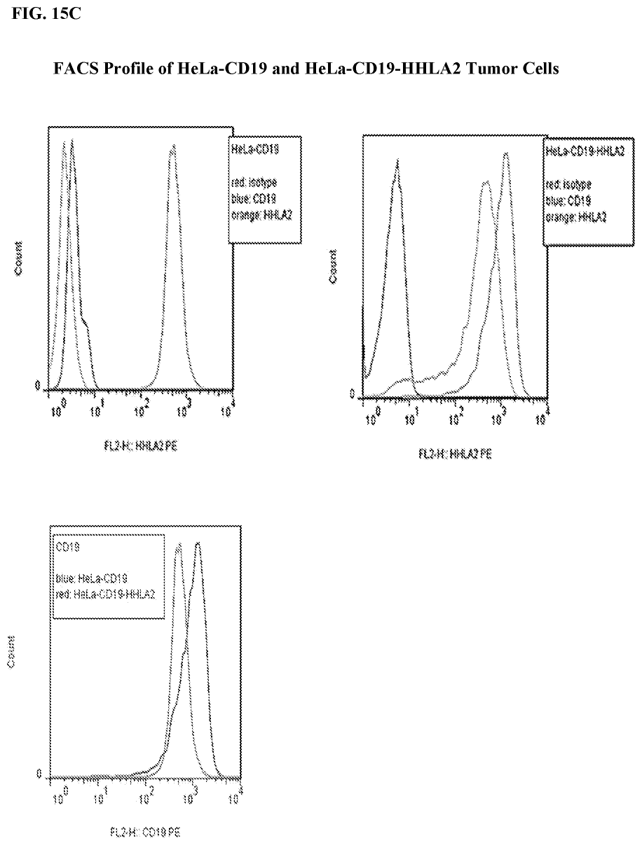

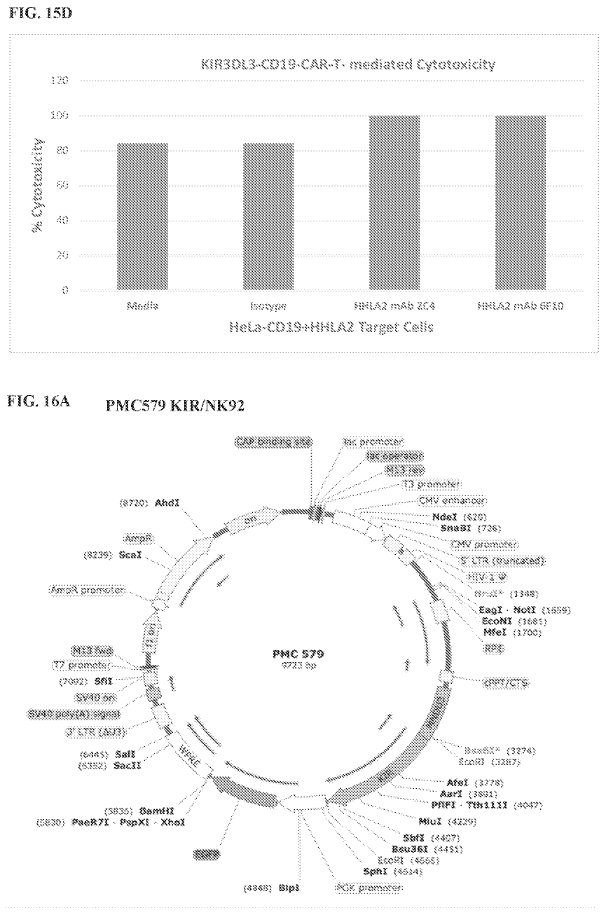

FIG. 15A shows the KIR3DL3/CAR-19 expression plasmid and lentivirus production. DNAs encoding KIR3DL3 and the CD19 CAR (containing the FMC63 mouse anti-CD19 scFv) were inserted into a second-generation CAR cassette containing EF-1a promoter, the signalingpeptide from GM-CSF, a hinge region, the transmembrane and co-stimulatory domains from CD28 and CD3zeta activation domains. The KIR3DL3 cDNAwas inserted after the CAR, separated by a T2A ribosome-skipping sequence that's self cleavable. The KIR3DL3/CD19 CAR DNA was subcloned into a third generation lentiviral vector to generate the PMC456 expression plasmid. HEK293 cells were transfected with the PMC456 plasmid and the pPACKH1 lentivector packaging mix using the calcium phosphate transfection kit (Takara, Mountain View, CA). Cell culture supernatants were harvested 48 hrs later and cleared of cell debris by centrifugation at 110K g for 10 minutes, then resuspended in AIM V-AlbuMAX® medium (Thermo Fisher) and aliquoted and frozen at −80° C. Viral titers were determined by quantitative RT-PCR using the Lenti-X™ qRT-PCR kit (Takara) and the 7099HT thermal cycler (Thermo Fisher).

FIG. 15B shows the generation and expansion of KIR3DL3/CD19-CAR-T cells and the FACS profile of KIR3DL3/CD19 CAR-T cells (PMC456 Cells). PBMCs were isolated from human peripheral blood buffy coats and suspended at 1×10⁶ cells/ml in AIM V™ medium containing 10% FBS and 100 IU/ml of IL-2. T cells were activated with CD3/CD28 Dynabeads™ (Thermo Fisher) overnight, then treated with DEAE dextran (5 μg/ml) and infected with KIR3DL3/CAR19 lentivirus 24 hrs and 48 hrs later (MOI of 10). Cells were counted every 2-3 days over the 8 days and supplemented with fresh medium containing IL-2 to maintain the cell density at 1-3×10⁶ cells/ml. On day 10, the CAR-T cells were isolated by sequential binding to magnetic beads coated with biotinylated anti-FMC63 antibody. Day-10 cells were stained before and after CAR-T cell isolation with a mixture of anti-KIR3DL3 mAb 1G7 and biotinylated anti-FMC63 antibody. After rinsing, the cells were stained with a mixture of APC-conjugated goat anti-mouse IgG and PE-conjugated streptavidin. The cells were rinsed and analyzed by flow cytometry.

FIG. 15C shows the generation of stable HeLa-CD19 and HeLa-CD19+HHLA2 expressing tumor cells and their FACS profile. HeLa cells were transduced with lentivirus vectors expressing human CD19 to generate HeLa-CD19 cells. Separately, HeLa cells were transfected with a plasmid encoding HHLA2 that contained the neomycin resitance gene and were selected for neomycin resistance using 1 mg/ml of G418. These cells were then transduced with the lentivirus encoding human CD19 to generate HeLa-CD19+HHLA2 expressing cells. Tumor cells were stained with PE-conjugated anti-HHLA2 6F10 mAb, anti-CD19 mAb or isotype control (mouse IgG1) mAb. Cells were rinsed and analyzed by flow cytometry.

FIG. 15D shows that blocking the HHLA2-KIR3DL3 interaction using HHLA2 mAb enhances KIR3DL3 CD19-CAR-T cell cytotoxicity against HHLA2+CD19 transfected HeLa tumor cells. Real-time cellular analysis (RTCAR) was used to measure the cytolytic activity of the enriched KIR3DL3/CAR19 T cells. Adherent HeLa-CD19-HHLA2 cells were seeded into 96-well E-plates® (Acea Biosciences) at 1×10⁴ cells per well and monitored in culture overnight with the impedance-based xCELLigence® system (Acea Biosciences). The next day, the medium was removed and replaced with medium containing 3×10⁴ or 1×10⁵ effector cells (KIR3DL3/CAR19 T cells or non-transduced T cells), and the indicated HHLA2 mAbs or isotype control in triplicate. The E-plates® were monitored for another day with the RTCA® system, and impedance of the tumor cell monolayer was plotted over time. Percent (%) cytotoxicity was calculated as (impedance of target cells without effector cells-impedance of target cells with effector cells)/impedance of target cells without effector cells x 100.

### Example 10: Blocking HHLA2-KIR3DL3 Interaction Enhances NK Cell Cytotoxicity Against Tumor Cells

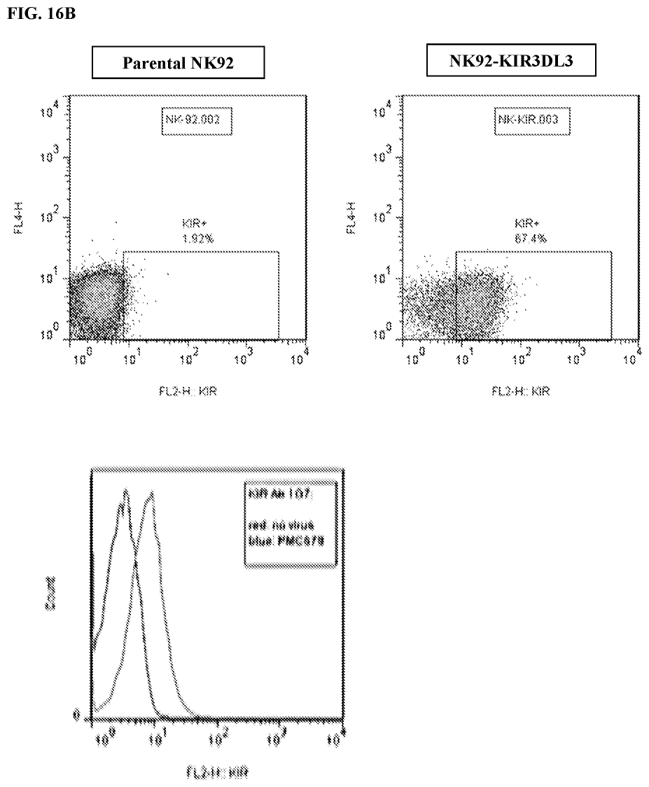

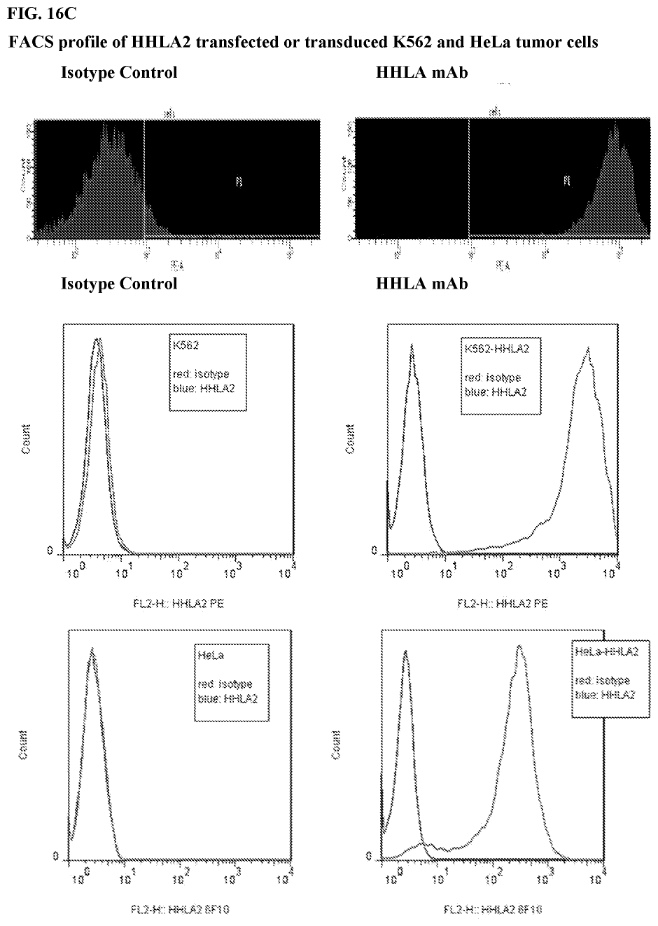

FIG. 16A shows the KIR3DL3 expression plasmid and lentivirus production. The KIR3DL3 cDNA was subcloned into a third-generation lentiviral vector to generate the PMC579 expression plasmid. The plasmid also contained the eGFP cDNA under the PGK promoter. Lentivirus was prepared as described in the preceding section and used to transduce the human NK cell line NK-92.

FIG. 16B shows derivation of KIR3DL3 transduced NK92 cells. Three million NK92 cells were transduced with KIR3DL3 expressing lentivirus (PMC579 expression plasmid containing the MNDU3 promoter) at an MOI of 10 and expanded in culture for 10 days in RPMI medium with 20% FBS and 50 ng/ml IL-2, after which a portion of the cells (19 million) were sorted by GFP expression level. These cells were put back into culture in RPMI medium with 20% FBS and 50 ng/ml IL-2 for another 10 days.

FIG. 16C shows derivation of HHLA2 transfected K562 and HeLa tumor cells. K562 cells were electroporated (300 volts, 1600 uFarads) with 50 μg of Mlu I linearized HHLA2 cDNA in the pEF-Puro vector, selected for puromycin resistance, stained with PE-conjugated HHLA2 mab 6F10, sorted and single cell cloned. HeLa tumor cells were transduced with an HHLA2 encoding lentivirus.

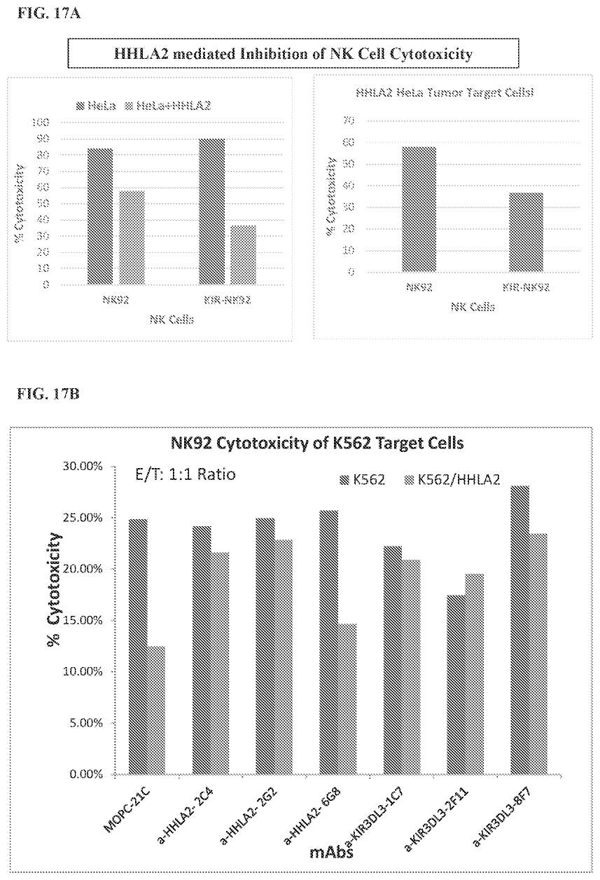

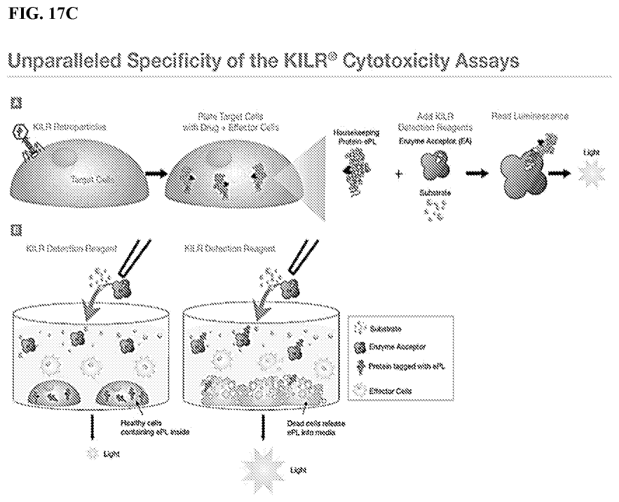

FIG. 17A shows inhibition of NK92 cytotoxicity by the KIR3DL3-HHLA interaction/pathway. NK92 parental cells or KIR3DL3 transduced NK92 cells were utilized in NK cytotoxicity assays against HeLa alone or HeLa+HHLA2 expressing cells. NK mediated cell killing was measured by culturing NK cells at an effector to target (E/T) ratio of 0.5:1 in the Acea xCelligence® impedance assay. Target cells were added to the 96-well E-plates® (20 thousand HeLa, HeLa-HHLA2) and impedance is monitored overnight. The next day the medium is removed and 150 μl of NK-KIR3DL3 or NK-NV (no viral vector) cells in NK-92 medium are added to each well. The plate is monitored overnight for chances in impedance.

FIG. 17B shows enhancement of NK92 cytotoxicity by HHLA2 mAbs and KIR3DL3 mAbs. Blocking HHLA2 mAbs 2G2, 2C4 and KIR3DL3 mAbs 1C7 and 2F11 reverse inhibition of NK92 cytotoxicity by HHLA2. Non-blocking HHLA2 mAb 6G8 and KIR3DL3 mAb 8F7 did not reverse inhibition of NK92 cytotoxicity by HHLA2. FIG. 17C shows a schematic diagram of the cytotoxicity assays.

### Example 11: The HHLA2-KIR3DL3 Interaction Inhibits NK Cell Cytolytic Activity, which is Reversible by HHLA2 or KIR3DL3 Antibodies

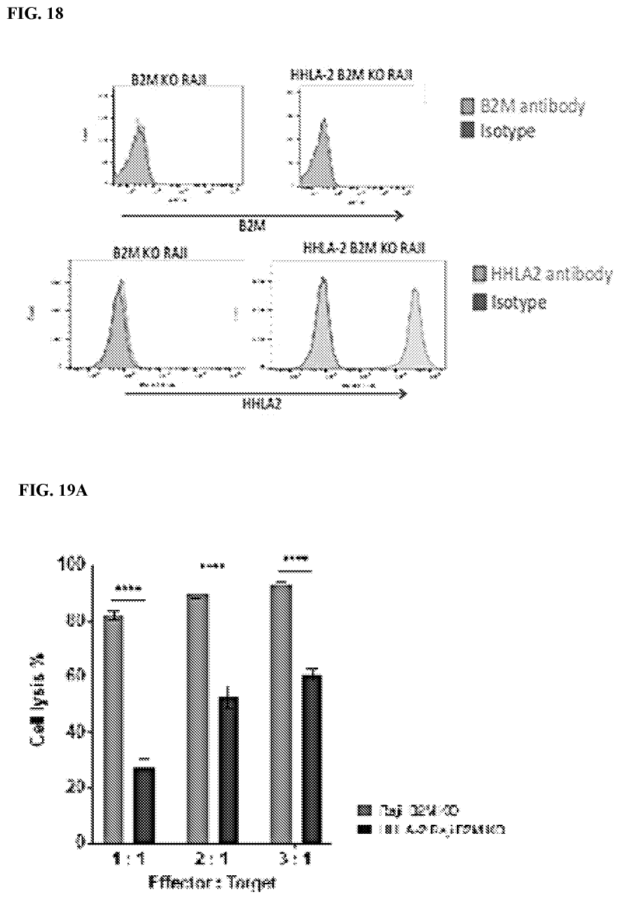

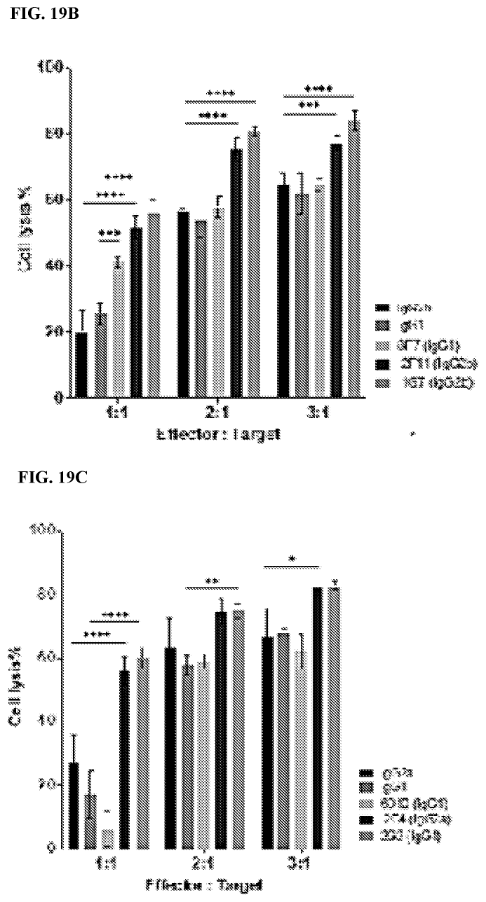

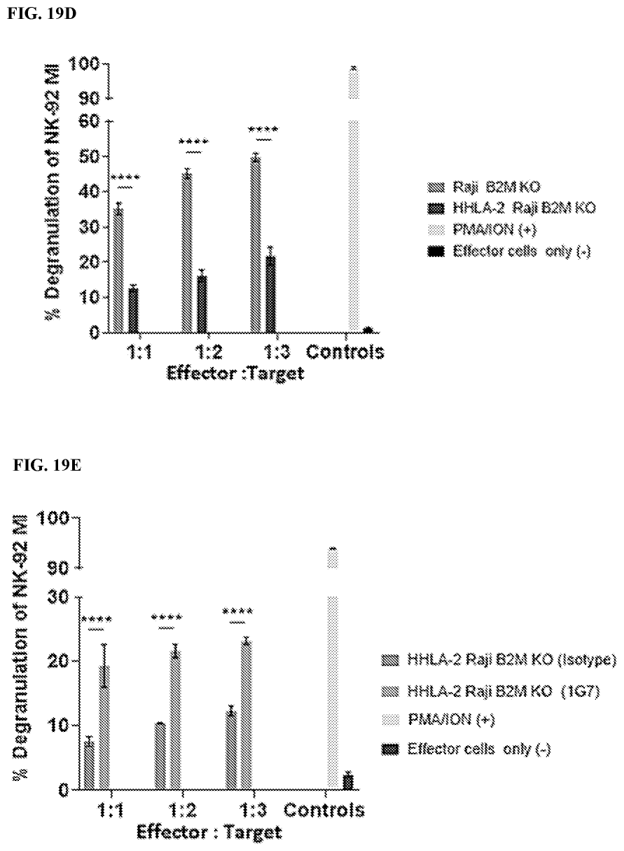

To assess the effects of the HHLA2-KIR3DL3 interaction on NK cytotoxicity, the NK cell line, NK92-MI, which endogenously produces IL2 and expresses KIR3DL3, was used (FIG. 10B). To confirm that HHLA2 expression inhibits NK cytotoxicity, Raji cells were first engineered to eliminate surface expression of MHC class I by CRISPR-mediated deletion of B2-microglobulin (B2M) to render them good targets for NK cells. NK cells are activated by the absence of MHC class I on target cells, and it was confirmed herein that Raji-B2M KO cells were efficiently lysed by NK92-MI cells (FIG. 18). Raji-B2M KO cells were engineered to express HHLA2 and FIG. 19A shows that HHLA2 expression on Raji-B2M KO cells inhibits cytotoxicity by NK-92-MI cells at all effector to target ratios.

To assess the effects of HHLA2 or KIR3DL3 antibodies on HHLA2 mediated inhibition of NK cytotoxicity, HHLA2-Raji-B2M KO model was incubated with NK92-MI cells at various E/T ratios and a fixed concentration of KIR3DL3 antibodies, 1G7 and 2F11. The KIR3DL3 blocking mAbs 1G7 and 2F11 enhanced NK cytotoxicity, as compared to isotype controls (FIG. 19B). Additionally, HHLA2 blocking antibodies 2C4 and 2G2 enhanced NK cytotoxicity (FIG. 19C). 8F7, a KIR3DL3 antibody, and 6D10, an HHLA2 antibody which are weak blockers of the KIR3DL3: HHLA2 interaction, did not enhance cytotoxicity (FIG. 19B and FIG. 19C).

To examine the role of KIR3DL3 in regulating NK cell lytic activity, a CD107a degranulation assay was performed. As measured by CD107a expression on the NK92-MI cell surface (degranulation), HHLA2 expression on the Raji B2M KO cells inhibits NK92-MI degranulation (FIG. 19D) and blockade of KIR3DL3 with the 1G7 mAb leads to enhanced degranulation of these NK cells (FIG. 19E).

### Example 12: HHLA2 is Expressed in RCC Tumors and is Distinct from PD-L1 Expression

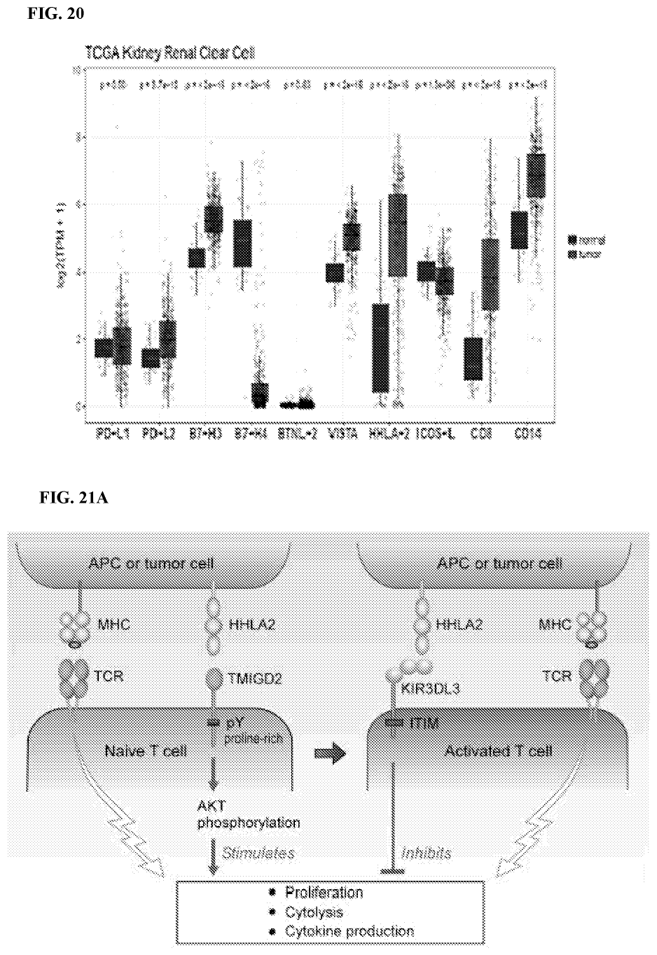

Pan-cancer transcriptomic analyses have demonstrated that HHLA2 is expressed in multiple tumor types, with renal cell carcinoma (RCC) showing highest levels of expression compared to normal tissue. In the analysis of mRNA expression of B7 family members in the TCGA, it was found herein that HHLA2 was the most highly upregulated B7 family member in ccRCC compared to normal kidney tissue (FIG. 20). PD-L1, PD-L2, B7-H3 and VISTA transcripts were only slightly higher in ccRCC compared to normal kidney and B7-H4 transcript was markedly reduced in ccRCC. As expected, CD8 transcripts were upregulated in ccRCC compared to normal kidney, reflecting the high immune infiltrate in ccRCC. There was a negative association between PD-L1 and HHLA2 expression, particularly within tumors.

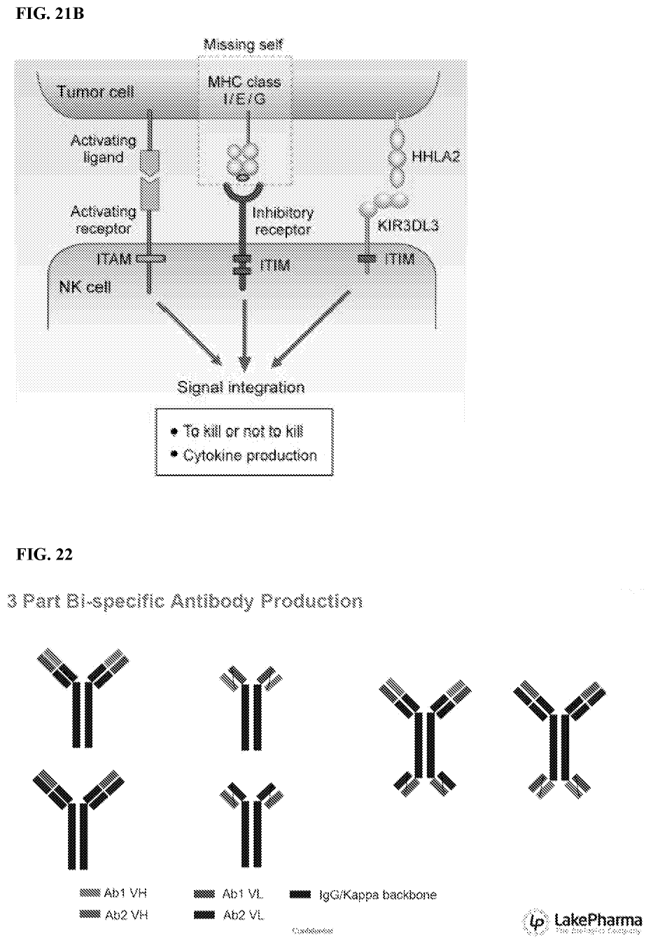

HHLA2 is a B7 family member with both immune stimulatory and inhibitory functions. TMIGD2 has previously been characterized as the immune stimulatory receptor for HHLA2 in both T and NK cells but the mechanisms by which HHLA2 could inhibit T and NK has been unknown (FIG. 21A and FIG. 21B). It was hypothesized herein the presence of an inhibitory receptor that could mediate this effect. A screen was performed herein for additional HHLA2 receptors using soluble recombinant HHLA2-mIgG2a on an array of cell surface receptors and identified KIR3DL3 as a novel receptor for HHLA2. KIR3DL3 is a member of the KIR family of receptors but to date its ligand was not known. It is demonstrated herein that the HHLA2-KIR3DL3 interaction is immune inhibitory in T and NK cells and that antibodies that block this interaction reverse the immune inhibition. Analysis for HHLA2 expression in RCC patient tumors shows a largely non-overlapping pattern of expression with PD-L1. Taken together, these findings demonstrate blockade of the KIR3DL3/HHLA2 interaction could represent a novel approach for cancer immunotherapy.

Killer-cell immunoglobulin-like receptors (KIRs) contribute to both innate and adaptive immune responses through their expression by NK cells and T cells. HLA class I ligands have been identified for 9 of the 13 KIR but no HLA ligand for KIR3DL3 has been identified. Members of the KIR family have a similar protein structure with 2 or 3 extracellular Ig domains and either a short (activating DS form) or long intracellular domain (inhibiting DL form) containing immunoreceptor tyrosine-based inhibitory motifs (ITIM). KIR3DL3 gene encodes three extracellular Ig domains, has only one ITIM and lacks the exon encoding the stem between the Ig domains and the transmembrane region. Although the structure of KIR3DL3 is suggestive of an inhibitory receptor, the function of KIR3DL3 has not been demonstrated and cognate ligands have not been identified.

The KIR gene family consists of 13 genes that encode either inhibitory or activating receptors. The expression of individual KIR genes is clonally distributed with only a fraction of NK and T cells expressing a given KIR repertoire. In individuals, the 13 KIR genes vary in their presence and copy number but KIR3DL3 is unique in the KIR family as it is present in all individuals. The KIR3DL3 promoter is the strongest of the KIR promoters but is generally silenced by methylation. KIR3DL3 is highly polymorphic; however, the majority of the 157 polymorphic residues map to sites distinct from known HLA ligand binding sites of other KIRs based on a KIR3DL3 structure model. This indicates that these polymorphisms are unlikely to affect HHLA2 ligand binding.

KIR3DL3 is rarely expressed in normal tissues with the exception of placental decidual NK cells and activated NK cells. HHLA2 is highly expressed on placental trophoblasts. This expression pattern is similar to PD-L1 expression on placental trophoblasts and other sites of immune privilege and consistent with a primary natural role in immune suppression. It was demonstrated herein for the first time that HHLA2 is a ligand for KIR3DL3 and functions as an inhibitory receptor in NK and T cells.

Consistent with the present observation that the HHLA2/KIR3DL3 interaction inhibits CD28 dependent CD3 signaling in T cells, Reider, et al. also showed that HHLA2-Fc inhibits T cell activation mediated by CD3 and CD28 signaling and ERK2 tyrosine phosphorylation. These observations are similar to results showing PD-L1/PD-1 interaction inhibits a CD3 signaling pathway dependent on tyrosine phosphorylation and MEK-1/ERK2 activation. Inhibition of CD3 signaling by PD-L1 is through tyrosine phosphorylation of the PD-1 ITSM motif and recruitment of SHP-2 phosphatase, which dephosphorylates proximal signaling molecules of the TCR and CD28 pathways. Taken together the above results indicate that PD-1 and KIR3DL3 may share similar pathways following T cell activation and inhibition of both pathways could be additive.

Expression of inhibitory immune receptors on T cells is dynamically modulated with activation. The HHLA2: KIR3DL3 pathway has parallels to the B7: CTLA-4 pathway. Resting T cells express immune stimulatory receptors such as CD28 (for CD80/86) and TMIGD2 (for HHLA2). TMIGD2 is primarily expressed on naive T cells and is down-modulated upon T cell activation and only expressed on 22% of memory CD4+ T cells and 29% of memory CD8+ T cells. Similarly, CD28 is expressed on only 50% of antigen-experienced human CD8 T cells. Upon activation, T cells upregulate expression of inhibitory receptors including CTLA-4, PD-1, and KIR3DL3 as presented herein. We show that KIR3DL3 expression is rare in non-activated T cells and is induced in a modest subpopulation of CD4 and CD8 T cells following stimulation with anti-CD3 and CD28 mAbs. Unlike the early PD-1 expression following T cell activation, KIR3DL3 is expressed later and peaks around day 21 following T cell activation. This pattern of regulation predicts that neoantigen-experienced T cells will express a preponderance of immunoinhibitory receptors at different times following activation. If the cognate ligands (B7 or HHLA2) are present, T cell inhibition can become the dominant outcome.

RCC is a malignancy with many recent therapeutic advances. RCC is highly immune infiltrated and has historically been immune responsive. IL-2 therapy has been an immune option for patients since 1992. More recently, inhibitors of the PD-1 pathway have not only shown activity as monotherapy but are also approved in combination with VEGFR TKI therapy or CTLA-4. However, resistance to therapy is seen in many patients and new treatment options are needed.

It is demonstrated herein that HHLA2 expression and PD-L1 expression are non-overlapping in RCC. This is consistent with a prior report on non-small cell lung cancer (NSCLC) in which 64% of tumor samples were HHLA2 positive and of these, 67% were PD-L1 negative. This study in NSCLC together with the HHLA2 and PD-L1 expression studies in ccRCC presented herein provide a rationale for targeting the HHLA2/KIR3DL3 immune checkpoint pathway alone or in combination with PD-1 inhibitors in these cancers and other cancers where the HHLA2 inhibitory pathway has been implicated.

One of the key accomplishments of this study is the identification of KIR3DL3 as the inhibitory receptor for HHLA2 and generation of checkpoint inhibitor antibodies that reverse HHLA2-KIR3DL3-mediated inhibition in T and NK cells. Prior to this study, it was not known if the binding of HHLA2 to TMIGD2 and KIR3DL3 occurred through one common overlapping epitope or via distinct sites of interaction. The present findings show that most of the HHLA2 antibodies generated block binding of HHLA2 to both TIMGD2 and KIR3DL3. Antibodies that block only one of these two receptor interactions were rare, thus the HHLA2 binding sites on TMIGD2 and KIR3DL3 appear to be overlapping but non-identical. It is demonstrated herein that HHLA2 antibodies that block the KIR3DL3-HHLA2 interaction but preserve the TMIGD2 stimulatory signal is one option for therapeutic development. Additionally, KIR3DL3 antibodies that selectively block KIR3DL3-HHLA2 interaction are also attractive candidates for therapeutic development.

Many immune combinations have been tested in preclinical models and multiple candidates have progressed to clinical trials. Most have not yet shown outstanding clinical activity. One hypothesis for this limited efficacy is that many pathways being co-targeted with PD-L1 share mechanisms for upregulation of expression. For example, Indoleamine 2,3-Dioxygenase (IDO) inhibition in cancer has been of interest but clinical trials of IDO-PD-1 combinations have not yielded promising results. IFN-γ upregulates both IDO and PD-L1 expression. It is demonstrated herein that HHLA2 expression is not regulated by IFN-γ and thus represents an independently regulated mechanism of tumor immune evasion. Additionally, HHLA2 immune signaling is important not only in T cells but in NK cells and inhibition of HHLA2 signaling through KIR3DL3 may affect a diverse set of lymphocytes in the immune microenvironment that mediate both innate and adaptive immune responses. In tumors, HHLA2 and PD-L1 expression appear to be non-overlapping and independently regulated.

Both the receptors and ligands of the HHLA2-KIR3DL3-TMIGD2 pathway are expressed in primates and not found in multiple other mammals including rodents, which is unique within the B7 and CD28 families. Thus, the functional studies of this pathway are limited to in vitro models using human cell lines and primary immune cells. To assess the in vivo role of this pathway, humanized models need to be developed. A triple knock in approach would be needed including genomic regulatory regions and complete genes as both the receptors and ligand have no murine orthologs.

Immune checkpoint inhibition of the PD-1 pathway is now the cornerstone for immune therapy of cancer. Despite the success of PD-1 inhibition, many patients develop resistance and identification of novel, non-redundant, immune inhibitory pathways is an important need in this field. Shifting the balance of immune inhibitory and stimulatory pathways away from inhibition may optimize the anti-tumor immune response. In summary, KIR3DL3 is identified herein as an immune inhibitory receptor for HHLA2. It is also developed herein the HHLA2 and KIR3DL3 antibodies that specifically block the immune inhibitory activity but spare the co-stimulatory activity of TMIGD2 (FIG. 21A and FIG. 21B). Phase I clinical trials testing the safety and preliminary efficacy of HHLA2 pathway inhibition are currently being developed.

### Example 13: Derivation of KIR3DL3 × PD-1 mAb IgG-scFV Bispecific Antibody

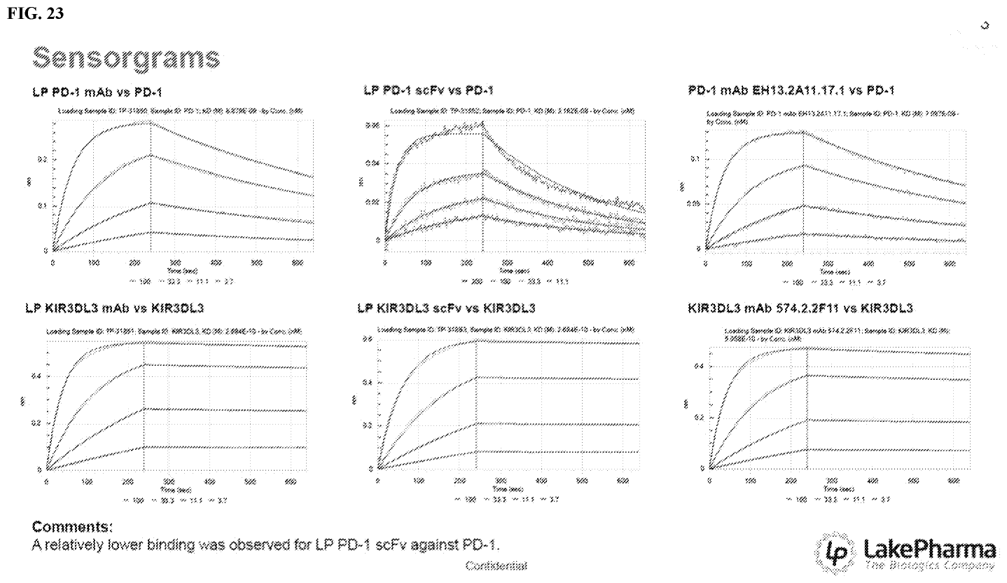

KIR3DL3 mAb 574.2.2F11 (referred to as 2F11) and PD-1 mAb EH13.2A11 (referred to as EH13) mouse hybridoma clones were selected for constructing a bispecific antibody. The 2F11 and EH13 clones were selected based on their high affinity against the target and potency for checkpoint inhibition in T cells. As the first step 2F11 and EH13 VH and V_{L} genes were expressed as either human IgG4 or scFv antibodies and tested for binding to their antigens KIR3DL3 and PD-1 respectively in an Octet binding assay as described in Table 6 methods. Binding affinities were compared to the respective parental hybridoma clones. As shown in Table 6 and FIG. 23, the KD's for KIR3DL3 mAb 2F11 human IgG4 and scFV formats were within 2-3 fold of the control parental hybridoma clone (0.29 nM to 0.51 nM). The PD-1 mAb EH13 human IgG4 showed similar binding within 1-2 fold compared to the control hybridoma (6.9 nM to 7.1 nM). However, the EH13 scFV showed a 4-5 fold lower affinity compared to the parental control hybridoma (31.6 nM vs 6.88 nM).

Based on these results a bispecific antibody format comprised of the PD-1 mAb EH13 human IgG4 and KIR3DL3 mAb 2F11 scFV was selected for construction of the bi-specific antibody.

The amino acid sequences for PD-1 and KIR3DL3 IgG4, scFV and the bi-specific antibody are shown in Tables 7, 8, and 9, respectively.

The agents that target both KIR3DL3 and PD-1 can be used to modulate immune response and/or treat cancer. For example, the KIR3DL3 × PD-1 bispecific antibodies are checkpoint immunotherapy that activate T and NK cells in tumors. In some instances, the KIR3DL3 × PD-1 bispecific antibody is additive or synergistic with PD-1 or PD-L1 or other checkpoint immunotherapy. Furthermore, HHLA2 or KIR3DL3 expression in the tumor is a useful biomarker of responsiveness to KIR3DL3 mAb and/or KIR3DL3 × PD-1 bispecific antibody checkpoint blockade.

TABLE 5
HHLA2 and KIR3DL3 mAb binding and receptor blocking characteristics

| | HHLA2 Binding | KIR3DL3 Blocking | TMIGD2 Blocking |
|---|---|---|---|
| HHLA2 mAb | EC50 (ug/ml) | IC50 (ug/ml) | IC50 (ug/ml) |
| 2C4 (IgG2a) | 0.28 | 1.22 | >20 |
| 2G2 (IgG1) | 0.24 | 0.88 | 1.18 |
| 6D10 (IgG1) | 2.3 | 3.74 | >20 |
| 6F10 (IgG1) | 0.3 | 1.11 | 1.43 |

| | KIR3DL3 Binding | KIR3DL3 Blocking | TMIGD2 Blocking |
|---|---|---|---|
| KIR3DL3 mAb | EC50 (ug/ml) | IC50 (ug/ml) | IC50 (ug/ml) |
| 1G7 (IgG2b) | 0.13 | 0.61 | — |
| 2F11 (IgG2b) | 0.43 | 0.42 | — |
| 8F7 (IgG1) | 1.0 | >10 | — |

In Table 5, HHLA2 (top) or KIR3DL3 (bottom) mAb binding to HHLA2 or KIR3DL3 transfected 300.19 cells is shown with EC50 values. The blockade of HHLA2-mIgG2a binding to KIR3DL3 and TMIGD2 transfected cells by HHLA2 and KIR3DL3 mAbs is shown with IC50 values.

TABLE 6
Binding affinities of KIR3DL3 and PD-1 human IgG4 and scFV antibodies
Methods & Kinetic Constants

| | |
|---|---|
| SR# | SR-18985 |
| Date | Aug. 6, 2020 |
| Scientist | YEE |
| Equipment | Octet RED96 |
| Assay Buffer | 0.1% BSA, 1XPBS, 0.02% Tween-20, 0.05% NaN3 |
| Regeneration Buffer | N/A |

### Procedures:

Binding experiments were performed on Octat Red af 25″° C. The human antibodies were loaded onto Anti-higG Fe Capture biosensors and the mouse antibodies were loaded onto Anti-migG Fe Capture biosensors both for 300 seconds. The ligand loaded sensors were dipped into a 3-fold series dilution (starting at 300 nad) of the antigens (PD-1 and KIR3DL3) for 240 seconds followed by dissociation for 400 seconds. Kinetic constanta were calculated using a monovalent (1:1) binding model Antigen: Haman PD1, Sino Biological Cat #: HPLC-10377-HOSH.

Antigen: Human KIR3DL3, Sino Biological Cat#: 16055-H08H

| Loading Sample ID | Sample ID | KD (M) | kon(1/Ms) | kdis(1/s) | Full X\hat{ }2 | Full R\hat{ }2 |
|---|---|---|---|---|---|---|
| LP PD-1 mAb (TP-31850) | PD-1 | 6.88E−09 | 1.99E+05 | 1.37E−03 | 0.0126 | 0.9993 |
| LP PD-1 scFv (TP-31852) | PD-1 | 3.16E−08 | 1.08E+05 | 3.41E−03 | 0.0082 | 0.9854 |
| PD-1 mAb EH13.2A11.17.1 | PD-1 | 7.10E−09 | 2.16E+05 | 1.54E−03 | 0.0053 | 0.9985 |
| LP KIR3DL3 mAb (TP-31851) | KIR3DL3 | 2.89E−10 | 2.48E+05 | 7.19E−05 | 0.0326 | 0.9996 |
| LP KIR3DL3 scFv (TP-31853) | KIR3DL3 | 2.68E−10 | 1.65E+05 | 4.42E−05 | 0.0531 | 0.9995 |
| KIR3DL3 mAb 574.2.2F11 | KIR3DL3 | 5.06E−10 | 2.33E+05 | 1.18E−04 | 0.0288 | 0.9995 |

Comments:
Non-specific binding between bare sensors (AHC, AMC) and the antigens was not observed during scouting.

TABLE 7
Representative exemplary amino acid sequences of PD-1 EH13 and
KIR3DL3 2F11 mAb human IgG4

Antibody: PD-1 EH13.2A11.17.1
Heavy Chain
Reference: MHC801HC.2
Isotype: Human IgG4 (S228P)
SignalPeptide_EVKLVESGGGLVQPGGSLKLSCAASGFTFSSFTLSWVRQTPEKRLEWVAYISYGG
ADTYYPDTVKGRFTIFRDNAKNTLYLQMSSLKSEDTARYYCARHGPYGKGDYYAMDYWGQG
TSVTVSSASTKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALHNHYTQKSLSLSLG*
Light Chain
Reference: MHC801LC.1
Isotype: Human Kappa (Km3 allotype)
SignalPeptide_DIVLTQSPASLAVSLGQRATISCRASESVDDFGISFMNWFQQKPGQPPKLLIFAAS
KQGSGVPARFSGSGSGTDFSLNIHPMEEDDTAMYFCQQSKEVPWTFGGVTKLEIKRTVAAPSVF
IFPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGEC*
Antibody: KIR3DL3 mAb 574.2.2F11.2.7.4
Heavy Chain
Reference: AVS-3703HCT.2
Isotype: Human IgG4 (S228P)
SignalPeptide_DVQLVESGGGLVQPGGSRKLSCAASGFTFSYFGMHWVRRAPEKGLEWVAFISSD
STTIYYTDTVKGRFTISRDNPKNTLFLQMTSLRSEDTAIYYCARFYDGAMDYWGQGTSVTVSTA
STKGPSVFPLAPCSRSTSESTAALGCLVKDYFPEPVTVSWNSGALTSGVHTFPAVLQSSGLYSLSSVV
TVPSSSLGTKTYTCNVDHKPSNTKVDKRVESKYGPPCPPCPAPEFLGGPSVFLFPPKPKDTLMISRTPE
VTCVVVDVSQEDPEVQFNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKSLSLSLG*
Light Chain
Reference: AVS-3703LCT.2
Isotype: Human Kappa (Km3 allotype)
SignalPeptide_DIVMTQSQKFMSTSVGDRVSITCKASQSVRTAVAWYQQKPGQSPKVLIYLASNRH
TGVPDRFTGSGSGTDFTLTISNVQSEDLADYFCLQHWNYPLTFGAGTKLELKRTVAAPSVFIFPP
SDEQLKSGTASVVCLLNNFYPREAKVQWKVDNALQSGNSQESVTEQDSKDSTYSLSSTLTLSKADYE
KHKVYACEVTHQGLSSPVTKSFNRGEC*

TABLE 8
Representative exemplary amino acid sequences of PD-1 EH13 and
KIR3DL3 2F11 mAb scFv's

Protein: PD-1 scFv
Reference: MHC801HC.2, MHC801LC.1
Isotype: Human IgG4 (S228P) Fc
SignalPeptide_ESKYGPPCPPCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGKGGGGAGGGGEVKLVESGGGLVQPGGSL
KLSCAASGFTFSSFTLSWVRQTPEKRLEWVAYISYGGADTYYPDTVKGRFTIFRDNAKNTLYLQ
MSSLKSEDTARYYCARHGPYGKGDYYAMDYWGQGTSVTVSSGGGGSGGGGSGGGGSDIVLTQ
SPASLAVSLGQRATISCRASESVDDFGISFMNWFQQKPGQPPKLLIFAASKQGSGVPARFSGSGS
GTDFSLNIHPMEEDDTAMYFCQQSKEVPWTFGGVTKLEIK*
Protein: KIR3DL3 scFv
Reference: AVS-3703HCT.2, AVS-3703LCT.2
Isotype: Human IgG4 (S228P) Fc
SignalPeptide_ESKYGPPCPPCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGKGGGGAGGGGDVQLVESGGGLVQPGGSR
KLSCAASGFTFSYFGMHWVRRAPEKGLEWVAFISSDSTTIYYTDTVKGRFTISRDNPKNTLFLQ
MTSLRSEDTAIYYCARFYDGAMDYWGQGTSVTVSTGGGGSGGGGSGGGGSDIVMTQSQKFMST
SVGDRVSITCKASQSVRTAVAWYQQKPGQSPKVLIYLASNRHTGVPDRFTGSGSGTDFTLTISN
VQSEDLADYFCLQHWNYPLTFGAGTKLELK*

In some embodiments, the heavy chain/Fc construct is designed with the C-terminal lysine removed to create a more homogeneous product, as referenced by Cai et al. (2011) Biotechnol Bioeng, 108 (2): 404-12. The sequences presented in Table 8 are the sequences in which the terminal lysine has been removed.

In some embodiments, sequences are codon optimized for mammalian expression.

In some embodiments, the gene inserts with the listed elements and tags will be included in the final construct for expression. In some embodiments, the full insert will be cloned into a high expression mammalian vector.

TABLE 9
Representative amino acid sequences of an exemplary PD-1 x KIR3DL3
bispecific antibody-PD-1 EH13 IgG4 mAb and KIR3DL3 2F11 scFv Bispecific
antibody

Antibody: PD-1-IgG4_KIR3DL3-ScFv
Heavy Chain
References:
• H12858, pLEV123-PD-1 EH13.2A11.17.1-hlgG4 (S228P, L235E)
• J2876, pLEV123-KIR3DL3 scFv
Isotype: Human IgG4 (S228P, L235E)
SignalPeptide_EVKLVESGGGLVQPGGSLKLSCAASGFTFSSFTLSWVRQTPEKRLEWVAYISYGG
ADTYYPDTVKGRFTIFRDNAKNTLYLQMSSLKSEDTARYYCARHGPYGKGDYYAMDYWGQG
TSVTVSSASTKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFEGGPSVFLFPPKPKD
TLMISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEALHNHYTQKSLSLSLGGG
GGAGGGGDVQLVESGGGLVQPGGSRKLSCAASGFTFSYFGMHWVRRAPEKGLEWVAFISSDST
TIYYTDTVKGRFTISRDNPKNTLFLQMTSLRSEDTAIYYCARFYDGAMDYWGQGTSVTVSTGG
GGSGGGGSGGGGSDIVMTQSQKFMSTSVGDRVSITCKASQSVRTAVAWYQQKPGQSPKVLIYL
ASNRHTGVPDRFTGSGSGTDFTLTISNVQSEDLADYFCLQHWNYPLTFGAGTKLEL*
Light Chain [Same LC AS L12858]
Reference: L12858, pLEV123-PD-1 EH13.2A11.17.1-hKappa
Isotype: Human Kappa (Km3 allotype)
SignalPeptide_DIVLTQSPASLAVSLGQRATISCRASESVDDFGISFMNWFQQKPGQPPKLLIFAAS
KQGSGVPARFSGSGSGTDFSLNIHPMEEDDTAMYFCQQSKEVPWTFGGVTKLEIKRTVAAPSVF
IFPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGEC*

In some embodiments, the heavy chain/Fc construct is designed with the C-terminal lysine removed to create a more homogeneous product, as referenced by Cai et al. (2011) Biotechnol Bioeng, 108 (2): 404-12. The sequences presented in Table 9 are the sequences in which the terminal lysine has been removed.

In some embodiments, sequences are codon optimized for mammalian expression. The sequences in Table 9 have been previously codon optimized and constructed in SR-18230.

In some embodiments, the gene inserts with the listed elements and tags will be included in the final construct for expression. In some embodiments, the full insert will be cloned into a high expression mammalian vector.

### REFERENCES

1 Zou W, Wolchok J D, Chen L. PD-L1 (B7-H1) and PD-1 pathway blockade for cancer therapy: Mechanisms, response biomarkers, and combinations. Sci Transl Med. 2016; 8 (328): 328rv4.
2. Baumeister S H, Freeman G J, Dranoff G, Sharpe A H. Coinhibitory Pathways in Immunotherapy for Cancer. Annu Rev Immunol. 2016; 34:539-73.
3. Sharma P, Allison J P. The future of immune checkpoint therapy. Science. 2015;348 (6230): 56-61.
4. Pitt J M, Vetizou M, Daillere R, Roberti M P, Yamazaki T, Routy B, et al. Resistance Mechanisms to Immune-Checkpoint Blockade in Cancer: Tumor-Intrinsic and-Extrinsic Factors. Immunity. 2016; 44 (6): 1255-69.
5. Zaretsky J M, Garcia-Diaz A, Shin D S, Escuin-Ordinas H, Hugo W, Hu-Lieskovan S, et al. Mutations Associated with Acquired Resistance to PD-1 Blockade in Melanoma. N Engl J Med. 2016; 375 (9): 819-29.
6. O'Donnell J S, Long G V, Scolyer R A, Teng M W, Smyth M J. Resistance to PD1/PDL1 checkpoint inhibition. Cancer Treat Rev. 2017; 52:71-81.
7. Crespo J, Vatan L, Maj T, Liu R, Kryczek I, Zou W. Phenotype and tissue distribution of CD28H (+) immune cell subsets. Oncoimmunology. 2017;6 (12): e1362529.
8. Janakiram M, Chinai J M, Fineberg S, Fiser A, Montagna C, Medavarapu R, et al. Expression, Clinical Significance, and Receptor Identification of the Newest B7 Family Member HHLA2 Protein. Clin Cancer Res. 2015;21 (10): 2359-66.
9. Zhou Q H, Li K W, Chen X, He H X, Peng S M, Peng S R, et al. HHLA2 and PD-L1 co-expression predicts poor prognosis in patients with clear cell renal cell carcinoma. J Immunother Cancer. 2020; 8 (1).
10. Wang B, Ran Z, Liu M, Ou Y. Prognostic Significance of Potential Immune Checkpoint Member HHLA2 in Human Tumors: A Comprehensive Analysis. Front Immunol. 2019; 10:1573.
11. Zhu Y, Yao S, Iliopoulou B P, Han X, Augustine M M, Xu H, et al. B7-H5 costimulates human T cells via CD28H. Nat Commun. 2013; 4:2043.
12. Zhao R, Chinai J M, Buhl S, Scandiuzzi L, Ray A, Jeon H, et al. HHLA2 is a member of the B7 family and inhibits human CD4 and CD8 T-cell function. Proc Natl Acad Sci USA. 2013; 110 (24) : 9879-84.
13. Wang J, Manick B, Wu G, Hao R. Biofunctions of three new B7 family members (IRM7P.486). J Immunol. 2014; 192 ((1 Supplement)): 126.11.
14. Xiao Y, Freeman G J. A New B7: CD28 Family Checkpoint Target for Cancer Immunotherapy: HHLA2. Clin Cancer Res. 2015;21 (10): 2201-3.
15. Zhuang X, Long E O. CD28 Homolog Is a Strong Activator of Natural Killer Cells for Lysis of B7H7 (+) Tumor Cells. Cancer Immunol Res. 2019; 7 (6): 939-51.
16. Pende D, Falco M, Vitale M, Cantoni C, Vitale C, Munari E, et al. Killer Ig-Like Receptors (KIRs): Their Role in NK Cell Modulation and Developments Leading to Their Clinical Exploitation. Front Immunol. 2019; 10:1179.
17. Bakker A B, Phillips J H, Figdor C G, Lanier L L. Killer cell inhibitory receptors for MHC class I molecules regulate lysis of melanoma cells mediated by NK cells, gamma delta T cells, and antigen-specific CTL. J Immunol. 1998;160 (11): 5239-45.
18. Guerra N, Guillard M, Angevin E, Echchakir H, Escudier B, Moretta A, et al. Killer inhibitory receptor (CD158b) modulates the lytic activity of tumor-specific T lymphocytes infiltrating renal cell carcinomas. Blood. 2000;95 (9): 2883-9.
19. Mingari M C, Schiavetti F, Ponte M, Vitale C, Maggi E, Romagnani S, et al. Human CD8+T lymphocyte subsets that express HLA class I-specific inhibitory receptors represent oligoclonally or monoclonally expanded cell populations. Proc Natl Acad Sci U S A. 1996; 93 (22): 12433-8.
20. Rieder S A, Wang J, White N, Qadri A, Menard C, Stephens G, et al. B7-H7 (HHLA2) inhibits T-cell activation and proliferation in the presence of TCR and CD28 signaling. Cell Mol Immunol. 2020.
21. Leaton L A, Shortt J, Kichula K M, Tao S, Nemat-Gorgani N, Mentzer A J, et al. Conservation, Extensive Heterozygosity, and Convergence of Signaling Potential All Indicate a Critical Role for KIR3DL3 in Higher Primates. Front Immunol. 2019; 10:24.
22. Trompeter H I, Gomez-Lozano N, Santourlidis S, Eisermann B, Wernet P, Vilches C, et al. Three structurally and functionally divergent kinds of promoters regulate expression of clonally distributed killer cell Ig-like receptors (KIR), of KIR2DL4, and of KIR3DL3. J Immunol. 2005;174 (7): 4135-43.
23. Vivian J P, Duncan R C, Berry R, O'Connor G M, Reid H H, Beddoe T, et al. Killer cell immunoglobulin-like receptor 3DL1-mediated recognition of human leukocyte antigen B. Nature. 2011; 479 (7373): 401-5.
24. Trundley A E, Hiby S E, Chang C, Sharkey A M, Santourlidis S, Uhrberg M, et al. Molecular characterization of KIR3DL3. Immunogenetics. 2006;57 (12): 904-16.
25. Brown J A, Dorfman D M, Ma F R, Sullivan E L, Munoz O, Wood C R, et al. Blockade of programmed death-1 ligands on dendritic cells enhances T cell activation and cytokine production. J Immunol. 2003; 170 (3): 1257-66.
26. Sheppard K A, Fitz L J, Lee J M, Benander C, George J A, Wooters J, et al. PD-1 inhibits T-cell receptor induced phosphorylation of the ZAP70/CD3zeta signalosome and downstream signaling to PKCtheta. FEBS Lett. 2004; 574 (1-3): 37-41.
27. Chemnitz J M, Parry R V, Nichols K E, June C H, Riley J L. SHP-1 and SHP-2 associate with immunoreceptor tyrosine-based switch motif of programmed death 1 upon primary human T cell stimulation, but only receptor ligation prevents T cell activation. J Immunol. 2004; 173 (2): 945-54.
28. Hui E, Cheung J, Zhu J, Su X, Taylor M J, Wallweber H A, et al. T cell costimulatory receptor CD28 is a primary target for PD-1-mediated inhibition. Science. 2017;355 (6332): 1428-33.
29. Patsoukis N, Duke-Cohan J S, Chaudhri A, Aksoylar H I, Wang Q, Council A, et al. Interaction of SHP-2 SH2 domains with PD-1 ITSM induces PD-1 dimerization and SHP-2 activation. Commun Biol. 2020; 3 (1): 128.
30. Boussiotis V A. Molecular and Biochemical Aspects of the PD-1 Checkpoint Pathway. N Engl J Med. 2016; 375 (18): 1767-78.
31. Sharpe A H, Freeman G J. The B7-CD28 superfamily. Nat Rev Immunol. 2002;2 (2): 116-26.
32. Tian Y, Sun Y, Gao F, Koenig M R, Sunderland A, Fujiwara Y, et al. CD28H expression identifies resident memory CD8+ T cells with less cytotoxicity in human peripheral tissues and cancers. Oncoimmunology. 2019; 8 (2): e1538440.
33. Zhong C, Lang Q, Yu J, Wu S, Xu F, Tian Y. Phenotypical and potential functional characteristics of different immune cells expressing CD28H/B7-H5 and their relationship with cancer prognosis. Clin Exp Immunol. 2020; 200 (1): 12-21.
34. Lum L G, Orcutt-Thordarson N, Seigneuret M C, Hansen J A. In vitro regulation of immunoglobulin synthesis by T-cell subpopulations defined by a new human T-cell antigen (9.3). Cell Immunol. 1982;72 (1): 122-9.
35. Linsley P S, Greene J L, Tan P, Bradshaw J, Ledbetter J A, Anasetti C, et al. Coexpression and functional cooperation of CTLA-4 and CD28 on activated T lymphocytes. J Exp Med. 1992; 176 (6): 1595-604.
36. Agata Y, Kawasaki A, Nishimura H, Ishida Y, Tsubata T, Yagita H, et al. Expression of the PD-1 antigen on the surface of stimulated mouse T and B lymphocytes. Int Immunol. 1996; 8 (5): 765-72.
37. Margolin K A, Rayner A A, Hawkins M J, Atkins M B, Dutcher J P, Fisher R I, et al. Interleukin-2 and lymphokine-activated killer cell therapy of solid tumors: analysis of toxicity and management guidelines. J Clin Oncol. 1989;7 (4): 486-98.
38. Atkins M B, Lotze M T, Dutcher J P, Fisher R I, Weiss G, Margolin K, et al. High-dose recombinant interleukin 2 therapy for patients with metastatic melanoma: analysis of 270 patients treated between 1985 and 1993. J Clin Oncol. 1999;17 (7): 2105-16.
39. Motzer R J, Penkov K, Haanen J, Rini B, Albiges L, Campbell M T, et al. Avelumab plus Axitinib versus Sunitinib for Advanced Renal-Cell Carcinoma. N Engl J Med. 2019; 380 (12): 1103-15.
40. Rini B I, Plimack E R, Stus V, Gafanov R, Hawkins R, Nosov D, et al. Pembrolizumab plus Axitinib versus Sunitinib for Advanced Renal-Cell Carcinoma. N Engl J Med. 2019; 380 (12): 1116-27.
41. Motzer R J, Tannir N M, McDermott D F, Aren Frontera O, Melichar B, Choueiri T K, et al. Nivolumab plus Ipilimumab versus Sunitinib in Advanced Renal-Cell Carcinoma. N Engl J Med. 2018; 378 (14): 1277-90.
42. Cheng H, Borczuk A, Janakiram M, Ren X, Lin J, Assal A, et al. Wide Expression and Significance of Alternative Immune Checkpoint Molecules, B7× and HHLA2, in PD-L1-Negative Human Lung Cancers. Clin Cancer Res. 2018; 24 (8): 1954-64.
43. Munn D H. Blocking IDO activity to enhance anti-tumor immunity. Front Biosci (Elite Ed). 2012; 4:734-45.
44. Gaule P, Smithy J W, Toki M, Rehman J, Patell-Socha F, Cougot D, et al. A Quantitative Comparison of Antibodies to Programmed Cell Death 1 Ligand 1. JAMA Oncol. 2017;3 (2): 256-9.
45. Mahoney K M, Sun H, Liao X, Hua P, Callea M, Greenfield E A, et al. PD-L1 Antibodies to Its Cytoplasmic Domain Most Clearly Delineate Cell Membranes in Immunohistochemical Staining of Tumor Cells. Cancer Immunol Res. 2015;3 (12): 1308-15.
46. Callea M, Albiges L, Gupta M, Cheng S C, Genega E M, Fay A P, et al. Differential Expression of PD-L1 between Primary and Metastatic Sites in Clear-Cell Renal Cell Carcinoma. Cancer Immunol Res. 2015;3 (10): 1158-64.
47. Perez J A, Goldsack E, Norambuena L. [Medullary carcinoma of the thyroid. Clinical case]. Rev Med Chil. 1989;117 (4): 431-4.
48. Pignon J C, Jegede O, Shukla S A, Braun D A, Horak C E, Wind-Rotolo M, et al. irRECIST for the Evaluation of Candidate Biomarkers of Response to Nivolumab in Metastatic Clear Cell Renal Cell Carcinoma: Analysis of a Phase II Prospective Clinical Trial. Clin Cancer Res. 2019; 25 (7): 2174-84.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih. gov.

### EQUIVALENTS

Those skilled in the art will recognize, or be able to ascertain using no more than routine experimentation, many equivalents to the specific embodiments encompassed by the present disclosure described herein. Such equivalents are intended to be encompassed by the following claims.

1. A monoclonal antibody, or antigen-binding fragment thereof, comprising:
a) a VH comprising a VH CDR1 amino acid sequence of SEQ ID NO: 67, a VH CDR2 amino acid sequence of SEQ ID NO: 69, a VH CDR3 amino acid sequence of SEQ ID NO: 71; and
VL comprising a VL CDR 1 amino acid sequence of SEQ ID NO: 73, a VL CDR2 amino acid of SEQ ID NO: 75, and a VL CDR3 amino acid sequence of SEQ ID NO: 77; or
b) one, two, or three heavy chain CDR sequences each selected from the group consisting of the sequences listed in Tables 2, 7, and 8; and one, two, or three light chain CDR sequences each selected from the group consisting the sequences listed in Tables 2, 7, and 8.

2. The monoclonal antibody, or antigen-binding fragment thereof, of claim 1, wherein the monoclonal antibody, or antigen-binding fragment thereof, comprises:
a) a VH comprising an amino acid sequence of SEQ ID NO: 79; and
b) a VL comprising an amino acid sequence of SEQ ID NO: 81.

3-4. (canceled)

5. The monoclonal antibody, or antigen-binding fragment thereof, of claim 1, wherein the monoclonal antibody, or antigen-binding fragment thereof,
i) is chimeric, humanized, composite, murine, or human;
ii) is (a) detectably labeled, (b) conjugated to a cytotoxic agent, optionally a chemotherapeutic agent, a biologic agent, a toxin, and/or a radioactive isotope, (c) comprises an effector domain, (d) comprises an Fc domain, and/or (e) is selected from the group consisting of Fv, Fav, F(ab′)2), Fab′, dsFv, scFv, sc (Fv)2, and diabodies fragments;
iii) inhibits the binding of HHLA2 to KIR3DL3; and/or
iv) specifically binds KIR3DL3.

6-9. (canceled)

10. A bispecific antibody, or antigen-binding fragment thereof, comprising:
a) VH comprising a VH CDR1 amino acid sequence of SEQ ID NO: 67, a VH CDR2 amino acid sequence of SEQ ID NO: 69, a VH CDR3 amino acid sequence of SEQ ID NO: 71; and
VL comprising a VL CDR 1 amino acid sequence of SEQ ID NO: 73, a VL CDR2 amino acid of SEQ ID NO: 75, and a VL CDR3 amino acid sequence of SEQ ID NO: 77; or
b) one, two, or three heavy chain CDR sequences each selected from the group consisting of the sequences listed in Tables 2 and 7-9; and one, two, or three light chain CDR sequences each selected from the group consisting the sequences listed in Tables 2 and 7-9.

11. The bispecific antibody, or antigen-binding fragment thereof, of claim 10 comprising:
a) a VH comprising an amino acid sequence of SEQ ID NO: 79; and
b) a VH comprising an amino acid sequence of SEQ ID NO: 81.

12-13. (canceled)

14. The bispecific antibody, or antigen-binding fragment thereof, of claim 10, wherein the bispecific antibody, or antigen-binding fragment thereof,
i) is chimeric, humanized, composite, murine, or human;
ii) is (a) detectably labeled, (b) conjugated to a cytotoxic agent, optionally a chemotherapeutic agent, a biologic agent, a toxin, and/or a radioactive isotope, (c) comprises an effector domain, (d) comprises an Fc domain, and/or (e) is selected from the group consisting of Fv, Fav, F(ab′)2), Fab′, dsFv, scFv, sc (Fv)2, and diabodies fragments;
iii) inhibits the binding of (a) HHLA2 to KIR3DL3, and (b) PD-1 to PD-L1 and/or PD-L2; and/or
iv) specifically binds KIR3DL3 and PD-1.

15-20. (canceled)

21. An isolated nucleic acid molecule that:
(a) encodes a monoclonal antibody, or antigen-binding fragment thereof, of claim 1;
(b) comprises a sequence with at least 95% homology to a nucleic acid encoding a monoclonal antibody, or antigen-binding fragment thereof of claim 1; and/or
c) hybridizes, under stringent conditions, with the complement of a nucleic acid encoding a monoclonal antibody, or antigen-binding fragment thereof of claim 1.

22. A vector comprising the isolated nucleic acid of claim 21.

23. A host cell which comprises the isolated nucleic acid of claim 21.

24. A device or kit comprising at least one monoclonal antibody, or antigen-binding fragment thereof, of claim 1.

25. A method of producing at least one monoclonal antibody, or antigen-binding fragment thereof, of claim 1, which method comprises the steps of:
(i) culturing a transformed host cell which has been transformed by a nucleic acid comprising a sequence encoding the at least one monoclonal antibody, or antigen-binding fragment thereof, of claim 1 under conditions suitable to allow expression of said monoclonal antibody, or antigen-binding fragment thereof; and (ii) recovering the expressed monoclonal antibody, or antigen-binding fragment thereof.

26. A pharmaceutical composition comprising (i) at least one monoclonal antibody, or antigen-binding fragment thereof, of claim 1 and (ii) a pharmaceutically acceptable excipient.

27-34. (canceled)

35. A method of treating a subject afflicted with cancer comprising administering to the subject at least one monoclonal antibody, or antigen-binding fragment thereof, of claim 1.

36. The method of claim 35, wherein the at least one monoclonal antibody, or antigen-binding fragment thereof, (a) reduces the number of proliferating cancer cells in the cancer; (b) reduces the volume or size of a tumor of the cancer; and/or (c) activates a T cell and/or an NK cell.

37-38. (canceled)

39. The method of claim 35, further comprising administering to the subject an additional therapy selected from the group consisting of immunotherapy, checkpoint blockade, cancer vaccines, chimeric antigen receptors, chemotherapy, radiation, target therapy, and surgery, optionally wherein the chimeric antigen receptor targets CD19.

40-41. (canceled)

42. The method of claim 35, wherein the cancer is selected from the group consisting of adenocarcinoma, chronic myelogenous leukemia (CML), lung cancer, renal cancer, pancreatic cancer, colorectal cancer, acute myeloid leukemia, head and neck carcinoma, liver cancer, ovarian cancer, prostate cancer, uterine cancer, gliomas, glioblastoma, neuroblastoma, breast cancer, pancreatic ductal carcinoma, thymoma, B-CLL, leukemia, B cell lymphoma, and a cancer infiltrated with immune cells expressing a receptor to HHLA2, optionally wherein the cancer is selected from the group consisting of lung cancer, renal cancer, pancreatic cancer, colorectal cancer, acute myeloid leukemia (AML), head and neck carcinoma, liver cancer, ovarian cancer, prostate cancer, and uterine cancer.

43. (canceled)

44. The method of claim 35, wherein the subject is an animal model of cancer, optionally herein the animal model is a mouse model or a humanized mouse model.

45. (canceled)

46. The method of claim 35, wherein the subject is a human.

47-69. (canceled)
